(19) United States
(12) Patent Application Publication
(10) Pub. No.: US 2014/0309424 A1
(43) Pub. Date: Oct. 16, 2014

(54) DIRECT SYNTHESIS OF 18F-FLUOROMETHOXY COMPOUNDS FOR PET IMAGING AND THE PROVISION OF NEW PRECURSORS FOR DIRECT RADIOSYNTHESIS OF PROTECTED DERIVATIVES OF O-([18F] FLUOROMETHYL) TYROSINE

(71) Applicants: Thomas Brumby, Berlin (DE); Keith Graham, Berlin (DE); Martin Kruger, Berlin (DE)

(72) Inventors: Thomas Brumby, Berlin (DE); Keith Graham, Berlin (DE); Martin Kruger, Berlin (DE)

(73) Assignee: PIRAMAL IMAGING SA, MATRAN (CH)

(21) Appl. No.: 14/129,174

(22) PCT Filed: Jun. 29, 2012
(86) PCT No.: PCT/EP2012/062786

§ 371 Date: Apr. 25, 2014

(30) Foreign Application Priority Data
Jun. 30, 2011 (EP) 11075153.4
Jun. 30, 2011 (EP) 11075154.2

(51) Int. Cl. A61K 51/04; C07D 249/18; C07C 227/16; C07C 269/06; C07D 249/04; C07C 229/36; C07D 471/04; C07C 271/22

(52) U.S. Cl. CPC A61K 51/0453; C07D 471/04; C07D 249/18; C07C 271/22; C07C 269/06; C07D 249/04; A61K 51/0455; C07C 229/36; C07C 227/16
USPC 546/117; 548/259; 560/29; 548/255; 560/36

(57) ABSTRACT

The invention describes novel direct synthesis methods for converting a precursor into a PET-tracer with a ¹⁸F-fluoromethoxy-group. The invention is also directed to novel and stable precursors for the direct radiosynthesis of protected derivatives of O—([¹⁸F]Fluoromethyl)tyrosines.

### FIELD OF INVENTION

The invention describes novel direct synthesis methods for converting a precursor into a PET-tracer with ¹⁸F-fluoromethoxy-groups. The invention further describes novel and stable precursors for the direct radiosynthesis of protected derivatives of O—([¹⁸F]Fluoromethyl)tyrosines, and methods for obtaining those compounds.

### BACKGROUND ART

The fluoromethoxy-group has been used for introduction of fluorine into a compound of biological interest for some time. It has the advantage of being very similar to the methoxy-group with respect to steric demand. Replacement of methoxy by fluoromethoxy in biologically active compounds can be done without loss of affinity to the target of interest, thus very often. Fluoromethoxy-despite being formally a formaldehyde acetal derivative—is quite a stable group in many molecules. Especially as substituents on aromatic rings, methoxy—by fluoromethoxy-substitution gives chemically stable compounds. Stability against metabolic degradation, however, is diminished, which is the reason, why this group is not frequently used in therapeutic drug discovery activities. Biological stability; however, may well be sufficient for use in PET, as very long plasma half lives are usually not desired for PET-tracers.

This would make the fluoromethoxy an ideal group to introduce an ¹⁸F-label into any biologically active molecule containing an aromatic methoxy group. However, in current labelling literature this group is not nearly as often used as the sterically more demanding fluoroethoxy group (search in Chemical Abstracts Service (CAS) reveals 21 [¹⁸F]-fluoromethoxy compounds (table 1) versus 335 [¹⁸F]-fluoroethoxy-compounds). This huge preference cannot be solely explained by the greater stability of the fluoroethoxy-group, as the risk of losing the biological activity is greater by using this group compared to fluoromethoxy. But taking into account the fundamental difference between the two labelling strategies the choice of fluoroethoxy over fluoromethoxy becomes completely rational.

For generation of the fluoroethoxy group, a wide choice of precursor groups are available (tosyloxyethoxy, mesyloxyethoxy or haloethoxy). These precursor molecules despite being reactive molecules, can be isolated and stored and allow an easy direct labelling access to their corresponding tracers.

By contrast, fluoromethoxy-labelled tracers are almost always made by a so called “indirect” labelling. To this end, a radioactive fluoromethylating agent is prepared. The radiochemical practise knows a variety of such labelling reagents as detailed in the table 1.

TABLE 1
Reagents for indirect synthesis of [¹⁸F]-Fluoromethyltracers

| Tracer | CAS Registry | | |
|---|---|---|---|
| (trivial name) | number | Literature | Reagent |
| | 1083103-28-9 | WO 2008141249 GE | F—CH₂OTs |
| | 1059188-91-8 | Journal of Medicinal Chemistry (2008), | F—CH₂—Br |
| | | 51(18), 5833-5842 | |
| | 947395-20-2 | WO 2007096193 A3 | F—CH₂—Br |
| [¹⁸F]FM- | 1004511-89-0 | Nuclear Medicine and Biology (2007), | Indirect |
| SA4503 | | 34(5), 571-577 | |
| | 934200-18-7 | WO 2007041025 A3 | F—CD₂-Br |
| [¹⁸F]SPA-RQ | 262598-99-2 | Journal of Labelled Compounds and | F—CH₂—Br |
| | | Radio pharmaceuticals (2006), 49(1), | |
| | | 17-31 and 49(11), 935-937 (correction) | |
| | | Synapse (2007), 61(4), 242-251 | |
| | 863887-82-5 | WO 2005079391 A3 | F—CH₂—Br |
| | 849469-05-2 | WO 2005030723 A1 | F—CH₂OTf |
| di-deutero | 848769-79-9 | Bioorganic & Medicinal Chemistry | FCH₂I |
| FMDAA1106 | | (2005), 13(5), 1811-1818 | |
| D-FMT | 870452-26-9 | WO 2005115971 A1 | indirect |
| L-FMT | 627092-21-1 | WO 2005009928 A3 | indirect |
| | 867281-18-3 | Journal of Labelled Compounds & | BrCH₂F |
| | | Radio pharmaceuticals (2005), 48(1), | |
| | | 1-10 | |
| | | Journal of Nuclear Medicine and | |
| | | Molecular Imaging (2006), | |
| | | 33(10), 1134-1139 | |
| FMDAA1106 | 505084-40-2 | U.S. Pat. No. 6,870,069 B2 | FCH₂I |
| | | Journal of Medicinal Chemistry (2004), | Indirect |
| | | 47(9), 2228-2235 | and direct |
| | | Bioorganic & Medicinal Chemistry | FCH₂I |
| | | Letters (2003), 13(2), 201-204 | |
| | 844446-45-3 | Synapse (New York, NY, US) (2004), | BrCD₂F |
| | | 53(2), 57-67 | |
| (S,S)-[¹⁸F] | 844446-44-2 | Synapse (2004), 53(2), 57-67 | BrCH₂F |
| FMeNER | | Psychopharmacology (2006), 188(1), | |
| | | 119-127 | |
| | 686768-01-4 | WO 2004038374 A3 | BrCD₂F |
| | 677000-29-2 | WO 2004029024 A3 | BrCD₂F |
| | 262598-99- | WO 2004029006 A3 | FCH₂I, |
| | 2P | WO 2000018403 A1 | FCH₂Br |
| | | Journal of Nuclear Medicine | |
| | | (2007), 48(1), 100-107 | |
| | 844446-45-3 | Nuclear Medicine and Biology (2008), | indirect |
| | | 35(7), 733-740 | |
| | 870452-26-9 | Journal of Nuclear Medicine (2006), | indirect |
| | | 47(4), 679-688 | |
| | | European Journal of Nuclear Medicine | |
| | | and Molecular Imaging (2006), 33(9), | |
| | | 1017-1024 | |
| | 851014-76-1 | Journal of Fluorine Chemistry (2004), | FCH₂I |
| | | 125(12), 1879-1886 | |

Generally, such indirect syntheses require more steps and give inferior yields when compared to their direct counterparts. Some of the reagents mentioned above are gaseous, thus requiring special equipment not present in every laboratory. The reagents for direct synthesis of fluoromethylethers should be available. For example, chloromethylethers of many phenols are commercially available. However, not all desired chloromethylethers are stable. The chloromethylether of boc tyrosine methylester was synthesized and found to be chemically not stable (Angew. Chem. Int. Ed. 2002, 3449). Other authors found such compounds stable, but very reactive when dissolved in solvents containing water (J. Appl. Chem. 1953, 266). Interestingly, there is only one report for use of a halo-methyl compound as a labelling precursor to make a fluoromethoxy-labelled tracer (Bioorganic & Medicinal Chemistry 2005, 13, 1811-1818). This report stated that the labelled fluoromethyl compound “could be obtained, but the radiochemical yield was not reproducible (0-35%)”. In the end, the authors relied for production of the tracer on the established indirect methods. Aromatic tosyloxymethoxy-compounds can be synthesized (e.g. Synthesis 1971, 150), but such compounds have not been used for synthesis of [¹⁸F]-tracers. Thus, it appears that methoxy-compounds substituted with leaving groups commonly used for aliphatic nucleophilic substitution reactions (e.g. OCH₂-Hal, OCH₂—OTs, OCH₂—OMs or OCH₂—OTf) are not of use for the synthesis of [¹⁸F]-fluoromethoxy-compounds (OCH₂—F).

So far, for direct synthesis of [¹⁸F]-fluoromethoxy compounds, precursors are lacking, which are stable compounds with a long shelf life, which do not decompose under standard labelling conditions and give reproducible results in the labelling reaction.

O—N activating groups have been known and are in use for a long time in amide-forming reactions. (e.g. N-Hydroxybenzotriazole (HOBt), 7-aza-N-Hydroxybenzotriazole (HOAt), 6-chloro-N-hydroxybenzotriazole, 3-Hydroxy-1,2,3-benzotriazin-4(3H)-one, Ethyl cyano(hydroximino)acetate, 1-Hydroxypyridinone, ethyl-1-hydroxy-1H-1,2,3-triazole-4-carboxylate) (e.g. Houben-Weyl E22, 2003, p 443ff and 522ff). Such groups have also been used as leaving groups in aromatic nucleophilic substitution reactions to form [¹⁸F]-substituted aromatic compounds, see WO 2008/104203.

O—N substituents attached to methoxy groups (OCH₂ON) have been described—over 7000 structures of this type are known in CAS. However, in combination with fluorine (F) only very few structures can be found. In several patents, C═NOCH₂F is described as a prodrug which could release the corresponding ketone (C═O) after hydrolysis (e.g. WO2008/143730, WO2008/106204, Bioorg. Med. Chem. Lett. 2002, 833). None of these documents teach the use of the ON-activating group as a leaving group to synthesize fluoromethoxy-groups.

The inventors surprisingly found that such O—N-activating groups can be used as leaving groups in aliphatic nucleophilic substitutions to form fluoromethoxy-groups. Moreover, they form stable precursors for reliable and reproducible synthesis of fluoromethoxy-compounds.

Further, both O-(Fluoromethyl)-D-tyrosine and O-(Fluoromethyl)-L-tyrosine have been described as PET-tracers for in vivo imaging of various tumour types (D-FMT: WO 2005115971; Eur. J. Nucl. Med. Mol. Imag. 2006, p 1017; J. Nucl. Med. 50, p 290, 2009; J. Nucl. Med. 47, p 679, 2006; Nuc. Med. Biol. 2009 p 295; L-FMT, WO 2005009928; J. Label. Comp. Radiopharm. 46, p 555, 2003). However, in all syntheses reported to date for these compounds, as stated above a so called “indirect” labelling has been employed, which consists of preparation of a labelled ¹⁸F-synthon (e.g. fluoromethyl bromide, fluoromethyl-tosylate, -mesylate or -triflate), which is reacted with tyrosine to give the desired tracer. The radiochemical practice knows a variety of such labelling reagents, but not only for synthesis of O-fluoromethyltyrosines, as detailed in table 1.

### SUMMARY OF THE INVENTION

The present invention is directed to radiolabelling methods to convert compounds of general formula I into compounds of general formula II, and further is directed to novel precursors according to the general formula I and Ia for direct radiosynthesis of protected [¹⁸F]Fluoromethyl derivatives, and in particular derivatives of O-([¹⁸F]Fluoromethyl) tyrosine.

### FIGURES

FIG. 1: HPLC Left γ-trace and Right UV-detector.

FIG. 2: HPLC Left γ-trace and Right UV detector.

FIG. 3: HPLC final product DFMT (QC).

FIG. 4: HPLC final product DFMT (QC)+co-injection with cold reference.

FIG. 5: HPLC final product DFMT (chiral).

FIG. 6: HPLC final product DFMT (chiral)+co-injection with cold reference.

### DETAILED DESCRIPTION OF THE INVENTION

### Direct Radiolabelling Methods

The present invention is directed to radiolabeling methods to convert compounds of formula I into compounds of formula II.

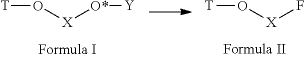

The radiolabeling methods for converting compounds of formula I into compounds of formula II comprise the steps of
Reacting compound of Formula I with a [¹⁸F]-Fluorination agent,
[optionally] deprotecting the obtained compound for obtaining deprotected compound of formula II and/or
[optionally] converting obtained compound into a suitable salts of inorganic or organic bases thereof, hydrates, complexes, and solvates thereof
   wherein:

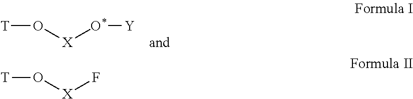

F is [¹⁸F] fluorine atom;
T is a small molecule;
X is CH₂, CHD or CD₂;
Y is a substituted heteroaromatic ring containing one to four nitrogen atoms with the proviso that the oxygen (O*) is directly bound to one of the nitrogens of the heteroaromatic ring and O*—Y acts as leaving group.

Throughout the specification the term “deprotecting” means removing the protecting groups PG1 and PG2. Deprotecting occurs under acid and basic conditions.

The invention further refers to suitable salts of inorganic or organic acids, hydrates and solvates of the compounds of Formula II and is also meant to comprise single isomers, diastereomers, enantiomers and mixtures thereof of Formula II.

The compound of formula II obtained from the first method step can be protected or non-protected depending on T.

The “small molecule or small molecule T” according to the present invention is a bioactive compound that interacts with or has an effect on cell tissue or biological elements of mammal body wherein the biological activity of said small molecule is well known in the art. The biological activity represents the “intrinsic” property of a small molecule depending only on its structure and physical-chemical characteristics.

Further the “small molecule or small molecule T” according to the present invention is defined as organic compounds, inorganic compounds, and the like but not limited to natural and un-natural amino acids and nucleotides.

Preferably, T is a small molecule having a molecular mass of from about 150 daltons to about 1,500 daltons and having a biological activity.

More preferably, the small molecule has a molecular mass of from about 150 daltons to about 600 daltons, from about 150 daltons to about 400 daltons, or from about 150 daltons to about 350 daltons.

More preferably, the small molecule has a molecular mass of from about 600 daltons to about 1,500 daltons, or from about 600 daltons to about 1,000 daltons.

More preferably, T is a small molecule as defined above encompassing an aromatic or heteroaromatic moiety.

Even more preferably referring to compound of Formula I, T is a small molecule as defined above encompassing an aromatic or heteroaromatic moiety wherein the —O—X—O*—-Y group is covalently bond to the aromatic or heteroaromatic moiety, preferably, the —O—X—O*—Y group is covalently bond to the aromatic or heteroaromatic moiety at the para position.

Preferably, “aromatic moiety” is an aryl e.g. phenyl, naphthyl or tetrahydronaphthyl and heteroaromatic moiety is e.g. pyrrole, imidazole, triazole.

### Preferred Features:

Preferably, X is CH₂, or CD₂.

Preferably, Y is 5 to 10 membered heteroaromatic ring containing one to four heteroatoms wherein the heteroatom is Nitrogen (N). The heteroaromatic ring is a single ring (preferably 5 or 6 membered with up to three nitrogens) or a fused ring (preferably 9 or 10 membered with up to four nitrogens). Preferably, the heteroaromatic ring comprises 2 to 4 heteroatoms, more preferably 3 to 4.

More preferably. Y is

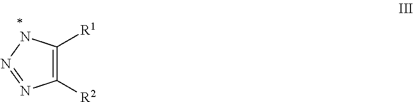

wherein
* indicates the position of the covalent bond to the Oxygen (O*) in formula I;
R¹ is H, CN, or COOR⁴, and R² is H, CN, or COOR⁴, or
R¹ and R² form together a 6 membered aromatic ring, which optionally comprise 1 Nitrogen (N) and 1 methine of the 6 membered ring is optionally substituted with Halogen, NO₂, CN, COOR³, SO₂R³ or CF₃,
R³ is C₁-C₃ alkyl, and
R⁴ is C₁-C₆ alkyl.

Preferably, R¹ and R² form together a 6 membered aromatic ring, which optionally comprise 1 Nitrogen (N) and 1 methine of the 6 membered ring is optionally substituted with Halogen, NO₂, or CF₃.

Preferably, R³ is C₁ alkyl(methyl).

Preferably, R⁴ is C₁ alkyl(methyl) or C₂ alkyl(ethyl).

Preferably, Halogen is chloro (Cl).

Even more preferably, Y is

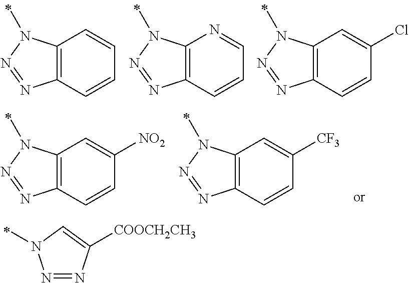

* indicates the position of the covalent bond to the Oxygen (O*) in formula I.

Even more preferably, Y is

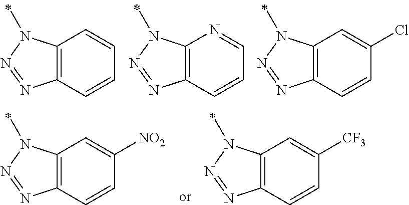

* indicates the position of the covalent bond to the Oxygen (O*) in formula I.

Preferably, O*—Y acts as a leaving group suitable for introducing a fluoride.

Even more preferably referring to compound of Formula I, T is a small molecule of formula below

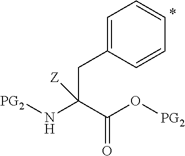

wherein
* indicates the position of the —O—X—O*—Y group in formula I;
Z is Hydrogen or methyl;
y is

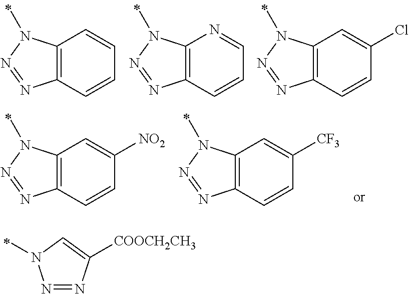

PG1 is carboxylic acid protecting group defined as
   Alkyl,
   Alkyl substituted with one phenyl,
   Alkyl substituted with one or two C₃-C₆cycloalkyl,
   Alkyl substituted with one phenyl and one C₃-C₆cycloalkyl, or
   fluorenylmethyl
   wherein
      Alkyl is a branched or linear C₁-C₆ alkyl, and optionally substituted with C₁-C₃ Alkoxy, and
      phenyl is optionally substituted with up to three C₁-C₃ Alkyl, C₁-C₃ Alkoxy or Halogen;
PG2 is an amino protecting group,
   preferably PG2 is a carbamate- or alkylaryl-amino protecting group, and even more preferred PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

Even more preferably referring to compound of Formula II, T is a small molecule as defined above encompassing an aromatic or heteroaromatic moiety, wherein the Fluoromethoxy group (—O—X—F) is covalently bond to the aromatic or heteroaromatic moiety preferably, the —O—X—F group is covalently bond to the aromatic or heteroaromatic moiety at the para position.

Even more preferably referring to compound of Formula II, T is a small molecule of formula below

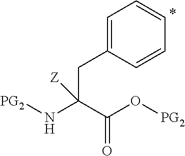

wherein
* indicates the position of the (—O—X—F) Oxygen forming the ester bond in formula II; Z, Y, PG1, and PG2 are as defined above.

Optionally, the small molecule (T) discloses functional groups (NH₂, COOH and OH) that interfere with fluorolabelling reaction. Thus, the functional groups are protected in a way known to the person skilled in the art. In particular, functional groups are amines, carboxylic acids, thiols, and alcohols that are protected with carbamates or arylalkylamines for amines, esters for carboxylic acids, thioethers for thiols and ethers or esters for alcohols.

The groups are chosen in a way to allow deprotection after the fluorine incorporation. General ways for protection are given in Greene and Wuts, Protecting groups in Organic Synthesis, Wiley Interscience, third edition, 1999 and fourth edition 2007.

### Preferred Methods:

Preferably, the radiolabeling methods for converting compounds of formula I into compounds of formula II comprise the steps of
Reacting compound of Formula I with a [¹⁸F]-Fluorination agent,
[optionally] deprotecting the obtained compound for obtaining deprotected compound of formula II and/or
[optionally] converting obtained compound into suitable salts of inorganic or organic bases thereof, hydrates, complexes, and solvates thereof,
   wherein:

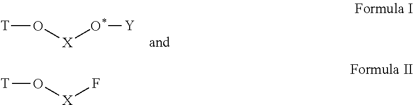

F is [¹⁸F] fluorine atom;
T is a small molecule;
X is CH₂, or CD₂
Y is

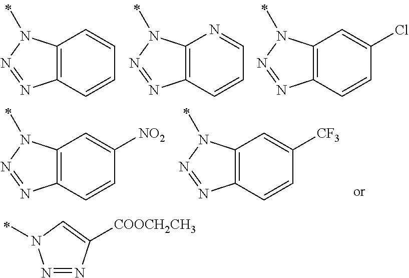

More preferably, the radiolabeling methods for converting compounds of formula I into compounds of formula II comprise the steps of
Reacting compound of Formula I with a [¹⁸F]-Fluorination agent,
[optionally] deprotecting the obtained compound for obtaining deprotected compound of formula II and/or
[optionally] converting obtained compound into a suitable salts of inorganic or organic bases thereof, hydrates, complexes, and solvates thereof,
   wherein:

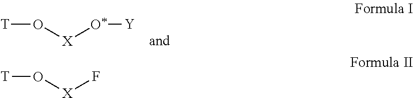

F is [¹⁸F] fluorine atom;
T is a small molecule having a molecular mass of from about 150 daltons to about 1,500 daltons and a biological activity;
X is CH₂, or CD₂
Y is

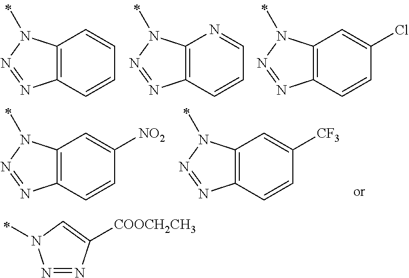

Even more preferably, the radiolabeling methods for converting compounds of formula I into compounds of formula II comprise the steps of
Reacting compound of Formula I with a [¹⁸F]-Fluorination agent,
[optionally] deprotecting the obtained compound for obtaining deprotected compound of formula II and/or
[optionally] converting obtained compound into a suitable salts of inorganic or organic bases thereof, hydrates, complexes, and solvates thereof,
   wherein:

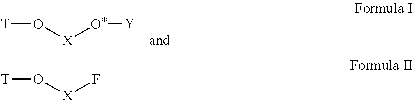

F is [¹⁸F] fluorine atom;
T is a small molecule having a molecular mass of from about 150 daltons to about 1,500 daltons, and a biological activity and encompassing an aromatic or heteroaromatic moiety wherein the —O—X—O*—Y and —O—X—F groups are covalently bond to the aromatic or heteroaromatic moiety, preferably, the —O—X—O*—Y and —O—X—F groups are covalently bond to the aromatic or heteroaromatic moiety at the para position;
X is CH₂, or CD₂
Y is

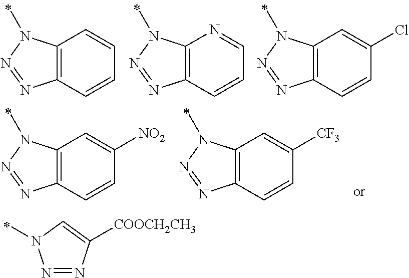

Even more preferably, the radiolabeling method is as following

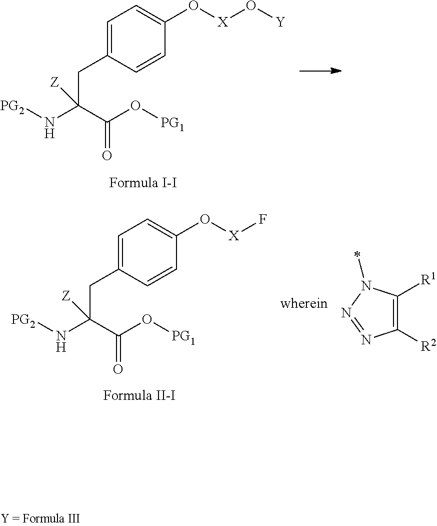

wherein Z, Y, R¹, R², PG1, and PG2 are as defined above.

### Fluorination Reagents and Conditions:

The ¹⁸F-Fluorination agent can be K¹⁸F, H¹⁸F, Rb¹⁸F, Cs¹⁸F, Na¹⁸F.

Optionally, the ¹⁸F-Fluorination agent comprises a chelating agent such as a cryptand (e.g.: 4,7,13,16,21,24-Hexaoxa-1,10-diazabicyclo[8.8.8]-hexacosane —Kryptofix®) or a crown ether (e.g.: 18-crown-6).

The ¹⁸F-Fluorination agent can also be a tetraalkylammonium salt of ¹⁸F or a tetraalkylphosphonium salt of ¹⁸F, known to those skilled in the art, e.g.: tetrabutylammonium[¹⁸F]fluoride, tetrabutylphosphonium[¹⁸F]fluoride.

Preferably, the ¹⁸F-Fluorination agent is Cs¹⁸F, K¹⁸F, tetrabutylammonium[¹⁸F]fluoride.

The reagents, solvents and conditions which can be used for this fluorination are common and well-known to the skilled person in the art, see, e.g., J. Fluorine Chem., 27 (1985):177-191; Coenen, Fluorine-18 Labeling Methods: Features and Possibilities of Basic Reactions, (2006), or Schubiger P. A., Friebe M., Lehmann L., (eds), PET-Chemistry—The Driving Force in Molecular Imaging. Springer, Berlin Heidelberg, pp. 15-50).

Preferably, the solvents used according to the present method are DMF, DMSO, acetonitrile, DMA, or mixtures thereof, more preferably the solvent is acetonitrile, or DMSO.

### Further Preferred Embodiments of Formula I:

Compounds of formula I are defined below, but not limited to
tert-Butyl O-[(1H-benzotriazol-1-yloxy)methyl]-N-(tert-butoxycarbonyl)-D-tyrosinate

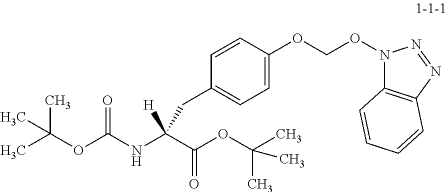 [SMILES: CC(C)(C)OC(=O)N[C@H](Cc1ccc(OCOn2nnc3ccccc32)cc1)C(=O)OC(C)(C)C]

tert-Butyl N-(tert-butoxycarbonyl)-O-[(1H-1,2,3-triazolo[5,4-b]pyridin-1-yloxy)methyl]-D-tyrosinate

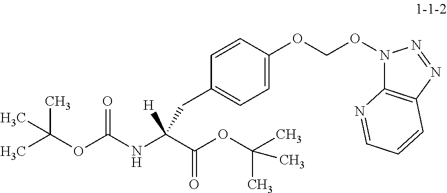 [SMILES: CC(C)(C)OC(=O)N[C@H](Cc1ccc(OCOn2nnc3cccnc32)cc1)C(=O)OC(C)(C)C]

Dicyclopropylmethyl O-[(1H-benzotriazol-1-yloxy)methyl]-N-(tert-butoxycarbonyl)-D-tyrosinate

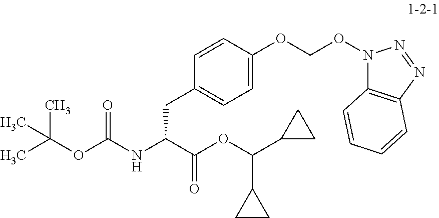 [SMILES: CC(C)(C)OC(=O)N[C@H](Cc1ccc(OCOn2nnc3ccccc32)cc1)C(=O)OC(C1CC1)C1CC1]

Dicyclopropylmethyl O-[(1H-benzotriazol-1-yloxy)methyl]-N-(tert-butoxycarbonyl)-L-tyrosinate

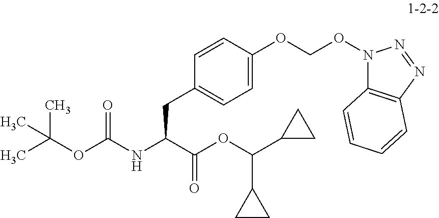 [SMILES: CC(C)(C)OC(=O)N[C@@H](Cc1ccc(OCOn2nnc3ccccc32)cc1)C(=O)OC(C1CC1)C1CC1]

Dicyclopropylmethyl N-(tert-butoxycarbonyl)-O-[(6-nitro-1H-benzotriazol-1-yloxy)methyl]-D-tyrosinate

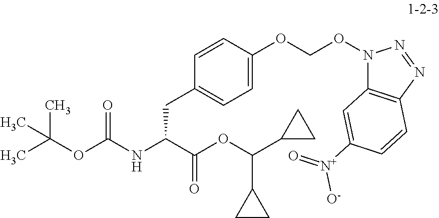 [SMILES: CC(C)(C)OC(=O)N[C@H](Cc1ccc(OCOn2nnc3ccc([N+](=O)[O-])cc32)cc1)C(=O)OC(C1CC1)C1CC1]

2,4-Dimethoxybenzyl O-[(1H-benzotriazol-1-yloxy)methyl]-N-(tert-butoxycarbonyl)-D-tyrosinate

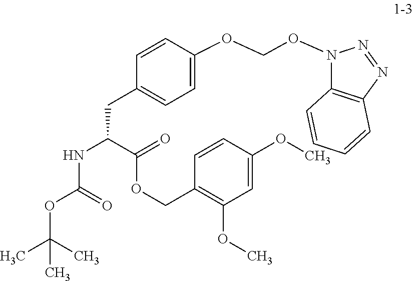 [SMILES: COc1ccc(COC(=O)[C@@H](Cc2ccc(OCOn3nnc4ccccc43)cc2)NC(=O)OC(C)(C)C)c(OC)c1]

Cyclopropylmethyl O-[(1H-benzotriazol-1-yloxy)methyl]-N-(tert-butoxycarbonyl)-D-tyrosinate

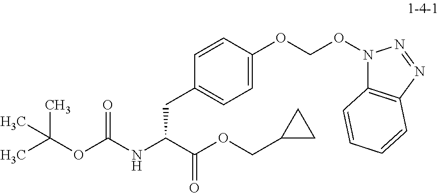 [SMILES: CC(C)(C)OC(=O)N[C@H](Cc1ccc(OCOn2nnc3ccccc32)cc1)C(=O)OCC1CC1]

Cyclopropylmethyl N-(tert-butoxycarbonyl)-O-({[4-(ethoxycarbonyl)-1H-1,2,3-triazol-1-yl]-oxy}methyl)-D-tyrosinate

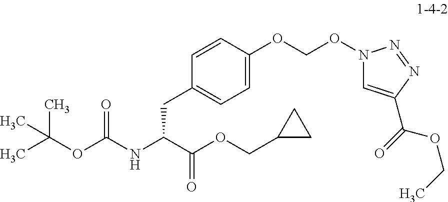 [SMILES: CCOC(=O)c1cn(OCOc2ccc(C[C@@H](NC(=O)OC(C)(C)C)C(=O)OCC3CC3)cc2)nn1]

4-Methoxybenzyl O-[(1H-benzotriazol-1-yloxy)methyl]-N-(tert-butoxycarbonyl)-D-tyrosinate

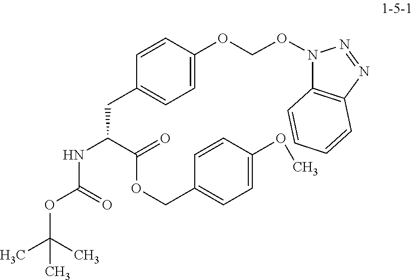 [SMILES: COc1ccc(COC(=O)[C@@H](Cc2ccc(OCOn3nnc4ccccc43)cc2)NC(=O)OC(C)(C)C)cc1]

4-Methoxybenzyl N-(tert-butoxycarbonyl)-O-{[(6-chloro-1H-benzotriazol-1-yl)oxy]methyl}-D-tyrosinate

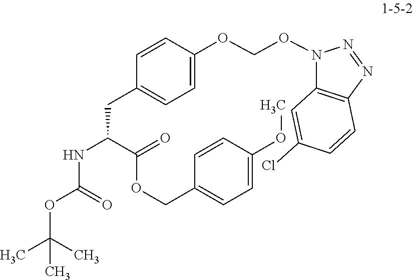 [SMILES: COc1ccc(COC(=O)[C@@H](Cc2ccc(OCOn3nnc4ccc(Cl)cc43)cc2)NC(=O)OC(C)(C)C)cc1]

4-Methoxybenzyl N-(tert-butoxycarbonyl)-O-[(6-trifluoromethyl-1H-benzotriazol-1-yloxy)methyl]-D-tyrosinate

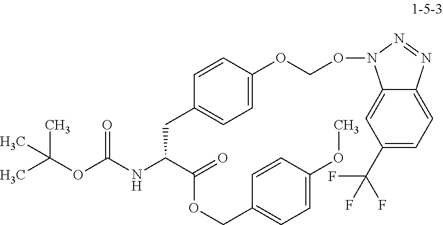 [SMILES: COc1ccc(COC(=O)[C@@H](Cc2ccc(OCOn3nnc4ccc(C(F)(F)F)cc43)cc2)NC(=O)OC(C)(C)C)cc1]

4-Methoxybenzyl O-[(6-trifluoromethyl-1H-benzotriazol-1-yloxy)methyl]-N-(tert-butoxycarbonyl)-L-tyrosinate.

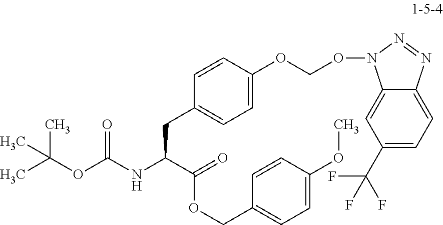 [SMILES: COc1ccc(COC(=O)[C@H](Cc2ccc(OCOn3nnc4ccc(C(F)(F)F)cc43)cc2)NC(=O)OC(C)(C)C)cc1]

alpha-Methylbenzyl O-[(1H-benzotriazol-1-yloxy)methyl]-N-(tert-butoxycarbonyl)-D-tyrosinate

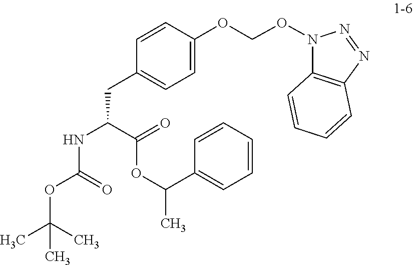 [SMILES: CC(OC(=O)[C@@H](Cc1ccc(OCOn2nnc3ccccc32)cc1)NC(=O)OC(C)(C)C)c1ccccc1]

alpha,alpha-Dimethylbenzyl O-[(1H-benzotriazol-1-yloxy)methyl]-N-(tert-butoxycarbonyl)-D-tyrosinate

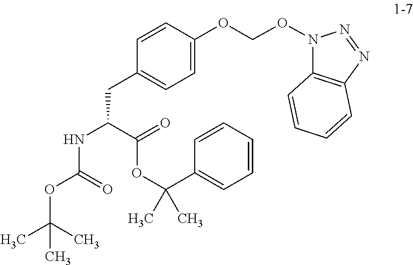 [SMILES: CC(C)(C)OC(=O)N[C@H](Cc1ccc(OCOn2nnc3ccccc32)cc1)C(=O)OC(C)(C)c1ccccc1]

tert-Butyl O-[(1H-benzotriazol-1-yloxy)methyl]-N-trityl-D-tyrosinate

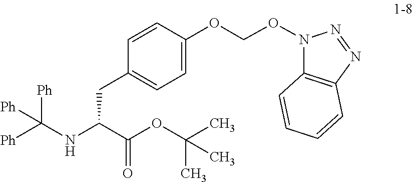 [SMILES: CC(C)(C)OC(=O)[C@@H](Cc1ccc(OCOn2nnc3ccccc32)cc1)NC(c1ccccc1)(c1ccccc1)c1ccccc1]

4-Methoxybenzyl O-[(1H-benzotriazol-1-yloxy)methyl]-N-trityl-D-tyrosinate

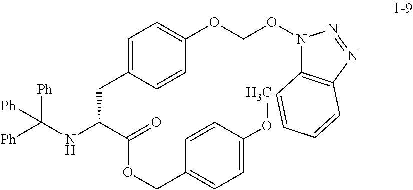 [SMILES: COc1ccc(COC(=O)[C@@H](Cc2ccc(OCOn3nnc4ccccc43)cc2)NC(c2ccccc2)(c2ccccc2)c2ccccc2)cc1]

Cyclopropylmethyl O-[(1H-benzotriazol-1-yloxy)[²H₂]methyl]-N-(tert-butoxycarbonyl)-D-tyrosinate

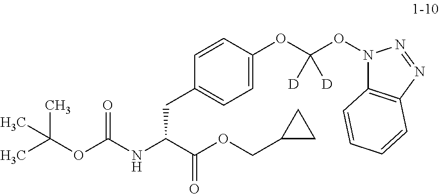 [SMILES: [2H]C([2H])(Oc1ccc(C[C@@H](NC(=O)OC(C)(C)C)C(=O)OCC2CC2)cc1)On1nnc2ccccc21]

2,4-Dimethoxybenzyl O-[(1H-benzotriazol-1-yloxy)methyl]-N-trityl-D-tyrosinate

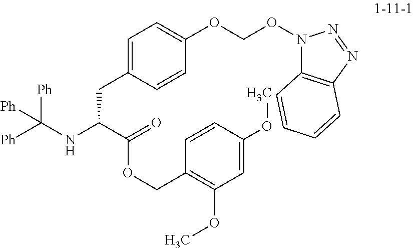 [SMILES: COc1ccc(COC(=O)[C@@H](Cc2ccc(OCOn3nnc4ccccc43)cc2)NC(c2ccccc2)(c2ccccc2)c2ccccc2)c(OC)c1]

2,4-Dimethoxybenzyl 0-{[(6-chloro-1H-benzotriazol-1-yl)oxy]methyl}-N-trityl-D-tyrosinate

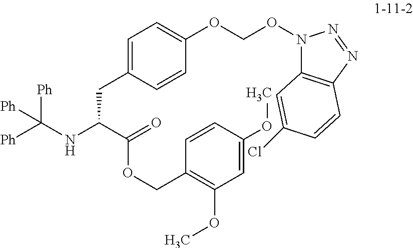 [SMILES: COc1ccc(COC(=O)[C@@H](Cc2ccc(OCOn3nnc4ccc(Cl)cc43)cc2)NC(c2ccccc2)(c2ccccc2)c2ccccc2)c(OC)c1]

2,4-Dimethoxybenzyl 0-{[(6-trifluoromethyl-1H-benzotriazol-1-yl)oxy]methyl}-N-trityl-D-tyrosinate

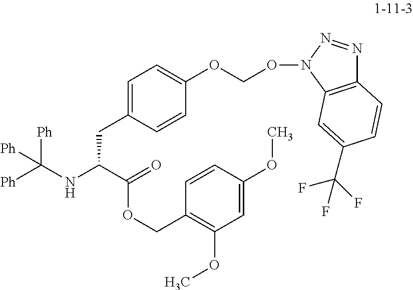 [SMILES: COc1ccc(COC(=O)[C@@H](Cc2ccc(OCOn3nnc4ccc(C(F)(F)F)cc43)cc2)NC(c2ccccc2)(c2ccccc2)c2ccccc2)c(OC)c1]

Methyl O-[(1H-benzotriazol-1-yloxy)methyl]-N-(tert-butoxycarbonyl)-alpha-methyl-tyrosinate

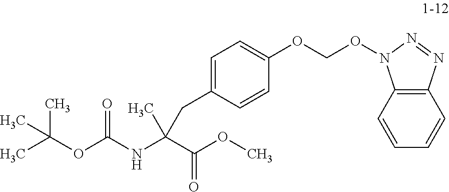 [SMILES: COC(=O)C(C)(Cc1ccc(OCOn2nnc3ccccc32)cc1)NC(=O)OC(C)(C)C]

Benzyl 7-[(1H-benzotriazol-1-yloxy)methoxy]-3,4-dihydroisoquinoline-2(1H)-carboxylate

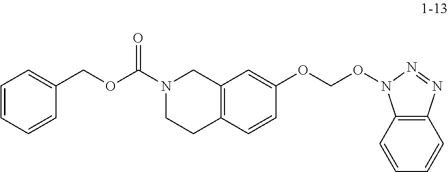 [SMILES: O=C(OCc1ccccc1)N1CCc2ccc(OCOn3nnc4ccccc43)cc2C1]

2-{2-[4-(1H-Benzotriazol-1-yloxymethoxy)phenyl]-5,7-dimethylpyrazolo[1,5-a]pyrimidin-3-yl}-N,N-diethyl-acetamide

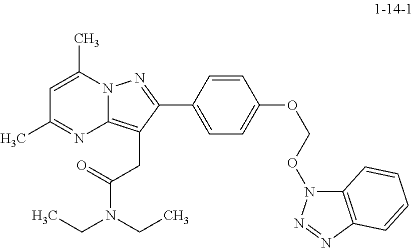 [SMILES: CCN(CC)C(=O)Cc1c(-c2ccc(OCOn3nnc4ccccc43)cc2)nn2c(C)cc(C)nc12]

2-[(1H-Benzotriazol-1-yloxy)methoxy]ethyl benzoate

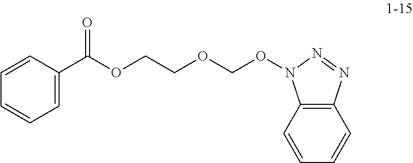 [SMILES: O=C(OCCOCOn1nnc2ccccc21)c1ccccc1]

1-[(Benzyloxy)methoxy]-1H-benzotriazole

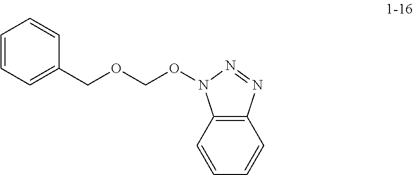 [SMILES: c1ccc(COCOn2nnc3ccccc32)cc1]

Further preferred embodiments of formula II:

Compounds of formula II are defined below, but not limited to
tert-Butyl N-(tert-butoxycarbonyl)-O-([¹⁸F]fluoromethyl)-D-tyrosinate.

### Labelling of 1-1-1 and 1-1-2

Dicyclopropylmethyl N-(tert-butoxycarbonyl)-O-([¹⁸F]-fluoromethyl)-D-tyrosinate.

### Labelling of 1-2-1

Dicyclopropylmethyl N-(tert-butoxycarbonyl)-O-([¹⁸F]fluoromethyl)-L-tyrosinate.

### Labelling of 1-2-2

2,4-Dimethoxybenzyl N-(tert-butoxycarbonyl)-O-([¹⁸F]fluoromethyl)-D-tyrosinate.

### Labelling of 1-3

Cyclopropylmethyl N-(tert-butoxycarbonyl)-O-([¹⁸F]fluoromethyl)-D-tyrosinate.

### Labelling of 1-4-1 and 1-4-2

4-Methoxybenzyl N-(tert-butoxycarbonyl)-O-([¹⁸F]fluoromethyl)-D-tyrosinate.

### Labelling of 1-5-1 and 1-5-2

1-Phenylethyl N-(tert-butoxycarbonyl)-O-([¹⁸F]fluoromethyl)-D-tyrosinate.

### Labelling of 1-6

1-Methyl-1-phenylethyl N-(tert-butoxycarbonyl)-O-([¹⁸F]fluoromethyl)-D-tyrosinate.

### Labelling of 1-7

tert-Butyl O-([¹⁸F]-fluoromethyl)-N-trityl-D-tyrosinate.

### Labelling of 1-8

4-Methoxybenzyl O-([¹⁸F]fluoromethyl)-N-trityl-D-tyrosinate.

### Labelling of 1-9

Cyclopropylmethyl N-(tert-butoxycarbonyl)-O-([¹⁸F]-fluoro[²H₂]methyl)-D-tyrosinate.

### Labelling of 1-10

2,4-Dimethoxybenzyl 0-([¹⁸F]-fluoromethyl)-N-trityl-D-tyrosinate

### Labelling of 1-11-1, 1-11-2 and 1-11-3

Methyl N-(tert-butoxycarbonyl)-O-([¹⁸F]-fluoromethyl)-alpha-methyl-DL-tyrosinate.

### Labelling of 1-12

7-[¹⁸F]Fluoromethoxy-3,4-dihydro-1H-isoquinoline-2-carboxylic acid benzyl ester

### Labelling of 1-13

N,N-Diethyl-2-[2-(4-[¹⁸F]-fluoromethoxyphenyl)-5,7-dimethylpyrazolo[1,5-a]pyrimidin-3-yl]-acetamide

### Labelling of 1-14-1

Benzoic acid 2-[¹⁸F]fluoromethoxy ethyl ester

### Labelling of 1-15

[¹⁸F]Fluoromethoxymethylbenzene

### Labelling of 1-16

Compounds according to Formula Ia and IIa

The present invention is further directed to novel and stable precursors for the direct radiosynthesis of protected derivatives of O-([¹⁸F]Fluoromethyl)tyrosines according to the general Formulae Ia and IIa.

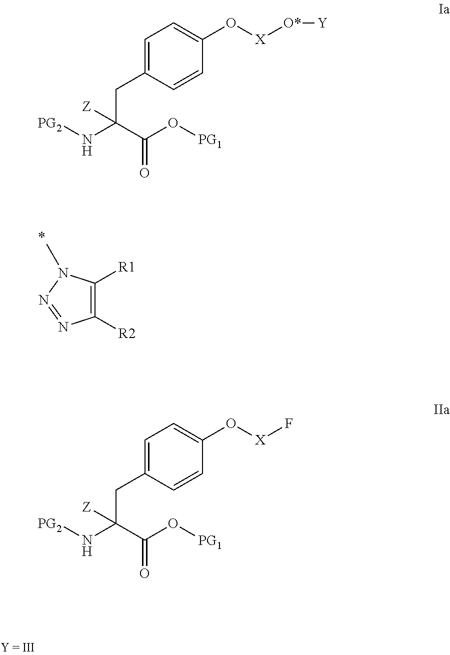

### Detailed Description of the Compound Inventions

In a first aspect, the present invention of novel precursors is directed to compounds of Formula Ia

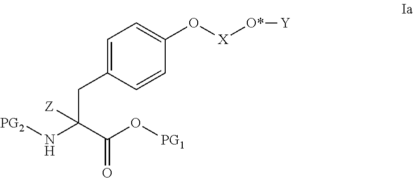

wherein:
X is CH₂, CHD or CD₂;
Y is a substituted or unsubstituted heteroaromatic ring containing one to four Nitrogen atoms (N) with the proviso that the oxygen (O*) is directly bound to one of the Nitrogen atoms (N) of the heteroaromatic ring and O*—Y acts as leaving group;
Z is Hydrogen or methyl;
PG1 is a carboxylic acid protecting group, containing up to 20 carbon atoms, optionally containing independently one or more O, N or S atoms; and
PG2 is an amino protecting group, containing up to 20 carbon atoms, optionally containing one or more O, N or S atoms and are optionally substituted with one to three halogens.

The invention further refers to suitable salts of inorganic or organic acids, hydrates and solvates of the compounds of Formula Ia and is also meant to comprise single isomers, diastereomers, enantiomers and mixtures thereof of Formula Ia.

### Preferred Features:

Preferably, Y is 5 to 10 membered heteroaromatic ring containing one to four Nitrogen atoms (N).

The heteroaromatic ring is a single ring (preferably 5 or 6 membered with up to three Nitrogen atoms (N)) or a fused ring (preferably 9 or 10 membered with up to four Nitrogen atoms (N)).

A substituted heteroaromatic ring is substituted with halogen, NO2, CN, COOR3, SO2R3 or CF₃ wherein R³ is defined below.

Preferably, the heteroaromatic ring comprises 2 to 4, Nitrogen atoms (N)) more preferably 3 to 4 or 3.

More preferably, Y is a moiety of Formula III

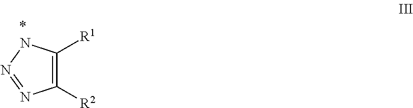

wherein
* indicates the position of the covalent bond to the Oxygen (O*) in Formula Ia;
R¹ is H, CN, or COOR⁴, and R² is H, CN, or COOR⁴, or
R¹ and R² form together a 6 membered aromatic ring, which optionally comprise 1 Nitrogen atom (N) and 1 methine of the 6 membered ring is optionally substituted with halogen, NO₂, CN, COOR³, SO₂R³ or CF₃,
R³ is C₁-C₃ alkyl, and
R⁴ is C₁-C₆ alkyl.

Preferably, R¹ and R² form together a 6 membered aromatic ring, which optionally comprise 1 Nitrogen atom (N) and 1 methine of the 6 membered ring is optionally substituted with halogen, NO₂, or CF₃.

Preferably, R³ is C₁ alkyl(methyl).

Preferably, R⁴ is C₁ alkyl(methyl) or C₂ alkyl(ethyl).

Preferably, halogen is chloro (Cl).

Even more preferably, Y is

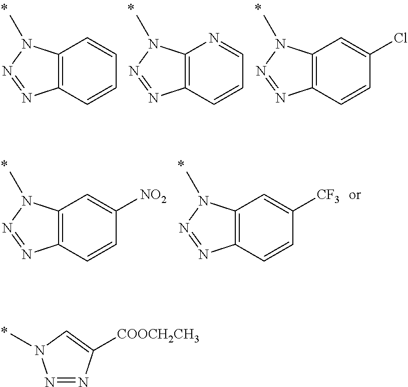

* indicates the position of the covalent bond to the Oxygen (O*) in Formula Ia.

Even more preferably, Y is

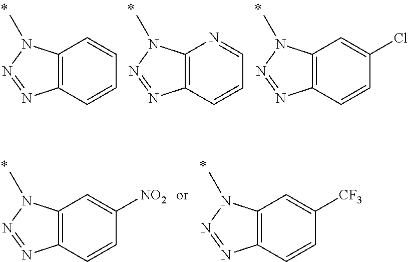

* indicates the position of the covalent bond to the Oxygen (O*) in Formula Ia.

Preferably, O*—Y acts as a leaving group suitable for introducing a fluoride.

PG1 is a carboxylic acid protecting group (forming an ester) containing up to 20 carbon atoms, optionally containing independently one or more O, N or S atoms; and compatible with radiolabeling conditions.

Preferably, PG1 is
Alkyl,
Alkyl substituted with one phenyl,
Alkyl substituted with one or two C₃-C₆cycloalkyl,
Alkyl substituted with one phenyl and one C₃-C₆cycloalkyl, or
fluorenylmethyl
wherein
   Alkyl is a branched or linear C₁-C₆ alkyl, and optionally substituted with C₁-C₃-alkoxy, and
   Phenyl is optionally substituted with up to three C₁-C₃ alkyl, C₁-C₃ alkoxy or halogen.

PG1 is defined with the proviso that PG1 contains up to 20 carbon atoms,

Preferably, branched or linear C₁-C₆ alkyl is a C₁-C₃ alkyl. More preferably, C₁-C₆ alkyl is C₁-alkyl(methyl) when substituted and C₄-alkyl (e.g. tert-Butyl) when unsubstituted. Preferably, branched or linear C₁-C₆ alkyl substituted with one phenyl is a branched or linear C₁-C₃ alkyl substituted with one phenyl. More preferably, branched or linear C₁-C₆ alkyl substituted with one phenyl is a methyl-phenyl(benzyl), ethyl-phenyl or i-Propyl-phenyl (e.g. Cumyl). Preferably, methyl-phenyl(benzyl), ethyl-phenyl and i-Propyl-phenyl (e.g. Cumyl) are substituted with up to two methoxy-groups.

Preferably, C₁-C₃ alkoxy is C₁-alkoxy(methoxy).

Preferably, branched or linear C₁-C₆ alkyl substituted with one or two C₃-C₆cycloalkyl is a branched or linear C₁-C₃ alkyl substituted with one or two cyclo-propyl.

Preferably, branched or linear C₁-C₆ alkyl substituted with one phenyl and one C₃-C₆ cycloalkyl is a branched or linear C₁-C₃ alkyl substituted with one phenyl and one C₃-C₆ cycloalkyl wherein the C₃-C₆ cycloalkyl is preferably C₃ cycloalkyl(cyclo-propyl),

Fluorenylmethyl is

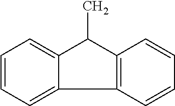

More preferably, PG1 is
Alkyl,
Alkyl substituted with one phenyl,
Alkyl substituted with one or two C₃ cycloalkyl,
Alkyl substituted with one phenyl and one C₃ cycloalkyl, or
fluorenylmethyl
wherein
   Alkyl is a branched or linear C₁-C₄ alkyl, and optionally substituted with C₁-C₃-alkoxy, and
   Phenyl is optionally substituted with up to three C₁-C₃ alkyl, C₁-C₃ alkoxy or halogen.

Even more preferably, PG1 is

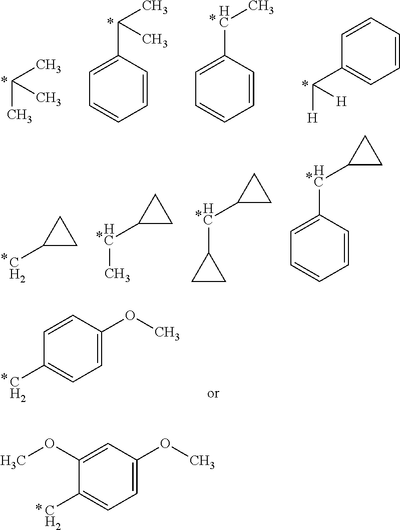

wherein * indicates the position of the Oxygen (O) forming the ester bond in Formula Ia.

Even more preferably, PG1 is

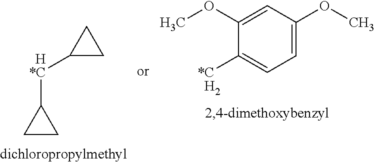

wherein * indicates the position of the Oxygen (O) forming the ester bond in Formula Ia.

PG2 is an amino protecting group containing up to 20 carbon atoms, optionally containing one or more O, N or S atoms and are optionally substituted with one to three halogen atoms, and compatible with radiolabeling conditions.

Preferably, PG2 is a carbamate or an arylalkyl protecting group containing up to 20 carbon atoms.

More preferably PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

Even more preferably, PG2 is tert-Butoxycarbonyl (BOC) or Triphenylmethyl (trityl).

Preferably, Z is Hydrogen.

### Preferred Compound of Formula Ia:

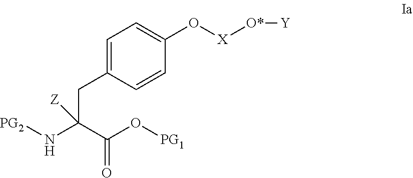

wherein:
X is CH₂ or CD₂;
Y is

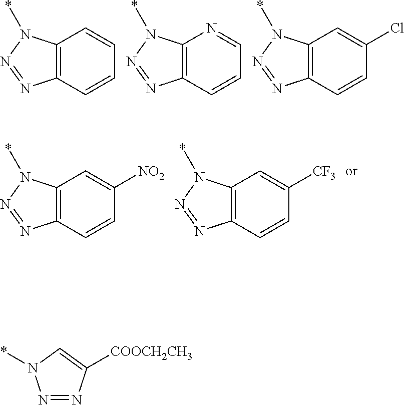

Z is Hydrogen or methyl;
PG1 is dicyclopropylmethyl or 2,4-dimethoxybenzyl; and
PG2 is tert-Butoxycarbonyl (BOC) or Triphenylmethyl (trityl).

In a first embodiment, the invention of novel precursors is directed to compounds of Formula Ia

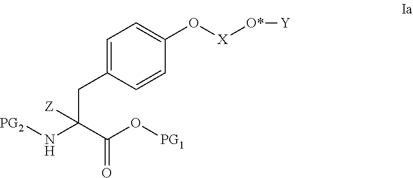

wherein:
X CH₂;
Y is a substituted or unsubstituted heteroaromatic ring containing one to four Nitrogen atoms (N) with the proviso that the oxygen (O*) is directly bound to one of the Nitrogen atoms (N) of the heteroaromatic ring and O*—Y acts as leaving group;
Z Hydrogen;
PG1 is a carboxylic acid protecting group, containing up to 20 carbon atoms, optionally containing independently one or more O, N or S atoms; and
PG2 is an amino protecting group, containing up to 20 carbon atoms, optionally containing one or more O, N or S atoms and are optionally substituted with one to three halogens.

The invention further refers to suitable salts of inorganic or organic acids, hydrates and solvates of the compounds of Formula Ia and is also meant to comprise single isomers, diastereomers, enantiomers and mixtures thereof of Formula Ia.

Formula (Ib) correspond to the Markush Formula below

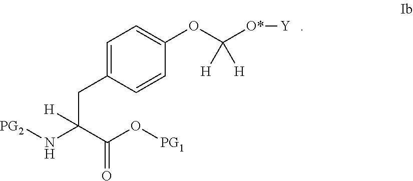

Preferred features disclosed above in respect of Y, PG1 and PG2 are incorporated herein.

Preferably, the invention is directed to compounds of Formula (Ib) wherein
Y is a moiety of Formula III

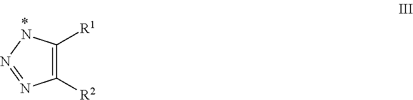

wherein
* indicates the position of the covalent bond to the Oxygen (O*) in Formula Ib;
R¹ is H, CN, or COOR⁴, and R² is H, CN, or COOR⁴, or
R¹ and R² form together a 6 membered aromatic ring, which optionally comprise 1

Nitrogen atom (N) and 1 methine of the 6 membered ring is optionally substituted with halogen, NO₂, CN, COOR³, SO₂R³ or CF₃,
R³ is C₁-C₃ alkyl,
R⁴ is C₁-C₆ alkyl;
PG1 is
   Alkyl,
   Alkyl substituted with one phenyl,
   Alkyl substituted with one or two C₃-C₆cycloalkyl,
   Alkyl substituted with one phenyl and one C₃-C₆cycloalkyl, or fluorenylmethyl
   wherein
      Alkyl is a branched or linear C₁-C₆ alkyl, and optionally substituted with C₁-C₃alkoxy, and
      Phenyl is optionally substituted with up to three C₁-C₃ alkyl, C₁-C₃ alkoxy or halogen; and
PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

More preferably, compounds of Formula (Ib) is wherein
Y is

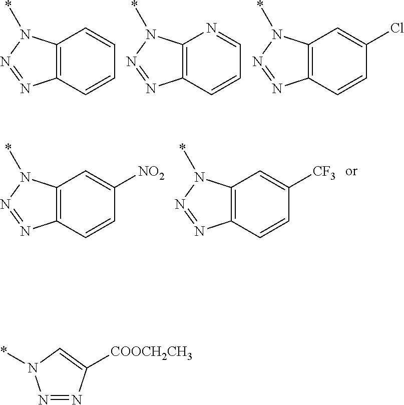

PG1 is dicyclopropylmethyl or 2,4-dimethoxybenzyl, and
PG2 is tert-Butoxycarbonyl (BOC) or Triphenylmethyl (trityl).

In a second embodiment, the invention of novel precursors is directed to compounds of Formula Ia

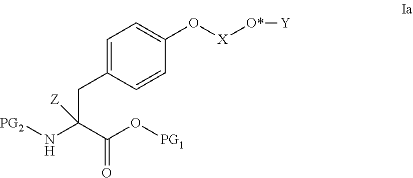

wherein:
X is CD₂;
Y is a substituted or unsubstituted heteroaromatic ring containing one to four

Nitrogen atoms (N) with the proviso that the oxygen (O*) is directly bound to one of the Nitrogen atoms (N) of the heteroaromatic ring and O*—Y acts as leaving group;
Z is Hydrogen;
PG1 is a carboxylic acid protecting group, containing up to 20 carbon atoms, optionally containing independently one or more O, N or S atoms; and
PG2 is an amino protecting group, containing up to 20 carbon atoms, optionally containing one or more O, N or S atoms and are optionally substituted with one to three halogens.

The invention further refers to suitable salts of inorganic or organic acids, hydrates and solvates of the compounds of Formula Ia and is also meant to comprise single isomers, diastereomers, enantiomers and mixtures thereof of Formula Ia.

Formula (Ic) corresponds to the Markush Formula below

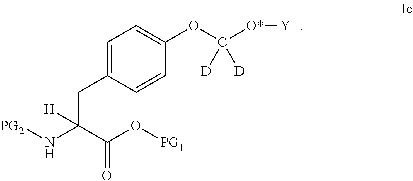

Preferred features disclosed above in respect of Y, PG1 and PG2 are incorporated herein.

Preferably, the invention is directed to compounds of Formula (Ic) wherein
Y is a moiety of Formula III

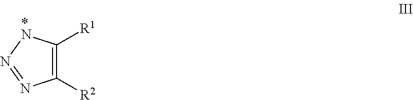

wherein
* indicates the position of the covalent bond to the Oxygen (O*) in Formula Ic;
R¹ is H, CN, or COOR⁴, and R² is H, CN, or COOR⁴, or
R¹ and R² form together a 6 membered aromatic ring, which optionally comprise 1 Nitrogen atom (N) and 1 methine of the 6 membered ring is optionally substituted with halogen, NO₂, CN, COOR³, SO₂R³ or CF₃,
R³ is C₁-C₃ alkyl,
R⁴ is C₁-C₆ alkyl;
PG1 is
   Alkyl,
   Alkyl substituted with one phenyl,
   Alkyl substituted with one or two C₃-C₆ cycloalkyl,
   Alkyl substituted with one phenyl and one C₃-C₆ cycloalkyl, or
   fluorenylmethyl
   wherein
      Alkyl is a branched or linear C₁-C₆ alkyl, and optionally substituted with C₁-C₃ alkoxy, and
      Phenyl is optionally substituted with up to three C₁-C₃ alkyl, C₁-C₃ alkoxy or halogen; and
PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

More preferably, compounds of Formula (Ic) is wherein
Y is

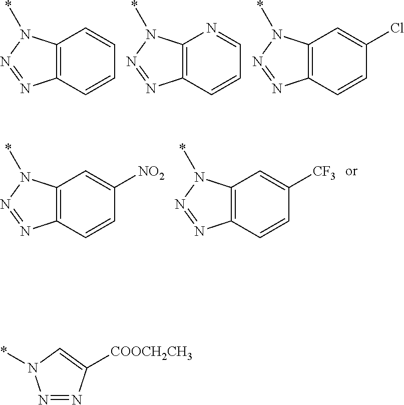

PG1 is dicyclopropylmethyl or 2,4-dimethoxybenzyl, and
PG2 is tert-Butoxycarbonyl (BOC) or Triphenylmethyl (trityl).

In a third embodiment, the invention of novel precursors is directed to compounds of Formula Ia

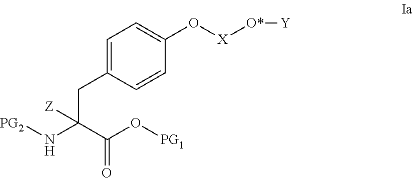

wherein:
X is CH₂;
Y is a substituted or unsubstituted heteroaromatic ring containing one to four Nitrogen atoms (N) with the proviso that the oxygen (O*) is directly bound to one of the Nitrogen atoms (N) of the heteroaromatic ring and O*—Y acts as leaving group;
Z is methyl;
PG1 is a carboxylic acid protecting group, containing up to 20 carbon atoms, optionally containing independently one or more O, N or S atoms; and
PG2 is an amino protecting group, containing up to 20 carbon atoms, optionally containing one or more O, N or S atoms and are optionally substituted with one to three halogens.

The invention further refers to suitable salts of inorganic or organic acids, hydrates and solvates of the compounds of Formula Ia and is also meant to comprise single isomers, diastereomers, enantiomers and mixtures thereof of Formula Ia.

Formula (Id) corresponds to the Markush Formula below

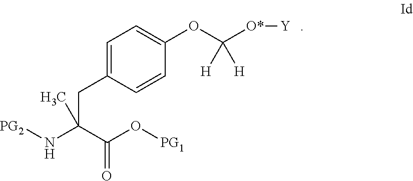

Preferred features disclosed above in respect of Y, PG1 and PG2 are incorporated herein.

Preferably, the invention is directed to compounds of Formula (Id) wherein
Y is a moiety of Formula III

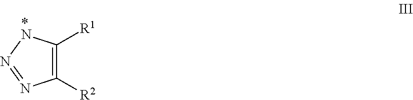

wherein
* indicates the position of the covalent bond to the Oxygen (O*) in Formula Id;
R¹ is H, CN, or COOR⁴, and R² is H, CN, or COOR⁴, or
R¹ and R² form together a 6 membered aromatic ring, which optionally comprise 1 Nitrogen atom (N) and 1 methine of the 6 membered ring is optionally substituted with halogen, NO₂, CN, COOR³, SO₂R³ or CF₃,
R³ is C₁-C₃ alkyl,
R⁴ is C₁-C₆ alkyl;
PG1 is
   Alkyl,
   Alkyl substituted with one phenyl,
   Alkyl substituted with one or two C₃-C₆ cycloalkyl,
   Alkyl substituted with one phenyl and one C₃-C₆ cycloalkyl, or
   fluorenylmethyl
   wherein
      Alkyl is a branched or linear C₁-C₆ alkyl, and optionally substituted with C₁-C₃ alkoxy, and
      Phenyl is optionally substituted with up to three C₁-C₃ alkyl, C₁-C₃ alkoxy or halogen; and
PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

More preferably, compounds of Formula (Id) is wherein
Y is

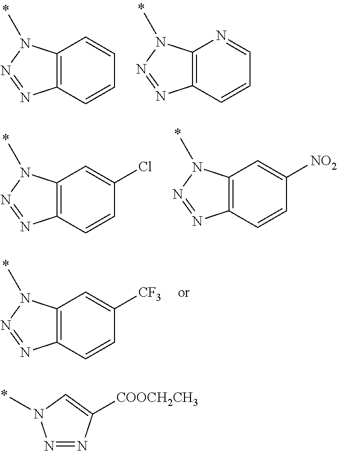

PG1 is dicyclopropylmethyl or 2,4-dimethoxybenzyl, and
PG2 is tert-Butoxycarbonyl (BOC) or Triphenylmethyl (trityl).

In a fourth embodiment, the invention of novel precursors is directed to compounds of Formula Ia

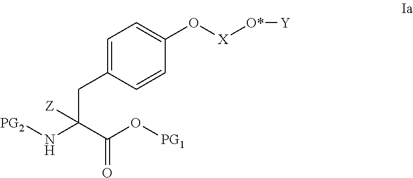

wherein:
X is CD₂;
Y is a substituted or unsubstituted heteroaromatic ring containing one to four Nitrogen atoms (N) with the proviso that the oxygen (O*) is directly bound to one of the Nitrogen atoms (N) of the heteroaromatic ring and O*—Y acts as leaving group;
Z is methyl;
PG1 is a carboxylic acid protecting group, containing up to 20 carbon atoms, optionally containing independently one or more O, N or S atoms; and
PG2 is an amino protecting group, containing up to 20 carbon atoms, optionally containing one or more O, N or S atoms and are optionally substituted with one to three halogens.

The invention further refers to suitable salts of inorganic or organic acids, hydrates and solvates of the compounds of Formula Ia and is also meant to comprise single isomers, diastereomers, enantiomers and mixtures thereof of Formula Ia.

Formula (Ie) corresponds to the Markush Formula below

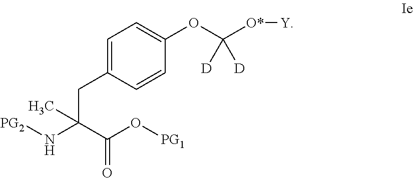

Preferred features disclosed above in respect of Y, PG1 and PG2 are incorporated herein. Preferably, the invention is directed to compounds of Formula (Ie) wherein
Y is a moiety of Formula III

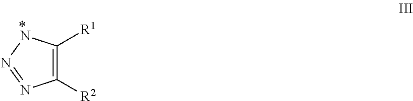

wherein
* indicates the position of the covalent bond to the Oxygen (O*) in Formula Ie;
R¹ is H, CN, or COOR⁴, and R² is H, CN, or COOR⁴, or
R¹ and R² form together a 6 membered aromatic ring, which optionally comprise 1 Nitrogen atom (N) and 1 methine of the 6 membered ring is optionally substituted with halogen, NO₂, CN, COOR³, SO₂R³ or CF₃,
R³ is C₁-C₃ alkyl,
R⁴ is C₁-C₆ alkyl;
PG1 is
   Alkyl,
   Alkyl substituted with one phenyl,
   Alkyl substituted with one or two C₃-C₆ cycloalkyl,
   Alkyl substituted with one phenyl and one C₃-C₆ cycloalkyl, or
   fluorenylmethyl
   wherein
      Alkyl is a branched or linear C₁-C₆ alkyl, and optionally substituted with C₁-C₃ alkoxy, and
      Phenyl is optionally substituted with up to three C₁-C₃ alkyl, C₁-C₃ alkoxy or halogen; and
PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

More preferably, compounds of Formula (Ie) is wherein
Y is

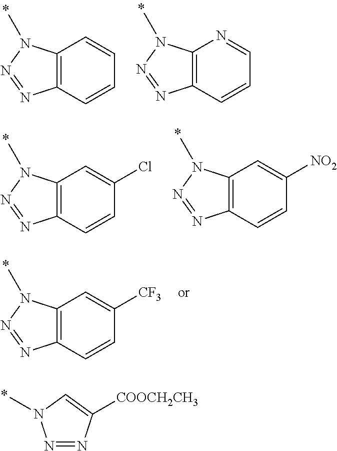

PG1 is dicyclopropylmethyl or 2,4-dimethoxybenzyl, and
PG2 is tert-Butoxycarbonyl (BOC) or Triphenylmethyl (trityl).

In a fifth embodiment, the invention of novel precursors is directed to compounds of Formula (D-Ia), (D-Ib), (D-Ic), (D-Id) or (D-Ie) wherein

(D-Ia), (D-Ib), (D-Ic), (D-Id) or (D-Ie)

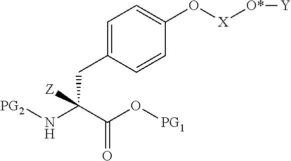

| Formula\Substituent | Z | X |
|---|---|---|
| D-Ia | H, CH₃ | CH₂, CD₂ |
| D-Ib | H | CH₂ |
| D-Ic | H | CD₂ |
| D-Id | CH₃ | CH₂ |
| D-Ie | CH₃ | CD₂ |

Y, PG1 and PG2 are disclosed as above and encompass preferred features as disclosed above.

The invention further refers to suitable salts of inorganic or organic acids, hydrates and solvates of the compounds of Formula (D-Ia), (D-Ib), (D-Ic), (D-Id) or (D-Ie) and is also meant to comprise single isomers, diastereomers, enantiomers and mixtures thereof of Formula (D-Ia), (D-Ib), (D-Ic), (D-Id) or (D-Ie).

Embodiments and preferred features can be combined together and are within the scope of the invention.

Invention compounds are but not limited to
tert-Butyl O-[(1H-benzotriazol-1-yloxy)methyl]-N-(tert-butoxycarbonyl)-D-tyrosinate

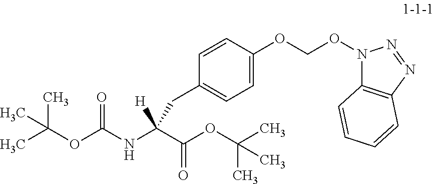 [SMILES: CC(C)(C)OC(=O)N[C@H](Cc1ccc(OCOn2nnc3ccccc32)cc1)C(=O)OC(C)(C)C]

tert-Butyl N-(tert-butoxycarbonyl)-O-[(1H-1,2,3-triazolo[5,4-b]pyridin-1-yloxy)methyl]-D-tyrosinate

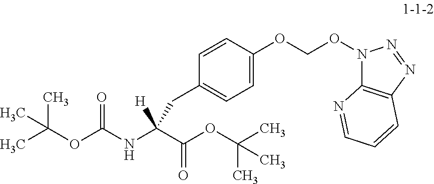 [SMILES: CC(C)(C)OC(=O)N[C@H](Cc1ccc(OCOn2nnc3cccnc32)cc1)C(=O)OC(C)(C)C]

Dicyclopropylmethyl O-[(1H-benzotriazol-1-yloxy)methyl]-N-(tert-butoxycarbonyl)-D-tyrosinate

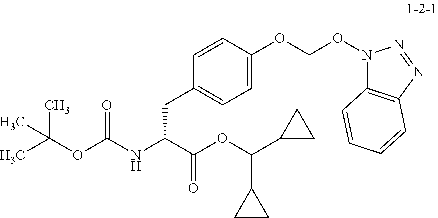 [SMILES: CC(C)(C)OC(=O)N[C@H](Cc1ccc(OCOn2nnc3ccccc32)cc1)C(=O)OC(C1CC1)C1CC1]

Dicyclopropylmethyl O-[(1H-benzotriazol-1-yloxy)methyl]-N-(tert-butoxycarbonyl)-L-tyrosinate

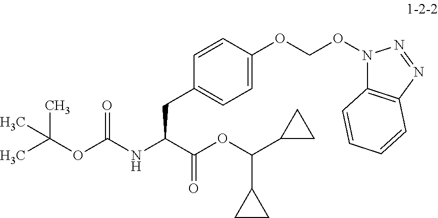 [SMILES: CC(C)(C)OC(=O)N[C@@H](Cc1ccc(OCOn2nnc3ccccc32)cc1)C(=O)OC(C1CC1)C1CC1]

Dicyclopropylmethyl N-(tert-butoxycarbonyl)-O-[(6-nitro-1H-benzotriazol-1-yloxy)methyl]-D-tyrosinate

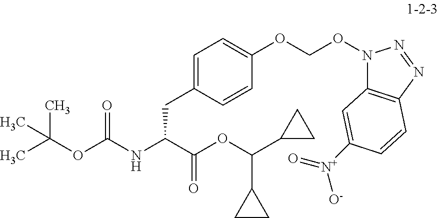 [SMILES: CC(C)(C)OC(=O)N[C@H](Cc1ccc(OCOn2nnc3ccc([N+](=O)[O-])cc32)cc1)C(=O)OC(C1CC1)C1CC1]

2,4-Dimethoxybenzyl O-[(1H-benzotriazol-1-yloxy)methyl]-N-(tert-butoxycarbonyl)-D-tyrosinate

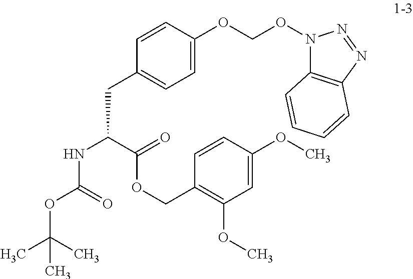 [SMILES: COc1ccc(COC(=O)[C@@H](Cc2ccc(OCOn3nnc4ccccc43)cc2)NC(=O)OC(C)(C)C)c(OC)c1]

Cyclopropylmethyl O-[(1H-benzotriazol-1-yloxy)methyl]-N-(tert-butoxycarbonyl)-D-tyrosinate

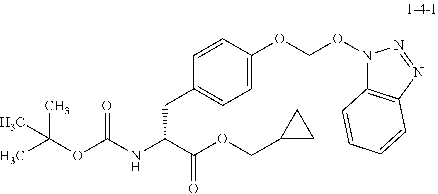 [SMILES: CC(C)(C)OC(=O)N[C@H](Cc1ccc(OCOn2nnc3ccccc32)cc1)C(=O)OCC1CC1]

Cyclopropylmethyl N-(tert-butoxycarbonyl)-O-({[4-(ethoxycarbonyl)-1H-1,2,3-triazol-1-yl]oxy}methyl)-D-tyrosinate

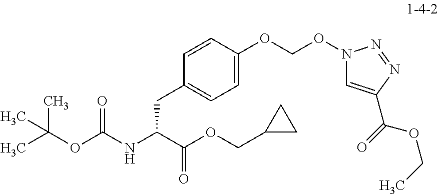 [SMILES: CCOC(=O)c1cn(OCOc2ccc(C[C@@H](NC(=O)OC(C)(C)C)C(=O)OCC3CC3)cc2)nn1]

4-Methoxybenzyl O-[(1H-benzotriazol-1-yloxy)methyl]-N-(tert-butoxycarbonyl)-D-tyrosinate

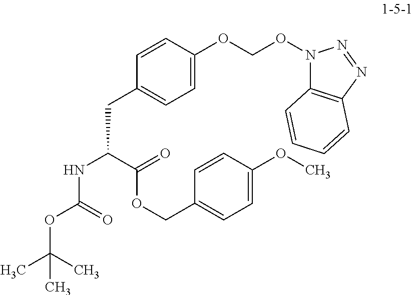 [SMILES: COc1ccc(COC(=O)[C@@H](Cc2ccc(OCOn3nnc4ccccc43)cc2)NC(=O)OC(C)(C)C)cc1]

4-Methoxybenzyl N-(tert-butoxycarbonyl)-O-{[(6-chloro-1H-benzotriazol-1-yl)oxy]methyl}-D-tyrosinate

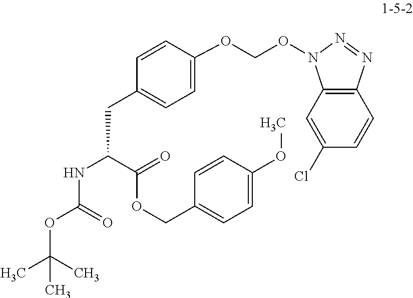 [SMILES: COc1ccc(COC(=O)[C@@H](Cc2ccc(OCOn3nnc4ccc(Cl)cc43)cc2)NC(=O)OC(C)(C)C)cc1]

4-Methoxybenzyl N-(tert-butoxycarbonyl)-O-[(6-trifluoromethyl-1H-benzotriazol-1-yloxy)methyl]-D-tyrosinate

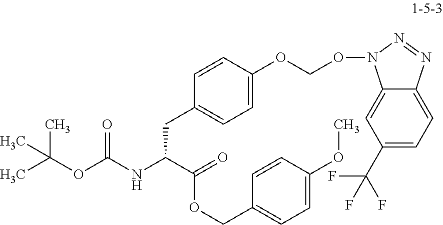 [SMILES: COc1ccc(COC(=O)[C@@H](Cc2ccc(OCOn3nnc4ccc(C(F)(F)F)cc43)cc2)NC(=O)OC(C)(C)C)cc1]

4-Methoxybenzyl O-[(6-trifluoromethyl-1H-benzotriazol-1-yloxy)methyl]-N-(tert-butoxycarbonyl)-L-tyrosinate.

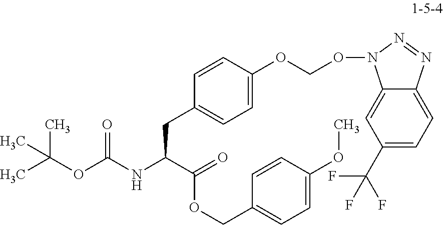 [SMILES: COc1ccc(COC(=O)[C@H](Cc2ccc(OCOn3nnc4ccc(C(F)(F)F)cc43)cc2)NC(=O)OC(C)(C)C)cc1]

alpha-Methylbenzyl O-[(1H-benzotriazol-1-yloxy)methyl]-N-(tert-butoxycarbonyl)-D-tyrosinate

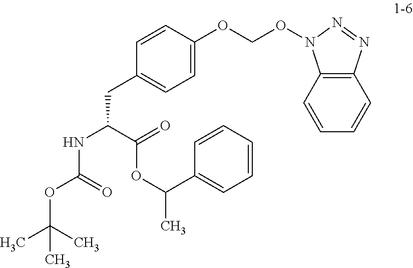 [SMILES: CC(OC(=O)[C@@H](Cc1ccc(OCOn2nnc3ccccc32)cc1)NC(=O)OC(C)(C)C)c1ccccc1]

alpha,alpha-Dimethylbenzyl O-[(1H-benzotriazol-1-yloxy)methyl]-N-(tert-butoxycarbonyl)-D-tyrosinate

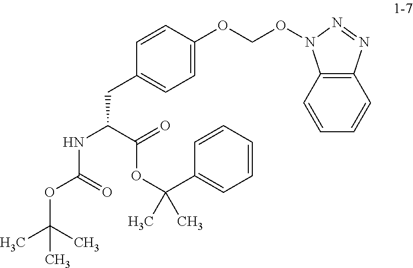 [SMILES: CC(C)(C)OC(=O)N[C@H](Cc1ccc(OCOn2nnc3ccccc32)cc1)C(=O)OC(C)(C)c1ccccc1]

tert-Butyl O-[(1H-benzotriazol-1-yloxy)methyl]-N-trityl-D-tyrosinate

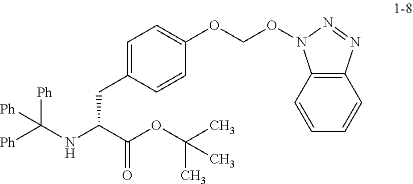 [SMILES: CC(C)(C)OC(=O)[C@@H](Cc1ccc(OCOn2nnc3ccccc32)cc1)NC(c1ccccc1)(c1ccccc1)c1ccccc1]

4-Methoxybenzyl O-[(1H-benzotriazol-1-yloxy)methyl]-N-trityl-D-tyrosinate

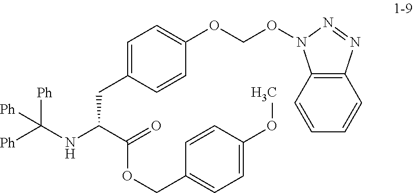 [SMILES: COc1ccc(COC(=O)[C@@H](Cc2ccc(OCOn3nnc4ccccc43)cc2)NC(c2ccccc2)(c2ccccc2)c2ccccc2)cc1]

Cyclopropylmethyl O-[(1H-benzotriazol-1-yloxy)[²H₂]methyl]-N-(tert-butoxy-carbonyl)-D-tyrosinate

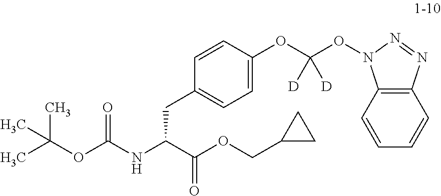 [SMILES: [2H]C([2H])(Oc1ccc(C[C@@H](NC(=O)OC(C)(C)C)C(=O)OCC2CC2)cc1)On1nnc2ccccc21]

2,4-Dimethoxybenzyl O-[(1H-benzotriazol-1-yloxy)methyl]-N-trityl-D-tyrosinate

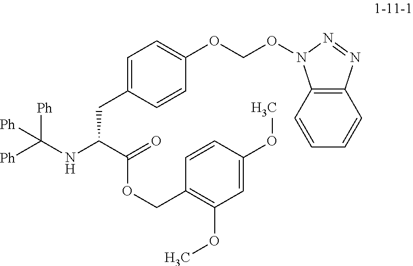 [SMILES: COc1ccc(COC(=O)[C@@H](Cc2ccc(OCOn3nnc4ccccc43)cc2)NC(c2ccccc2)(c2ccccc2)c2ccccc2)c(OC)c1]

2,4-Dimethoxybenzyl 0-{[(6-chloro-1H-benzotriazol-1-yl)oxy]methyl}-N-trityl-D-tyrosinate

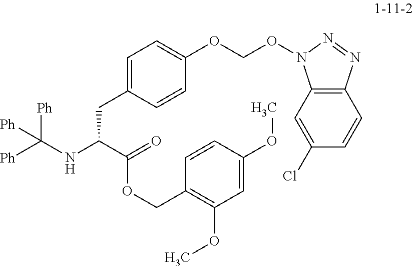 [SMILES: COc1ccc(COC(=O)[C@@H](Cc2ccc(OCOn3nnc4ccc(Cl)cc43)cc2)NC(c2ccccc2)(c2ccccc2)c2ccccc2)c(OC)c1]

2,4-Dimethoxybenzyl 0-{[(6-trifluoromethyl-1H-benzotriazol-1-yl)oxy]methyl}-N-trityl-D-tyrosinate

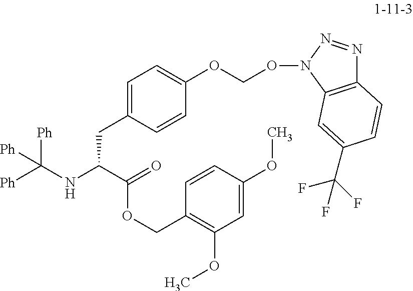 [SMILES: COc1ccc(COC(=O)[C@@H](Cc2ccc(OCOn3nnc4ccc(C(F)(F)F)cc43)cc2)NC(c2ccccc2)(c2ccccc2)c2ccccc2)c(OC)c1]

Methyl O-[(1H-benzotriazol-1-yloxy)methyl]-N-(tert-butoxycarbonyl)-alpha-methyltyrosinate

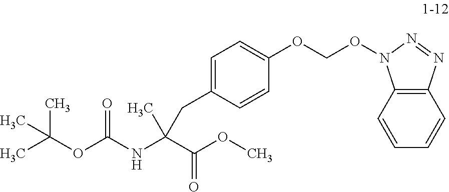 [SMILES: COC(=O)C(C)(Cc1ccc(OCOn2nnc3ccccc32)cc1)NC(=O)OC(C)(C)C]

In a second aspect, the present invention of novel precursors is directed to compounds of Formula IIa

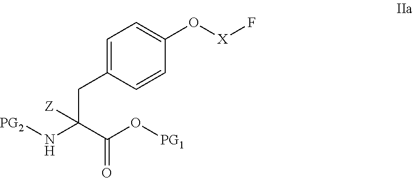

wherein:
X is CH₂, CHD or CD₂;
F is ¹⁸F or ¹⁹F;
Z is Hydrogen or methyl;
PG1 is a carboxylic protecting group, containing up to 20 carbon atoms, optionally containing independently one or more O, N or S atoms; and
PG2 is an amino protecting group, containing up to 20 carbon atoms, optionally containing one or more O, N or S atoms and are optionally substituted with one or two halogens.

The invention further refers to suitable salts of inorganic or organic acids, hydrates and solvates of the compounds of Formula IIa and is also meant to comprise single isomers, diastereomers, enantiomers and mixtures thereof of Formula IIa.

### Preferred Features:

PG1 is a carboxylic acid protecting group (forming an ester) containing up to 20 carbon atoms, optionally containing independently one or more O, N or S atoms; and compatible with radiolabeling conditions.

Preferably, PG1 is
Alkyl,
Alkyl substituted with one phenyl,
Alkyl substituted with one or two C₃-C₆cycloalkyl,
Alkyl substituted with one phenyl and one C₃-C₆cycloalkyl, or
fluorenylmethyl
wherein
   Alkyl is a branched or linear C₁-C₆ alkyl, and optionally substituted with C₁-C₃ alkoxy, and
   Phenyl is optionally substituted with up to three C₁-C₃ alkyl, C₁-C₃ alkoxy or halogen.

Preferably, branched or linear C₁-C₆ alkyl is a C₁-C₃ alkyl. More preferably, C₁-C₆ alkyl is C₁-alkyl(methyl) when substituted and C₄-alkyl (e.g. tert-Butyl) when unsubstituted.

Preferably, branched or linear C₁-C₆ alkyl substituted with one phenyl is branched or linear C₁-C₃ alkyl substituted with one phenyl. More preferably, branched or linear C₁-C₆ alkyl substituted with one phenyl is methyl-phenyl (benzyl), ethyl-phenyl or i-Propyl-phenyl (e.g. Cumyl). Preferably, methyl-phenyl(benzyl), ethyl-phenyl and i-Propyl-phenyl (e.g. Cumyl) are substituted with up to two methoxy-groups.

Preferably, C₁-C₃alkoxy is C₁ alkoxy(methoxy).

Preferably, branched or linear C₁-C₆ alkyl substituted with one or two C₃-C₆ cycloalkyl is branched or linear C₁-C₃ alkyl substituted with one or two cyclo-propyl.

Preferably, branched or linear C₁-C₆ alkyl substituted with one phenyl and one C₃-C₆ cycloalkyl is branched or linear C₁-C₃ alkyl substituted with one phenyl and one C₃-C₆ cycloalkyl wherein the C₃-C₆ cycloalkyl is preferably C₃ cycloalkyl(cyclo-propyl),

Fluorenylmethyl is

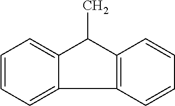

More preferably, PG1 is
Alkyl,
Alkyl substituted with one phenyl,
Alkyl substituted with one or two C₃ cycloalkyl,
Alkyl substituted with one phenyl and one C₃ cycloalkyl, or
fluorenylmethyl
wherein
   Alkyl is a branched or linear C₁-C₄ alkyl, and optionally substituted with C₁-C₃ alkoxy, and
   Phenyl is optionally substituted with up to three C₁-C₃ alkyl, C₁-C₃ alkoxy or halogen.

Even more preferably, PG1 is

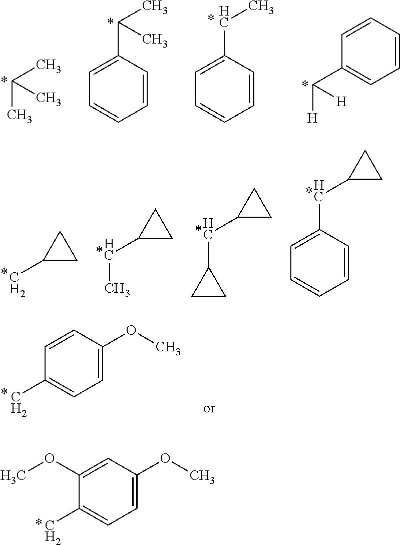

wherein * indicates the position of the Oxygen (O) forming the ester bond in Formula IIa;

Even more preferably, PG1 is

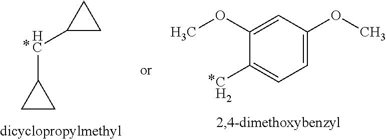

wherein * indicates the position of the Oxygen (O) forming the ester bond in Formula IIa.

PG2 is an amino protecting group containing up to 20 carbon atoms, optionally containing one or more O, N or S atoms and are optionally substituted with one to three halogens, and compatible with radiolabeling conditions.

Preferably, PG2 is a carbamate or an arylalkyl protecting group.

More preferably PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

Even more preferably, PG2 is tert-Butoxycarbonyl (BOC) or Triphenylmethyl (trityl).

Preferably, F is ¹⁸F.

Preferably, F is ¹⁹F.

Preferably, Z is Hydrogen.

### Preferred Compounds of Formula IIa:

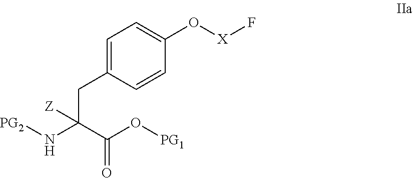

wherein:
X is CH₂ or CD₂;
F is ¹⁸F;
Z is Hydrogen or methyl;
PG1 is dicyclopropylmethyl or 2,4-dimethoxybenzyl; and
PG2 is tert-Butoxycarbonyl (BOC) or Triphenylmethyl (trityl).

Hereto, in a first embodiment, the invention of novel precursors is directed to compounds of Formula IIa

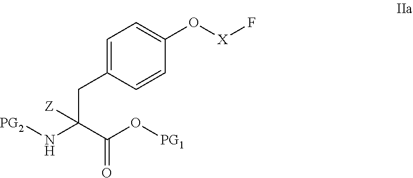

wherein:
X is CH₂;
F is ¹⁸F or ¹⁹F;
Z is Hydrogen;
PG1 is a carboxylic acid protecting group, containing up to 20 carbon atoms, optionally containing independently one or more O, N or S atoms; and
PG2 is an amino protecting group, containing up to 20 carbon atoms, optionally containing one or more O, N or S atoms and are optionally substituted with one to three halogens.

The invention further refers to suitable salts of inorganic or organic acids, hydrates and solvates of the compounds of Formula IIa and is also meant to comprise single isomers, diastereomers, enantiomers and mixtures thereof of Formula IIa.

Formula (IIb) corresponds to the Markush Formula below

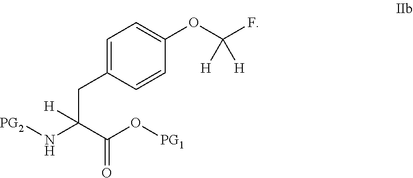

Preferred features disclosed above in respect of F, PG1 and PG2 are incorporated herein.

Preferably, the invention is directed to compounds of Formula (IIb) wherein
F is ¹⁸F or ¹⁹F;
PG1 is
   Alkyl,
   Alkyl substituted with one phenyl,
   Alkyl substituted with one or two C₃-C₆cycloalkyl,
   Alkyl substituted with one phenyl and one C₃-C₆cycloalkyl, or
   fluorenylmethyl
   wherein
      Alkyl is a branched or linear C₁-C₆ alkyl, and optionally substituted with C₁-C₃ alkoxy, and
      Phenyl is optionally substituted with up to three C₁-C₃ alkyl, C₁-C₃ alkoxy or halogen; and
PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

More preferably, compounds of Formula (IIb) is wherein
F is ¹⁸F;
PG1 is dicyclopropylmethyl or 2,4-dimethoxybenzyl, and
PG2 is tert-Butoxycarbonyl (BOC) or Triphenylmethyl (trityl).

Hereto, in a second embodiment, the invention of novel precursors is directed to compounds of Formula IIa

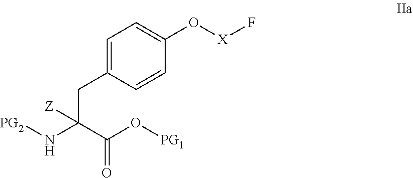

wherein:
X is CD₂;
F is ¹⁸F or ¹⁹F;
Z is Hydrogen;
PG1 is a carboxylic acid protecting group, containing up to 20 carbon atoms, optionally containing independently one or more O, N or S atoms; and
PG2 is an amino protecting group, containing up to 20 carbon atoms, optionally containing one or more O, N or S atoms and are optionally substituted with one to three halogens.

The invention further refers to suitable salts of inorganic or organic acids, hydrates and solvates of the compounds of Formula IIa and is also meant to comprise single isomers, diastereomers, enantiomers and mixtures thereof of Formula IIa.

Formula (IIc) corresponds to the Markush Formula below

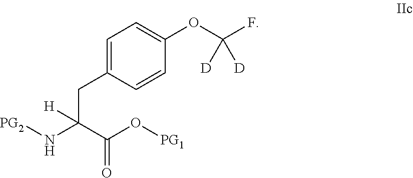

Preferred features disclosed above in respect of F, PG1 and PG2 are incorporated herein.

Preferably, the invention is directed to compounds of Formula (IIc) wherein
Y is ¹⁸F or ¹⁹F;
PG1 is
   Alkyl,
   Alkyl substituted with one phenyl,
   Alkyl substituted with one or two C₃-C₆cycloalkyl,
   Alkyl substituted with one phenyl and one C₃-C₆cycloalkyl, or
   fluorenylmethyl
   wherein
      Alkyl is a branched or linear C₁-C₆ alkyl, and optionally substituted with C₁-C₃ alkoxy, and
      Phenyl is optionally substituted with up to three C₁-C₃ alkyl, C₁-C₃ alkoxy or halogen; and
PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

More preferably, compounds of Formula (IIc) is wherein
Y is ¹⁸F;
PG1 is dicyclopropylmethyl or 2,4-dimethoxybenzyl, and
PG2 is tert-Butoxycarbonyl (BOC) or Triphenylmethyl (trityl).

Hereto, in a third embodiment, the invention of novel precursors is directed to compounds of Formula IIa

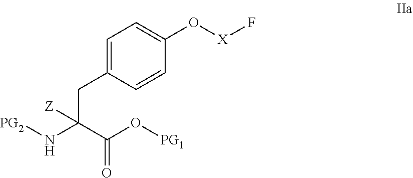

wherein:
X is CH₂;
F is ¹⁸F or ¹⁹F;
Z is methyl;
PG1 is a carboxylic acid protecting group, containing up to 20 carbon atoms, optionally containing independently one or more O, N or S atoms; and
PG2 is an amino protecting group, containing up to 20 carbon atoms, optionally containing one or more O, N or S atoms and are optionally substituted with one to three halogens.

The invention further refers to suitable salts of inorganic or organic acids, hydrates and solvates of the compounds of Formula IIa and is also meant to comprise single isomers, diastereomers, enantiomers and mixtures thereof of Formula IIa.

Formula (IId) corresponds to the Markush Formula below

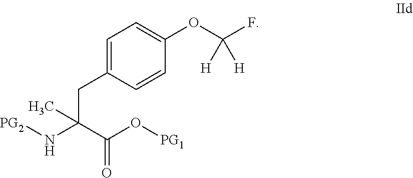

Preferred features disclosed above in respect of F, PG1 and PG2 are incorporated herein.

Preferably, the invention is directed to compounds of Formula (IId) wherein
Y is ¹⁸F or ¹⁹F;
PG1 is
   Alkyl,
   Alkyl substituted with one phenyl,
   Alkyl substituted with one or two C₃-C₆cycloalkyl,
   Alkyl substituted with one phenyl and one C₃-C₆cycloalkyl, or
   fluorenylmethyl
   wherein
      Alkyl is a branched or linear C₁-C₆ alkyl, and optionally substituted with C₁-C₃alkoxy, and
      Phenyl is optionally substituted with up to three C₁-C₃ alkyl, C₁-C₃ alkoxy or halogen; and
PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

More preferably, compounds of Formula (IId) is wherein
Y is ¹⁸F;
PG1 is dicyclopropylmethyl or 2,4-dimethoxybenzyl, and
PG2 is tert-Butoxycarbonyl (BOC) or Triphenylmethyl (trityl).

Hereto, in a fourth embodiment, the invention of novel precursors is directed to compounds of Formula IIa

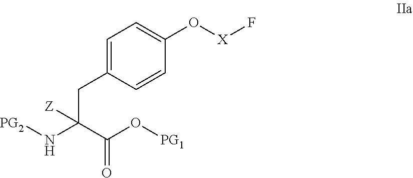

wherein:
X is CD₂;
F is ¹⁸F or ¹⁹F;
Z is methyl;
PG1 is a suitable carboxylic acid protecting group, containing up to 20 carbon atoms, optionally containing independently one or more O, N or S atoms; and
PG2 is a suitable amino protecting group, containing up to 20 carbon atoms, optionally containing one or more O, N or S atoms and are optionally substituted with one to three halogens.

The invention further refers to suitable salts of inorganic or organic acids, hydrates and solvates of the compounds of Formula IIa and is also meant to comprise single isomers, diastereomers, enantiomers and mixtures thereof of Formula IIa.

Formula (IIe) corresponds to the Markush Formula below

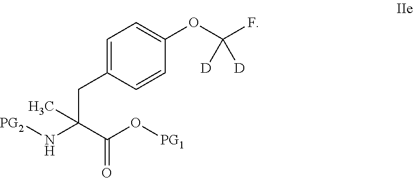

Preferred features disclosed above in respect of F, PG1 and PG2 are incorporated herein.

Preferably, the invention is directed to compounds of Formula (IIe) wherein
Y is ¹⁸F or ¹⁹F;
PG1 is
   Alkyl,
   Alkyl substituted with one phenyl,
   Alkyl substituted with one or two C₃-C₆cycloalkyl,
   Alkyl substituted with one phenyl and one C₃-C₆cycloalkyl, or
   fluorenylmethyl
   wherein
      Alkyl is a branched or linear C₁-C₆ alkyl, and optionally substituted with C₁-C₃ alkoxy, and
      Phenyl is optionally substituted with up to three C₁-C₃ alkyl, C₁-C₃ alkoxy or halogen; and
PG2 is selected from the group comprising Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) and 4-Methoxyphenyldiphenylmethyl (MMTr).

Preferably, compounds of Formula (IIe) is wherein
Y is ¹⁸F;
PG1 is dicyclopropylmethyl or 2,4-dimethoxybenzyl, and
PG2 is tert-Butoxycarbonyl (BOC) or Triphenylmethyl (trityl).

Hereto, in a fifth embodiment, the invention of novel precursors is directed to compounds of Formula (D-IIa), (D-IIb), (D-IIc), (D-IId) or (D-IIe)

(D-IIa), (D-IIb), (D-IIc), (D-IId) or (D-IIe)

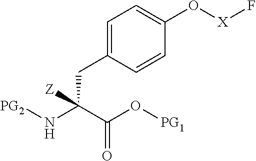

wherein

| Formula\Substituent | Z | X |
|---|---|---|
| D-IIa | H, CH₃ | CH₂, CD₂ |
| D-IIb | H | CH₂ |
| D-IIc | H | CD₂ |
| D-IId | CH₃ | CH₂ |
| D-IIe | CH₃ | CD₂ |

F, PG1 and PG2 are disclosed as above and encompass preferred features as disclosed above.

The invention further refers to suitable salts of inorganic or organic acids, hydrates and solvates of the compounds of Formula (D-IIa), (D-IIb), (D-IIc), (D-IId) or (D-IIe) and is also meant to comprise single isomers, diastereomers, enantiomers and mixtures thereof of Formula (D-IIa), (D-IIb), (D-IIc), (D-IId) or (D-IIe).

Embodiments and preferred features can be combined together and are within the scope of the invention.

¹⁹F-Invention compounds are but not limited to
tert-Butyl N-(tert-butoxycarbonyl)-O-(fluoromethyl)-D-tyrosinate

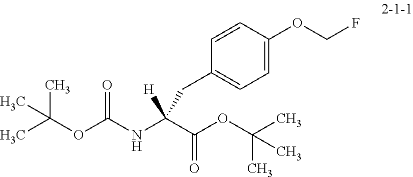 [SMILES: CC(C)(C)OC(=O)N[C@H](Cc1ccc(OCF)cc1)C(=O)OC(C)(C)C]

Dicyclopropylmethyl N-(tert-butoxycarbonyl)-O-(fluoromethyl)-D-tyrosinate

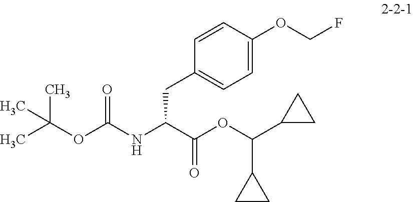 [SMILES: CC(C)(C)OC(=O)N[C@H](Cc1ccc(OCF)cc1)C(=O)OC(C1CC1)C1CC1]

Dicyclopropylmethyl N-(tert-butoxycarbonyl)-O-(fluoromethyl)-L-tyrosinate

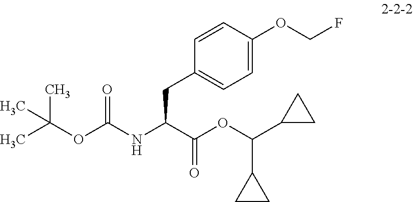 [SMILES: CC(C)(C)OC(=O)N[C@@H](Cc1ccc(OCF)cc1)C(=O)OC(C1CC1)C1CC1]

tert-Butyl O-(fluoromethyl)-N-trityl-D-tyrosinate

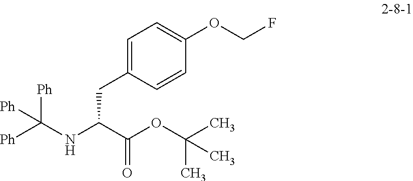 [SMILES: CC(C)(C)OC(=O)[C@@H](Cc1ccc(OCF)cc1)NC(c1ccccc1)(c1ccccc1)c1ccccc1]

2,4-Dimethoxybenzyl O-(fluoromethyl)-N-trityl-D-tyrosinate

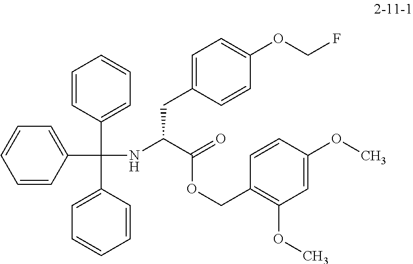 [SMILES: COc1ccc(COC(=O)[C@@H](Cc2ccc(OCF)cc2)NC(c2ccccc2)(c2ccccc2)c2ccccc2)c(OC)c1]

Methyl N-(tert-butoxycarbonyl)-O-(fluoromethyl)-alpha-methyl-D-tyrosinate

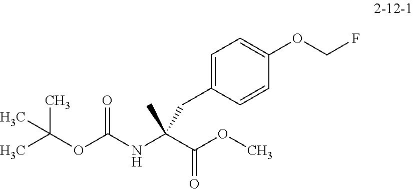 [SMILES: COC(=O)[C@@](C)(Cc1ccc(OCF)cc1)NC(=O)OC(C)(C)C]

Methyl N-(tert-butoxycarbonyl)-O-(fluoromethyl)-alpha-methyl-L-tyrosinate

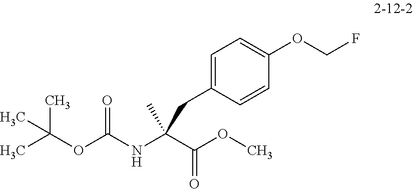 [SMILES: COC(=O)[C@](C)(Cc1ccc(OCF)cc1)NC(=O)OC(C)(C)C]

¹⁸F-Invention compounds are but not limited to
tert-Butyl N-(tert-butoxycarbonyl)-O-([¹⁸F]fluoromethyl)-D-tyrosinate.

### Labelling of 1-1-1 and 1-1-2

Dicyclopropylmethyl N-(tert-butoxycarbonyl)-O-([¹⁸F]-fluoromethyl)-D-tyrosinate.

### Labelling of 1-2-1 and 1-2-3

Dicyclopropylmethyl N-(tert-butoxycarbonyl)-O-([¹⁸F]fluoromethyl)-L-tyrosinate.

### Labelling of 1-2-2

2,4-Dimethoxybenzyl N-(tert-butoxycarbonyl)-O-([¹⁸F]fluoromethyl)-D-tyrosinate.

### Labelling of 1-3

Cyclopropylmethyl N-(tert-butoxycarbonyl)-O-([¹⁸F]fluoromethyl)-D-tyrosinate.

### Labelling of 1-4-1 and 1-4-2

4-Methoxybenzyl N-(tert-butoxycarbonyl)-O-([¹⁸F]fluoromethyl)-D-tyrosinate.

### Labelling of 1-5-1, 1-5-2 and 1-5-3

4-Methoxybenzyl N-(tert-butoxycarbonyl)-O-([¹⁸F]fluoromethyl)-L-tyrosinate.

### Labelling of 1-5-4

alpha-Methylbenzyl N-(tert-butoxycarbonyl)-O-([¹⁸F]-fluoromethyl)-D-tyrosinate.

### Labelling of 1-6

alpha,alpha-Dimethylbenzyl N-(tert-butoxycarbonyl)-O-([¹⁸F]-fluoromethyl)-D-tyrosinate.

### Labelling of 1-7

tert-Butyl O-([¹⁸F]-fluoromethyl)-N-trityl-D-tyrosinate.

### Labelling of 1-8

4-Methoxybenzyl O-([¹⁸F]fluoromethyl)-N-trityl-D-tyrosinate.

### Labelling of 1-9

Cyclopropylmethyl N-(tert-butoxycarbonyl)-O-([¹⁸F]-fluoro[²H₂]methyl)-D-tyrosinate.

### Labelling of 1-10

2,4-Dimethoxybenzyl O-([¹⁸F]fluoromethyl)-N-trityl-D-tyrosinate

### Labelling of 1-11-1, 1-11-2 and 1-11-3

Methyl N-(tert-butoxycarbonyl)-O-([¹⁸F]-fluoromethyl)-alpha-methyl-DL-tyrosinate.

### Labelling of 1-12

In a third aspect, the present invention is directed to compositions comprising compound(s) of the Formula IIa, IIb, IIc, IId, IIe, (D-IIa), (D-IIb), (D-IIc), (D-IId) or (D-IIe) independently or mixtures thereof and reagents suitable for deprotection of the amino group and the ester function of the tyrosine, as exemplified in Greene, Wuts, Protecting Groups in Organic synthesis (third edition 1999 and Fourth Edition, Wiley 2007). The person skilled in the art is familiar with auxiliaries, vehicles, excipients, diluents, carriers or adjuvants which are suitable for the desired deprotecting reaction leading to the unprotected fluoromethyl-tyrosines on account of his/her expert knowledge.

In a fourth aspect, the present invention is directed to compositions comprising compound(s) of the Formula Ia, Ib, Ic, Id, Ie, (D-Ia), (D-Ib), (D-Ic), (D-Id) or (D-Ie) independently or mixtures thereof and reagents suitable for fluoro labelling. The reagents, solvents and conditions which can be used for this fluorination are known to the person skilled in the field. See, e.g., J. Fluorine Chem., 27 (1985):177-191.

In a fifth aspect, the present invention provides a kit comprising a sealed vial containing a predetermined quantity of a compound of Formula Ia, Ib, Ic, Id, Ie, (D-Ia), (D-Ib), (D-Ic), (D-Id) or (D-Ie) independently or mixtures thereof and suitable salts of inorganic or organic acids, hydrates and solvates. Optionally the kit comprises reagents for labelling, deprotection and a pharmaceutically acceptable carrier, diluent, excipient or adjuvant.

In a sixth aspect, the present invention is directed to methods for obtaining compounds of Formula Ia.

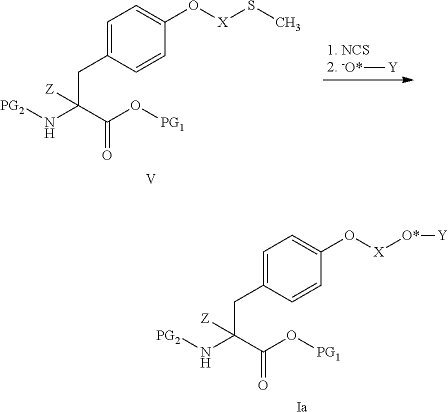

The methods for obtaining compounds of Formula Ia comprises the step of
Reacting compound of Formula V first with N-Chloro-succinimide (NCS) and then
with anion of H—O*—Y for obtaining compounds of Formula Ia,
   wherein
   compound of Formula V is compound of Formula Ia is

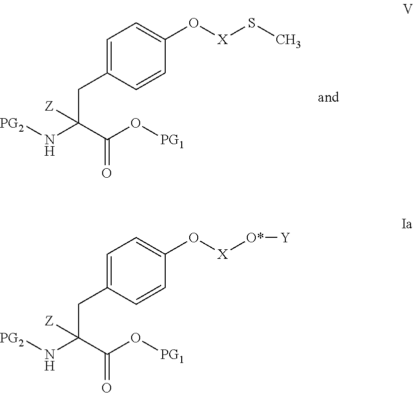

wherein Z, PG1, PG2, X, and Y are as defined above in first aspect.

Optionally, the method step is preceded by alkylation of a compound of Formula IV with Cl—X—SCH₃ for obtaining intermediate of Formula V,

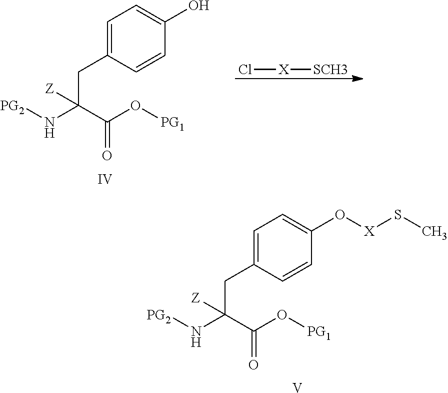

wherein Z, PG1, PG2, and X are as defined above in first aspect.

Preferred features disclosed above in respect of Z, PG1, PG2, X, and Y are incorporated herein.

Preferably, the method for obtaining compounds of Formula Ia is defined as such that
X is CH₂ or CD₂;
Y is

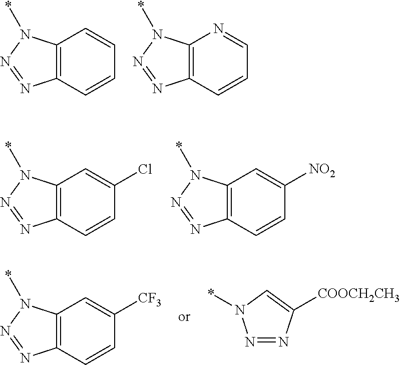

Z is Hydrogen or methyl; and
PG1 is dicyclopropylmethyl or 2,4-dimethoxybenzyl, and
PG2 is tert-Butoxycarbonyl (BOC) or Triphenylmethyl (trityl).

In a seventh aspect, the present invention is directed to a method for obtaining compounds of Formula IIa.

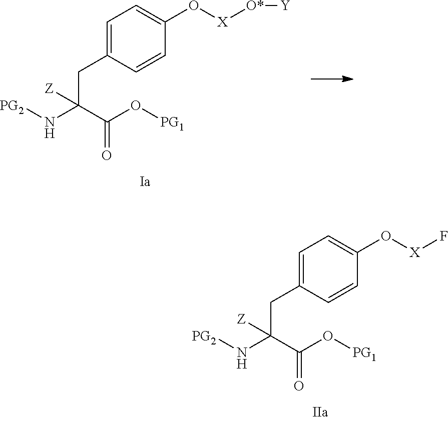

The method for obtaining compounds of Formula IIa comprises the step of
Reacting compound of Formula Ia with a ¹⁸F-Fluorination agent, and
[Optionally] converting obtained compound into a suitable salts of inorganic or organic bases thereof, hydrates, complexes, and solvates thereof
   wherein
   compound of Formula Ia is compound of Formula IIa is

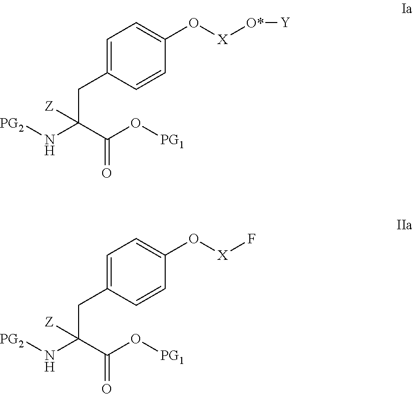

and F, Z, PG1, PG2, X, and Y are as defined above in first aspect.

Preferred features disclosed above in respect of F, Z, PG1, PG2, X, and Y are incorporated herein.

Preferably, the method for obtaining compounds of Formula IIa is defined as such that
X is CH₂ or CD₂;
Y is

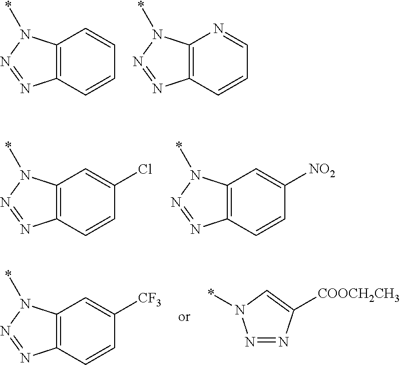

Z is Hydrogen or methyl;
F is ¹⁸F;
PG1 is dicyclopropylmethyl and 2,4-dimethoxybenzyl; and
PG2 is tert-Butoxycarbonyl (BOC) or Triphenylmethyl (trityl).

The ¹⁸F-Fluorination agent can be K¹⁸F, H¹⁸F, Rb¹⁸F, Cs¹⁸F, Na¹⁸F.

Optionally, the ¹⁸F-Fluorination agent comprises a chelating agent such as a cryptand (e.g.: 4,7,13,16,21,24-Hexaoxa-1,10-diazabicyclo[8.8.8]-hexacosane—Kryptofix®) or a crown ether (e.g.: 18-crown-6).

The ¹⁸F-Fluorination agent can also be a tetraalkylammonium salt of ¹⁸F or a tetraalkylphosphonium salt of ¹⁸F, known to those skilled in the art, e.g.: tetrabutylammonium[¹⁸F]fluoride, tetrabutylphosphonium[¹⁸F]fluoride.

Preferably, the ¹⁸F-Fluorination agent is Cs¹⁸F, K¹⁸F, tetrabutylammonium[¹⁸F]fluoride.

The reagents, solvents and conditions which can be used for this fluorination are common and well-known to the skilled person in the field. See, e.g., J. Fluorine Chem., 27 (1985):177-191; Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). Preferably, the solvents used in the present method are DMF, DMSO, acetonitrile, DMA, or mixtures thereof, preferably the solvent is acetonitrile, DMSO.

In an eighth aspect, the present invention is directed to a method for obtaining compounds of Formula VI.

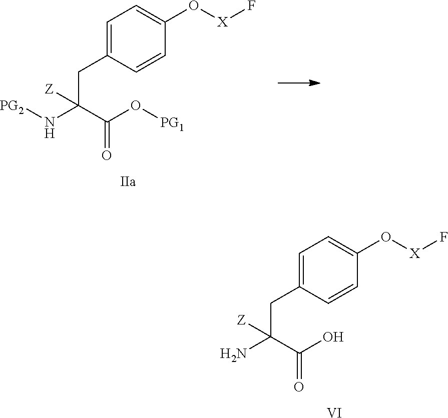

The method for obtaining compounds of Formula VI comprises the steps of
deprotecting the compound of Formula IIa for obtaining deprotected compound of Formula VI, and
[Optionally] converting obtained compound into a suitable salts of inorganic or organic bases thereof, hydrates, complexes, and solvates thereof
   wherein
   compound of Formula IIa is compound of Formula VI is

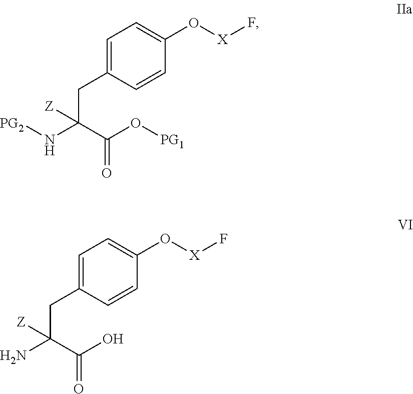

and F, Z, PG1, PG2, X, and Y are as defined above in first and second aspects.

Preferred features disclosed above in respect of F, Z, PG1, PG2, X, and Y are incorporated herein.

Deprotecting means removing the protecting groups PG1 and PG2. Preferably, deprotecting occurs under acid conditions, wherein more preferably the acid is HCl in organic- or aqueous-solvents or TFA with or without additives.

Preferably, the method for obtaining compounds of Formula VI is defined as such that
X is CH₂ or CD₂;
Z is Hydrogen or methyl;
F is ¹⁸F;
PG1 is dicyclopropylmethyl or 2,4-dimethoxybenzyl; and
PG2 is tert-Butoxycarbonyl (BOC) or Triphenylmethyl (trityl).

### DEFINITIONS

“D” means Deuterium.

The acronym “PET” abbreviates positron emission tomography, which reflects a nuclear medicine imaging technique producing a three-dimensional image or picture of functional processes within the body. The system detects pairs of gamma rays emitted indirectly by a positron emitting radionuclide of the tracer or PET-tracer, which is introduced into the body on a biologically active molecule. Three-dimensional images of tracer concentration within the body are then constructed by computer analysis. PET-imaging can be combined by magnetic resonance imaging (MRI) or CT.

The term “stable” in accordance with the present invention specifies the provided precursor compounds in which the chemical structure is not altered when the compound is stored at a temperature from about −80° C. to about +40° C., preferably from about −80° C. to +25° C., more preferably from about −20° C. to +20° C., even more preferably from about −20° C. to 0° C. for at least one week, preferably at least one month, more preferably at least six month, even more preferably at least one year and/or the provided presursor compounds which under IUPAC standard conditions maintains its structural integrity long enough to be useful for the synthesis of PET-tracers pursuant to the invention.

Fluorenylmethyl is

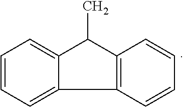

As used herein, the term “alkyl” refers to a C₁-C₆ straight chain or branched chain alkyl group such as, for example methyl, ethyl, propyl, isopropyl, butyl, isobutyl, tert-butyl, pentyl, isopentyl, and neopentyl. Preferably, alkyl is C₁-C₃ straight chain or branched chain alkyl.

As used herein, the term “cycloalkyl”, refers to a C₃-C₆ cyclic alkyl group such as, for example, cyclopropyl, cyclobutyl, cyclopentyl, and cyclohexyl.

As used herein, the term “alkoxy” refers to alkyl groups respectively linked to the respective scaffold by an oxygen atom, i.e. —O—, with the alkyl portion being as defined above, such as for example methoxy, ethoxy, isopropoxy, tert-butoxy, hexyloxy.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl.

The term “heteroaryl” as employed herein by itself or as part of another group refers to monocyclic or bicyclic aromatic groups containing from 5 to 12 carbons in the ring and where up to 4 carbons are replaced by nitrogens in a way, that the resulting heteroaromatic system contains one N—H group. Typical examples are pyrrole, imidazole, triazole; their benzannelated analogs, indol, benzimidazoles, benzotriazoles, pyridyl fused analogs like azabenzotriazole and other fused systems like imidazopyrroles or imidazotriazoles.

The term “halo” refers to fluoro, chloro, bromo, and iodo.

The term “amine-protecting group” as employed herein by itself or as part of another group is known or obvious to someone skilled in the art, which is chosen from but not limited to a class of protecting groups namely carbamates, amides, imides, N-alkyl amines, N-aryl amines, enamines, N-sulfonyl and which is chosen from but not limited to those described in the textbook Greene and Wuts, Protecting groups in Organic Synthesis, third edition, (third edition 1999, page 494-653, which is hereby incorporated herein by reference. Preferred amine protecting groups are carbamates (e.g. Boc) and Aralkyl (e.g. Trityl).

The term “carboxylic acid-protecting group” as employed herein refers to a protecting group employed to block or protect the carboxylic acid functionality while the reactions involving other functional sites of the compound are carried out. Carboxy protecting groups are disclosed in Greene, Wuts, Protective Groups in Organic Synthesis, third edition, 1999, page 372-453, which is hereby incorporated herein by reference. Such protecting groups are well known to those skilled in the art, having been extensively used in the protection of carboxylic acids. Representative carboxy protecting groups are alkyl (e.g., methyl, ethyl or tertiary butyl and the like); arylalkyl, for example, phenethyl or benzyl and substituted derivatives thereof such as alkoxybenzyl or nitrobenzyl groups and the like; alkylcycloalkyl (e.g. cyclopropylmethyl or dicyclopropylmethyl); alkoxyalkyl (e.g. methoxymethyl (MOM) or benzyloxymethyl (BOM).

Preferred O-protected compounds of the invention are compounds wherein the protected carboxy group is a lower alkyl (for example, methyl ester, ethyl ester, propyl ester, isopropyl ester, butyl ester, sec-butyl ester, isobutyl ester, tert. butylester, amyl ester, isoamyl ester), alkyl-cycloalkyl (for example cycloalkylmethyl, dicycloalkylmethyl, 1-cycloalkylethyl) or arylalkyl (for example, benzyl, 4-methoxybenzyl, 2,4-dimethoxybenzyl) ester.

As used hereinafter in the description of the invention and in the claims, the terms “salts of inorganic or organic acids”, “inorganic acid” and “organic acid” refer to mineral acids, including, but not being limited to: acids such as carbonic, nitric, phosphoric, hydrochloric, perchloric or sulfuric acid or the acidic salts thereof such as potassium hydrogen sulphate, or to appropriate organic acids which include, but are not limited to: acids such as carboxylic and sulfonic acids, examples of which are trifluoracetic, methansulfonic, ethanesulfonic, benzenesulfonic, toluenesulfonic and trifluormethanesulfonic acid, respectively.

Suitable salts of the compounds according to the invention include salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalene disulfonic acid, acetic acid, trifluoroacetic acid, propionic acid, lactic acid, tartaric acid, malic acid, citric acid, fumaric acid, maleic acid and benzoic acid.

Suitable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

If chiral centres or other forms of isomeric centres are present in a compound according to the present invention, all forms of such stereoisomers, including enantiomers and diastereoisomers, are intended to be covered herein. Compounds containing chiral centres may be used as racemic mixture or as an enantiomerically enriched mixture or as a diastereomeric mixture or as a diastereomerically enriched mixture, or these isomeric mixtures may be separated using well-known techniques, and an individual stereoisomer maybe used alone. In cases wherein compounds may exist in tautomeric forms, such as keto-enol tautomers, each tautomeric form is contemplated as being included within this invention whether existing in equilibrium or predominantly in one form.

The terms “halide, halo” (halogen) as employed herein by itself or as part of another group is known or obvious to someone skilled in the art, and means fluoro, chloro, bromo, and iodo.

As used hereinafter in the description of the invention and in the claims, the term “fluorine isotope” (F) refers to all isotopes of the fluorine atomic element unless explicitly otherwise indicated. Fluorine isotope (F) is selected from radioactive or non-radioactive isotope. The radioactive fluorine isotope is [¹⁸F]. The non-radioactive “cold” fluorine isotope is [¹⁹F].

The stereochemistry can be denoted in several ways. For the amino acids often D/L is used for the alpha-position referring to position of the residues when drawn in the Fischer-projection. Stereochemically D corresponds to the stereodescriptor “R” and L corresponds to the stereodescriptor “S” for all of the compounds of the invention.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The entire disclosure[s] of all applications, patents and publications, cited herein are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

### Abbreviations

| | |
|---|---|
| 18-c-6 | 1,4,7,10,13,16-hexaoxacyclooctadecane |
| br | broad signal (in NMR data) |
| CI | chemical ionisation |
| d | doublet |
| CH₂Cl₂ | dichloromethane |
| Cs₂CO₃ | cesium carbonate |
| DAD | diode array detector |
| dd | doublet of doublet |
| ddd | doublet of doublet of doublet |
| dt | doublet of triplet |
| DMF | N,N-dimethylformamide |
| DMSO | dimethylsulfoxide |
| EI | electron ionisation |
| ELSD | evaporative light scattering detector |
| ESI | electro spray ionisation |
| EtOAc | ethyl acetate |
| EtOH | ethanol |
| Fmoc | fluorenylmethyloxycarbonyl |
| HCOOH | Formic acid |
| HPLC | high performance liquid chromatography |
| GBq | Giga Becquerel |
| h | hour |
| K_{2.2.2} | 4,7,13,16,21,24-hexaoxa-1,10- |
| | diazabicyclo[8.8.8]-hexacosane |
| K₂CO₃ | potassium carbonate |
| K₂HPO₄ | dipotassium phosphate |
| KOH | potassium hydroxide |
| MBq | Mega Becquerel |
| MeCN | acetonitrile |
| MeOH | methanol |
| MS | mass spectrometry |
| MTB | methyl tert-butyl ether |
| m | multiplet |
| mc | centred multiplet |
| min | minute |
| NaH | sodium hydride |
| NMR | nuclear magnetic resonance |
| | spectroscopy: chemical shifts (δ) are |
| | given in ppm. |
| q | quadruplet (quartet) |
| PMB | para-methoxybenzyl |
| PET | positron Emission Tomography |
| RT | room temperature |
| s | singlet |
| t | triplet |
| TBAOH | tetrabutylammonium hydroxide |
| TBS | tert-butyldimethyl silyl |
| THF | tetrahydrofuran |
| THP | tetrahydropyran |
| UPLC | ultra performance liquid chromatography |

### Experimental Section

The following examples can be repeated with similar success by substituting the generically or specifically described reactants and/or operating conditions of this invention for those used in the preceding examples.

### Detailed Description of the Synthesis of Compound of Formula I and Ia

### 1. N-Protected Tyrosine Esters

N-protected tyrosines can be directly esterified by alkylation of the carboxylic acid without protection of the phenol function (e.g. Jung M. E. Tetrahedron 1997, 8815). The reaction of the salts of N-protected tyrosines with suitable alkylation agents also gives the protected tyrosine esters.

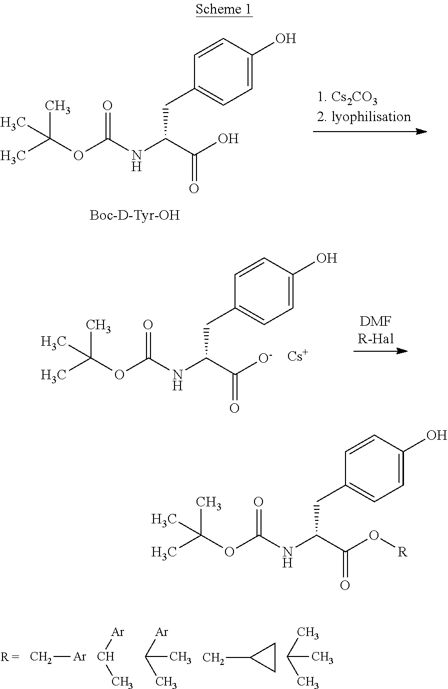

Alternatively, a tyrosine ester can be reacted with a dialkyldicarbonate to introduce a carbamate or a trityl-group as N-protection.

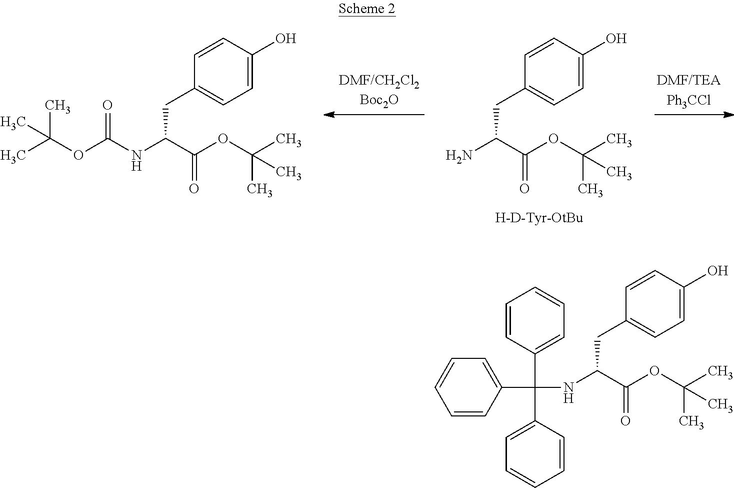 [SMILES: CC(C)(C)OC(=O)N[C@H](Cc1ccc(O)cc1)C(=O)OC(C)(C)C ; CC(C)(C)OC(=O)[C@H](N)Cc1ccc(O)cc1 ; CC(C)(C)OC(=O)[C@@H](Cc1ccc(O)cc1)NC(c1ccccc1)(c1ccccc1)c1ccccc1]

Finally, if no suitable alkylating agent is available or the tyrosine ester is not easily available, direct esterification methods can be employed using the corresponding alcohol. This is exemplary demonstrated in the synthesis of the dicyclopropylmethylester. In such case it is advisable to protect the phenol prior to esterification as shown in scheme 3.

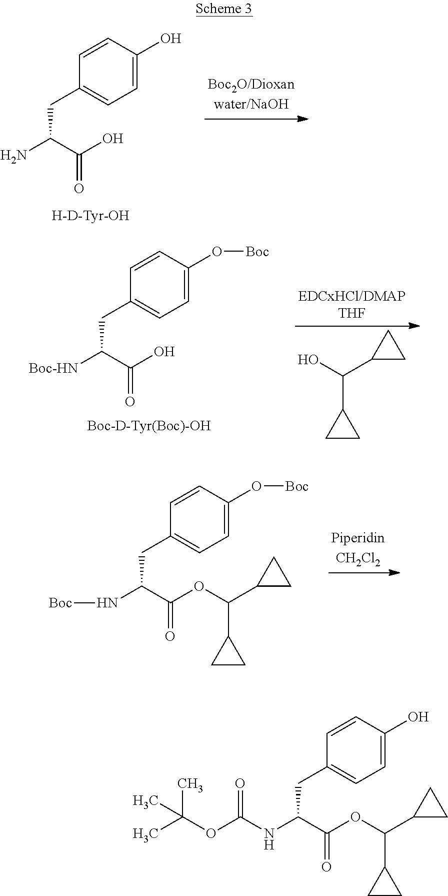

D-Tyrosine is bisbocylated according to Pozdnev, V. F.; Chemistry of Natural Compounds; English; 18; 1; 1982; 125-126 and then be esterified with a standard DMAP/Carbodiimide coupling method. Selective deprotection is done according to Nakamura K., Tetrahedron Lett. 2004, 495.

These examples show, that a wide choice of protected tyrosines can be easily synthesized.

### 2. Methylthiomethylethers of N-protected tyrosine esters

The phenol group of the protected tyrosine esters is converted into a methylthiomethyl-ether by alkylation with methylthiomethylchloride in a DMF/THF mixture using potassium-tert.-butylate as base and sodium iodide to enhance the reactivity of the alkylation agent.

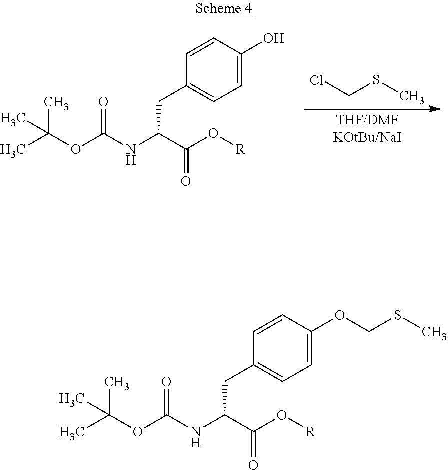

To a person skilled in the art it is obvious, that other chlorothiomethylethers can be employed, like for example 1-[(chloromethyl)sulfanyl]-4-methylbenzene or 1-[(chloromethyl)sulfanyl]-4-chlorobenzene.

### 3. Conversion of the methylthiomethylethers into compounds of formula I

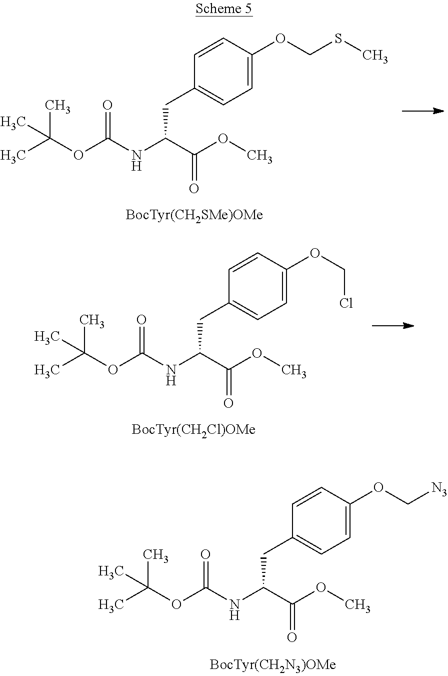

The basic strategy has been described in Angew. Chem. Int. Ed. 2002, 3449 for the synthesis of BocTyr(azidomethyl)OMe. The chloromethylether can be made in 73% using N-chlorosuccinimide and trimethylsilylchloride as activation agent in dichloromethane. The compound could be isolated although some hydrolysis was reported. When this reaction was conducted with the more acid labile tert-butylester, the yield dropped to 24% and with the even more labile dicyclopropylmethylester, the chloromethylether could not be isolated. It proved advantageous not to isolate this labile intermediate. In an improved protocol, no activation is employed and the reaction mixture is immediately after workup reacted with the ON-nucleophile in an anhydrous environment.

The chloromethylether can also be obtained by reaction with sulfuryl chloride in dichloromethane at 0° C. as described in Journal of Medicinal Chemistry, 2005, Vol. 48, No. 10, 3586-3604.

For this it is advantageous to employ the ON-nucleophiles like HOBt in anhydrous form. However, HOBt is commercial supplied only as the hydrate. Anhydrous salts of HOBt are not commercial as well. It was found however, that tetrabutylammonium OBt can easily be prepared in anhydrous form. Commercial HOBt hydrate was dissolved in anhydrous tetrabutylammoniumhydroxide (Commercial 1M in MeOH) and the solvent evaporated to give a yellow solid. This was stripped twice with toluene to give anhydrous Bu₄NOBt. This compound can safely be dried at 40° C. This method can be used for all O—N-nucleophiles described in this patent. Alternatively, KOBt can be made by reaction of HOBt*H₂O with KOH in Methanol and dried by stripping with toluene and evaporation in the vacuum at 40° C.

Reaction of the raw mixture from the reaction of the methylthiomethylether with N-chlorosuccinimide with the tetrabutylammonium salt of the O—N-nucleophile, gives the inventive compounds of formula I.

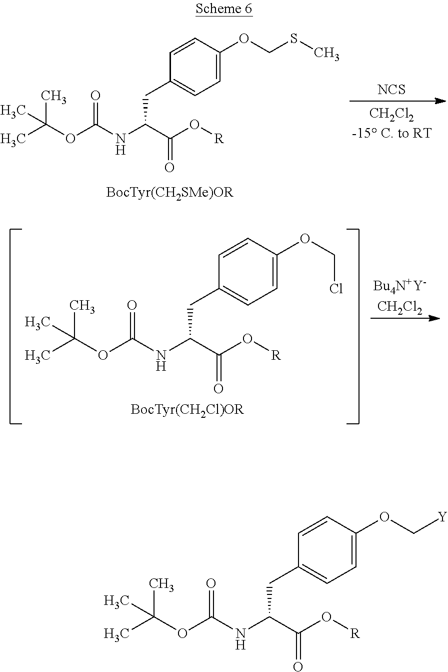

It is understood, that this method is also applicable to tyrosine esters with other N-protection and other aromatic systems as shown in Scheme 7 for the synthesis of a precursor for an analog to DPA-714 which is a well known PBR-Ligand (Journal of Labelled Compounds and Radiopharmaceuticals 2008, Vol 51(7), 286-292.; Journal of Nuclear Medicine 2008, Vol 49(5), 814-822).

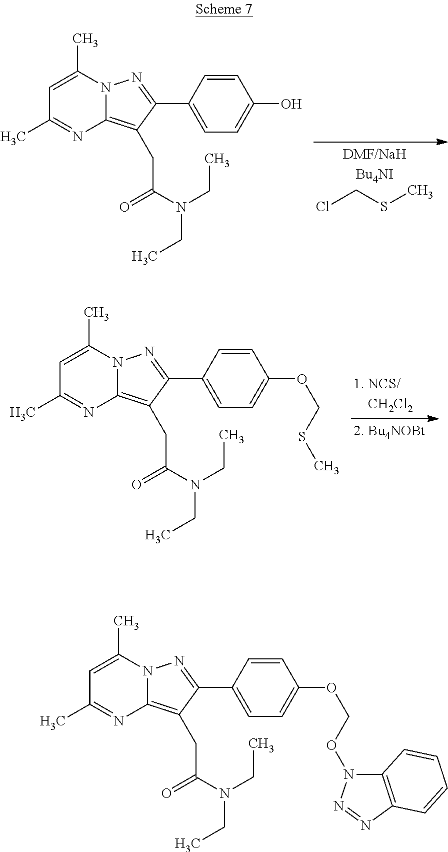

### Exemplary Description of the Synthesis of Compounds of Formula I and Ia

Halo- or tosyloxy-compounds can be directly reacted N-Hydroxybenzotriazoles under basic conditions. In anhydrous conditions, the tetrabutylammonium salt of a N-Hydroxybenzotriazole or the potassium salt of a hydroxybenzotriazole is advantageously employed as shown in Scheme 8 and 9.

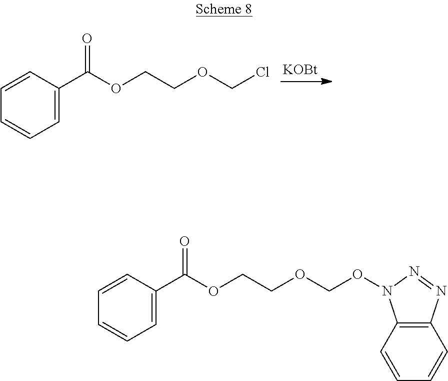 [SMILES: O=C(OCCOCCl)c1ccccc1 ; O=C(OCCOCOn1nnc2ccccc21)c1ccccc1 ; "KOBt"]

### 1. Synthesis of Non Radioactive Compounds

First number denotes Precursor (1), that is compounds of formula (I), cold standard (2), that is compounds of formula (III) or intermediates (3), second number denotes example, third number to distinguish compounds within the example. (1-3-2=second compound of formula (I) in example 3).

### Detailed Description of the Synthesis of Compound of Formula Ic

For the synthesis of deuterated compounds Ic a slightly different synthesis route is employed (Scheme 7) which has the advantage, that perdeuterated dimethylsulf oxide is a readily available reagent. It is reacted according to a published procedure (J. Chem. Soc. Perkin I, 1983, 1141-44) with the protected tyrosine to give the deuterated methylthiomethylether. The last reaction step is the same as described above.

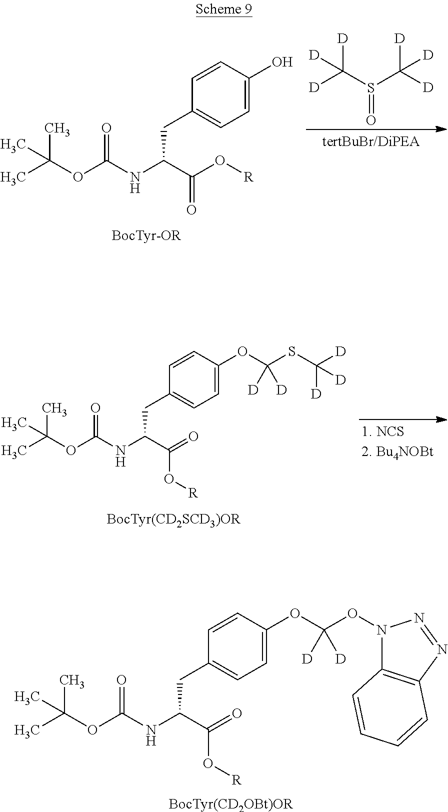

### Detailed Description of the Synthesis of Compound of Formula Id

Synthesis of the αMethyl tyrosine derivatives Id and Ie is done by combination of the methods described for the synthesis of Ib and Ic respectively. For synthesis of the compounds Id commercially available racemic αMethyl-tyrosine-methylester is used as starting material (Scheme 8). Bocylation proceeds as described (J. Med. Chem. 2004, 47, 1223-33). Alkylation with ClCH₂SCH₃ is done as described above. The benzotriazolyl-methylether can be obtained by the same reaction as described for compounds Ib above.

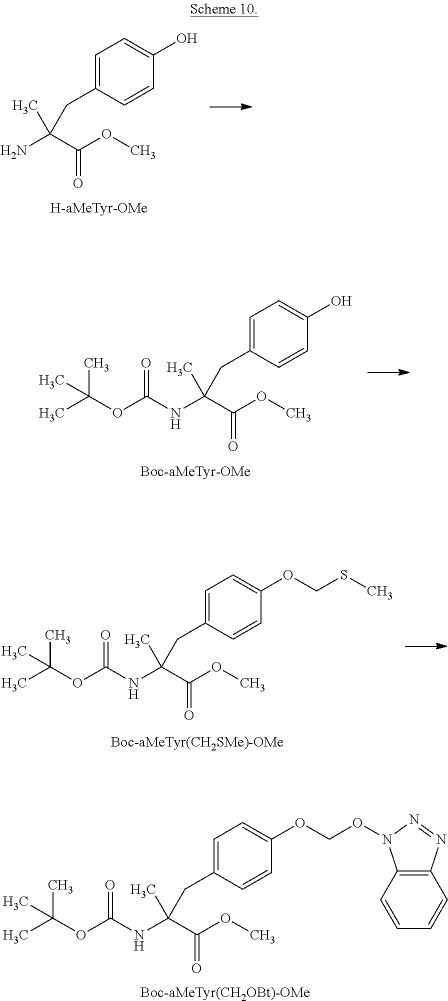

### Detailed Description of the Synthesis of Compound of Formula Ie

The compounds of Formula Ie can be obtained by combination of the methods depicted in Schemes 9 and 10.

### Detailed Description of the Synthesis of Compound of Formula IIa

Synthesis of the [¹⁹F]-fluoromethylethers IIb or IId is usually done by reaction of the N-protected tyrosine ester with Bromofluoromethane as shown in Scheme 11 for IIb.

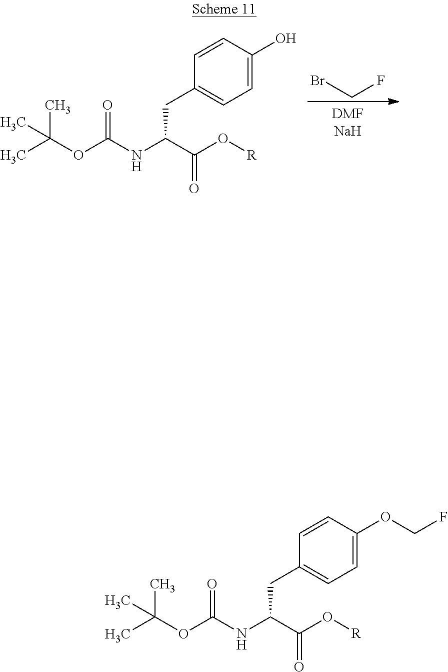

Compounds of Formula IIc and IIe could be synthesized in analogy using Bromo-fluoro[²H₂]methane. In our experimental work, the compounds IIIa and IIIc were used as cold reference in the radiosynthesis of [¹⁸F]-IIc and [¹⁸F]-IIe.

The radiosynthesis of the [¹⁸F]-fluoromethylethers IIa can be carried out in a two-step process using a reactive intermediate synthon, e.g. [¹⁸F]Fluoromethyl bromide (Iwata et al., Appl. Radiat. Isot., 2002, 57, 347-352), [¹⁸F]Fluoromethyl iodide (Zhang et al., J. Med. Chem., 2004, 47, 2228-2235, Zhang et al., J. Fluorine Chem., 2004, 125, 1879-1886), [¹⁸F]Fluoromethyl tosylate (Neal et al., J. Label. Compd. Radiopharm., 2005, 48, 557-568), [¹⁸F]Fluoromethyl triflate (Iwata et al., Appl. Radiat. Isot., 2002, 57, 347-352) or [¹⁸F]Fluoromethyl mesylate (Neal et al., J. Label. Compd. Radiopharm., 2005, 48, 557-568), and reacting it with the a hydroxyl functional under basic conditions. These methods are known to those skilled in the art. The reactions can be carried out, for example in a typical reaction vessel (e.g. Wheaton vial) which is known to someone skilled in the art or in a microreactor. The reaction can be heated by typical methods, e.g. oil bath, heating block or microwave. The radiofluorination reactions are carried out in acetonitrile with potassium carbonate as base and “kryptofix” as crown-ether. But also other solvents can be used which are well known to experts. These possible conditions include, but are not limited to: dimethyl sulfoxide and dimethylformamide as solvent and tetraalkyl ammonium and tetraalkyl phosphonium carbonate as base. Water and/or alcohol can be involved in such a reaction as co-solvent. The radiofluorination reactions are conducted for one to 60 minutes. Preferred reaction times are five to 50 minutes. Further preferred reaction times are 10 to 40 min. This and other conditions for such radiofluorination are known to experts (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50; Ametamey et al., Chem. Rev., 2008, 108, 1501-1516). The radiofluorination can be carried out in a “hot-cell” and/or by use of a module (review: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated or semi-automated synthesis.

### EXAMPLES

### 1.1 Example 1

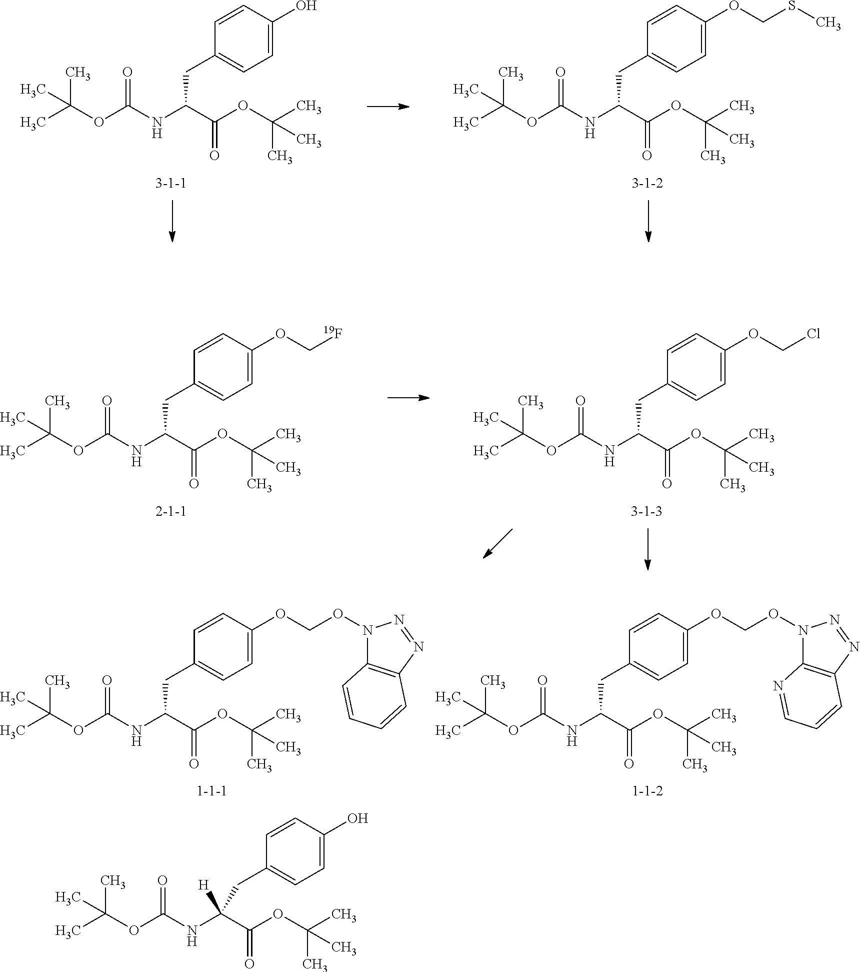

### tert-Butyl N-(tert-butoxycarbonyl)-D-tyrosinate. 3-1-1

To a stirred solution of tert-butyl D-tyrosinate (47.46 g, 200 mmol) in dichloromethane (600 ml) and N,N-dimethylformamide (60 ml) was added triethylamine (22 g, 220 mmol) and di-tert-butyl dicarbonate (43.65 g, 200 mmol). The mixture was stirred at r.t. for 2 h and then subsequently washed with aqueous 1N hydrochloric acid (3×100 ml), saturated sodium hydrogen carbonate (100 ml), brine (100 ml), dried (magnesium sulfate) and concentrated to give 3-1-1 as a light yellow oil, which solidified on standing. Yield 64 g (95%).

MS (C₁, NH₃): m/z=355 (M+NH₄), 388 (M+H), 399 (M+NH₄—C₄H₈), 382 (M+H—C₄H₈), 238 (M+H−C₄H₈−CO₂).

¹H-NMR (400 MHz, CD₂Cl₂): δ=7.00 (d, J=8.3 Hz, 2H, Ar), 6.74 (d, J=8.1 Hz, 2H, Ar), 5.31 (d, J=8.1 Hz, 1H, NH), 4.33 (mc, 1H, 2-H), 2.94 (mc, 2H, 3-H), 1.41 ppm (s, 18H, 1-tBu, 2-tBu).

¹³C-NMR (100 MHz, CD₂Cl₂): δ (ppm) 171.13 (C1), 155.59 (br., 2-C1), 155.22 (Ar—C4), 130.52 (Ar—C2), 127.59 (Ar—C1), 115.18 (Ar—C3), 81.93 (1-C1), 79.64 (br., 2-C2), 55.20 (C-2), 37.38 (br. C-3), 27.70 (2-C3), 27.40 (1-C2).

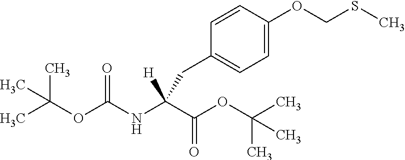 [SMILES: CSCOc1ccc(C[C@@H](NC(=O)OC(C)(C)C)C(=O)OC(C)(C)C)cc1]

### tert-Butyl N-(tert-butoxycarbonyl)-O-[(methylsulfanyl)methyl]-D-tyrosinate. 3-1-2

A solution of 3-1-1 (4.62 g, 13.7 mmol) and sodium iodide (0.21 g, 1.4 mmol) in N,N-dimethylformamide (30 ml) was cooled in an ice water bath. A solution of potassium tert-butoxide (1.73 g, 15.4 mmol) in tetrahydrofuran (15 ml) was added. Then chloromethyl methyl sulfide (1.3 ml, 1.50 g, 15.8 mmol) was added. The mixture was stirred at r.t. for 3 h after which TLC showed complete conversion. Ethyl acetate (60 ml) was added and the mixture was washed with water (50 ml). The water layer was extracted with ethyl acetate (50 ml). The combined organic layers were consecutively washed with 10% citric acid, brine, dried (magnesium sulfate) and concentrated to give a thick yellow oil (smelly), 6.0 g. Purification on column chromatography (SiO₂, heptane/ethyl acetate 3/1) gave pure 3-1-2 as an oil, 3.3 g (81%).

The reaction was repeated with 23.1 g of 3-1-1 to give 19.6 g of 3-1-2 (72%)

MS (ES+): m/z=420 (M+Na), 398 (M+H), 242 (M−2 C₄H₈−CO₂).

MS (C₁, NH₃): m/z=415 (M+NH₄), 398 (M+H), 359 (M+NH₄−C₄H₈), 342 (M−C₄H₈), 303 (M+NH₄−2 C₄H₈).

¹H-NMR (400 MHz, CD₂Cl₂): δ=7.08 (d, J=8.6 Hz, 2H, Ar), 6.87 (d, J=8.3 Hz, 2H, Ar), 5.13 (s, 2H, O—CH₂—O), 4.98 (db, J=6.8 Hz, 1H, NH), 4.34 (mc, 1H, 2-H), 2.98 (mc, 2H, 3-H), 2.15 (s, 3H, SCH₃), 1.41, 1.40 ppm (s, 18H, 1-tBu, 2-tBu).

¹³C-NMR (100 MHz, CD₂Cl₂): δ (ppm) 170.93 (C1), 154.97 (Ar—C4), 154.97 (br., 2-C1), 130.61 (Ar—C1), 130.50 (Ar—C2), 115.81 (Ar—C3), 81.85 (1-C1), 79.38 (br., 2-C2), 77.48 (O—CH₂—S), 55.08 (C-2), 37.43 (br., C-3), 28.05 (2-C3), 27.72 (1-C2), 14.29 (SCH₃).

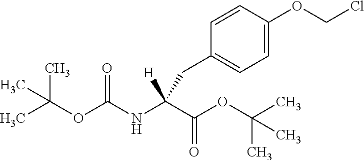 [SMILES: CC(C)(C)OC(=O)N[C@H](Cc1ccc(OCCl)cc1)C(=O)OC(C)(C)C]

### tert-Butyl N-(tert-butoxycarbonyl)-O-(chloromethyl)-D-tyrosinate. 3-1-3

To a solution of 3-1-2 (18.2 g, 46 mmol) in dichloromethane (200 ml) at r.t. was added N-chlorosuccinimide (7.34 g, 55 mmol). After stirring for 10 min was added trimethylsilyl chloride (7.60 g, 70 mmol). The mixture was stirred at r.t. overnight. The mixture was consecutively washed with saturated sodium hydrogen carbonate, water, dried (magnesium sulfate) and concentrated to a yellow oil, 18 g (quantitative). Purification on column chromatography (450 g SiO₂, heptane/ethyl acetate 3/1) gave 3-1-3 as light yellow oil (4.3 g, 24%).

¹H-NMR (400 MHz, CD₂Cl₂): δ=7.17 (d, J=8.6 Hz, 1H, Ar), 7.02 (d, J=8.6 Hz, 1H, Ar), 5.90 (s, 2H, O—CH₂—O), 4.98 (db, J=6.6 Hz, 1H, NH), 4.36 (mc, 1H, 2-H), 3.00 (mc, 2H, 3-H), 1.41, 1.40 ppm (s, 18H, 1-tBu, 2-tBu).

¹³C-NMR (100 MHz, CD₂Cl₂): δ (ppm) 170.80 (C1), 154.91 (br. 2-C1), 154.48 (Ar—C4), 131.75 (Ar—C1), 130.78 (Ar—C2), 115.93 (Ar—C3), 81.96 (1-C1), 79.41 (br. 2-C2), 77.61 (O—CH₂—O), 54.95 (C-2), 37.53 (br. C-3), 28.03 (2-C3), 27.71 (1-C2).

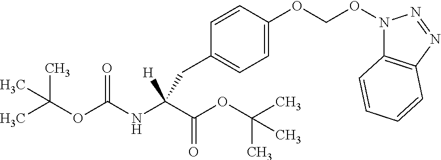 [SMILES: CC(C)(C)OC(=O)N[C@H](Cc1ccc(OCOn2nnc3ccccc32)cc1)C(=O)OC(C)(C)C]

### tert-Butyl O-[(1H-benzotriazol-1-yloxy)methyl]-N-(tert-butoxycarbonyl)-D-tyrosinate. 1-1-1

To a stirred solution of 1H-benzotriazol-1-ol hydrate (1.0 g, 8.5 mmol, stripped free of water with toluene) in N,N-dimethylformamide (2 ml) and dichloromethane (20 ml) was added at r.t. a solution of 3-1-3 (1.0 g, 2.6 mmol) in dichloromethane (5 ml), followed by 4-(dimethylamino)pyridine (0.4 g, 3.2 mmol). The mixture was stirred at r.t. for 30 min after which time TLC indicated complete consumption of starting material. Water (50 ml) was added and the mixture was extracted with tert-butyl methyl ether (3×50 ml). The organic layers were combined and washed with water (2×30 ml), dried (magnesium sulfate) and concentrated to give 1.3 g of a solid/oil mixture. Purification on column chromatography (30 g SiO₂, heptane/ethyl acetate 3/1) gave pure 1-1-1 as white solid (0.75 g, 60%).

MS (ES+): m/z=507 (M+Na), 485 (M+H), 429 (M+H−C₄H₈), 385 (M+H−C₄H₈−CO₂.

¹H-NMR (300 MHz, CD₂Cl₂): δ=7.97 (db, J=8.6 Hz, 1H, Bt), 7.38 (mc, 2H, Bt), 7.25-7.06 (m, 5H, 1 Bt, Ar—H), 6.03 (s, 2H, O—CH₂—O), 5.05 (db, J=7.7 Hz, 1H, NH), 4.41 (m, 1H, 2-H), 3.09 (dd, J=13.8 Hz, J=6.0 Hz, 1H, 3-H), 3.00 (dd, J=13.8 Hz, J=6.0 Hz, 1H, 3-H), 1.43, 1.41 ppm (s, 18H, 1-tBu, 2-tBu).

¹³C-NMR (75 MHz, CD₂Cl₂): δ (ppm) 170.72 (C1), 155.12 (Ar—C4), 154.88 (br., 2-C1), 143.44 (Bt C3a), 131.94 (Ar—C1), 130.95 (Ar—C2), 128.68 (Bt C7a), 128.18 (Bt C6), 124.55 (Bt C5), 119.83 (Bt C4), 115.94 (Ar—C3), 108.99 (Bt C7), 99.06 (O—CH₂—O), 81.93 (1-C1), 79.40 (br., 2-C2), 55.00 (C-2), 37.52 (br., C-3), 28.01 (2-C3), 27.71 (1-C2).

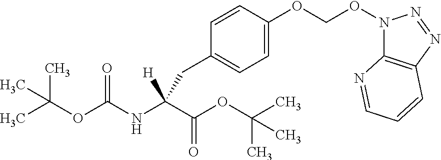 [SMILES: CC(C)(C)OC(=O)N[C@H](Cc1ccc(OCOn2nnc3cccnc32)cc1)C(=O)OC(C)(C)C]

### tert-Butyl N-(tert-butoxycarbonyl)-O-[(1H-1,2,3-triazolo[5,4-b]pyridin-1-yloxy)methyl]-D-tyrosinate. 1-1-2

To a stirred solution of 1H-1,2,3-triazolo[5,4-b]pyridine-1-ol (0.5 g, 3.7 mmol) in N,N-dimethylformamide (1 ml) and dichloromethane (10 ml) was added at r.t. a solution of 3-1-3 (1.0 g, 2.6 mmol) in dichloromethane (5 ml) followed by 4-(dimethylamino)pyridine (0.4 g, 3.2 mmol). The mixture was stirred at r.t. for 3 days after which time TLC indicated only trace of starting material. Water (20 ml) was added and the mixture was extracted with tert-butyl methyl ether (3×25 ml). The organic layers were combined and consecutively washed with water (20 ml), 0.5 N hydrochloric acid (10 ml), brine (10 ml), dried (magnesium sulfate) and concentrated to a sticky solid, 0.85 g. Purification on column chromatography (25 g SiO₂, heptane/ethyl acetate 1/1) gave pure 1-1-2 as white solid (0.40 g, 32%).

MS (ES+): m/z=508 (M+Na), 486 (M+H), 430 (M+H−C₄H₈), 374 (M+H−2×C₄H₈), 366 (M+H−C₄H₈−CO₂), 330 (M+H−2×C₄H₈—CO₂).

¹H-NMR (400 MHz, CD₂Cl₂): δ=8.67 (dd, J=4.5 Hz, J=1.0 Hz, 1H, At 6-H), 8.37 (dd, J=8.5 Hz, J=1.0 Hz, 1H, At 5-H), 7.41 (dd, J=8.5 Hz, J=4.5 Hz, 1H, At 4-H), 7.19 (s, 4H, Ph-H), 6.04 (s, 2H, O—CH₂—O), 5.03 (db, J=7.6 Hz, 1H, NH), 4.38 (m, 1H, 2-H), 3.08 (dd, j 0 13:7 Hz, J=5.9 Hz, 1H, 3-H), 3.00 (dd, J=13.7 Hz, J=6.1 Hz, 1H, 3-H). 1.43, 1.41 ppm (s, 18H, 1-tBu, 2-tBu).

¹³C-NMR (100 MHz, CD₂Cl₂): δ (ppm) 170.82 (C1), 155.69 (Ar—C4), 154.97 (br., 2-C1), 151.50 (At C6), 140.25 (At C7a), 135.14 (At C3a), 132.21 (Ar—C1), 130.82 (Ar—C2), 129.12 (At C4), 120.76 (At C5), 117.13 (Ar—C3), 99.65 (O—CH₂—O), 81.96 (1-C1), 79.42 (br., 2-C2), 55.02 (C-2), 37.51 (C-3), 28.06 (2-C3), 27.75 (1-C2).

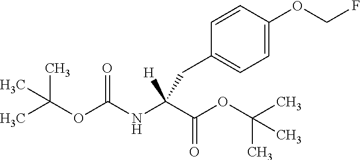 [SMILES: CC(C)(C)OC(=O)N[C@H](Cc1ccc(OCF)cc1)C(=O)OC(C)(C)C]

### tert-Butyl N-(tert-butoxycarbonyl)-O-(fluoromethyl)-D-tyrosinate. 2-1-1

A: 1.50 g (4.45 mmol) 3-1-1 were dissolved in 30 ml N,N-dimethylformamide, cooled to 10° C. and 194 mg (4.45 mmol) sodium hydride (60% in mineral oil) were added in one portion. The mixture was stirred for 30 min.

B: 30 ml N,N-dimethylformamide were cooled to 0° C. and bromofluoromethane was bubbled into the solution. By weighing of the flask and of the steel container the amount of gas dissolved was determined.

30 ml of N,N-dimethylformamide containing 1 g (8.89 mmol) bromofluoromethane were added slowly at 0° C. to the solution prepared in A and the reaction was stirred at 0° C. for 2 h. The mixture was allowed to warm to r.t. and was stirred another 2 h, after which the reaction mixture was poured in to water and extracted with dichloromethane twice. The combined organic phases were dried over magnesium sulfate, and evaporated to give the raw product as an oil. Chromatography (Silica gel, Gradient hexane to hexane/ethyl acetate 3:1) gave 1.3 g (79%) of the title compound as clear oil. An analytical sample was purified by preparative HPLC.

HPLC (Chiralpak AD-H 5μ150×4.6 mm, Hexane/Ethanol 9:1, 1.0 ml/min, (1 mg/ml EtOH, 5 μl injected), DAD 210 nm, 25° C.): tᵣ=4.8 min (96.35%).

MS (Cl, NH₃): m/z=387 (M+NH₄⁺), 370 (M+H⁺), 331 (M+NH₄⁺−C₄H₈), 314 (M+H⁺−C₄H₈).

MS (ES⁺): m/z=761 (2M+Na⁺), 739 (2M+H⁺), 683 (M+H⁺−C₄H₈), 639 (2M+H⁺−C₄H₈−CO₂). 392 (M+Na⁺), 370 (M+H⁺).

¹H-NMR (400 MHz, CD₂Cl₂): δ=7.14 (d, J=8.6 Hz, 2H, Ar), 7.00 (d, J=8.6 Hz, 2H, Ar), 5.69 (d, ²J_{HF}=54.8 Hz, 2H, F—CH₂—O), 4.99 (d, J=7.6 Hz, 1H, NH), 4.36 (mc, 1H, 2-H), 3.00 (mc, 2H, 3-H), 1.41 ppm (s, 9H, 1-tBu), 1.40 (s, 9H, 2-tBu).

¹³C-NMR (100 MHz, CD₂Cl₂): δ (ppm) 170.79 (C1), 155.72 (d, ³J_{CF}=3.2 Hz, Ar—C4), 154.90 (2-C1), 131.82 (Ar—C1), 130.80 (Ar—C2), 116.36 (Ar—C3), 100.01 (d, ¹J_{CF}=217.3 Hz, O—CH₂—F), 81.92 (1-C1), 79.38 (br,. 2-C2), 54.99 (C-2), 37.48 (C-3), 28.03 (2-C3), 27.71 (1-C2).

### 1.2 Example 2

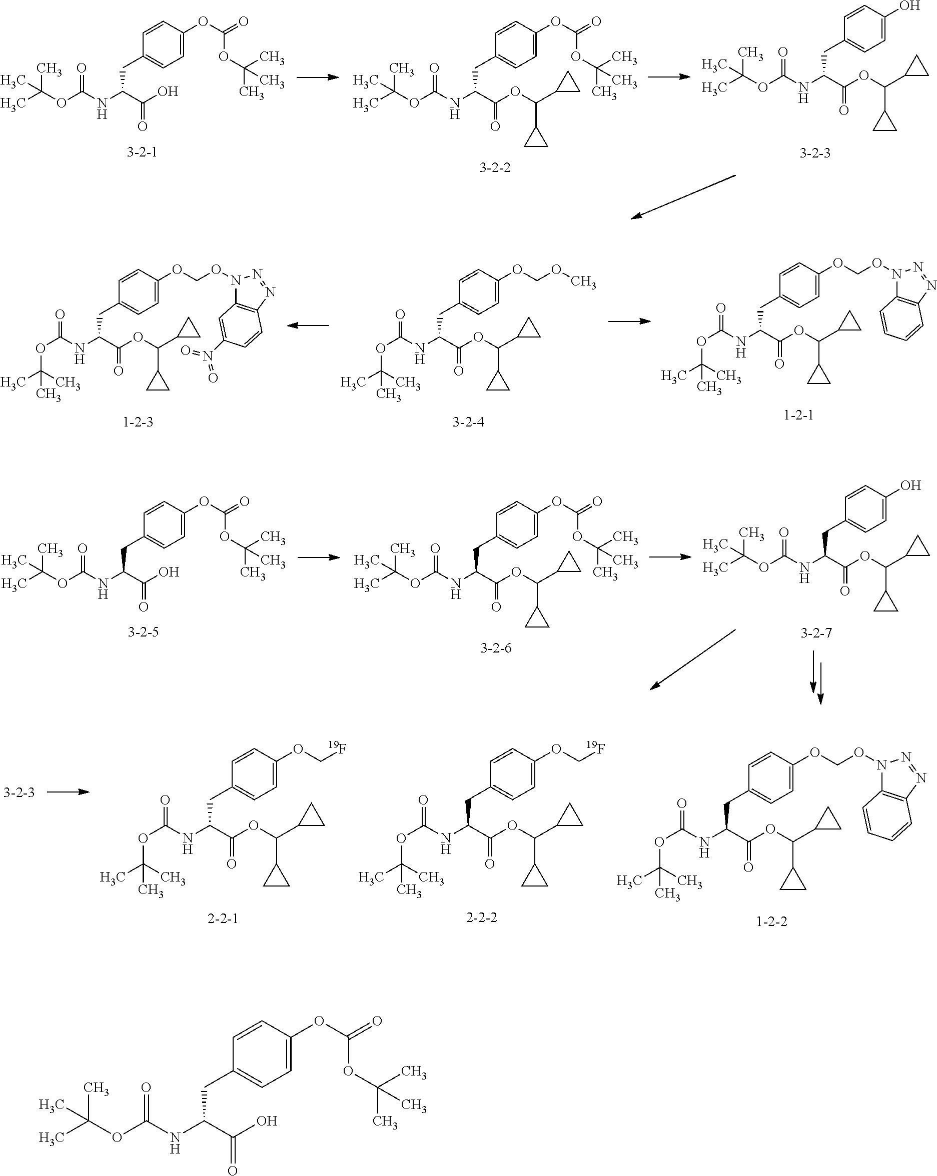

### N,O-Bis(tert-butoxycarbonyl)-D-tyrosine. 3-2-1

18.1 g (100.0 mmol) D-tyrosine were suspended in 250 ml water and a solution of 65.4 g (300.0 mmol) di-tert-butyl dicarbonate in 150 ml 2-propanol was added. The pH was adjusted to 11.5-12 by repeated addition of sodium hydroxide (32% in water). The reaction warmed slightly to about 37° C. and was brought to 20° C. by cooling. Then 250 ml water were added and the mixture extracted with ether. The combined organic phases were washed with water and dried over sodium sulfate. Evaporation of the solvent gave a gummy residue, which was taken up in ethyl acetate. The solution was filtered and hexane added. Upon evaporation, white crystals formed, which were dried i. vac. at 30° C. The yield was 39.1 g (>100%).

α_{D}=−27.9 (c=1, dioxane).

MS (ESI⁺): m/e=785 (2M+Na⁺), 763 (2M+H⁺), 663 (2M+H⁺−C₄H₈−CO₂), 404 (M+Na⁺).

MS (ESI⁻): m/e=761 (2M−Na⁺), 661 (2M−H⁺−C₄H₈−CO₂), 380 (M−H⁺).

¹H NMR (DMSO-d₆, 400 MHz): δ (ppm) 7.25 (d, J=8.6 Hz, 2H, H-2′), 7.06 (d, J=8.6 Hz, 2H. H-3′), 6.88 (d, J=8.1 Hz, 1H, NH), 4.03 (ddd, J=9.3, 8.3, 4.6 Hz, 1H, H-2), 3.03 (dd, J=13.6, 4.5 Hz, 1H, H-3), 2.83 (dd, J=13.6, 9.9 Hz, 1H, H3), 1.48 (s, 9H), 1.32 (s, 9H).

¹³C NMR (DMSO-d₆, 101 MHz): δ (ppm) 173.4 (C-1), 155.3 (2C-1), 151.3 (4′C-1), 149.1 (C-4′), 135.9 (C-1′), 130.1 (C-2′), 120.9 (C-3′), 83.0 (4′C-2), 77.9 (2C-2), 55.3 (C-2), 35.9 (C-3), 28.1 (2C-3), 27.2 (4′C-3).

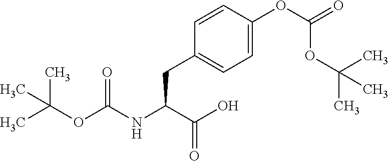 [SMILES: CC(C)(C)OC(=O)N[C@@H](Cc1ccc(OC(=O)OC(C)(C)C)cc1)C(=O)O]

### N,O-Bis(tert-butoxycarbonyl)-L-tyrosine. 3-2-5

In the same way as for 3-2-1, 18.1 g L-tyrosine were reacted to give 35.9 g (94%) of 3-2-5 as a white solid.

α_{D}=+14.6° (c=1, dioxane).

MS (ESI⁻): m/e=785 (2M+Na⁺), 763 (2M+H⁺), 663 (2M+H⁺−C₄H₈−CO₂), 404 (M+Na⁺).

MS (ESI⁻): m/e=761 (2M−H⁺), 661 (2M−H⁺−C₄H₈—CO₂), 380 (M−H⁺).

¹H NMR (400 MHz, DMSO-d₆): δ (ppm) 7.09 (d, J=8.3 Hz, 2H), 6.94 (d, J=8.3 Hz, 2H), 5.79 (d, J=6.1 Hz, 1H), 3.69 (dt, J=5.1, 5.8 Hz, 1H), 3.01 (dd, J=5.3, 13.1 Hz, 1H), 2.90 (dd, J=5.6, 13.4 Hz, 1H), 1.44 (s, 9H), 1.31 (s, 9H).

¹³C NMR (DMSO-d₆, 101 MHz): δ (ppm) 173.3 (C-1), 155.0 (2C-1), 151.9 (4′C-1), 149.2 (3C-4), 137.5 (3C-1), 130.9 (3C-2), 120.8 (3C-3), 83.3 (4′C-2), 77.6 (2C-2), 56.6 (C-2), 37.2 (C-3), 28.7 (2C-3), 27.7 (4′C-3).

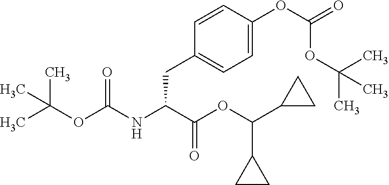 [SMILES: CC(C)(C)OC(=O)N[C@H](Cc1ccc(OC(=O)OC(C)(C)C)cc1)C(=O)OC(C1CC1)C1CC1]

### Dicyclopropylmethyl N,O-bis(tert-butoxycarbonyl)-D-tyrosinate. 3-2-2

10.0 g (26.2 mmol) 3-2-1 and 320 mg (2.62 mmol) 4-(dimethylamino)pyridine were dissolved in 30 ml dichloromethane. 3.82 g (34.1 mmol) dicyclopropylmethanol and 653 mg (34.11 mmol) N-ethyl-N′-3-dimethylaminopropyl carbodiimide hydrochloride were added and the mixture stirred at ambient temperature. The reaction was stirred over night. Ethyl acetate was added and the insolubles were filtered off. The filtrate was concentrated i. vac. Chromatography in two batches on a Biotage system (Flash40+M cartridge, 40 ml/min, n-hexane to n-hexane/ethyl acetate 1:4 in 30 min) gave 6.99 g (56%) of 3-2-2.

MS (ESI⁺): m/e=514 (M+K⁺), 498 (M+Na⁺), 476 (M+H⁺), 458 (M+H⁺−H₂O), 420 (M+H⁺−C₄H₈), 376 (M+H⁺−C₄H₈−CO₂).

¹H NMR (CHLOROFORM-d, 300 MHz):): δ (ppm) 7.21 (d, J=8.7 Hz, 2H, Ar—H), 7.09 (d, J=8.7 Hz, 2H, Ar—H), 5.00 (d, J=7.3 Hz, 1H, NH), 4.52-4.64 (m, 1H, 2-H), 3.89 (t, J=8.9 Hz, 1H, OCH), 3.03-3.23 (m, 2H, 3H₂), 1.56 (s, 9H, OBoc), 1.43 (s, 9H, NBoc), 1.00-1.16 (m, 2H, cyclopropyl CH), 0.41-0.64 (m, 4H, cyclopropyl CH₂), 0.25-0.41 (m, 4H, cyclopropyl CH₂).

¹³C NMR (CHLOROFORM-d, 75 MHz): δ (ppm): 171.4 (C-1), 155.0 (2C-1), 151.8 (4′C-1), 150.0 (3C-4), 133.6 (3C-1), 130.5 (3C-2), 121.1 (3C-3), 83.8 (1C-1), 83.5 (4′C-2), 79.8 (2C-2), 54.4 (C-2), 37.5 (C-3), 28.3 (2C-3), 27.7 (4′C-3), 14.6, 14.6 (cyclopropyl CH), 3.1, 3.0, 2.9, 2.7 (cyclopropyl CH₂).

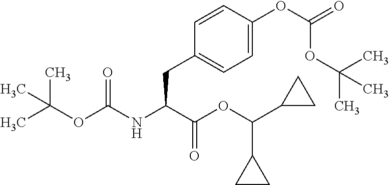 [SMILES: CC(C)(C)OC(=O)N[C@@H](Cc1ccc(OC(=O)OC(C)(C)C)cc1)C(=O)OC(C1CC1)C1CC1]

### Dicyclopropylmethyl N,O-bis(tert-butoxycarbonyl)-L-tyrosinate. 3-2-6

In the same way as for 3-2-2, 10 g 3-2-5 were reacted to give 5.52 g (44%) 3-2-6.

MS (Cl⁺, NH₃): m/e=493 (M+NH₄⁺), 476 (M+H⁺), 437 (M+NH₄⁺−C₄H₈), 420 (M+H⁺−C₄H₈), 376 (M+H⁺−C₄H₈−CO₂), 95 (C₇H₁₁⁺).

¹H NMR (CHLOROFORM-d, 300 MHz): δ (ppm) 7.20 (d, J=8.7 Hz, 2H, Ar—H), 7.08 (d, J=8.5 Hz, 2H, Ar—H), 4.99 (d, J=7.9 Hz, 1H, NH), 4.49-4.65 (m, 1H, 2-H), 3.87 (t, J=8.5 Hz, 1H, OCH), 3.03-3.20 (m, 2H, 3-H₂), 1.55 (s, 9H, OBoc), 1.42 (s, 9H, NBoc), 0.98-1.14 (m, 2H, Cyclopropyl CH), 0.40-0.62 (m, 4H, Cyclopropyl CH₂), 0.25-0.39 (m, 4H, Cyclopropyl CH₂).

¹³C NMR (CHLOROFORM-d, 75 MHz): δ (ppm) 171.4 (C-1), 155.0 (2C-1), 151.8 (OBoc C-1), 150.0 (3C-4), 133.6 (3C-1), 130.5 (3C-2), 121.1 (3C-3), 83.8 (1C-1), 83.5 (OBoc C-2), 79.8 (2C-2), 54.4 (C-2), 37.5 (C-3), 28.3 (3C-3), 27.7 (OBoc C-3), 14.7, 14.6 (cyclopropyl CH), 3.2, 3.0, 2.9, 2.7 (Cyclopropyl CH).

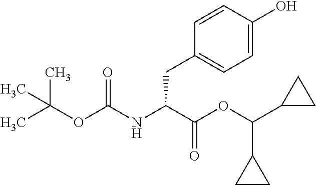 [SMILES: CC(C)(C)OC(=O)N[C@H](Cc1ccc(O)cc1)C(=O)OC(C1CC1)C1CC1]

### Dicyclopropylmethyl N-(tert-butoxycarbonyl)-D-tyrosinate. 3-2-3

5.0 g (10.5 mmol) 3-2-2 was dissolved in 150 ml dichloromethane and 150 ml piperidine added. The mixture was stirred at r.t. for 3 h, after which HPLC/MS indicated complete conversion. Ethyl acetate was added the insolubles were filtered off. The filtrate was concentrated i. vac. and taken up in ethyl acetate. Again the insolubles were separated and the solvent removed i. vac. Chromatography in two batches on a Biotage system (Flash40+M cartridge, 40 ml/min, hexane to ethyl acetate in 30 min) gave 3.74 g (95%) of 3-2-3.

¹H NMR (CHLOROFORM-d, 400 MHz): δ (ppm) 7.05 (d, J=8.0 Hz, 2H, Ar—H), 6.73 (d, J=8.0 Hz, 2H, Ar—H), 5.71 (br. s., 1H, NH), 5.02 (d, J=8.0 Hz, 1H), 4.49-4.60 (m, 1H, 2-H), 3.90 (t, J=8.3 Hz, 3H, OCH), 2.96-3.13 (m, 2H, 3-H₂), 1.44 (s, 9H, Boc), 1.03-1.16 (m, 2H, cyclopropyl CH), 0.53-0.64 (m, 2H, cyclopropyl CH₂), 0.43-0.52 (m, 2H, cyclopropyl CH₂), 0.28-0.42 (m, 4H, cyclopropyl CH₂).

¹³C NMR (CHLOROFORM-d, 101 MHz): δ (ppm) 171.8 (C-1), 155.2 (2C-1), 154.9 (3C-4), 130.6 (3C-2), 127.8 (3C-1), 115.3 (3C-3), 83.7 (1C-1), 79.9 (2C-2), 54.7 (C-2), 37.4 (C-3), 28.3 (2C-3), 14.7, 14.6 (cyclopropyl CH), 3.1, 3.0, 2.9, 2.7 (cyclopropyl CH₂).

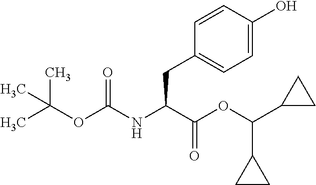 [SMILES: CC(C)(C)OC(=O)N[C@@H](Cc1ccc(O)cc1)C(=O)OC(C1CC1)C1CC1]

### Dicyclopropylmethyl N-(tert-butoxycarbonyl)-L-tyrosinate. 3-2-7

In the same way as for 3-2-3, 2.5 g of 3-2-6 were reacted to give 1.53 g (77%) of slightly impure 3-2-7.

450 mg were purified by preparative HPLC:

Dionex: Pump P 580, Gilson: Liquid Handler 215, Knauer: UV-Detector K-2501, Chiralpak IC 5 μm 250×30 mm, hexane/ethanol 95:5, 40 ml/min, r.t., 450 mg/3.0 ml ethanol, 8×0.35 ml, UV 220 nm. 298 mg of 3-2-7 were obtained with 99.6% purity.

MS (ESI+): m/e=773 (2M+Na+), 751 (2M+H+), 473.

MS (ESI−): m/e=795 (2M+HCOO−), 749 (2M−H+), 420, (M+HCOO−).

¹H NMR (CHLOROFORM-d, 400 MHz): δ (ppm) 7.04 (d, J=8.1 Hz, 2H, Ar—H), 6.72 (d, J=8.3 Hz, 2H, Ar—H), 4.94-5.07 (m, 1H, NH), 4.46-4.60 (m, 1H, 2-H), 3.88 (t, J=8.3 Hz, 1H, OCH), 2.96-3.12 (m, 2H, 3-H2), 1.42 (s, 9H, Boc), 1.02-1.15 (m, 2H, cyclopropyl CH), 0.52-0.64 (m, 2H, cyclopropyl CH₂), 0.41-0.52 (m, 2H, cyclopropyl CH₂), 0.26-0.41 (m, 4H, cyclopropyl CH₂).

¹³C NMR (CHLOROFORM-d, 101 MHz): δ (ppm) 171.8 (C-1), 155.3 (3C-4), 155.1 (2C-1), 130.7 (3C-2), 127.7 (3C-1), 115.4 (3C-3), 83.8 (1C-1), 79.9 (2C-2), 54.8 (C-2), 37.4 (C-3), 28.4 (2C-3), 14.7, 14.7 (cyclopropyl CH), 3.2, 3.0, 3.0, 2.8, 2.7 (cyclopropyl CH₂).

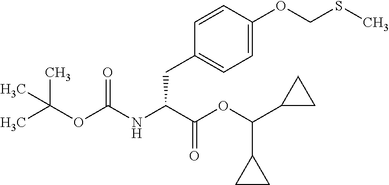 [SMILES: CSCOc1ccc(C[C@@H](NC(=O)OC(C)(C)C)C(=O)OC(C2CC2)C2CC2)cc1]

### Dicyclopropylmethyl N-(tert-butoxycarbonyl)-O-[(methylsulfanyl)methyl]-D-tyrosinate. 3-2-4

A solution of 2.5 g (6.66 mmol) 3-2-3, 100 mg (0.67 mmol) sodium iodide and 17 ml N,N-dimethylformamide was cooled to 0° C. in an ice bath. A suspension of 822 mg (7.23 mmol) potassium tert-butylate in 10 ml tetrahydrofuran was added, resulting in a greenish solution. 510 μl chloro dimethyl sulfide were added. The mixture was allowed to come to r.t., after 2 h HPLC/MS indicated complete consumption of the starting material. Ethyl acetate was added and the insolubles were filtered off. The filtrate was concentrated i. vac. Chromatography on a Biotage system (Flash40+M cartridge, 40 ml/min, n-hexane to n-hexane/ethyl acetate 1:4 in 30 min) gave 1.60 g (55%) of 3-2-4.

MS (ESI⁺): m/e=771 (2M+H⁺−C₄H₈−CO₂), 590 (771-Tyr), 530 (M+C₇H₁₁), 474 (M+K⁺), 436 (M+H⁺), 380 (M+H⁺−C₄H₈), 336 (M+H⁺−C₄H₈−CO₂).

¹H NMR (CHLOROFORM-d, 400 MHz): δ (ppm) 7.10-7.17 (m, 2H, Ar—H), 6.84-6.91 (m, 2H, Ar—H), 5.12 (s, 2H, OCH₂S), 4.98 (d, J=7.8 Hz, 1H, NH), 4.50-4.62 (m, 1H, 2-H), 3.90 (t, J=8.3 Hz, 1H, OCH), 2.99-3.17 (m, 2H, 3-H₂), 2.25 (s, 3H, S—CH₃), 1.43 (s, 9H, Boc), 1.02-1.14 (m, 2H, cyclopropyl CH), 0.52-0.63 (m, 2H, cyclopropyl CH₂), 0.42-0.53 (m, 2H, cyclopropyl CH₂), 0.27-0.42 (m, 4H, cyclopropyl CH₂).

¹³C NMR (CHLOROFORM-d, 101 MHz): δ (ppm) 171.5 (C-1), 156.1 (3C-4), 155.0 (2C-1), 130.6 (3C-2), 129.3 (3C-1), 115.9 (3C-3), 83.6 (1C-1), 79.7 (2C-2), 72.5 (OCH₂S), 54.6 (C-2), 37.3 (C-3), 28.3 (2C-3), 14.6, 14.6 (cyclopropyl CH), 14.6 (SCH₃), 3.1, 3.0, 2.9, 2.7 (cyclopropyl CH₂).

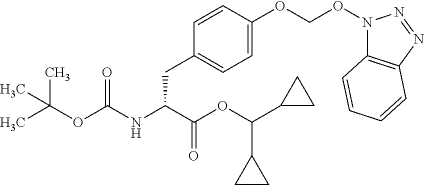 [SMILES: CC(C)(C)OC(=O)N[C@H](Cc1ccc(OCOn2nnc3ccccc32)cc1)C(=O)OC(C1CC1)C1CC1]

### Dicyclopropyl methyl O-[(1H-benzotriazol-1-yloxy)methyl]-N-(tert-butoxycarbonyl)-D-tyrosinate. 1-2-1

1. A solution of 500 mg (1.15 mmol) Dicyclopropylmethyl N-(tert-butoxycarbonyl)-O-[(methylsulfanyl)methyl]-D-tyrosinate 3-2-4 in 4.5 ml dichloromethane was cooled to −15° C. and 169 mg (1.26 mmol) N-chlorosuccinimide added. After 4 h during which the mixture was allowed to come to ambient temperature, UPLC-MS indicated formation of about 50% chloromethyl ether.

2. 495.9 mg (3.66 mmol) 1H-benzotriazol-1-ol hydrate were stirred with 3.67 ml tetrabutylammonium hydroxide (anhydrous, 1 M in methanol). After 30 min at ambient temperature, the solution was carefully evaporated i. vac. at max. 40° C. The residue was dissolved twice in anhydrous toluene and evaporated as described above. A yellow solid resulted, which was used without further purification.

3. The tetrabutylammonium 1H-benzotriazol-1-olate prepared above was dissolved in 5 ml dichloromethane and molecular sieve (4 Å) added. To this solution the chloromethyl ether prepared under 1.) was added at ambient temperature and stirred over night. The solution was directly chromatographed on a Biotage system (Flash40+M cartridge, 40 ml/min, n-hexane to n-hexane/ethyl acetate 1:4 in 20CV=2640 ml) gave 71.5 mg (12%) of 1-2-1.

The compound was further purified by preparative HPLC: Waters Autopurification system: Pump 254, Sample Manager 2767, CFO; DAD 2996, ELSD 2424, SQD 3001; XBrigde C18 5 μm 150×19 mm; A=water+0.1% formic acid; B=acetonitrile; 0-1 min 40% B, 1-8 min 40-100% B, 8-10 min 100% B; 25 ml/min; r.t.; 71 mg/2 ml dimethyl sulfoxide/methanol 1:1; 2×1 ml, DAD scan range 210-400 nm. Peak at 7.2-7.5 min was collected to give 36 mg of the title material (purity: 95.0% by DAD).

The acid solvent apparently caused some decomposition during evaporation. The compound was repurified: Agilent: Prep 1200, 2×Prep Pump, DLA, MWD, Prep FC, ESA: Corona; Chiralpak IC 5 μm 250×20 mm; hexane/ethanol 80:20; 20 ml/min; r.t.; 36 mg/1.5 ml ethanol/methanol 1:1; 2×0.75 ml; UV 254 nm. The peak at 10.6-12.6 min was collected to give 22 mg of the title material (purity: 99.7% by UV).

¹H NMR (DICHLOROMETHANE-d2, 300 MHz): δ (ppm) 7.93-8.05 (m, 1H), 7.31-7.47 (m, 2H), 7.24 (d, J=8.7 Hz, 2H) 7.17-7.25 (m, 1H), 7.08 (d, J=8.7 Hz, 2H), 6.03 (s, 2H, OCH₂O), 5.05 (d, J=7.7 Hz, 1H, NH), 4.46-4.63 (m, 1H, 2-H), 3.90 (t, J=8.7 Hz, 1H, OCH), 2.95-3.30 (m, 2H, 3-H₂), 1.41 (s, 9H, tBu), 1.02-1.19 (m, 2H, CH, cyclopropyl), 0.41-0.66 (m, 4H, CH₂, cyclopropyl), 0.26-0.41 (m, 4H, CH₂, cyclopropyl).

¹³C NMR (DICHLOROMETHANE-d₂, 75 MHz): δ (ppm) 171.4 (C-1), 155.2 (2C-1), 154.9 (3C-4), 143.5 (BtC-4), 131.8 (3C-1), 131.0 (3C-2), 128.7 (BtC-7a), 128.2 (BtC-6), 124.6 (BtC-5), 119.9 (BtC-4), 116.1 (3C-3), 109.0 (BtC-7), 99.1 (OCH₂O), 83.6 (1C-1), 79.5 (2C-2), 54.7 (C-2), 37.3 (C-3), 28.1 (2C-3), 14.6, 14.6 (cyclopropyl CH), 3.0, 2.7, 2.7, 2.5 (Cyclopropyl CH₂).

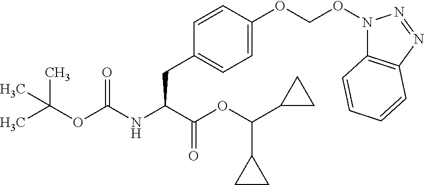 [SMILES: CC(C)(C)OC(=O)N[C@@H](Cc1ccc(OCOn2nnc3ccccc32)cc1)C(=O)OC(C1CC1)C1CC1]

### Dicyclopropyl methyl O-[(1H-benzotriazol-1-yloxy)methyl]-N-(tert-butoxycarbonyl)-L-tyrosinate. 1-2-2

The compound can be prepared in analogy to 1-2-1 from 3-2-7. In the fact, it was isolated from a preparation of 1-2-1, where a stereochemically impure tyrosine derivative had been inadvertedly used as starting material.

307 mg of a mixture were purified by preparative HPLC: Dionex: Pump P 580, Gilson: Liquid Handler 215, Knauer: UV-Detector K-2501; Chiralpak IC 5 μm 250×30 mm; hexane/ethanol 80:20; 30 ml/min; r.t.; 307 mg/1.5 ml ethanol; 6×0.25 ml; UV 254 nm. The peak at 15.7 to 17.5 min was collected to give 128 mg of 1-2-1 with 98% purity. The peak at 20.0 to 21.3 min was collected to give 30 mg of 1-2-2 with 98% purity.

MS (ESI⁻): m/e=524 (M+H⁺).

¹H NMR (DICHLOROMETHANE-d₂, 300 MHz): δ (ppm) 7.97 (dt, J=7.5, 0.9 Hz, 1H, Bt-H), 7.31-7.47 (m, 2H, Bt-H), 7.24 (d, J=8.7 Hz, 2H, Ar—H), 7.21 (d, J=7.5 Hz, 1H, Bt-H), 7.08 (d, J=8.7 Hz, 2H, Ar—H), 6.03 (s, 2H, OCH₂O), 5.06 (d, J=7.5 Hz, 1H, NH), 4.53 (dt, J=7.5, 5.8 Hz, 1H, 2-H), 3.90 (t, J=8.5 Hz, 1H, CHO), 3.17 (dd, J=13.8, 5.8 Hz, 1H, 3-H), 3.06 (dd, J=13.8, 5.7 Hz, 1H, 3-H), 1.41 (s, 9H, Boc), 1.04-1.18 (m, 2H, cyclopropyl CH), 0.42-0.65 (m, 4H, cyclopropyl CH₂), 0.30-0.41 (m, 4H, cyclopropyl CH₂).

¹³C NMR (DICHLOROMETHANE-d₂, 75 MHz): δ (ppm) 171.3 (C-1), 155.1 (3C-4), 154.9 (2C-1), 143.4 (Bt C-3a), 131.7 (3C-1), 131.0 (3C-2), 128.6 (Bt C-7a), 128.2 (Bt C-6), 124.6 (Bt C-5), 119.8 (Bt C-4), 116.0 (3C-3), 109.0 (Bt C-7), 99.0 (OCH₂O), 83.6 (1C-1), 79.5 (2C-2), 54.7 (C-2), 37.3 (C-3), 28.0 (2C-3), 14.6, 14.5 (cyclopropyl CH), 3.0, 2.7, 2.4 (cyclopropyl CH₂).

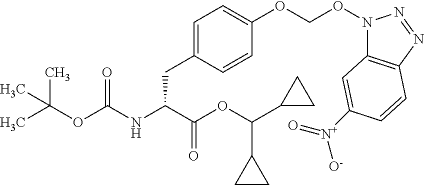 [SMILES: CC(C)(C)OC(=O)N[C@H](Cc1ccc(OCOn2nnc3ccc([N+](=O)[O-])cc32)cc1)C(=O)OC(C1CC1)C1CC1]

### Dicyclopropylmethyl O-[(6-nitro-1H-benzotriazol-1-yloxy)methyl]-N-(tert-butoxy carbonyl)-D-tyrosinate. 1-2-3

In the same way as described for 1-2-1, 150 mg (0.30 mmol) 3-2-4 in 2.5 ml dichloromethane were reacted to give after chromatography (Biotage system SNAP 25 cartridge, 25 ml/min, n-hexane to n-hexane/ethyl acetate 6:4 in 10CV, then isocratic 4 CV) 74 mg (43%) of 1-2-3, which was further purified by preparative HPLC: (Dionex: Pump P 580, Gilson: Liquid Handler 215, Knauer: UV-Detector K-2501; Chiralpak IC 5 μm 250×20 mm; Hexane/Ethanol 50:50; 30 ml/min; RT; 74 mg/2.0 ml EtOH; 2×1.0 ml: UV 210 nm). The fraction eluting at 10.6-11.8 min were collected to give 45 mg (26%) of 1-2-3 with a purity of 99.5%.

MS (ESI⁺): m/e=662 (M+C₇H₁₁⁺), 590 (M+Na⁺), 568 (M+H⁺), 512 (M+H⁺—C₄H₈), 468 (M+H⁺−CO₂−C₄H₈).

¹H NMR (DICHLOROMETHANE-d₂, 500 MHz): δ (ppm) 8.25 (dd, J=9.1, 1.3 Hz, 1H, Bt H-5), 8.18 (d, J=9.1 Hz, 1H, Bt H-4), 8.19 (d, J=1.3 Hz, 1H, Bt H-7), 7.31 (d, J=8.2 Hz, 2H, Ar—H), 7.11 (d, J=8.2 Hz, 2H, Ar—H), 6.14 (s, 2H, OCH₂O), 5.12 (d, J=7.6 Hz, 1H, NH), 4.57 (dt, J=7.6, 5.4 Hz, 1H, 2-H), 3.95 (t, J=8.2 Hz, 1H, OCH), 3.24 (dd, J=13.9, 5.4 Hz, 1H, 3-H), 3.14 (dd, J=13.9, 5.4 Hz, 1H, 3-H), 1.46 (s, 9H, Boc), 1.10-1.21 (m, 2H, cyclopropyl CH), 0.47-0.67 (m, 4H, cyclopropyl CH₂), 0.35-0.45 (m, 4H, cyclopropyl CH₂).

¹³C NMR (DICHLOROMETHANE-d₂, 126 MHz): δ (ppm) 171.3 (C-1), 154.9 (2C-1), 154.6 (3C-4), 147.4 (Bt C-6), 145.4 (Bt C-3a), 132.3 (3C-1), 131.3 (3C-2), 128.2 (Bt C-7a), 121.2 (Bt C-4), 119.5 (Bt C-5), 115.9 (3C-3), 106.7 (Bt C-7), 99.4 (OCH₂O), 83.6 (1C-1), 79.5 (2C-2), 54.7 (C-2), 37.2 (C-3), 28.0 (2C-3), 14.6, 14.5 (1C-2), 3.0, 2.7, 2.5 (1C-3/4).

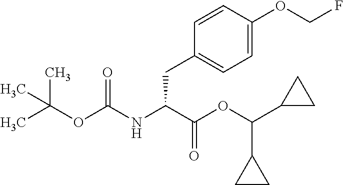 [SMILES: CC(C)(C)OC(=O)N[C@H](Cc1ccc(OCF)cc1)C(=O)OC(C1CC1)C1CC1]

### Dicyclopropylmethyl N-(tert-butoxycarbonyl)-O-(fluoromethyl)-D-tyrosinate. 2-2-1

As described in the preparation of 2-1-1, 250 mg (0.67 mmol) 3-2-3 were reacted to give 290 mg of raw product, which was purified by preparative HPLC.

Dionex: Pump P 580, Gilson: Liquid Handler 215, Knauer: UV-Detector K-2501, Chiralpak IC 5 μm 250×30 mm, hexane/ethanol 95:5, 40 ml/min, r.t., 290 mg/3 ml ethanol, 10×0.3 ml, UV 220 nm. The peak at 7.7-8.2 min was collected to give 85 mg (33%) 2-2-1 with 99.8% purity.

¹H NMR (CHLOROFORM-d, 400 MHz): δ (ppm) 7.16 (d, J=8.5 Hz, 2H, Ar—H), 6.99 (d, J=8.5 Hz, 2H, Ar—H), 5.68 (d, ²J_{HF}=54.7 Hz, 2H, OCH₂F), 4.98 (d, J=7.8 Hz, 1H, NH), 4.50-4.62 (m, 1H, 2-H), 3.89 (t, J=8.5 Hz, 2H, CHO), 3.13 (dd, J=14.1, 6.0 Hz, 1H, 3-H), 3.05 (dd, J=13.8, 5.5 Hz, 1H, 3-H), 1.42 (s, 9H, Boc), 0.99-1.16 (m, 2H, Cyclopropyl CH), 0.52-0.63 (m, 2H, cyclopropyl CH₂), 0.41-0.52 (m, 2H, cyclopropyl CH₂), 0.27-0.40 (m, 4H, cyclopropyl CH₂).

¹³C NMR (101 MHz, CHLOROFORM-d): δ ppm 171.5 (C-1) 155.8 (d, ³J_{CF}=3, 1 Hz, 3C-4) 155.0 (2C-1) 131.31 (30-1) 130.8 (3C-2) 116.6 (3C-3) 100.8 (d, ¹J_{CF}=218.5 Hz, OCH₂F) 83.7 (1C-1) 79.76 (2C-2) 54.56 (C-2) 37.41 (C-3) 28.31 (2C-3) 14.66, 14.62 (cyclopropyl CH) 3.13, 2.96, 2.92, 2.71 (cyclopropyl CH₂).

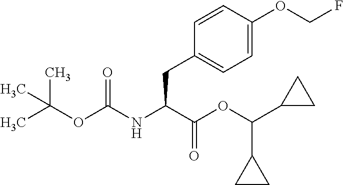 [SMILES: CC(C)(C)OC(=O)N[C@@H](Cc1ccc(OCF)cc1)C(=O)OC(C1CC1)C1CC1]

### Dicyclopropylmethyl N-(tert-butoxycarbonyl)-O-(fluoromethyl)-L-tyrosinate. 2-2-2

As described in the preparation of 2-1-1, 250 mg (0.67 mmol) 3-2-7 were reacted to give 244 mg of raw product, which was purified by preparative HPLC.

Dionex: Pump P 580, Gilson: Liquid Handler 215, Knauer: UV-Detector K-2501, Chiralpak IC 5 μm 250×30 mm, hexane/ethanol 95:5, 40 ml/min, r.t., 244 mg/1.8 ml ethanol/methanol 1:1, 3×0.6 ml, UV 220 nm. The peaks at 8.3-9.2 min were collected to give 83 mg (38%) of 2-2-2 in >99% purity.

¹⁹F NMR (DMSO-d₆, 376 MHz): δ (ppm)-149.8 (t, ¹J_{HF}=55.1 Hz).

¹H NMR (DMSO-d₆, 300 MHz): δ (ppm) 7.21 (d, J=8.5 Hz, 2H, Ar—H), 6.98 (d, J=8.5 Hz, 2H, Ar—H), 5.78 (d, ¹J_{HF}=54.3 Hz, 2H, OCH₂O), 3.97-4.12 (m, 1H, NH), 3.82 (t, J=8.1 Hz, 1H, OCH), 2.90 (dd, J=13.8, 5.7 Hz, 1H, 3-H), 2.80 (dd, J=13.6, 10.0 Hz, 1H, 3-H), 1.30 (s, 9H, Boc), 0.92-1.12 (m, 2H, cyclopropyl CH), 0.09-0.53 (m, 8H, cyclopropyl CH₂).

¹³C NMR (DMSO-d₆, 75 MHz): δ (ppm) 172.1 (C-1), 155.7 (3C-4), 155.2 (2C-1), 132.8 (3C-1), 130.9 (3C-2), 116.4 (3C-3), 101.0 (d, ¹J_{CF}=215.8 Hz, OCH₂O), 81.8 (1C-1), 78.6 (2C-2), 56.2 (C-2), 36.1 (C-3), 28.6 (2C-2), 15.0 (cyclopropyl CH), 3.1, 2.9, 2.8, 2.8 (cyclopropyl CH₂).

### 1.3 Example 3

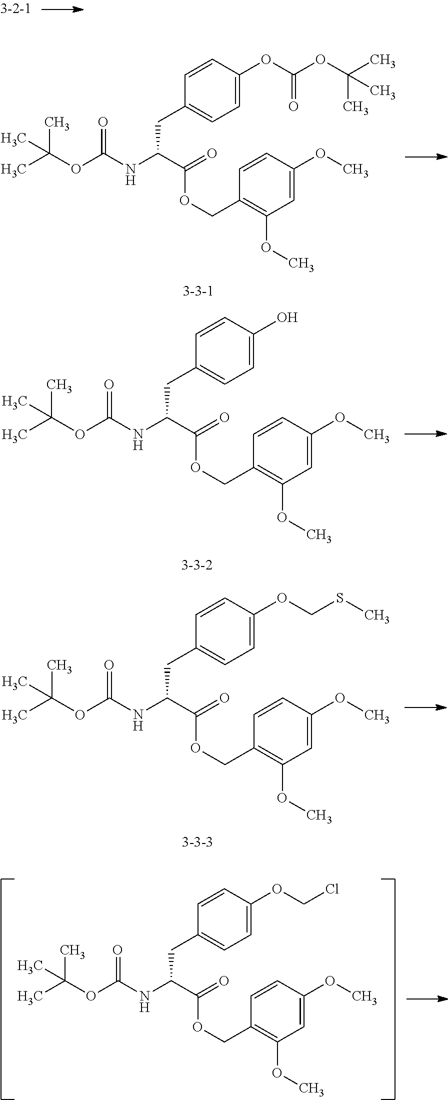

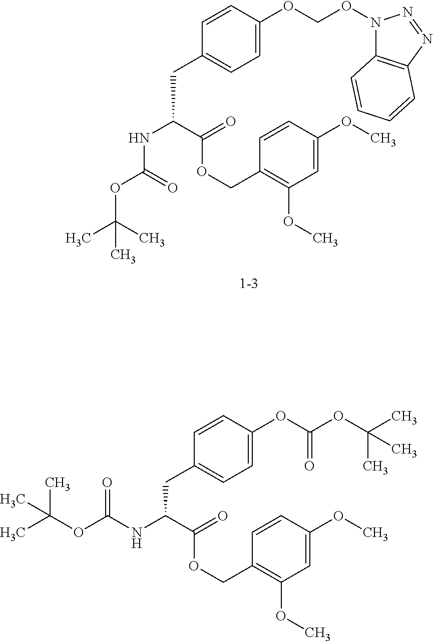 [SMILES: COc1ccc(COC(=O)[C@@H](Cc2ccc(OCOn3nnc4ccccc43)cc2)NC(=O)OC(C)(C)C)c(OC)c1 ; COc1ccc(COC(=O)[C@@H](Cc2ccc(OC(=O)OC(C)(C)C)cc2)NC(=O)OC(C)(C)C)c(OC)c1]

### 2,4-Dimethoxybenzyl N,O-bis(tert-butoxycarbonyl)-D-tyrosinate. 3-3-1

5.0 g (13.1 mmol) 3-2-1 and 160 mg (1.31 mmol) 4-(dimethylamino)pyridine were dissolved in 30 ml dichloromethane (previously dried over 4 Å molecular sieve). 2.87 g (17.0 mmol) 2,4-Dimethoxybenzyl alcohol and 3.27 g (17.0 mmol) N-ethyl-N′-3-dimethylaminopropyl carbodiimide hydrochloride were added and the mixture stirred at ambient temperature over night. Ethyl acetate was added and the insolubles were filtered off. The filtrate was concentrated i. vac. Column chromatography over 500 g silica gel with stepwise gradient (1 L hexane, hexane/ethyl acetate 9:1, hexane/ethyl acetate 8:2, hexane/ethyl acetate 7:3, hexane/ethyl acetate 6:4, respectively) gave 2.15 g (31%) of 3-3-1. (smaller scale reactions gave 49-55% yield).

MS (ESI⁺): m/e=549 (M+H⁺+OH), 532 (M+H⁺), 151 (C₉H₁₁C₂⁺).

¹H NMR (CHLOROFORM-d, 400 MHz): δ (ppm) 7.19 (d, J=9.1 Hz, 2H, Dmb H-7), 6.98-7.10 (m, 4H, Dmb H6, H-4, Tyr H-4/8), 6.42-6.51 (m, 2H, Tyr H-5/7), 5.19 (d, J=11.1 Hz, 1H, Dmb H-1), 5.07 (d, J=11.1 Hz, 1H, Dmb H-1), 4.99 (d, J=8.1 Hz, 1H, NH), 4.54-4.64 (m, 1H, Tyr H-2), 3.83 (s, 3H, Dmb OMe), 3.82 (s, 3H, Dmb OMe), 3.00-3.15 (m, 2H, Tyr H-3), 1.56 (s, 9H, tBu), 1.41 (s, 9H, t-Bu).

¹³C NMR (CHLOROFORM-d, 101 MHz): δ (ppm) 171.7 (C-1), 161.6 (Dmb C-5), 159.2 (Dmb C-3), 155.1 (2C-1), 151.9 (OBoc C-1), 150.0 (3C-4), 133.6 (3C-1), 132.0 (Dmb C-7), 130.4 (3C-2), 121.2 (3C-3), 116.0 (Dmb C-2), 104.1 (Dmb C-6), 98.6 (Dmb C-4), 83.5 (OBoc C-2), 79.9 (2C-2), 62.8 (Dmb C-1), 55.5 (Dmb-OMe), 55.5 (Dmb-OMe), 54.3 (C-2), 37.5 (C-3), 28.4 (2C-3), 27.7 (OBoc C-3).

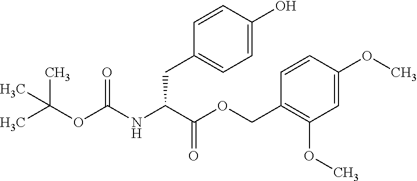 [SMILES: COc1ccc(COC(=O)[C@@H](Cc2ccc(O)cc2)NC(=O)OC(C)(C)C)c(OC)c1]

### 2,4-Dimethoxybenzyl N-(tert-butoxycarbonyl)-D-tyrosinate. 3-3-2

2.10 g (3.95 mmol) 3-3-1 was dissolved in 40 ml dichloromethane (dried over 4 Å molecular sieve) and 40 ml piperidine added. The mixture was stirred at r.t. for 2 h, after which HPLC/MS over 80% conversion. The reaction mixture was partitioned between ethyl acetate and water, The organic phase was separated and dried over sodium sulfate. The residue obtained upon evaporation i. vac. (3.8 g) was purified by chromatography on a Biotage system (Flash 40+M, n-hexane to ethyl acetate in 15CV=1980 ml) to give 1.10 g (64.5%) 3-3-2.

¹H NMR (400 MHz, DICHLOROMETHANE-d₂): δ (ppm) 7.19 (d, J=7.8 Hz, 1H, DMB 6-H), 6.92 (m, d, J=8.6 Hz, 2H, Ar—H), 6.68 (d, J=8.1 Hz, 2H, Ar—H), 6.43-6.51 (m, 2H, DMB 3-H, 5-H), 5.14 (d, J=11.9 Hz, 1H, DMB 1-H), 5.00 (d, J=8.3 Hz, 1H, NH), 5.05 (d, J=11.6 Hz, 1H, DMB 1-H), 4.41-4.54 (m, 1H, 2-H), 3.82 (s, 3H, DMB OMe), 3.81 (s, 3H, DMB OMe), 2.83-3.08 (m, 2H, 3-H), 1.39 (s, 9H, Boc).

¹³C NMR (101 MHz, DICHLOROMETHANE-d₂): δ (ppm) 171.9 (C-1), 161.6 (DMB C-5), 159.2 (DMB C-3), 155.1 (3C-4), 155.1 (2C-1), 131.7 (DMB C-7), 130.5 (3C-2), 127.8 (3C-1), 116.0 (DMB C-2), 115.2 (3C-3), 104.1 (DMB C-6), 98.4 (DMB C-4), 79.6 (2C-2), 62.6 (DMB C-1), 55.5 (DMB 5-OMe), 55.4 (DMB 3-OMe), 54.7 (C-2), 37.2 (C-3), 28.0 (2C-3).

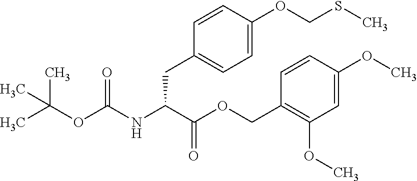 [SMILES: COc1ccc(COC(=O)[C@@H](Cc2ccc(OCSC)cc2)NC(=O)OC(C)(C)C)c(OC)c1]

### 2,4-Dimethoxybenzyl N-(tert-butoxycarbonyl)-O-[(methylsulfanyl)methyl]-D-tyrosinate. 3-3-3

1.1 g (2.556 mmol) 3-3-2 were reacted as described for 3-2-4. Chromatography of the raw product on a Biotage system (Flash 40+M, gradient hexane to ethyl acetate/hexane 1:3, 15CV=1980 ml) gave 490 mg (39%) of 3-3-3.

MS (ESI⁻): m/e=514 (M+Na⁺), 301 (C₁₈H₂₁O₄⁺). 151 (C₉H₁₁O₂⁺).

MS (ESI⁻): m/e=536 (M+HCOO⁻).

¹H NMR (400 MHz, DICHLOROMETHANE-d₂) δ (ppm) 7.20 (d, J=8.1 Hz, 1H, Dmb 7-H), 7.00 (d, J=8.3 Hz, 2H, Ar—H), 6.82 (d, J=8.6 Hz, 2H, Ar—H), 6.49 (d, J=2.3 Hz, 1H, Dmb 4-H), 6.47 (dd, J=2.3, 8.3 Hz, 1H, Dmb 6-H), 5.12 (s, 2H, S—CH₂), 5.14 (d, J=11.9 Hz, 1H), 5.06 (d, J=11.9 Hz, 1H), 4.98 (d, J=8.1 Hz, 1H, NH), 4.43-4.55 (m, 1H. 2-H), 3.82 (s, 3H, Dmb-OCH₃), 3.81 (s, 3H, Dmb-OCH₃), 3.03 (dd, J=5.3, 13.9 Hz, 1H, 3-H), 2.96 (dd, J=5.8, 13.9 Hz, 1H, 3-H), 2.23 (s, 3H, SCH₃), 1.39 (s, 9H, Boc).

¹³C NMR (101 MHz, DICHLOROMETHANE-d₂): δ (ppm) 171.8 (C-1), 161.6 (Dmb C-5), 159.2 (Dmb C-3), 156.1 (3C-4), 154.9 (2C-1), 131.8 (Dmb C-7), 130.4 (3C-2), 129.3 (3C-1), 116.0 (Dmb C-2), 115.8 (3C-3), 104.1 (Dmb C-6), 98.4 (Dmb C-4), 79.5 (2C-2), 72.4 (OCH₂S), 62.6 (Dmb C-1), 55.5 (Dmb OCH₃), 55.4 (Dmb OCH₃), 54.6 (C-2), 37.2 (C-3), 28.0 (3C-3), 14.3 (SCH₃).

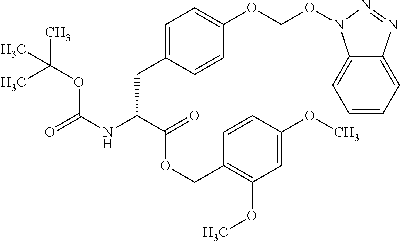 [SMILES: COc1ccc(COC(=O)[C@@H](Cc2ccc(OCOn3nnc4ccccc43)cc2)NC(=O)OC(C)(C)C)c(OC)c1]

### 2,4-Dimethoxybenzyl O-[(1H-benzotriazol-1-yloxy)methyl]-N-(tert-butoxycarbonyl)-D-tyrosinate 1-3

240 mg (0.49 mmol) 3-3-3 were reacted as described for 1-2-1. The raw product was directly chromatographed on a Biotage system (Flash40+M cartridge, 40 ml/min, n-hexane to n-hexane/ethyl acetate 65:35 in 18CV=2376 ml) gave 75 mg (27%) of 1-3. The compound was further purified by preparative HPLC: Dionex: Pump P 580, Gilson: Liquid Handler 215, Knauer: UV-Detector K-2501; Chiralpak IC 5 μm 250×20 mm; hexane/ethanol 80:20; 20 ml/min; r.t.; 71 mg/1 ml ethanol/methanol 1:1; 2×0.5 ml, UV 254 nm. The peak at 20.0-22.5 min was collected to give 43 mg (15%) of the title material (purity: 98.5%) as white solid.

MS (Cl⁺, NH₃): m/e=578 (M⁺), 151 (C₉H₁₁O₂⁺).

¹H NMR (DICHLOROMETHANE-d₂, 400 MHz): δ (ppm) 8.03 (d, J=8.3 Hz, 1H, Bt), 7.38-7.49 (m, 2H, Bt), 7.26 (d, J=8.3 Hz, 1H, Dmb 7-H), 7.24 (d, J=8.5 Hz, 1H, Bt), 7.15 (d, J=8.5 Hz, 2H, Ar—H), 7.07 (d, J=8.5 Hz, 2H, Ar—H), 6.54 (d, J=2.3 Hz, 1H, Dmb 4-H), 6.51 (dd, J=8.3, 2.3 Hz, 1H, Dmb 6-H), 6.07 (s, 2H, OCH₂O), 5.22 (d, J=11.8 Hz, 1H, Dmb-1-H), 5.14 (d, J=11.5 Hz, 1H, Dmb 1-H), 5.09 (d, J=7.3 Hz, 1H, NH), 4.52-4.65 (m, 1H, 2-H), 3.88 (s, 3H, Dmb OMe), 3.84 (s, 3H, Dmb OMe), 3.16 (dd, J=13.8, 5.5 Hz, 1H, 3-H), 3.07 (dd, J=13.8, 5.3 Hz, 1H, 3-H), 1.46 (s, 9H, Boc).

¹³C NMR (101 MHz, DICHLOROMETHANE-d₂): δ (ppm) 172.0 (C-1), 162.1 (Dmb C-5), 159.6 (Dmb C-3), 155.5 (3C-4), 155.3 (2C-1), 143.9 (Bt C-3a), 132.2 (Dmb C-7), 132.0 (3C-1), 131.3 (3C-2), 129.1 (Bt C-7a), 128.6 (Bt C-6), 125.0 (Bt C-5), 120.3 (Bt C-4), 116.5 (3C-3), 116.4 (Dmb C-2), 109.5 (Bt C-7), 104.6 (Dmb C-6), 99.4 (OCH₂O), 98.8 (Dmb C-4), 80.0 (2C-2), 63.1 (Dmb C-1), 55.9 (Dmb OMe), 55.8 (Dmb OMe), 55.0 (C-2), 37.8 (C-3), 28.4 (2C-3).

### 1.4 Example 4

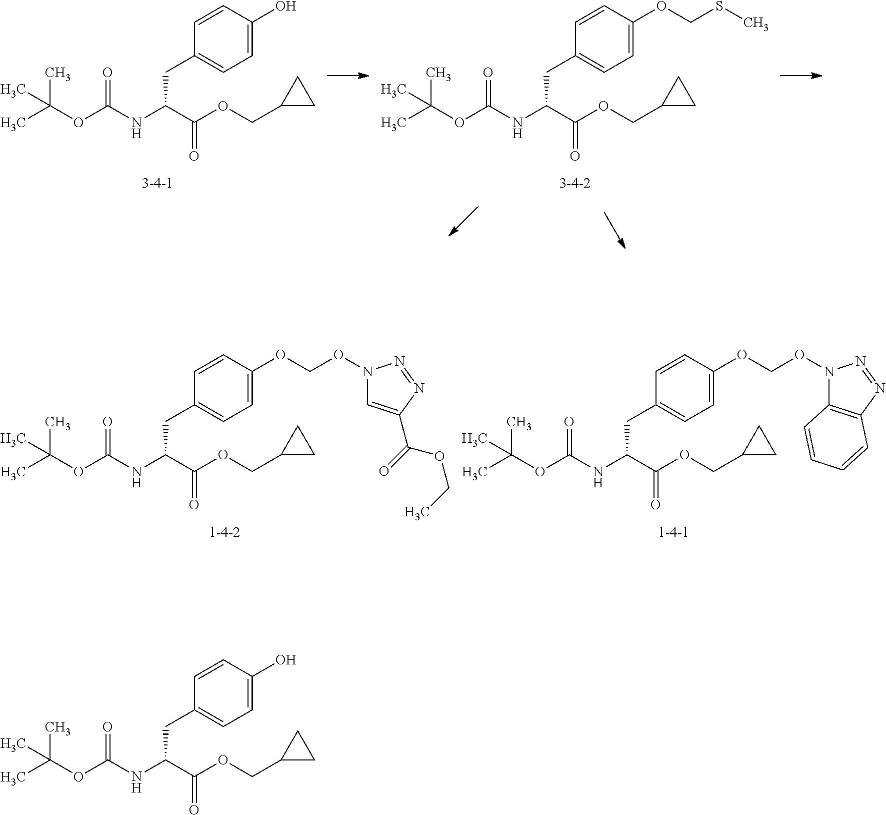

### Cyclopropylmethyl N-(tert-butoxycarbonyl)-D-tyrosinate. 3-4-1

5.00 g (17.8 mmol) Boc-D-tyrosine and 2.90 g (8.89 mmol) cesium carbonate were stirred in 150 ml water for 30 min at r.t. and then lyophilized. The resulting white powder was dissolved in 100 ml N,N-dimethylformamide (dried over 4 Å molecular sieve), 1,724 ml (17.8 mmol) (Bromomethyl)cyclopropane were added and the mixture stirred at r.t. over night. The mixture was partitioned between ethyl acetate and water, the aqueous phase was extracted with ethyl acetate, the combined organic phases dried over sodium sulfate and the solvent evaporated i. vac. The residue was dissolved in ethyl acetate and extracted twice with water. After drying and evaporation 5.28 g (89%) 3-4-1 were obtained as a white solid.

¹H NMR (400 MHz, CHLOROFORM-d) d ppm 7.00 (d, J=8.1 Hz, 2H, Ar—H) 6.73 (d, J=7.8 Hz, 2H, Ar—H) 5.54 (br. s, 1H, OH) 5.02 (d, J=7.6 Hz, 1H, NH) 4.48-4.61 (m, 1H, 2-H) 3.88-3.99 (m, 2H, OCH₂) 2.95-3.10 (m, 2H, 3-H₂) 1.42 (s, 9H, Boc) 1.03-1.18 (m, 1H, cyclopropyl CH) 0.52-0.63 (m, 2H, cyclopropyl CH₂) 0.22-0.31 (m, 2H, cyclopropyl CH₂). Spectrum is identical with an earlier preparation via a different route.

MS (ESI⁺): m/e=693 (2M+Na⁺), 671 (2M+H⁺), 336 (M+H⁺), 280 (M+H⁺−C₄H₈), 236 (M+H⁺−C₄H₈−CO₂).

¹³C NMR (CHLOROFORM-d, 101 MHz): δ (ppm) 172.2 (C-1), 155.7 (3C-4), 155.2 (2C-1), 130.4 (3C-2), 127.1 (3C-1), 115.5 (3C-3), 79.9 (2C-2), 70.2 (1C-1), 54.6 (C-2), 37.5 (C-3), 28.4 (2C-3), 9.7 (cyclopropyl CH), 3.5, 3.4 (cyclopropyl CH₂).

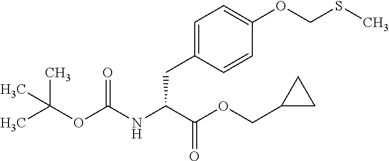 [SMILES: CSCOc1ccc(C[C@@H](NC(=O)OC(C)(C)C)C(=O)OCC2CC2)cc1]

### Cyclopropylmethyl N-(tert-butoxycarbonyl)-O-[(methylsulfanyl)methyl]-D-tyrosinate. 3-4-2

A solution of 1.19 g (3.55 mmol) 3-4-1, 53 mg (0.36 mmol) sodium iodide and 8 ml N,N-dimethylformamide was cooled to 0° C. in an ice bath. A suspension of 358 mg (3.19 mmol) potassium tert-butylate in 3 ml tetrahydrofuran was added, resulting in a greenish solution. 337 μl (4.08 mmol) chloro dimethyl sulfide were added. The mixture was allowed to come to r.t., stirred for 2 h and stored at 5° C. over night. Ethyl acetate was added and the insolubles were filtered off. The filtrate was concentrated i. vac. Chromatography on a Biotage system (Flash40+M cartridge, 40 ml/min, n-hexane to n-hexane/ethyl acetate 1:4, 15 CV=1980 ml) gave 660 mg (47%) of 3-2-4 and 200 mg (17%) starting material.

MS (ESI⁺): m/e=791 (2M+H⁺), 396 (M+H⁺), 340 (M+H⁺−C₄H₈), 296 (M+H⁺−C₄H₈−CO₂).

¹H NMR (DICHLOROMETHANE-d₂, 400 MHz): δ (ppm) 7.09 (d, J=8.6 Hz, 2H, Ar—H), 6.87 (d, J=8.6 Hz, 2H, Ar—H), 5.13 (s, 2H, OCH₂S), 4.99 (d, J=7.8 Hz, 1H, NH), 4.42-4.54 (m, 1H, 2-H), 3.87-3.99 (m, 2H, OCH₂), 2.95-3.12 (mc, 2H, 3-H), 2.22 (s, 3H, SCH₃), 1.40 (s, 9H, Boc), 1.04-1.18 (m, 1H, cyclopropyl CH), 0.53-0.61 (m, 2H, cyclopropyl CH₂), 0.22-0.31 (m, 2H, cyclopropyl CH₂).

¹³C NMR (101 MHz, DICHLOROMETHANE-d₂): δ (ppm) 171.9 (C-2), 156.1 (3C-4), 154.9 (2C-1), 130.4 (3C-2), 129.4 (3C-1), 115.9 (3C-3), 79.5 (2C-2), 72.4 (OCH₂S), 70.1 (1C-1), 54.6 (C-2), 37.4 (C-3), 28.0 (3C-3), 14.3 (SCH₃), 9.6 (1C-2), 3.2 (1C-3), 3.1 (1C-4).

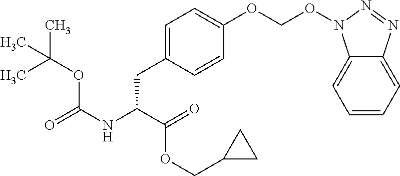 [SMILES: CC(C)(C)OC(=O)N[C@H](Cc1ccc(OCOn2nnc3ccccc32)cc1)C(=O)OCC1CC1]

### Cyclopropylmethyl O-[(1H-benzotriazol-1-yloxy)methyl]-N-(tert-butoxycarbonyl)-D-tyrosinate. 1-4-1

650 mg (1.64 mmol) 3-4-2 were reacted as described for 1-2-1. The reaction mixture was directly applied to Isolute and chromatographed on a Biotage system (Flash40+M cartridge, 40 ml/min, n-hexane to n-hexane/ethyl acetate 1:2 in 15CV=1980 ml) gave 400 mg (50%) of 1-4-1. The whole reaction including purification was done in one day. Storage of the raw product adversely affects the yield.

The compound was further purified by preparative HPLC: Dionex: Pump P 580, Gilson: Liquid Handler 215, Knauer: UV-Detector K-2501; Chiralpak IC 5 μm 250×20 mm; hexane/ethanol 80:20; 40 ml/min; r.t.; 400 mg/3.2 ml ethanol; 8×0.4 ml, UV 254 nm. The peak at 15.6-18.1 min was collected to give 308 mg (39%) of 1-4-1 as white solid with 99.9% purity.

MS (ESI⁺): m/e=505 (M+Na⁺), 483 (M+H⁺), 427 (M+H⁺−C₄H₈), 383 (M+H⁺−C₄H₈−CO₂).

¹H NMR (DICHLOROMETHANE-d₂, 400 MHz): δ (ppm) 8.03 (dt, J=8.3, 1.0 Hz, 1H, Bt H-7), 7.47 (ddd, J=8.0, 7.0, 1.0 Hz, 1H, Bt 5-H*), 7.41 (ddd, J=8.0, 7.0, 1.0 Hz, 1H, Bt 6-H*), 7.27 (d, br., J=8.0 Hz, 1H, Bt 4-H), 7.25 (d, J=8.5 Hz, 2H, Ar—H), 7.13 (d, J=8.5 Hz, 2H, Ar—H), 6.08 (s, 2H, OCH₂O), 5.11 (d, J=7.5 Hz, 1H, NH), 4.53-4.64 (m, 1H, 2-H), 3.94-4.07 (mc, 2H, OCH₂), 3.20 (dd, J=14.1, 5.5 Hz, 1H, 3-H), 3.11 (dd, J=13.6, 5.5 Hz, 1H, 3-H), 1.47 (s, 9H, Boc), 1.13-1.22 (m, 1H, cyclopropyl CH), 0.59-0.66 (m, 2H, cyclopropyl CH₂), 0.31-0.37 (m, 2H, cyclopropyl CH₂).

¹³C NMR (DICHLOROMETHANE-d₂, 101 MHz): δ (ppm) 172.2 (C-1), 155.6 (3C-4), 155.3 (2C-1), 143.9 (Bt C-3a), 132.1 (3C-1), 131.3 (3C-2), 129.1 (Bt C-7a), 128.6 (Bt C-6), 125.0 (Bt C-5), 120.3 (Bt C-4), 116.6 (3C-3), 109.4 (Bt C-7), 99.5 (OCH₂O), 80.0 (2C-2), 70.6 (1C-1), 55.0 (C-2), 37.9 (C-3), 28.4 (2C-3), 10.1 (1C-2), 3.6 (1C-3), 3.5 (1C-4).

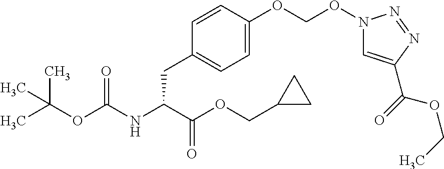 [SMILES: CCOC(=O)c1cn(OCOc2ccc(C[C@@H](NC(=O)OC(C)(C)C)C(=O)OCC3CC3)cc2)nn1]

### Cyclopropylmethyl N-(tert-butoxycarbonyl)-O-({[4-(ethoxycarbonyl)-1H-1,2,3-triazol-1-yl]oxy}methyl)-D-tyrosinate. 1-4-2

360 mg (0.91 mmol) 3-4-2 were reacted as described for 1-4-1. The reaction mixture was directly applied to Isolute and chromatographed on a Biotage system (Flash40+M cartridge, 40 ml/min, n-hexane to n-hexane/ethyl acetate 1:2 in 15CV=1980 ml) gave 230 mg (50%) of 1-4-2. The compound was further purified by preparative HPLC. Agilent: Prep 1200, 2×Prep Pump, DLA, MWD, Prep FC, ESA: Corona, Chiralpak IC 5 μm 250×20 mm, hexane/ethanol 50:50, 15 ml/min, r.t., 230 mg/3.5 ml ethanol/methanol 1:1, 7×0.5 ml, UV 210 nm. The peak at 7.0-8.9 min was collected to give 190 mg (41%) of 1-4-2 with 98.5% purity.

MS (ESI⁺): m/e=527 (M+Na⁺), 505 (M+H⁺), 449 (M+H⁺−C₄H₈), 405 (M+H⁺−C₄H₈−CO₂).

MS (ESI⁻): m/e=549 (M+HCOO).

¹H NMR (DICHLOROMETHANE-d₂, 400 MHz): δ (ppm) 8.00 (s, 1H, T H-5), 7.19 (d, J=8.6 Hz, 2H, Ar—H), 7.06 (d, J=8.6 Hz, 2H, Ar—H), 5.90 (s, 2H, OCH₂O), 5.04 (d, J=7.8 Hz, 1H, NH), 4.52 (ddd, J=7.8, 6.1, 5.6 Hz, 1H, 2-H), 4.36 (q, J=7.3 Hz, 2H, T OCH₂), 3.92 (dd, J=11.4, 7.6 Hz, 1H, OCH₂), 3.96 (dd, J=11.4, 7.3 Hz, 2H, OCH₂), 3.13 (dd, J=13.6, 5.6 Hz, 1H, 3-H), 3.04 (dd, J=13.6, 6.1 Hz, 1H, 3-H), 1.36 (t, J=7.1 Hz, 3H), 1.40 (s, 9H, Boc), 1.08-1.16 (m, 1H, cyclopropyl CH), 0.53-0.62 (m, 2H, cyclopropyl CH₂), 0.24-0.32 (m, 2H, cyclopropyl CH₂).

¹³C NMR (101 MHz, DICHLOROMETHANE-d₂): δ (ppm) 171.7 (C—), 159.9 (T COOEt), 155.0 (3C-4), 154.9 (2C-1), 138.2 (T C-4), 132.2 (3C-1), 131.0 (3C-2), 123.3 (T C-5), 116.3 (3C-3), 99.2 (OCH₂O), 79.6 (2C-2), 70.2 (1C-1), 61.4 (T OCH₂), 54.6 (C-2), 37.4 (C-3), 28.0 (2C-3), 14.0 (T CH₃), 9.7 (cyclopropyl CH), 3.2 (cyclopropyl CH₂), 3.1 (cyclopropyl CH₂).

### Example 5

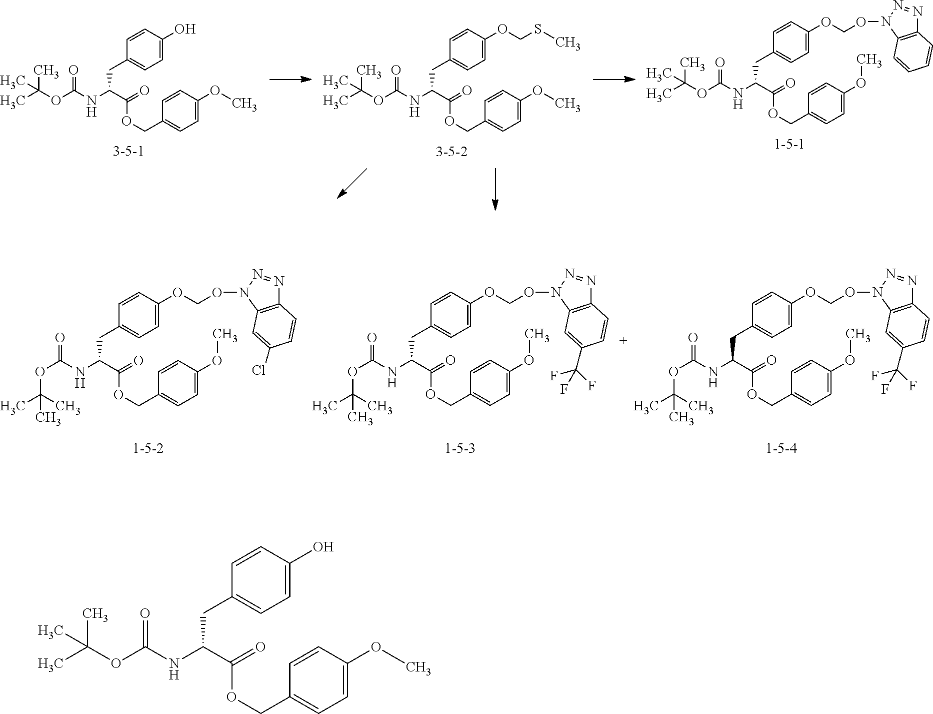

### 4-Methoxybenzyl N-(tert-butoxycarbonyl)-D-tyrosinate. 3-5-1

To 1,763 g (6.27 mmol) Boc-D-Tyr-OH in 52 ml N,N-dimethylformamide were added 1,041 g (3.2 mmol) cesium carbonate and the mixture stirred at r.t. for 1.5 h. 1,260 g (6.27 mmol) 4-methoxybenzyl bromide were added and the mixture stirred at r.t. over night. It was diluted with ethyl acetate and water. The pH was adjusted to 5 with 250 μl of 5% acetic acid. The aqueous phase was separated and extracted with ethyl acetate. The combined extracts were dried and evaporated in the vacuum at 50° C. to yield 2.79 g (100%) of 3-5-1.

MS (ESI⁺): m/e=402.53 (M+H⁺), 803.72 (2M+H⁺).

¹H NMR (CHLOROFORM-d, 400 MHz): δ (ppm) 7.24 (d, J=8.5 Hz, 2H, Mbn-H), 6.82-6.92 (m, 4H, Mbn-H, Ar—H), 6.67 (d, J=8.3 Hz, 2H, Ar—H), 5.11 (d, J=12.0 Hz, 1H, Mbn 1-H), 5.02 (d, J=12.5 Hz, 1H, Mbn 1-H), 4.99 (d, J=8.5 Hz, 1H, NH), 4.49-4.59 (m, 1H, 2-H), 3.81 (s, 3H, Mbn OCH₃), 2.93-3.03 (m, 2H, 3-H), 1.41 (s, 9H, Boc).

¹³C NMR (CHLOROFORM-d, 101 MHz): δ (ppm) 171.9 (C-1), 159.8 (Mbn C-5), 155.2 (3C-4), 155.1 (2C-1), 130.4 (3C-2), 127.4 (3C-1), 127.4 (Mbn C-2), 115.4 (3C-3), 114.0 (Mbn C-4), 80.0 (2C-1), 66.9 (Mbn C-1), 55.3 (Mbn OCH₃), 54.6 (C-3), 37.4 (C-3), 28.3 (2C-3).

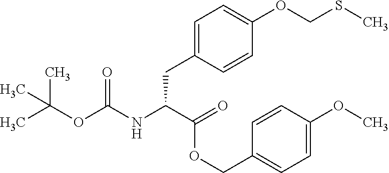 [SMILES: COc1ccc(COC(=O)[C@@H](Cc2ccc(OCSC)cc2)NC(=O)OC(C)(C)C)cc1]

### 4-Methoxybenzyl N-(tert-butoxycarbonyl)-O-[(methylsulfanyl)methyl]-D-tyrosinate. 3-5-2

1.60 g (3.99 mmol) 3-5-1 were dissolved in 32 ml N,N-dimethylformamide. 2.60 g (7.97 mmol) cesium carbonate were added and the mixture stirred for 30 min. 0.4 ml (4.78 mmol) Chloromethyl methyl sulfide were added and the mixture stirred at r.t. for 48 h. Further 0.1 ml (1.20 mmol) Chloromethyl methyl sulfide were added and the mixture stirred for 24 h. The solvent was distilled off and the residue distributed between water and ethyl acetate. The organic solvent was extracted with sodium chloride solution, dried and evaporated. The residue was chromatographed on a Biotage system (Isolera Four, SNAP 25 g, 25 ml/min, n-hexane to n-hexane/ethyl acetate 1:5) to give 682 mg (33%) 3-5-2.

A previous preparation using 500 mg 3-5-1 gave 440 mg (77%) of 3-5-2.

MS (ESI⁺): m/e=462.55 (M+H⁺), 923.69 (2M+H⁺).

¹H NMR (CHLOROFORM-d, 300 MHz): δ (ppm) 7.25 (d, J=8.3 Hz, 2H, Mbn-H), 6.93 (d, J=8.3 Hz, 2H, Ar—H), 6.89 (d, J=8.7 Hz, 2H, Ar—H), 6.80 (d, J=8.5 Hz, 2H, Mbn-H), 5.10 (s, 2H, OCH₂S), 5.12 (d, J=12.1 Hz, 1H, Mbn 1-H), 5.03 (d, J=11.9 Hz, 1H, Mbn 1-H), 4.96 (d, J=8.5 Hz, 1H, NH), 4.48-4.63 (m, 1H, 2-H), 3.82 (s, 3H, OCH₃), 2.92-3.10 (m, 2H, 3-H), 2.25 (s, 3H, SCH₃), 1.41 (s, 9H, Boc).

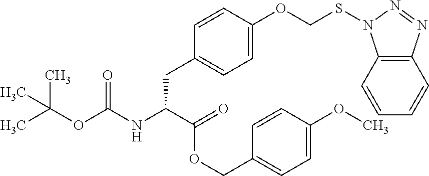 [SMILES: COc1ccc(COC(=O)[C@@H](Cc2ccc(OCSn3nnc4ccccc43)cc2)NC(=O)OC(C)(C)C)cc1]

### 4-Methoxybenzyl O-[(1H-benzotriazol-1-yloxy)methyl]-N-(tert-butoxycarbonyl)-D-tyrosinate. 1-5-1

520 mg (1.13 mmol) 3-5-2 were reacted as described for 1-2-1. The reaction mixture was directly applied to Isolute and chromatographed on a Biotage system (Isolera Four, SNAP 50 g, 40 ml/min, n-hexane to n-hexane/ethyl acetate 1:4) to give 492 mg.

The compound was further purified by preparative HPLC. Waters Autopurificationsystem: Pump 254, Sample Manager 2767, CFO, DAD 2996, ELSD 2424, SQD 3001, XBrigde C18 5 μm 150×19 mm, A=water+0.2% ammonia, B=acetonitrile, 0-1 min 40% B, 1-8 min 40-100% B, 25 ml/min, r.t., 500 mg/7 ml dimethyl sulfoxide/methanol 1:1, 7×1 ml, DAD scan range 210-400 nm, MS ESI+, ESI−, scan range 160-1000 m/z, ELSD. The peak at 6.6-7.0 min was collected to give 213 mg (33%) of 1-5-1 as white solid with >99% purity.

MS (ESI⁺): m/e=549.62 (M+H⁺).

¹H NMR (CHLOROFORM-d, 400 MHz): δ (ppm) 8.00 (d, J=7.6 Hz, 1H), 7.32-7.43 (m, 2H), 7.25-7.31 (m, 2H), 7.12 (d, J=8.1 Hz, 1H), 6.97 (d, J=8.6 Hz, 1H), 6.93-7.07 (d, J=8.6 Hz, 2H), 6.88 (d, J=8.6 Hz, 2H, Bn), 6.01 (s, 2H, OCH₂O), 5.16 (d, J=11.4 Hz, 1H), and 5.05 (d, J=11.4 Hz, 2H, OCH₂Ar), 5.02 (d, J=8.8 Hz, 1H, NH), 4.55-4.66 (m, 1H, 2-H), 3.79 (s, 3H. OCH₃), 3.11 (dd, J=13.9, 5.8 Hz, 1H, 3-H), 3.03 (dd, J=14.1, 5.6 Hz, 1H, 3-H), 1.43 (s, 9H, Boc).

¹³C NMR (CHLOROFORM-d, 101 MHz): δ (ppm) 171.6 (C-1), 159.9 (Mbn C-5), 155.2 (3C-4), 155.1 (2C-1), 143.5 (Bt C-3a), 131.2 (3C-1), 130.9 (3C2), 130.7 (Mbn C-3), 128.8 (Bt C-7a), 128.3 (Bt C-6), 127.3 (Mbn C-2), 124.7 (Bt C-5), 120.1 (Bt C-4), 116.1 (3C-3), 114.0 (Mbn C-4), 109.1 (Bt C-7), 99.0 (OCH₂O), 80.1 (2C-1), 67.1 (Mbn C-1), 55.4 (Mbn C-6), 54.5 (C-2), 37.5 (C-3), 28.4 (2C-3).

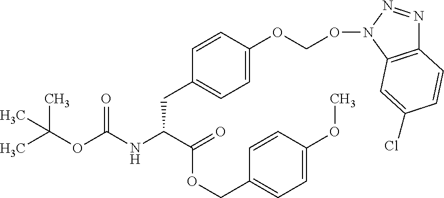 [SMILES: COc1ccc(COC(=O)[C@@H](Cc2ccc(OCOn3nnc4ccc(Cl)cc43)cc2)NC(=O)OC(C)(C)C)cc1]

### 4-Methoxybenzyl N-(tert-butoxycarbonyl)-O-{[(6-chloro-1H-benzotriazol-1-yl)oxy]methyl}-D-tyrosinate. 1-5-2

100 mg (0.22 mmol) 3-5-2 were reacted as described for 1-2-1, where the 1H-benzotriazol-1-ol hydrate was replaced by 6-chloro-1H-benzotriazol-1-ol. The reaction mixture was directly applied to Isolute and chromatographed on a Biotage system (Isolera Four, SNAP 10 g, 12 ml/min, n-hexane to n-hexane/ethyl acetate 4:1) to give 75 mg.

MS (ESI⁻): m/e=583.17 (M+H⁺).

MS (ESI⁻): m/e=627.10 (M+HCOO).

¹H NMR (CHLOROFORM-d, 300 MHz): δ (ppm) 7.92 (dd, J=8.9, 0.4 Hz, 1H Bt 4-h), 7.31 (dd, J=8.9, 1.9 Hz, 1H, Bt 5-H), 7.28 (d, J=8.5 Hz, 2H, PMB 2-H), 7.03 (d, J=8.7 Hz, 2H, Ar 2-H), 7.06 (br. s., 1H, Bt 7-H), 6.94 (d, J=8.5 Hz, 2H, PMB 3-H), 6.87 (d, J=8.7 Hz, 2H, Ar 3-H), 5.96-6.04 (m, 2H, OCH₂O), 5.15 (d, J=11.7 Hz, 1H, PMB CH₂), 5.05 (d, J=12.1 Hz, 1H, PMB CH₂), 5.01 (br. s., 1H, NH), 4.53-4.66 (m, 1H, 2-H), 3.79 (s, 3H, PMB OMe), 3.08 (br. s., 2H, 3-H), 1.43 (s, 9H, Boc).

¹³C NMR (CHLOROFORM-d, 75 MHz): δ (ppm) 171.6 (C-1), 159.8 (1C-5), 155.0 (2C-1), 154.8 (3C-4), 142.0 (Bt C-7a), 134.8 (Bt C-3a), 131.4 (3C-1), 131.0 (1C-2), 130.6 (3C-2), 129.3 (1C-3), 127.3 (Bt C-6), 126.0 (Bt C-5), 121.1 (Bt C-7), 116.0 (3C-3), 113.9 (1C-4), 108.9 (Bt C-4), 98.8 (OCH₂O), 80.0 (2C-2), 67.1 (1C-1), 55.3 (OCH₃), 54.4 (C-2), 37.4 (C-3), 28.3 (2C-3).

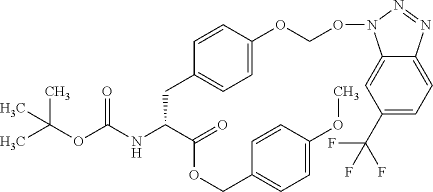 [SMILES: COc1ccc(COC(=O)[C@@H](Cc2ccc(OCOn3nnc4ccc(C(F)(F)F)cc43)cc2)NC(=O)OC(C)(C)C)cc1]

### 4-Methoxybenzyl O-[(6-trifluoromethyl-1H-benzotriazol-1-yloxy)methyl]-N-(tert-butoxycarbonyl)-D-tyrosinate. 1-5-3

150 mg (0.33 mmol) 3-5-2 were reacted as described for 1-2-1, where the 1H-benzotriazol-1-ol hydrate was replaced by 6-trifluoromethyl-1H-benzotriazol-1-ol. The reaction mixture was directly applied to Isolute and chromatographed on a Biotage system (Isolera Four, SNAP 25 g, 25 ml/min, n-hexane 1 CV, n-hexane to n-hexane/ethyl acetate 6:4 10CV, then isocratic 4CV) to give 221 mg (>100%). The material was further purified by HPLC: (Dionex: Pump P 580, Gilson: Liquid Handler 215, Knauer: UV-Detector K-2501; Chiralpak IC 5 μm 250×30 mm; Hexane/Ethanol 80:20; 40 ml/min; RT; 221 mg/3 ml EtOH/Dichlormethan 1:1; 6×500 ml; UV 210 nm). Two peaks, 13.1-14.1 min (77 mg (38%), 99.5% purity, 1-5-3) and 14.1-15.5 min (48 mg (22%), 93.2% purity, 1-5-4) were collected.

Both peaks had the same mass. The stereochemistry was putatively assigned in comparison with 1-2-1 and 1-2-2.

MS (ESI⁻): m/e=639 (M+Na⁺), 617 (M+H⁺), 561 (M+H⁺−C₄H₈), 121 (C₈H₃O⁺).

¹H NMR (DICHLOROMETHANE-d₂, 400 MHz): δ (ppm) 8.13 (d, J=8.6 Hz, 1H, Bt 4-H), 7.59 (dd, J=8.8, 1.3 Hz, 1H, Bt 5-H), 7.48 (s, 1H, Bt 7-H), 7.24-7.32 (m, 2H, Mbn 3-H), 7.07 (d, J=8.3 Hz, 2H, Ar—H), 6.98 (d, J=8.6 Hz, 2H, Ar—H), 6.83-6.92 (m, 2H, Mbn 4-H), 6.01-6.09 (m, 2H, OCH₂O), 5.12 (d, J=11.9 Hz, 1H, Mbn 1-H), 5.06 (d, J=11.9 Hz, 1H, Mbn 1-H), 5.02 (d, J=7.8 Hz, 1H, NH), 4.54 (dt, J=7.8, 5.8 Hz, 1H, 2-H), 3.77 (s, 1H, OMe), 3.11 (dd, J=13.9, 5.8 Hz, 1H, 3-H), 3.03 (dd, J=13.9, 5.8 Hz, 1H, 3H), 1.40 (s, 9H, Boc).

¹⁹F NMR (DICHLOROMETHANE-d₂, 376 MHz): δ (ppm) −62.3.

¹³C NMR (101 MHz, DICHLOROMETHANE-d₂) δ ppm 171.6 (C-1), 159.9 (Mbn C-5), 155.0 (br., 2C1), 154.6 (3C-4), 144.6 (Bt C-3a), 131.8 (3C-1), 131.0 (3C-2), 130.5 (Mbn C-3) 130.1 (q, ²J_{CF}=32.8 Hz, Bt C-6), 128.2 (Bt C-7a), 127.5 (Mbn C-2), 123.7 (q, ¹J_{CF}=272.4 Hz, CF₃), 121.2 (Bt C-4, Bt C-5), 115.9 (3C-3) 113.8 (Mbn C-4), 107.8 (q, ³J_{CF}=4.8 Hz, Bt C-7), 99.0 (OCH₂O), 79.6 (2C-2), 66.9 (Mbn C-1), 55.2 (Mbn OMe), 54.6 (C-2), 37.2 (C-3), 28.0 (2C-3).

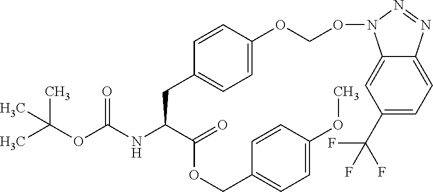 [SMILES: COc1ccc(COC(=O)[C@H](Cc2ccc(OCOn3nnc4ccc(C(F)(F)F)cc43)cc2)NC(=O)OC(C)(C)C)cc1]

### 4-Methoxybenzyl O-[(6-trifluoromethyl-1H-benzotriazol-1-yloxy)methyl]-N-(tert-butoxycarbonyl)-L-tyrosinate. 1-5-4

The compound can be prepared in analogy to 1-5-3 from L-tyrosine. In the event it was isolated from the preparation of 1-5-4, where a partial racemisation had occurred during the synthesis or stereochemically impure tyrosine had been used.

MS (ESI⁻): m/e=639 (M+Na⁺), 617 (M+H⁺), 561 (M+H⁺−C₄H₈), 121 (C₈H₉O⁺).

¹H NMR (DICHLOROMETHANE-d₂, 400 MHz): δ (ppm) 8.13 (d, J=8.8 Hz, 1H, Bt 4-H), 7.59 (dd, J=8.8, 1.3 Hz, 1H, Bt 5-H), 7.48 (s, 1H, Bt 7-H), 7.28 (d, J=8.6 Hz, 2H, Mbn 3-H), 7.07 (d, J=8.3 Hz, 2H, Ar—H), 6.98 (d, J=8.6 Hz, 2H, Ar—H), 6.84-6.90 (m, 2H, Mbn 4-H), 6.02-6.08 (m, 2H, OCH₂O), 5.12 (d, J=11.6 Hz, 1H, Mbn 1-H), 5.06 (d, J=11.6 Hz, 1H, Mbn 1-H), 5.02 (d, J=8.1 Hz, 1H, NH), 4.54 (dt, J=8.1, 5.8 Hz, 1H, 2-H), 3.77 (s, 3H, Mbn OMe), 3.11 (dd, J=13.9, 5.8 Hz, 1H, 3-H), 3.03 (dd, J=13.6, 5.8 Hz, 1H, 3-H), 1.40 (s, 9H, Boc).

¹⁹F NMR (DICHLOROMETHANE-d₂, 376 MHz): δ (ppm)-62.3.

¹³C NMR (DICHLOROMETHANE-d₂, 101 MHz): δ ppm 171.6 (C-1), 159.9 (Mbn C-5), 155.0 (2C1), 154.6 (3C-4), 144.6 (Bt C-3a), 131.8 (3C-1), 131.0 (3C-2), 130.5 (Mbn C-3), 130.1 (q, ²J_{CF}=32.8 Hz, Bt C-6), 128.2 (Bt C-7a), 127.5 (Mbn C5), 123.7 (q, ¹J_{CF}=273.2 Hz, CF₃), 121.2 (Bt C-4, Bt C-5), 115.9 (3C-3), 113.8 (Mbn C-4), 107.8 (q, ³J_{CF}=4.8 Hz, Bt C-7), 99.0 (OCH₂O) 79.7 (2C-2), 66.9 (Mbn C-1), 55.2 (Mbn OMe), 54.6 (C-2), 37.2 (C-3) 28.0 (2C-3).

### Example 6

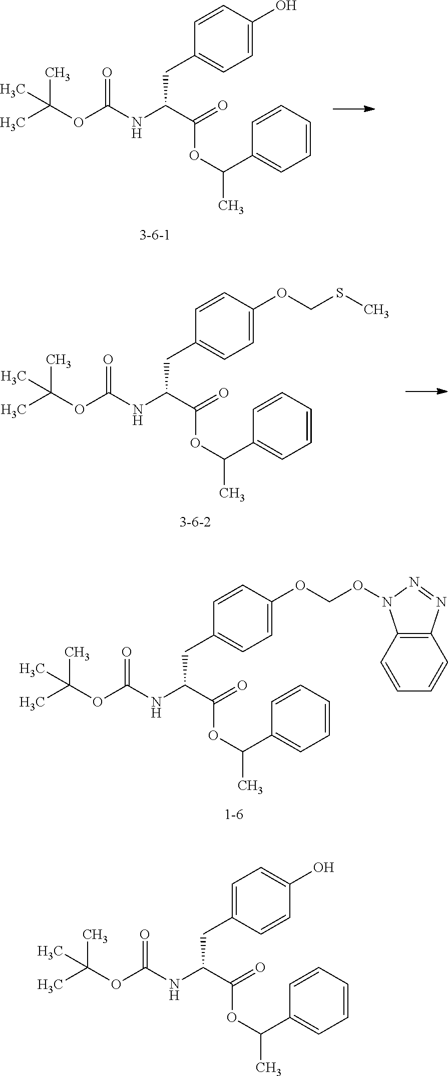

### alpha-Methyl benzyl N-(tert-butoxycarbonyl)-D-tyrosinate. 3-6-1

To 500 mg (1.78 mmol) Boc-D-Tyr-OH in 15 ml N,N-dimethylformamide were added 295.4 mg (0.91 mmol) caesium carbonate and the mixture stirred at r.t. for 0.5 h. 328.9 mg (1.78 mmol) 1-phenylethyl bromide were added and the mixture stirred at r.t. over night. The mixture was evaporated in the vacuum at 50° C. The residue was dissolved in ethyl acetate and water. The aqueous phase was separated and extracted with ethyl acetate. The combined extracts were dried and evaporated in the vacuum at 50° C. to yield 725 mg (106%) of 3-6-1 as mixture of diastereomers.

MS (ES+): m/e=386.55 (M+H⁺), 771.71 (2M+H⁺).

¹H-NMR (400 MHz, CHLOROFORM-d): δ (ppm) 1.37-1.47 (m, 9H), 1.51 (d, 1.5H), 1.57 (d, 1.5H), 3.02-3.13 (m, 2H), 4.48-4.63 (m, 1H), 4.84-5.05 (m, 1H), 5.82-5.99 (m, 1H), 6.55-6.66 (m, 1H), 6.68-6.84 (m, 2H), 7.00 (m, 1H), 7.29-7.41 (m, 5H).

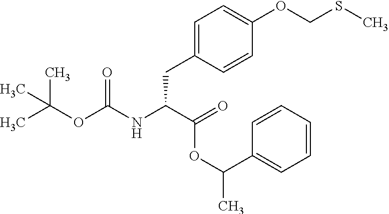 [SMILES: CSCOc1ccc(C[C@@H](NC(=O)OC(C)(C)C)C(=O)OC(C)c2ccccc2)cc1]

### alpha-Methyl benzyl N-(tert-butoxycarbonyl)-O-[(methylsulfanyl)methyl]-D-tyrosinate

### 3-6-2

101 mg (0.26 mmol) 3-6-1 and 4.05 mg (0.03 mmol) sodium iodide were dissolved in 2 ml N,N-dimethylformamide and cooled for 10 minutes with ice. 0.30 ml (0.30 mmol) potassium tert-butoxide, 1.0M in tetrahydrofuran were added and the mixture stirred for 60 min. 0.03 ml (0.30 mmol) chloromethyl methyl sulfide were added and the mixture stirred at r.t. for 48 h and at 66° C. for 5 h. The mixture was diluted with ethyl acetate, extracted with sodium chloride solution, dried and evaporated. The residue was chromatographed (SNAP 5 g, n-hexane/ethyl acetate 85:15) to give 45 mg (27%) 3-6-2 as mixture of diastereomers.

The reaction was repeated with 400 mg of 3-6-1 to give 350 mg (76%) of 3-6-2.

MS (ESI⁺): m/e=446.54 (M+H⁺), 891.69 (2M+H⁺).

¹H-NMR (400 MHz, CHLOROFORM-d): δ (ppm) 7.28-7.42 (m, 6H), 7.04-7.16 (m, 1H), 6.84-6.93 (m, 1H), 6.70-6.83 (m, 1H), 5.84-5.97 (m, 1H), 5.13 (s, 2H), 4.90-5.03 (m, 1H), 4.58 (m, 1H), 3.02-3.13 (m, 2H), 2.24-2.26 (m, 3H), 1.55-1.60 (m, 1.5H), 1.51 (m, 1.5H), 1.38-1.47 (m, 9H).

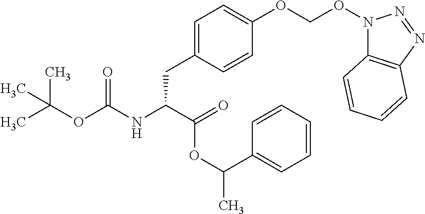 [SMILES: CC(OC(=O)[C@@H](Cc1ccc(OCOn2nnc3ccccc32)cc1)NC(=O)OC(C)(C)C)c1ccccc1]

### alpha-Methyl benzyl O-[(1H-benzotriazol-1-yloxy)methyl]-N-(tert-butoxycarbonyl)-D-tyrosinate. 1-6

120 mg (0.27 mmol) 3-6-2 were reacted as described for 1-2-1. The reaction mixture was directly applied to Isolute and chromatographed on a Biotage system (Isolera Four, SNAP 10 g, n-hexane to n-hexane/ethyl acetate 1:4) to give 55 mg (34.5%) 1-6 as mixture of diastereomers.

MS (ESI⁺): m/e=533.64 (M+H⁺).

¹H NMR (CHLOROFORM-d, 300 MHz): δ (ppm) 7.96-8.04 (m, 1H), 7.28-7.43 (m, 9H), 7.00-7.21 (m, 4H), 6.81-6.93 (m, 2H, Ar—H), 5.83-6.09 (m, 3H, OCH₂O, OCH), 4.91-5.09 (m, 1H, NH), 4.51-4.68 (m, 1H, 2-H), 2.89-3.23 (m, 2H, 3-H), 1.53 and 1.59 (d, J=6.8 Hz, CH₃), 1.43 (s, 9H).

¹³C NMR (CHLOROFORM-d, 75 MHz): δ (ppm) 171.0, 171.0 (C-1), 1551, 155.1 (3C-4), 155.0 (2C-1), 143.5 (Bt C-3a), 140.8, 140.6 (1 ipso), 131.4, 131.0 (3C-1), 130.9, 130.9 (3C-2), 128.8 (Bt C-3a), 128.6, 128.6 (1 meta), 128.3, 128.3 (1 para), 128.2 (Bt C-6), 126.6, 126.1 (1 ortho), 124.7 (Bt C-5), 120.1, 120.1 (Bt C-4), 116.1, 116.0 (3C-3), 109.1, 109.0 (Bt C-7), 99.0, 98.9 (OCH₂O), 80.0 (2C-2), 73.7, 73.7 (1C-1), 54.6, 54.3 (C-2), 37.7, 37.3 (C-3), 28.3 (2C-3), 22.1, 21.9 (1 CH₃).

### Example 7

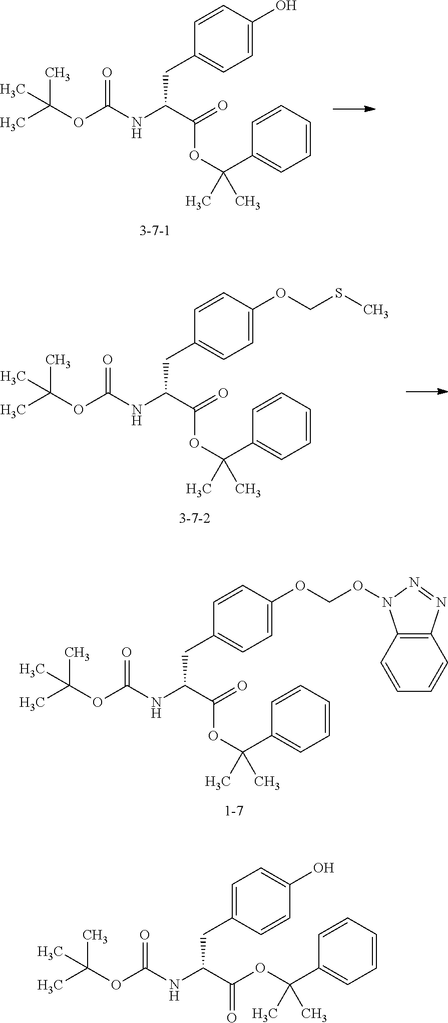

### alpha,alpha-Dimethylbenzyl N-(tert-butoxycarbonyl)-D-tyrosinate. 3-7-1

To 511.3 mg (1.82 mmol) Boc-D-Tyr-OH in 51 ml dichloromethane were added 1020.0 mg (3.6 mmol) 2-phenylisopropyltrichloroacetamidate (Tetrahedron Lett. 1993, 34, 323-326; WO 2008/048970, 66) in 10 ml cyclohexane. After stirring for 20 h, the mixture was concentrated and separated by chromatography (10 g, n-hexane to n-hexane/ethyl acetate 2:3) to give 772 mg (106%) 3-7-1.

MS (ESI⁺): m/e=400.54 (M+H⁺).

¹H-NMR (300 MHz, CHLOROFORM-d): δ (ppm) 7.28-7.34 (m, 5H), 7.04 (d, 2H), 6.75 (d, 2H), 4.85-4.97 (m, 1H), 4.42-4.55 (m, 1H), 2.92-3.05 (m, 2H), 1.70 (s, 3H), 1.60 (s, 3H), 1.42 (s, 9H).

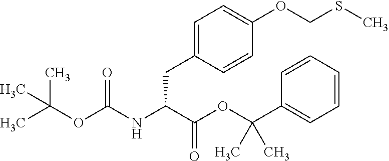 [SMILES: CSCOc1ccc(C[C@@H](NC(=O)OC(C)(C)C)C(=O)OC(C)(C)c2ccccc2)cc1]

### alpha,alpha-Dimethylbenzyl N-(tert-butoxycarbonyl)-O-[(methylsulfanyl)methyl)]-D-tyrosinate. 3-7-2

A solution of 1,228 g (3.07 mmol) 3-7-1, 47 mg (0.32 mmol) sodium iodide and 7 ml N,N-dimethylformamide was cooled to 0° C. in an ice bath. A suspension of 388 mg (3.46 mmol) potassium tert-butylate in 3 ml tetrahydrofuran was added. 293 μl (3.54 mmol) chloro dimethyl sulfide were added. The mixture was allowed to come to r.t. and stirred for 3 h. ethyl acetate was added. The mixture was extracted with water and sodium chloride solution, dried and concentrated in the vacuum. Chromatography over 10 g basic silica gel (n-hexane to n-hexane/ethyl acetate 2:3) gave 139 mg (8%) of 3-7-2 and 208 mg (14%) starting material.

MS (CI⁺): m/e=477.61 (M+NH₄⁺), 936.71 (2M+NH₄⁺).

¹H-NMR (300 MHz, CHLOROFORM-d): δ (ppm) 7.30-7.37 (m, 5H), 7.11 (d, 2H), 6.88 (d, 2H), 5.13 (s, 2H), 4.49 (d, 1H), 2.98-3.12 (m, 2H), 2.26 (s, 3H), 1.76 (m, 3H), 1.73 (m, 3H), 1.42 (s, 9H).

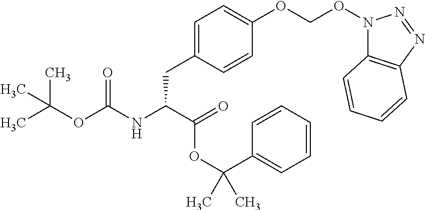 [SMILES: CC(C)(C)OC(=O)N[C@H](Cc1ccc(OCOn2nnc3ccccc32)cc1)C(=O)OC(C)(C)c1ccccc1]

### alpha,alpha-Dimethyl benzyl O-[(1H-benzotriazol-1-yloxy)methyl]-N-(tert-butoxy-carbonyl)-D-tyrosinate. 1-7

120 mg (0.26 mmol) 3-7-2 were reacted as described for 1-2-1. The reaction mixture was directly applied to Isolute and chromatographed on a Biotage system (Isolera Four, SNAP 10 g, n-hexane to n-hexane/ethyl acetate 1:4) to give 35 mg (23.3%) 1-7.

MS (ESI⁺): m/e=547.36 (M+H⁺).

¹H NMR (CHLOROFORM-d, 300 MHz): δ (ppm) 7.95-8.04 (m, 1H), 7.11-7.41 (m, 11H), 7.06 (d, J=8.7 Hz, 2H, Ar—H), 6.03 (s, 2H, OCH₂O), 4.98 (d, J=8.1 Hz, 2H. NH), 4.47-4.63 (m, 1H, 2-H), 3.15 (dd, J=13.9, 6.4 Hz, 1H, 3-H), 3.04 (dd, J=13.9, 6.0 Hz, 1H), 3-H, 1.78 (s, 3H, CH₃), 1.75 (s, 3H, CH₃), 1.42 (s, 9H, Boc).

¹³C NMR (CHLOROFORM-d, 75 MHz): δ (ppm) 170.4 (C-1), 155.2 (3C-4), 155.1 (2C-1), 144.9 (Cu ipso), 143.5 (Bt C-3a), 131.6 (3C-1), 131.0 (3C-2), 128.8 (Bt C-7a), 128.3 (Cu meta), 128.3 (Bt C-6), 127.3 (Cu para), 124.7 (Bt C-5), 124.4 (Cu ortho), 120.1 (Bt C-4), 116.0 (3C-3), 109.0 (Bt C-7), 99.0 (OCH₂O), 83.4 (Cu C), 79.9 (2C-2), 54.8 (C-2), 37.6 (C-3), 28.8 (Cu CH₃), 28.3 (2C-3), 27.9 (Cu CH₃).

### 1.8 Example 8

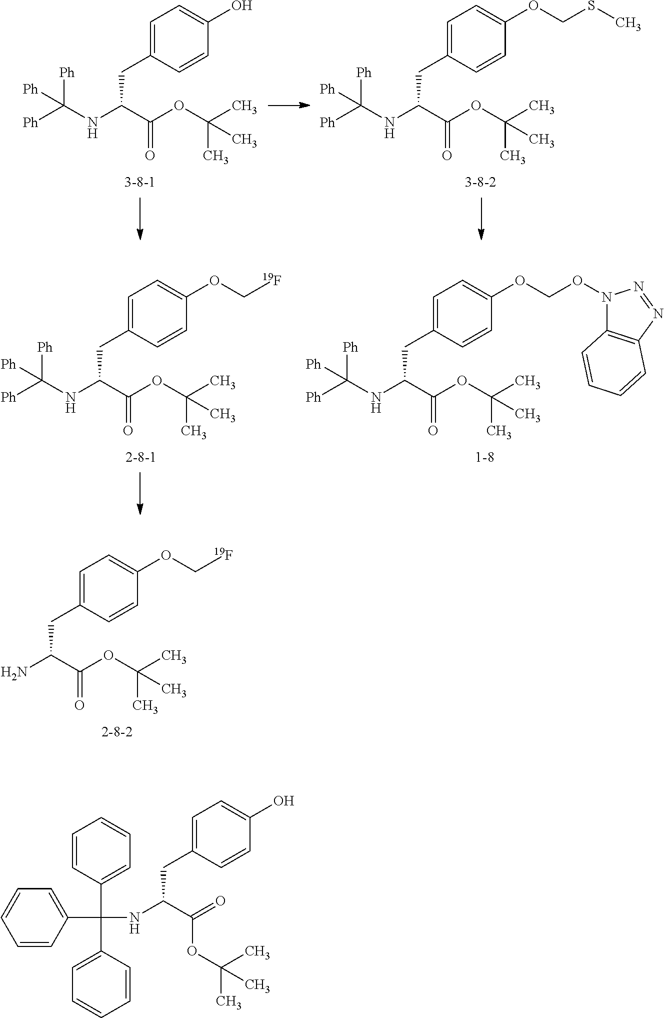 [SMILES: CC(C)(C)OC(=O)[C@@H](Cc1ccc(O)cc1)NC(c1ccccc1)(c1ccccc1)c1ccccc1 ; CSCOc1ccc(C[C@@H](NC(c2ccccc2)(c2ccccc2)c2ccccc2)C(=O)OC(C)(C)C)cc1 ; CC(C)(C)OC(=O)[C@@H](Cc1ccc(OC[19F])cc1)NC(c1ccccc1)(c1ccccc1)c1ccccc1 ; CC(C)(C)OC(=O)[C@@H](Cc1ccc(OCOn2nnc3ccccc32)cc1)NC(c1ccccc1)(c1ccccc1)c1ccccc1 ; CC(C)(C)OC(=O)[C@H](N)Cc1ccc(OC[19F])cc1 ; CC(C)(C)OC(=O)[C@@H](Cc1ccc(O)cc1)NC(c1ccccc1)(c1ccccc1)c1ccccc1]

### tert-Butyl N-trityl-D-tyrosinate. 3-8-1

This compound was synthesized as described (Journal of Labelled Compounds and Radiopharmaceuticals 2004; 47, 477-483) using D-Tyrosine.

MS (ESI⁺): m/e=243.47 (Ph₃C⁺).

MS (ES⁻): m/e=524.66 (M+HCOO⁻), 957.71 (2M−H⁺).

¹H NMR (DMSO-d6, 300 MHz): δ (ppm) 9.16 (s, 1H, OH), 7.29-7.37 (m, 6H), 7.18-7.27 (m, 6H), 7.10-7.18 (m, 3H), 6.83 (d, J=8.3 Hz, 2H), 6.60 (d, J=8.5 Hz, 2H), 3.09-3.21 (m, 1H, 2-H), 2.64 (d, J=9.2 Hz, 1H, NH), 2.56 (dd, J=13.6, 7.7 Hz, 1H, 3-H), 2.36 (dd, J=13.6, 6.0 Hz, 1H, 3-H), 1.01 (s, 9H, OtBu).

¹³C NMR (101 MHz, CHLOROFORM-d): δ (ppm) 173.9 (C-1), 154.4 (3C-4), 146.4 (Tr C-2), 131.2 (3C-2), 129.8 (3C-1), 128.9 (Tr C-3), 127.8 (Tr C-4), 126.4 (Tr C-5), 114.9 (3C-3), 80.5 (1C-1), 71.2 (Tr C-1), 58.2 (C-2), 41.3 (C-3), 27.9 (1C-2).

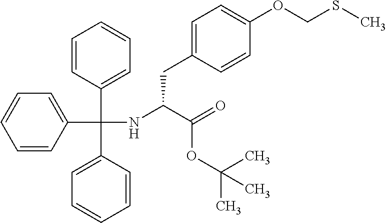 [SMILES: CSCOc1ccc(C[C@@H](NC(c2ccccc2)(c2ccccc2)c2ccccc2)C(=O)OC(C)(C)C)cc1]

### tert-Butyl O-[(methylsulfanyl)methyl]-N-trityl-D-tyrosinate. 3-8-2

A solution of 1.20 g (2.50 mmol) 3-8-1, 39 mg (0.26 mmol) sodium iodide and 5.5 ml N,N-dimethylformamide was cooled to 0° C. in an ice bath. A suspension of 365 mg (3.25 mmol) potassium tert-butylate in 3 ml tetrahydrofuran was added. After 10 min 238 μl (2.89 mmol) chloro dimethyl sulfide were added. The mixture was allowed to come to r.t. and stirred for 20 h. Ethyl acetate was added. The mixture was extracted with water, dried and concentrated in the vacuum. This gave 1.46 g (92%) of 3-8-2 with 85% purity.

MS (ESI⁺): m/e=243.47 (Ph₃C⁺), 540.66 (M+H⁺).

¹H NMR (CHLOROFORM-d, 400 MHz): δ (ppm) 7.41-7.48 (m, 6H), 7.18-7.25 (m, 6H), 7.11-7.18 (m, 5H), 6.88 (d, J=8.6 Hz, 2H), 5.13 (s, 2H, OCH₂S), 3.42-3.52 (m, 1H, 2-H), 2.83 (dd, J=13.4, 6.8 Hz, 1H, 3-H), 2.76 (dd, J=13.4, 6.1 Hz, 1H, 3-H), 2.58 (d, J=7.6 Hz, 1H, NH), 2.24 (s, 3H, SCH₃), 1.06 ppm (s, 9H, tBu).

¹³C NMR (CHLOROFORM-d, 75 MHz,): δ (ppm) 173.7 (C-1), 155.7 (3C-4), 146.3 (Tr C-2), 131.1 (3C-1), 131.0 (3C-2), 128.8 (Tr C-3), 127.8 (Tr C-4), 126.3 (Tr C-5), 115.6 (3C-3), 80.4 (1C-1), 72.5 (SCH₂O), 71.2 (Tr C-1), 58.1 (C-2), 41.3 (C-3), 27.8 (1C-2), 14.5 (SCH₃).

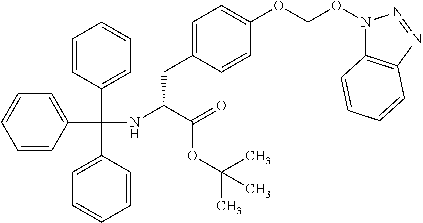 [SMILES: CC(C)(C)OC(=O)[C@@H](Cc1ccc(OCOn2nnc3ccccc32)cc1)NC(c1ccccc1)(c1ccccc1)c1ccccc1]

### tert-Butyl O-[(1H-benzotriazol-1-yloxy)methyl]-N-trityl-D-tyrosinate. 1-8

240 mg (0.45 mmol) 3-8-2 were reacted as described for 1-2-1. The reaction mixture was directly applied to Isolute and chromatographed on a Biotage system (Isolera Four, SNAP 25 g, n-hexane to n-hexane/ethyl acetate 1:4) to give 65 mg (23.3%) 1-8.

Waters Autopurificationsystem Pump 254, Sample Manager 2767, CFO, DAD 2996, ELSD 2424, SQD 3001, Luna C18(2) 5 μm 150×21.2 mm, A=water+0.1% formic acid, B=acetonitrile, 0-1 min 70% B, 1-12 min 70-100% B, 25 ml/min, r.t., 54 mg/1 ml dimethyl sulfoxide/methanol 1:1, 1×1 ml, DAD scan range 210-400 nm, MS ESI+, ESI−, scan range 160-1000 m/z, ELSD. The peak at 13.0-13.2 min was collected to give 12 mg (3.9%) of 1-8 with 97.4% purity.

MS (ESI⁺): m/e=243.47 (Ph₃C⁺), 627.63 (M+H⁺).

¹H NMR (CHLOROFORM-d, 400 MHz): δ (ppm) 7.99 (d, J=8.1 Hz, 1H, Bt-H), 7.47 (d, J=7.1 Hz, 6H, Tr-H), 7.28-7.34 (m, 2H, Bt-H), 7.21-7.24 (m, 6H, Tr-H), 7.13-7.20 (m, 4H, Bt-H, Ar—H), 7.09 (d, J=8.6 Hz, 2H, Ar—H), 6.01-6.08 (m, 2H, OCH₂O), 3.54 (dd, J=7.1, 5.8 Hz, 1H, 2-H), 2.90 (dd, J=13.4, 7.1 Hz, 1H, 3-H), 2.82 (dd, J=13.9, 5.8 Hz, 1H, 3-H), 2.62 (br. s, 1H, NH), 1.11 (s, 9H, tBu-H).

¹³C NMR (DICHLOROMETHANE-d2, 151 MHz): δ=173.6 (C-1), 155.5 (3C-4), 146.8 (Tr C-2), 143.9 (Bt C-3a), 133.6 (3C-1), 131.9 (3C-2), 129.2 (Tr C-3), 129.1 (Bt C-7a), 128.6 (Bt C-6), 128.2 (Tr C-4, 126.8 (Tr C-5), 125.0 (Bt C-5), 120.2 (Bt C-4), 116.1 (3C-3), 109.5 (Bt C-7), 99.7 (OCH₂O), 80.8 (1C-1), 71.7 (Tr C-1), 58.4 (C-2), 41.4 (C-3), 28.1 (1C-2).

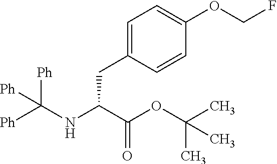 [SMILES: CC(C)(C)OC(=O)[C@@H](Cc1ccc(OCF)cc1)NC(c1ccccc1)(c1ccccc1)c1ccccc1]

### tert-Butyl O-(fluoromethyl)-N-trityl-D-tyrosinate. 2-8-1

To 200.0 mg (0.42 mmol) 3-8-1 in 4 ml N,N-dimethylformamide cooled to 5° C. were added 16.7 mg (0.42 mmol) sodium hydride (60%). The mixture was stirred for 30 min at 5-10° C. A solution of 167 mg (1.48 mmol) bromofluoromethane in 4 ml N,N-dimethylformamide was added and the mixture stirred for 2 h at 5-10° C. and 2 h at r.t. The mixture was partitioned between dichloromethane and water, the aqueous phase was extracted with dichloromethane, the combined organic phases dried and the solvent evaporated to give 214 mg (90%) 2-8-1.

MS (ESI⁺): m/e=243.47 (Ph₃C⁺) only.

¹⁹F NMR (376 MHz, CHLOROFORM-d): δ (ppm)-148.02 (t, J=55.1 Hz).

¹H NMR (CHLOROFORM-d, 300 MHz): δ (ppm) 7.44 (d, J=7.2 Hz, 6H), 7.11-7.25 (m, 11H), 7.00 (d, J=8.5 Hz, 2H), 5.69 (d, ¹J_{HF}=55.0 Hz, 2H), 3.42-3.55 (m, 1H, 2-H), 2.85 (dd, J=13.9, 6.4 Hz, 1H, 3-H), 2.78 (dd, J=13.6, 5.8 Hz, 1H, 3-H), 2.52-2.64 (m, 1H, NH), 1.07 (s, 9H, tBu).

¹³C NMR (101 MHz, CHLOROFORM-d) δ ppm 173.6 (C-1), 162.6 (s, 1C), 155.6 (d, ³J_{CF}=2.4 Hz, 3C-4), 146.3 (Tr C2), 133.0 (3C-1), 131.3 (3C-2), 128.8 (Tr C-3), 127.9 (Tr C4), 126.4 (Tr C-5), 116.4 (3C-3), 101.0 (d, ¹J_{CF}=218.9 Hz, OCH₂F), 80.6 (1C-1), 71.3 (Tr C-1), 58.0 (C-2), 41.3 (C-3), 36.5 (s, 1C), 29.8 (s, 1C), 27.9 (1C-2).

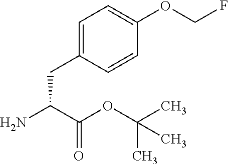 [SMILES: CC(C)(C)OC(=O)[C@H](N)Cc1ccc(OCF)cc1]

### tert-Butyl O-(fluoromethyl)-D-tyrosinate. 2-8-2

To a solution of 80 mg (0.16 mmol) 2-8-1 in 0.8 ml acetic acid were added 0.2 ml water and the solution stirred for 2 h at r.t. Water was added and the precipitate was filtered off. The filtrate was neutralized with sodium hydrogen-carbonate solution and extracted with ethyl acetate. The combined organic phases were dried over sodium sulfate and the solvent evaporated to give 30 mg (71%) 2-8-2.

Agilent: Prep 1200, 2×Prep Pump, DLA, MWD, Prep FC, XBrigde C18 5 μm 150×19 mm, A=water+0.2% ammonia, B=methanol, 0-1 min 10% B, 1-8 min 10-80% B, 8-8.1 min 80-100% B, 8.1-10 min 100% B, 25 ml/min, r.t., 30 mg/1 ml dimethyl sulfoxide/methanol 1:1, 1×1 ml, UV 219 nm. The Peak at 5-1.33 min was collected to give 17 mg (36%) of 2-8-2 with 99.3% purity.

MS (ES+): m/e=214.42 (M+H⁺−O₄H₈), 270.51 (M+H⁺), 539.62 (2M+H⁺).

¹⁹F NMR (376 MHz, CHLOROFORM-d): δ (ppm)-148.29 (t, J=53.9 Hz).

¹H NMR (CHLOROFORM-d, 400 MHz): δ (ppm) 7.10 (d, J=8.5 Hz, 2H, Ar—H), 6.94 (d, J=8.5 Hz, 2H, Ar—H), 5.61 (d, ²J_{HF}=54.7 Hz, 2H), 3.50 (dd, J=7.5, 5.5 Hz, 1H, 2-H), 2.92 (dd, J=13.6, 5.8 Hz, 1H, 3-H), 2.74 (dd, J=13.8, 7.5 Hz, 1H, 3-H), 1.65 (br. s, 2H, NH), 1.36 (s, 9H, tBu-H).

¹³C NMR (101 MHz, CHLOROFORM-d) δ (ppm) 174.25 (C-1) 155.67 (3C-4) 155.64 (2C-1) 132.71 (3-10) 130.60 (3C-2) 116.70 (d, ⁴J_{CF}=1.3 Hz, 3 C-3), 100.88 (d, ¹J_{CF}=218.9 Hz, OCH₂O) 81.21 (1C-1) 56.32 (C-2) 40.32 (C-3) 28.02 (1C-2).

### 1.9 Example 9

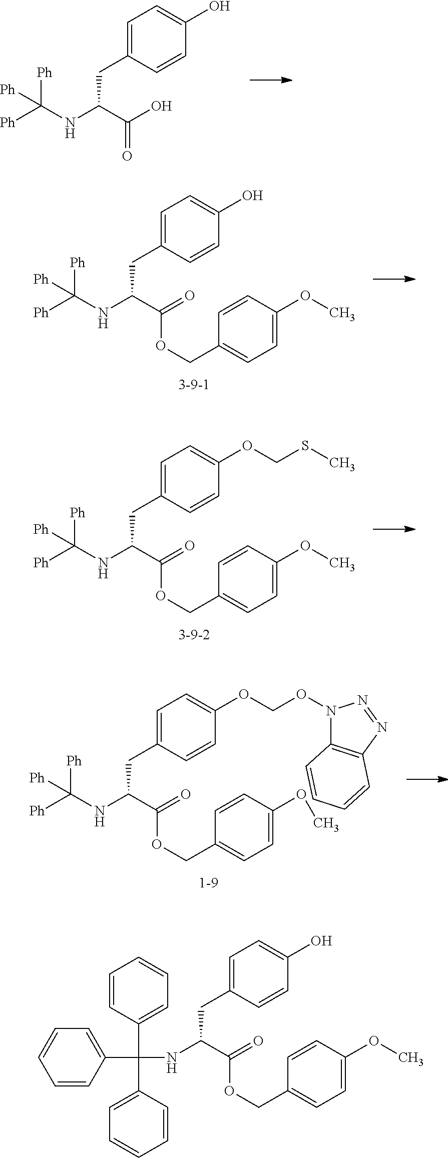 [SMILES: O=C(O)[C@@H](Cc1ccc(O)cc1)NC(c1ccccc1)(c1ccccc1)c1ccccc1 ; COc1ccc(COC(=O)[C@@H](Cc2ccc(O)cc2)NC(c2ccccc2)(c2ccccc2)c2ccccc2)cc1 ; COc1ccc(COC(=O)[C@@H](Cc2ccc(OCSC)cc2)NC(c2ccccc2)(c2ccccc2)c2ccccc2)cc1 ; COc1ccc(COC(=O)[C@@H](Cc2ccc(OCOn3nnc4ccccc43)cc2)NC(c2ccccc2)(c2ccccc2)c2ccccc2)cc1 ; COc1ccc(COC(=O)[C@@H](Cc2ccc(O)cc2)NC(c2ccccc2)(c2ccccc2)c2ccccc2)cc1]

### 4-Methoxybenzyl N-trityl-D-tyrosinate. 3-9-1

265 mg (0.53 mmol) N-trityl-D-tyrosine (Liebigs Ann. Chem. 1988, 1083-1084) were dissolved in 4.4 ml N,N-dimethylformamide. 89 mg (0.27 mmol) cesium carbonate were added and the mixture stirred for 30 min. 107 mg (0.54 mmol) 4-methoxybenzyl bromide were added and the mixture stirred for 16 h. Further 54 mg (0.27 mmol) 4-methoxybenzyl bromide were added and the mixture stirred at 40° C. for 4 h. The mixture was diluted with ethyl acetate and extracted with water. The aqueous phase was neutralized with acetic acid to pH 5 and extracted with ethyl acetate. The organic solutions were combined, dried over sodium sulfate and concentrated. The residue was purified by chromatography over 10 g silica gel with hexane/ethyl acetate 100-80/20-60/40 to give 215 mg (67%) 3-9-1.

A previous preparation using 200 mg N-trityl-D-tyrosine gave 83 mg (34%) 3-9-1.

MS (ESI⁻): m/e=544.33 (M+H⁺).

MS (ESI⁻): m/e=588.18 (M+HCOO).

¹H-NMR (400 MHz, CHLOROFORM-d): δ (ppm) 7.40-7.49 (m, 6H), 7.13-7.26 (m, 9H), 6.91-7.02 (m, 4H), 6.79-6.84 (m, 2H), 6.68-6.73 (m, 2H), 4.92 (br. s., 1H), 4.42 (d, 1H), 4.20 (d, 1H), 3.81 (s, 3H), 3.51-3.61 (m, 1H), 2.94 (dd, 1H), 2.84 (dd, 1H), 2.59-2.69 (m, 1H).

¹³C-NMR (101 MHz, CHLOROFORM-d): δ (ppm) 174.4, 159.5, 154.4, 145.9, 130.9, 127.9, 128.8, 129.4, 127.5, 130.0, 126.3, 113.7, 115.0, 71.0, 66.1, 58.3, 55.3, 41.4.

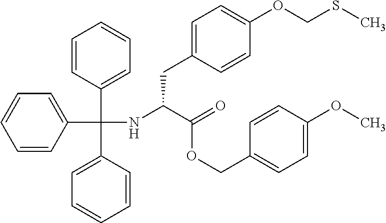 [SMILES: COc1ccc(COC(=O)[C@@H](Cc2ccc(OCSC)cc2)NC(c2ccccc2)(c2ccccc2)c2ccccc2)cc1]

### 4-Methoxybenzyl O-[(methylsulfanyl)methyl]-N-trityl-D-tyrosinate. 3-9-2

298 mg (0.55 mmol) 4-Methoxybenzyl N-trityl-D-tyrosinate 3-9-1 were dissolved in 4.5 ml N,N-dimethylformamide. 357 mg (1.1 mmol) cesium carbonate were added and the mixture stirred for 16 h. 64 mg (0.66 mmol) chloro dimethyl sulfide were added and the mixture stirred for 16 h. Further 23 mg (0.24 mmol) chloro dimethyl sulfide were added and the mixture stirred for 2 h. Further 23 mg (0.24 mmol) chloro dimethyl sulfide and 200 mg cesium carbonate were added and the mixture stirred for 20 h. The mixture was concentrated, diluted with ethyl acetate and extracted with water. The organic solutions were washed with saturated sodium chloride solution, dried over sodium sulfate and concentrated to give 335 mg.

MS (ESI⁻): m/e=604.23 (M+H⁺).

MS (ESI⁻): m/e=648.00 (M+HCOO).

¹H-NMR (300 MHz, CHLOROFORM-d): δ (ppm) 7.38-7.50 (m, 6H), 7.12-7.26 (m, 9H), 7.06 (d, 2H), 6.94 (d, 2H), 6.77-6.88 (m, 4H), 5.12 (s, 2H), 4.41 (d, 1H), 4.20 (d, 1H), 3.78-3.83 (m, 3H), 3.52-3.61 (m, 1H), 2.86 (dd, 1H), 2.76 (dd, 1H), 2.68 (d, 1H), 2.23-2.30 (m, 3H).

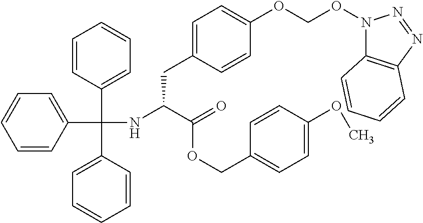 [SMILES: COc1ccc(COC(=O)[C@@H](Cc2ccc(OCOn3nnc4ccccc43)cc2)NC(c2ccccc2)(c2ccccc2)c2ccccc2)cc1]

### 4-Methoxybenzyl O-[(1H-benzotriazol-1-yloxy)methyl]-N-trityl-D-tyrosinate. 1-9

160 mg (0.27 mmol) 3-9-2 were reacted as described for 1-2-1. The reaction mixture was directly applied to Isolute and chromatographed on a Biotage system (Isolera Four, SNAP 10 g, n-hexane to n-hexane/ethyl acetate 4:1) to give 73 mg (40%) 1-9.

The material was further purified by HPLC (Waters Autopurificationsystem: Pump 254, Sample Manager 2767, CFO, DAD 2996, ELSD 2424, SQD 3001, XBrigde C18 5 μm 100×30 mm, A=water+0.1% formic acid, B=acetonitrile, 0-1 min 50% B, 1-8 min 50-100% B, 50 ml/min, r.t., 69 mg/2.1 ml dimethyl sulfoxide/methanol 1:1, 3×0.7 ml, DAD scan range 210-400 nm, MS ESI+, ESI−, scan range 160-1000 m/z, ELSD). The peak at 7.8-8.1 min was collected to give 16 mg (9%) of 1-9 with >99% purity.

MS (ESI⁻): m/e=691.26 (M+H⁺).

MS (ESI⁻): m/e=736.15 (M+HCOO).

¹H NMR (CHLOROFORM-d, 300 MHz): δ (ppm) 7.98 (d, J=8.3 Hz, 1H, Bt 7-H), 7.44 (d, J=7.0 Hz, 6H, Tr o-H), 7.09-7.33 (m, 14H, Tr m-H, p-H, Ar 2-H, Bt H-4,5,6), 7.02 (d, J=8.3 Hz, 2H, Mbn 2-H), 6.99 (d, J=8.3 Hz, 2H, Ar 3-H), 6.78 (d, J=8.7 Hz, 2H, Mbn 3-H), 6.01 (br. s, 2H, OCH₂O), 4.46 (d, J=12.1 Hz, 1H, Mbn 1-H), 4.26 (d, J=11.9 Hz, 1H, Mbn 1-H), 3.75 (s, 3H, Mbn OMe), 3.55-3.65 (m, 1H, 2-H), 2.95 (s, 2H).

¹³C NMR (CHLOROFORM-d, 101 MHz): δ (ppm) 174.2 (C-1), 159.6 (Mbn C-5), 155.1 (3C-4), 145.9 (Tr C-2), 143.5 (Bt C-3a), 132.6 (3C-1), 131.4 (3C-2), 130.2 (Mbn C-3), 128.8 (Tr C-3), 128.4 (Bt C-6), 128.0 (Bt C-7a), 127.9 (Tr C-4), 127.5 (Mbn C-2), 126.5 (Tr C-5), 124.7 (Bt C-5), 120.0 (Bt C-4), 115.9 (3C-3), 113.8 (Mbn C-4), 109.1 (Bt C-7), 99.2 (OCH₂O), 71.2 (Tr C-1), 66.3 (Mbn C-1), 58.1 (C-2), 55.3 (Mbn OMe), 41.4 (C-3).

### 1.10 Example 10

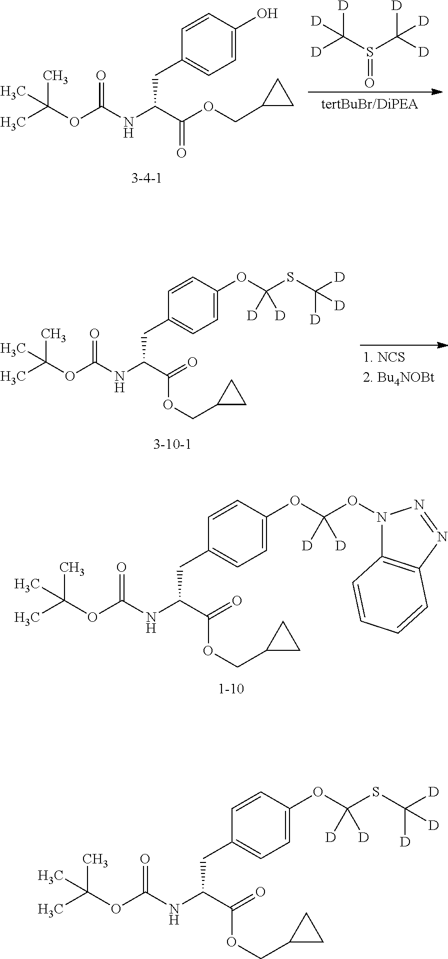

### Cyclopropylmethyl N-(tert-butoxycarbonyl)-O—[([²H₃]methylsulfanyl)[²H₂]methyl]-D-tyrosinate. 3-10-1

A solution of 1.00 g (2.98 mmol) 3-4-1 was dissolved in 10 ml [²H₆]dimethyl sulfoxide and 5.1 ml (29.8 mmol) ethyl-diisopropyl amine added. The mixture was heated to 45° C. under Argon atmosphere and the reaction started by addition of 3.46 ml 4.09 mmol) tert-butyl bromide. It was kept at this temperature for 72 h and then filtered. The filtrate was diluted with dichloromethane and washed with sat. sodium hydrogen carbonate. The organic phase was evaporated and the residue chromatographed on a Biotage system: (Flash40+M cartridge, 40 ml/min, 3CV dichloromethane, dichloromethane to dichloromethane/methanol 4:1 in 12CV, 15CV=1980 ml) gave 1.08 g which was further purified on an Autopurification System (Waters: 2525 Binary Gradient Module, Detector: MS Micromass ZQ, UV Photo Diode Array 2996, 210-350 nm; X-Bridge Prep 50×50 mm, C18 5 μm; Gradient: acetonitrile from 50% acetonitrile to 80%, water 0.1% formic acid; 9 Min, 60 ml/min) to give 153 mg (12%) of 3-10-1 as a clear oil. Re-chromatography of an impure fraction gave another 8 mg of 3-10-1.

MS (ESI⁺): m/e=423 (M+Na⁺), 401 (M+H⁺), 345 (M+H⁺−C₄H₈), 301 (M+H⁺−C₄H₈−CO₂).

¹H NMR (DICHLOROMETHANE-d₂, 600 MHz): δ (ppm) 7.14 (d, J=8.7 Hz, 2H, Ar—H), 6.92 (d, J=8.7 Hz, 2H, Ar—H), 5.15-5.17 (m, 0.09H, OCHDS), 4.99-5.09 (m, 1H, NH), 4.48-4.57 (m, 1H, 2-H), 3.97 (mc, 2H, OCH₂), 2.97-3.15 (m, 2H, 3-H), 2.22-2.25 (m, 0.09H, SCHD₂), 1.45 (s, 9H, Boc), 1.11-1.20 (m, 1H, cyclopropyl CH), 0.58-0.65 (m, 2H, cyclopropyl CH₂), 0.27-0.37 (m, 2H, cyclopropyl CH₂) [>90% deuteration in both positions].

¹³C NMR (DICHLOROMETHANE-d₂, 151 MHz): δ (ppm) 172.3 (C-1), 156.5 (3C-4), 155.3 (2C-1), 130.8 (3C-2), 129.8 (3C-1), 116.3 (3C-3), 79.9 (2C-2), 72.3 (quint., ¹J_{CD}=24.2 Hz, OCD₂S), 70.4 (1C-1), 55.0 (C-2), 37.7 (C—), 28.4 (2C-3), 14.0 (sept., ¹J_{CD}=21.6 Hz, SCD₃), 14.1 (quint, ¹J_{CD}=22.3 Hz, SCHD₂), 10.0 (1C-2), 3.6 (1C-3), 3.5 (1C-4).

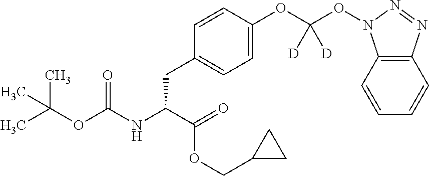 [SMILES: [2H]C([2H])(Oc1ccc(C[C@@H](NC(=O)OC(C)(C)C)C(=O)OCC2CC2)cc1)On1nnc2ccccc21]

### Cyclopropylmethyl O-[(1H-benzotriazol-1-yloxy)[²H₂]methyl]-N-(tert-butoxy-carbonyl)-D-tyrosinate. 1-10

160 mg (0.40 mmol) of 3-10-1 were reacted as described for 1-2-1 with the exception that the reaction time in step 3 is shortened to 10 min. The solution was directly chromatographed on a Biotage system (Flash25+M cartridge, 25 ml/min, n-hexane to n-hexane/ethyl acetate 1:1 in 15CV=780 ml) gave 88 mg of 1-10. The compound was further purified by preparative HPLC (Dionex: Pump P 580, Gilson: Liquid Handler 215, Knauer: UV-Detector K-2501, Chiralpak IC 5 μm 250×30 mm, hexane/ethanol 80:20, 40 ml/min, r.t., 88 mg/1.6 ml ethanol 2×0.8 ml, UV 254 nm). Collection of the eluate from 16.0 to 17.2 min gave after evaporation 43 mg of 1-10 with a purity of 97.3%. After thorough drying under high vacuum, 15.4 mg (8%) 1-10 was obtained.

MS (ESI⁺): m/e=485 (M+H⁺), 429 (M+H⁺−C₄H₈), 385 (M+H⁺−C₄H₈−CO₂).

¹H NMR (DICHLOROMETHANE-d₂, 600 MHz): δ (ppm) 7.95 (d, J=8.3 Hz, 1H, Bt-H), 7.39 (ddd, J=8.3, 6.8, 0.8 Hz, 1H, Bt-H), 7.34 (ddd, J=8.3, 6.8, 1.1 Hz, 1H, Bt-H), 7.16-7.23 (m, 3H, Bt-H, Ar—H), 7.06 (d, J=8.3 Hz, 2H, Ar—H), 5.38 (d, J=7.9 Hz, 1H, NH), 4.48 (ddd, J=7.9, 6.8, 5.6 Hz, 1H, 2-H), 3.98-3.88 (m, 2H, COOCH₂), 3.12 (dd, J=13.9, 5.6 Hz, 1H, 3-H), 3.02 (dd, J=13.9, 6.8 Hz, 1H, 3-H), 1.39 (s, 9H, Boc), 1.07-1.13 (m, 1H, Cyclopropyl CH), 0.51-0.59 (m, 2H, cyclopropyl CH₂), 0.23-0.29 (m, 2H, cyclopropyl CH₂). 6.02 (s, 0.08H, OCH₂O) corresponds to 4 mol % undeuterated compound, 6.01 (d, J=1.1 Hz, 0.12H, OCDHO) corresponds to 12 mol % monodeuterated compound.

¹³C NMR (DICHLOROMETHANE-d₂, 151 MHz): δ (ppm) 172.2 (C-1), 155.4 (3C-4, 2C-1), 143.7 (Bt C-3a), 132.1 (3C-1), 131.1 (3C-2), 128.9 (Bt C-7a), 128.5 (Bt C-6), 124.9 (Bt C-5), 120.1 (Bt C-4), 116.4 (3C-3), 109.3 (Bt C-7), 98.8 (p, ¹J_{CD}=25 Hz, OCD2O), 79.7 (2C-2), 70.3 (1C-1), 55.0 (C-2), 37.5 (C-3), 28.3 (2C-3), 9.9 (1C-2), 3.5 (1C-3), 3.4 (1C-4).

### 1.11 Example 11

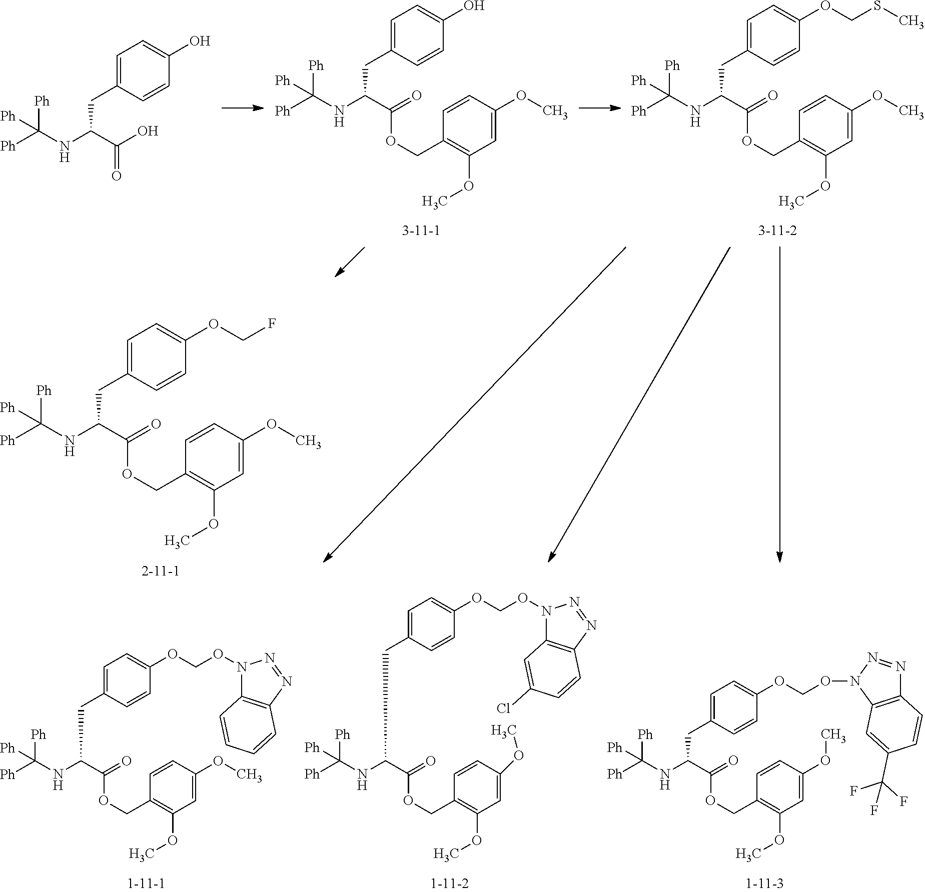

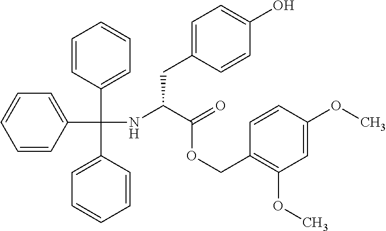 [SMILES: COc1ccc(COC(=O)[C@@H](Cc2ccc(O)cc2)NC(c2ccccc2)(c2ccccc2)c2ccccc2)c(OC)c1]

### 2,4-Dimethoxybenzyl N-trityl-D-tyrosinate. 3-11-1

5.00 g (11.81 mmol) N-trityl-D-tyrosine (Liebigs Ann. Chem. 1988, 1083-1084) were dissolved in 97.7 ml N,N-dimethylformamide. 2.31 g (7.08 mmol) cesium carbonate were added and the mixture stirred for 15 min. 3.14 g (13.58 mmol) 2,4-dimethoxybenzyl bromide in toluene (U.S. Pat. No. 5,663,200, 1997, Example 49a) were added and the mixture stirred for 16 h. The mixture was concentrated, diluted with ethyl acetate and extracted with water. The organic solutions were washed with saturated sodium chloride solution, dried over sodium sulfate and concentrated. The residue was purified by chromatography over a 55 g SNAP KP-NH cartridge (Biotage) with dichloromethane/ethanol 100/0-97/3-94/6-91/9 to give 4, 22 (50%) g.

The reaction was repeated with 4.37 g and 5.92 g N-trityl-D-tyrosine to give 5.55 g (94%) and 3.24 g (40%) 3-11-1 respectively.

MS (ESI⁺): m/e=574.42 (M+H⁺).

MS (ESI⁻): m/e=572.29 (M−H), 618.42 (M+HCOO).

¹H NMR (CHLOROFORM-d, 300 MHz): δ (ppm) 7.40-7.45 (m, 6H, Tr-H), 7.12-7.24 (m, 9H, Tr-H), 6.99 (d, 2H, Ar—H), 6.92 (d, 1H, Dmb H-6), 6.68 (d, 2H, Ar—H), 6.49 (d, 1H, Dmb

H-3), 6.40 (dd, 1H, Dmb H-5), 4.94 (br. s, 1H, OH), 4.58 (d, 1H, OCH₂Ar), 4.34 (d, 1H), 3.81 (s, 3H, Dmb OMe), 3.76 (s, 3H, Dmb OMe), 3.52-3.61 (m, 1H, 2-H), 2.85 (br. s, 1H, 3-H), 2.84 (br. s, 1H, 3-H), 2.59 (d, 1H, NH).

¹³C NMR (CHLOROFORM-d, 101 MHz): δ (ppm) 174.4 (C-1), 161.1 (Dmb C-5), 158.8 (Dmb C-3), 154.3 (3C-4), 146.0 (Tr C-2), 131.6 (Dmb C-7), 131.0 (3C-2), 129.5 (3C-1), 128.8 (Tr C-3), 127.8 (Tr C-4), 126.3 (Tr C-5), 116.4 (Dmb C-2), 115.0 (3C-3), 103.9 (Dmb C-6), 98.4 Dmb C-4), 71.1 (Tr C-1), 61.8 (Dmb C-1), 58.2 (C-2), 55.5 (Dmb OMe), 55.3 (Dmb OMe), 41.2 (C-3).

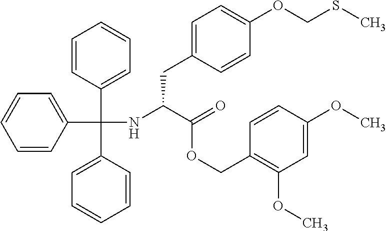 [SMILES: COc1ccc(COC(=O)[C@@H](Cc2ccc(OCSC)cc2)NC(c2ccccc2)(c2ccccc2)c2ccccc2)c(OC)c1]

### 2,4-Dimethoxybenzyl O-[(methylsulfanyl)methyl]-N-trityl-D-tyrosinate. 3-11-2

8.791 g (15.32 mmol) 2,4-Dimethoxybenzyl N-trityl-D-tyrosinate 3-11-1 were dissolved in 123 ml N,N-dimethylformamide. 9.99 g (30.65 mmol) cesium carbonate were added and the mixture stirred for 30 min. 1.78 g (18.39 mmol) chloro dimethyl sulfide were added and the mixture stirred for 20 h. The mixture was concentrated, diluted with ethyl acetate and extracted with water. The organic solutions were washed with saturated sodium chloride solution, dried over sodium sulfate and concentrated. The residue was purified by chromatography over a 110 g SNAP KP-NH cartridge (Biotage) with n-hexane/ethyl acetate 100/0-80/20-60/40 to give 5.59 g (52%) of 3-11-2.

¹H NMR (CHLOROFORM-d, 300 MHz): δ (ppm) 7.37-7.46 (m, 6H, Tr-H), 7.11-7.25 (m, 9H, Tr-H), 7.07 (d, J=8.7 Hz, 2H, Ar—H), 6.92 (d, J=8.9 Hz, 1H, Dmb H-6), 6.83 (d, J=8.7 Hz, 2H, Ar—H), 6.40 (d, J=2.1 Hz, 1H, Dmb H-3), 6.39 (dd, J=7.0, 2.3 Hz, 1H, Dmb H-5), 5.11 (s, 2H, OCH₂S), 4.58 (d, J=12.1 Hz, 1H, OCH₂Ar), 4.34 (d, J=12.1 Hz, 1H, OCH₂Ar), 3.80 (s, 3H, Dmb OMe), 3.76 (s, 3H, Dmb OMe), 3.51-3.63 (m, 1H, 2-H), 2.86 (br. s, 1H, 3-H), 2.84 (br. s, 1H, 3-H), 2.59 (d, J=10.5 Hz, 1H, NH), 2.25 (s, 3H, SCH₃).

¹³C NMR (CHLOROFORM-d, 75 MHz): δ (ppm) 174.2 (C-1), 161.1 (Dmb C-5), 158.8 (Dmb C-3), 155.8 (3C-4), 146.0 (Tr C-2), 131.5 (Dmb C-7), 130.9 (3C-2), 130.7 (3C-1), 128.8 (Tr C-3), 127.8 (Tr C-4), 126.3 (Tr C-5), 116.3 (Dmb C-2), 115.6 (3C-3), 103.8 (Dmb C-6), 98.3 (Dmb C-4), 72.4 (OCH₂S, 71.0 (Tr C-1), 61.6 (Dmb C-1), 58.1 (C-2), 55.4 (Dmb OMe), 55.3 (Dmb OMe), 41.2 (C-3), 14.6 (SCH₃).

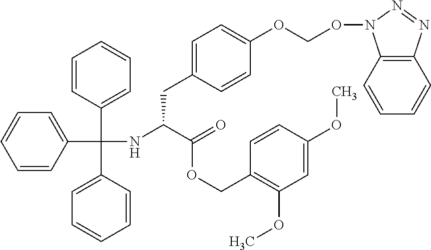 [SMILES: COc1ccc(COC(=O)[C@@H](Cc2ccc(OCOn3nnc4ccccc43)cc2)NC(c2ccccc2)(c2ccccc2)c2ccccc2)c(OC)c1]

### 2,4-Dimethoxybenzyl O-[(1H-benzotriazol-1-yloxy)methyl]-N-trityl-D-tyrosinate. 1-11-1

To a solution of 3-11-2 (356 mg, 0.56 mmol) in dichloromethane (5 ml) at −15° C. was added N-chlorosuccinimide (82.5 mg, 0.62 mmol). The cooling bath was removed and the solution stirred for 4 h. A solution of tetrabutylammonium 1-hydroxybenzotriazolat (253.8 mg, 0.67 mmol) in dichloromethane (2+0.5 ml) was added. The solution was stirred for 1 h. The reaction mixture was directly applied to Isolute and chromatographed on a Biotage system (Isolera Four, SNAP 10 g, dichloromethane/ethyl acetate 100/0-95/5) to give 248 mg. The compound was purified by preparative HPLC (Dionex: Pump P 580, Gilson: Liquid Handler 215, Knauer: UV-Detector K-2501, Chiralpak IB 5 μm 250×30 mm, Hexane/Ethanol 80:20, 40 ml/min, RT, 248 mg/3.5 ml Ethanol/Dichloromethane, 5×0.7 ml, UV 254 nm, 8.5-12.2 min, 94.2%, 120 mg, Peak 6-5.17 min) to give 116 mg of 1-11-1 with 94° A) purity.

MS (ESI⁺): m/e=721.39 (M+H⁺), 243.11 (C₁₉H₁₅⁺).

¹H NMR (DICHLOROMETHANE-d₂, 300 MHz): δ (ppm) 7.96 (d, J=8.3 Hz, 1H, Bt H-4), 7.37-7.51 (m, 6H, Tr H), 7.10-7.36 (m, 17H, Tr-H, Ar—H), 7.04 (d, J=8.7 Hz, 2H, Ar—H), 6.96 (d, J=8.1 Hz, 1H, Dmb 6-H), 6.40 (d, J=2.4 Hz, 1H, Dmb 3-H), 6.38 (dd, J=8.1, 2.4 Hz, 1H, Dmb 5-H), 5.98-6.07 (mc, 2H, OCH₂O), 4.59 (d, J=11.9 Hz, 1H, Dmb 1-H), 4.34 (d, J=11.9 Hz, 1H, Dmb 1-H), 3.75 (s, 3H, Dmb OMe), 3.75 (s, 3H, Dmb OMe), 2.80-2.99 (m, 2H, 3-H).

¹³C NMR (CHLOROFORM-d, 75 MHz): δ (ppm) 173.7 (C-1), 161.2 (Dmb C-5), 158.8 (Dmb C-3), 154.9 (3C-4), 146.0 (2C-2), 143.4 (Bt C-3a), 132.8 (3C-1), 131.4 (Dmb C-7), 131.3 (3C-3), 128.7 (Tr C-3), 128.7 (Bt C-7a), 128.2 (Bt C-6), 127.8 (Tr C-4), 126.3 (Tr C-5), 124.5 (Bt C-5), 119.7 (Bt C-4), 116.2 (Dmb C-2), 115.7 (3C-3), 109.0 (Bt C-7), 103.9 (Dmb C-6), 99.1 (OCH₂O), 98.1 (Dmb C-4), 71.1 (Tr C-1), 61.6 (Dmb C-1), 57.9 (C-2), 55.3 (Dmb OMe), 55.3 (Dmb OMe), 41.0 (C-3).

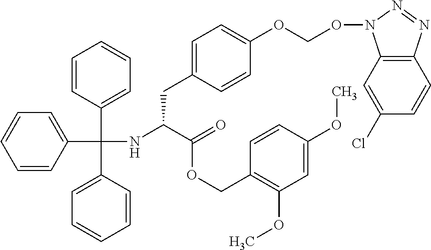 [SMILES: COc1ccc(COC(=O)[C@@H](Cc2ccc(OCOn3nnc4ccc(Cl)cc43)cc2)NC(c2ccccc2)(c2ccccc2)c2ccccc2)c(OC)c1]

### 2,4-Dimethoxybenzyl 0-{[(6-chloro-1H-benzotriazol-1-yl)oxy]methyl}-N-trityl-D-tyrosinate. 1-11-2

To a solution of 3-11-2 (80 mg, 0.13 mmol) in dichloromethane (1.1 ml) at −15° C. was added N-chlorosuccinimide (18.54 mg, 0.14 mmol). The cooling bath was removed and the solution stirred for 4 h. A solution of tetrabutylammonium 6-chloro-1-hydroxybenzotriazolat (62.3 mg, 0.15 mmol) in dichloromethane (0.6 ml) was added. The solution was stirred for 1 h. The reaction mixture was directly applied to Isolute and chromatographed (SNAP 5 g, dichloromethane to dichloromethane/ethyl acetate 95:5). The compound was purified by preparative HPLC (Waters Autopurificationsystem: Pump 254, Sample Manager 2767, CFO, DAD 2996, ELSD 2424, SQD 3001, XBrigde C18 5 μm 100×30 mm, A=water+0.2% ammonia, B=acetonitrile, 0-1 min 70% B, 1-8 min 70-100% B, 50 ml/min, r.t. 14 mg/1.5 ml dimethyl sulfoxide/methanol 1:1, 1×1.5 ml, DAD scan range 210-400 nm, MS ESI+, ESI−, scan range 160-1000 m/z, ELSD). The fractions eluting at 6.6-7.0 min were collected to give 6 mg (6%) of 1-11-2 with >99% purity (DAD).

¹H NMR (DICHLOROMETHANE-d₂, 400 MHz): δ (ppm) 7.90 (d, J=8.8 Hz, 1H, Bt 4-H), 7.41-7.46 (m, 6H, Tr-H), 7.27-7.33 (m, 2H, Bt 5-H, H-7), 7.14-7.26 (m, 11H, Tr-H, Ar—H), 7.01 (d, J=8.8 Hz, 2H, Ar—H), 6.94 (d, J=8.1 Hz, 1H, Dmb 6-H), 6.38 (d, J=2.3 Hz, 1H, Dmb 3-H), 6.36 (dd, J=8.1, 2.5 Hz, 1H, Dmb 5-H), 6.00 (s, 2H, OCH₂O), 4.59 (d, J=11.9 Hz, 1H, Dmb 1-H), 4.33 (d, J=11.9 Hz, 1H, Dmb 1-H), 3.75 (s, 3H, Dmb OMe), 3.74 (s, 3H, Dmb OMe), 3.53-3.60 (m, 1H, 2-H), 2.85-2.97 (m, 2H, 3-H).

¹³C NMR (CHLOROFORM-d, 101 MHz): δ (ppm) 173.8 (C-1), 161.2 (Dmb C-5), 158.9 (Dmb C-3), 154.7 (3C-4), 146.0 (2C-2), 142.1 (Bt C-3a), 134.6 (Bt C-6), 133.0 (3C-1), 131.5 (Dmb C-7), 131.4 (3C-2), 129.2 (Bt C-7a), 128.8 (Tr C-3), 127.9 (Tr C-4), 126.4 (Tr C-5), 125.9 (Bt C-5), 121.0 (Bt C-4), 116.2 (Dmb C-2), 115.8 (3C-3), 109.0 (Bt C-7), 103.9 (Dmb C-6), 99.2 (OCH₂O), 98.1 (Dmb C-4), 71.2 (Tr C-1), 61.6 (Dmb C-1), 58.0 (C-2), 55.3 (Dmb OMe), 55.3 (Dmb OMe), 41.0 (C-3).

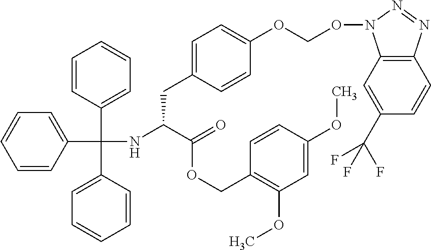 [SMILES: COc1ccc(COC(=O)[C@@H](Cc2ccc(OCOn3nnc4ccc(C(F)(F)F)cc43)cc2)NC(c2ccccc2)(c2ccccc2)c2ccccc2)c(OC)c1]

### 2,4-Dimethoxybenzyl 0-{[(6-trifluoromethyl-1H-benzotriazol-1-yl)oxy]methyl}-N-trityl-D-tyrosinate. 1-11-3

To a solution of 3-11-2 (824.6 mg, 1.30 mmol) in dichloromethane (12 ml) at −15° C. was added N-chlorosuccinimide (191.1 mg, 1.43 mmol). The cooling bath was removed and the solution stirred for 5 h. A solution of tetrabutylammonium 6-trifluoromethyl-1-hydroxy-benzotriazolat (694.1 mg, 1.56 mmol) in dichloromethane (6 ml) was added. The solution was stirred for 1 h. The reaction mixture was directly applied to Isolute and chromatographed (SNAP 25 g, n-hexane/ethyl acetate 100/0-85/15-60/40) to give 213 mg of 1-11-3 with >95% purity.

MS (ESI⁺): m/e=789.37 (M+H⁺).

MS (ESI⁻): m/e=833.07 (M+HCOO).

¹H NMR (CHLOROFORM-d, 400 MHz): δ (ppm) 8.12 (d, J=8.8 Hz, 1H, Bt 4-H), 7.57 (d, J=8.8 Hz, 1H, Bt 5-H), 7.40-7.50 (m, 6H, Tr o-H), 7.11-7.25 (m, 12H, Tr m-H, p-H, Bt 7-H, Ar—H), 6.95 (m, 3H, Ar—H, Dmb 6-H), 6.32-6.42 (m, 2H, Dmb 3-H, 5-H), 6.04 (s, 2H, OCH₂O), 4.62 (d, J=11.9 Hz, 1H, Dmb 1-H), 4.35 (d, J=11.9 Hz, 1H, Dmb 1-H), 3.76 (s, 3H, Dmb OMe), 3.75 (s, 3H, Dmb OMe), 3.62 (br. s., 1H, 2-H), 2.93 (m, 2H, 3-H), 2.62 (br. s, 1H, NH).

¹³C NMR (101 MHz, CHLOROFORM-d) δ ppm 174.0 (C-1), 161.2 (Dmb C-5), 158.9 (Dmb C-3), 154.3 (3C-4), 146.0 (2C-2), 144.5 (Bt C-3a), 133.1 (3C-1), 131.6 (Dmb C-7), 131.5 (3C-2), 130.4 (q, ²J_{CF}=32.0 Hz, Bt C-6), 128.8 (Tr C-3), 128.2 (Bt C-7a), 127.8 (Tr C-4), 126.4 (Tr C-5), 123.6 (q, ¹J_{CF}=273.2 Hz, CF₃), 121.4 (q, ³J_{CF}=3.2 Hz, Bt C-5), 121.2 (Bt C-4), 116.3 (Dmb C-2), 115.6 (3C-3), 107.9 (q, J=4.8 Hz, Bt C-7), 103.9 (Dmb C-6), 99.0 (OCH₂O), 98.4 (Dmb C-4), 71.2 (Tr C-1), 61.8 (Dmb C-1), 57.9 (C-2), 55.4 (Dmb OMe), 55.3 (Dmb OMe), 41.1 (C-3).

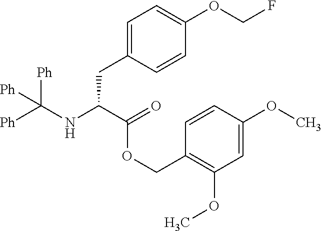 [SMILES: COc1ccc(COC(=O)[C@@H](Cc2ccc(OCF)cc2)NC(c2ccccc2)(c2ccccc2)c2ccccc2)c(OC)c1]

### 2,4-Dimethoxybenzyl 0-(fluoromethyl)-N-trityl-D-tyrosinate. 2-11-1

84 mg (0.15 mmol) 2,4-dimethoxybenzyl N-trityl-D-tyrosinate 3-11-1 were dissolved in 1 ml tetrahydrofuran. The solution was cooled to 0° C. 16.5 mg (0.41 mmol) sodium hydride (60% in mineral oil) were added and the mixture stirred for 1 hour. 1.05 ml of tetrahydro-furan containing bromofluoromethane were added slowly at 0° C. and the mixture was stirred at 0° C. for 12 h. 1 ml methanol was added and the mixture diluted with ethyl acetate and extracted with water. The organic solutions were dried over sodium sulfate and concentrated. The residue was applied to Isolute and chromatographed (SNAP 10 g, n-hexane to n-hexane/ethyl acetate 6:4) to give 60 mg of 2-11-1 with 90% purity.

MS (ESI⁺): m/e=606.24 (M+H⁺).

¹⁹F NMR (376 MHz, CHLOROFORM-d): δ (ppm)-147.9 (t, J=55.1 Hz).

¹H NMR (CHLOROFORM-d, 300 MHz): δ (ppm) 7.39-7.47 (m, 6H, Tr-H), 7.11-7.25 (m, 9H, Tr-H), 7.07 (d, J=8.5 Hz, 2H, Ar—H), 6.93 (d, J=8.5 Hz, 2H, Ar—H), 6.89 (d, J=8.9 Hz, 1H, Dmb 6-H), 6.35-6.43 (m, 2H, Dmb 5-H, 3-H), 5.67 (d, ²J_{HF}=55.0 Hz, 2H, OCH₂F), 4.58 (d, J=11.9 Hz, 1H, Dmb 1-H), 4.34 (d, J=11.9 Hz, 1H, Dmb 1-H), 3.80 (s, 3H, Dmb OMe), 3.75 (s, 3H, Dmb OMe), 3.52-3.64 (m, 1H, 2-H), 2.84-2.92 (m, 2H, 3-H, NH), 2.60 (d, br., J=9.6 Hz, 1H, 3-H).

¹³C NMR (75 MHz, CHLOROFORM-d) δ (ppm) 174.1 (C-1), 161.1 (Dmb C-5), 158.8 (Dmb C-3), 155.6 (d, ³J_{CF}=3.0 Hz, 3C-4), 145.9 (Tr C-2), 132.5 (Dmb C-7), 131.5 (3C-1), 131.0 (3C-2), 128.8 (Tr C-3), 127.8 (Tr C-4), 126.3 (Tr C-5), 116.3 (d, ⁴J_{CF}=1.2 Hz, 3C-3), 116.3 (Dmb C-2), 103.9 (Dmb C-6), 100.9 (d, ¹J_{CF}=218.4 Hz, OCH₂F), 98.3 (Dmb C-4), 71.1 (Tr C-1), 61.6 (Dmb C-1), 58.0 (C-2), 55.4 (Dmb OMe), 55.3 (Dmb OMe), 41.3 (C-3).

### 1.12 Example 12

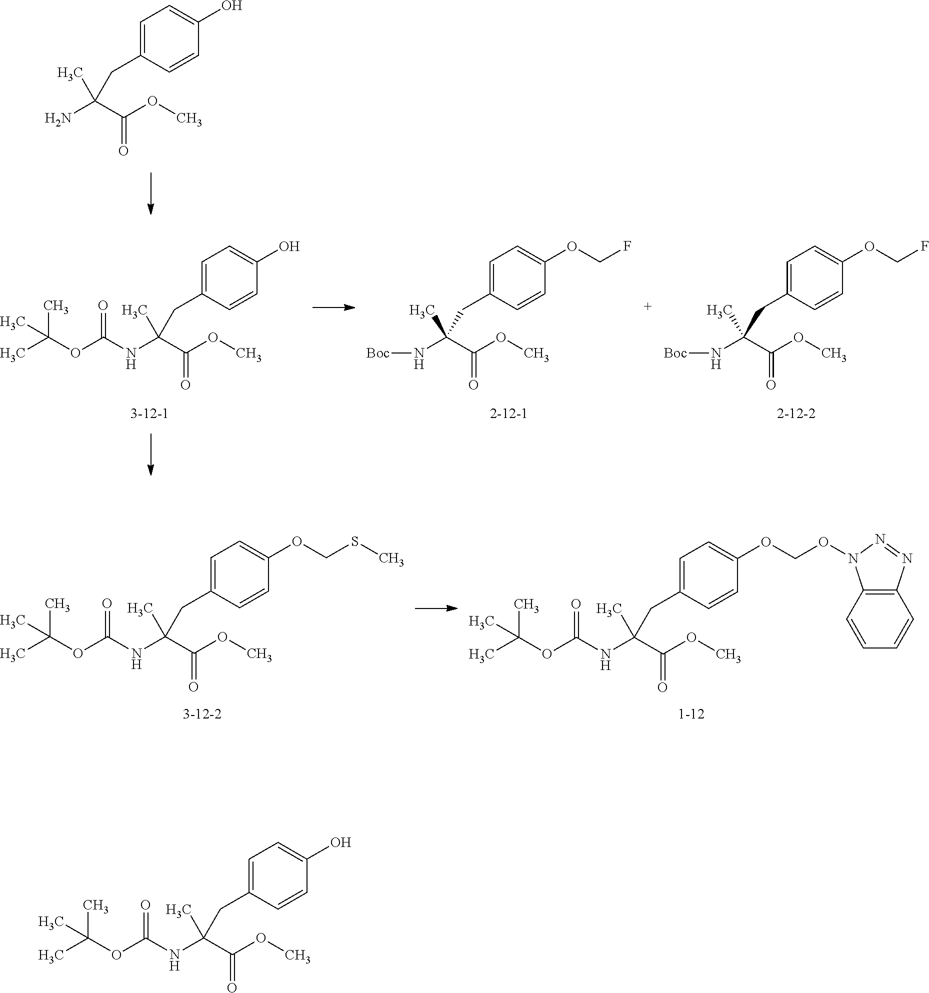

### Methyl N-(tert-butoxycarbonyl)-alpha-methyltyrosinate 3-12-1

9.25 g (37.6 mmol) Methyl alpha-methyltyrosinate hydrochloride were suspended in 100 ml dioxane and 100 ml 1N sodium hydrogen carbonate The pH of the reaction mixture was adjusted to 8-9 with 1N sodium hydroxide. 28.8 g (131 mmol) di-tert-butyl dicarbonate were added in portions and the mixture stirred for 3 d at r.t., while the pH was controlled and kept between 8 and 9. The reaction mixture was brought to pH 2 with 1N sodium hydrogen sulfate and extracted with ethyl acetate. The organic phase was washed with water and brine, after evaporation of the solvent 14.8 g of raw material were obtained. Chromatography on a Biotage Isolera system (SNAP 340 cartridge, 100 ml/min, n-hexane to n-hexane/ethyl acetate 59:41 in 12 CV=4080 ml, Fractions 83-100) gave 10 g (86%) 3-12-1 as white solid.

MS (ESI⁻): m/e=618 (2M−H⁺), 354 (M+HCOO⁻), 308 (M−H⁺).

MS (ESI⁺): m/e=641 (2M+Na⁺), 619 (2M+H⁺), 332 (M+Na⁺), 310 (M+H⁺), 254 (M+H⁺—C₄H₈), 210 (M+H⁺−CO₂−C₄H₈).

¹H NMR (CHLOROFORM-d, 500 MHz): δ (ppm) 6.96 (d, J=8.5 Hz, 2H, Ar—H), 6.77 (d, J=8.2 Hz, 2H, Ar—H), 5.91 (br. s., 1H, OH), 5.20 (br. s., 1H, NH), 3.79 (s, 3H, OMe), 3.30 (br. s., 1H, 3-H), 3.15 (d, J=13.6 Hz, 1H, 3-H), 1.59 (br. s., 3H, 2-CH₃), 1.51 (s, 9H, Boc).

¹³C NMR (CHLOROFORM-d, 126 MHz): δ (ppm) 174.6 (C-1), 155.1 (3C-4), 154.5 (2C-1), 131.2 (3C-2), 128.0 (br. 3C-1), 115.2 (3C-3), 79.6 (br. 2C-2), 60.5 (br. C-2), 52.5 (1C-1), 41.2 (br. C-3), 28.4 (3C-3), 23.6 (2-CH₃).

In fraction 62-68, 1.48 g (10%) of the bisbocylated compound were isolated.

Methyl N,O-bis(tert-butoxycarbonyl)-alpha-methyltyrosinate

MS (ESI⁺): m/e=432 (M+Na⁺), 427 (M⁺+H₂O), 410 (M+H⁺), 354 (M+H⁺−C₄H₈), 310 (M+H⁺−CO₂−C₄H₈), 254 (M+H⁺−CO₂₋₂C₄H₈).

¹H NMR (CHLOROFORM-d, 500 MHz): δ (ppm) 7.08 (s, 4H, Ar—H), 5.11 (br. s., 1H, NH), 3.76 (s, 3H, OCH₃), 3.36 (br. d, J=12.3 Hz, 1H, 3-H), 3.23 (d, J=13.6 Hz, 1H, 3-H), 1.56 (s, 9H, OBoc), 1.54 (br. s., 3H, 2-CH₃), 1.47 (s, 9H, NBoc).

¹³C NMR (CHLOROFORM-d, 126 MHz): δ (ppm) 174.3 (C-1), 154.3 (2C-1), 151.9 (OBoc C-1), 150.1 (3C-4), 134.0 (br., 3C-1), 131.0 (3C-2), 120.9 (3C-3), 83.5 (OBoc C-2), 79.6 (br., 2C-2), 60.2 (br., C-2), 52.6 (OCH₃), 40.8 (br., C-3), 28.4 (2C-3), 27.8 (OBoc C-3), 23.7 (br., 2-CH₃).

### Methyl (R) and (S)-2-[(tert-Butoxycarbonyl)amino]-3-(fluoromethoxy)phenyl-2-methylpropionate

As described in the preparation of 2-1-1, 250 mg (0.81 mmol) 3-12-1 were reacted to give 221 mg of raw product, which was purified by preparative HPLC (Dionex: Pump P 580, Gilson: Liquid Handler 215, Knauer: UV-Detector K-2501, Chiralpak AD-H 5 μm 250×20 mm, hexane/ethanol 80:20, 20 ml/min, r.t., 221 mg/4 ml ethanol, 10×0.4 ml, UV 210 nm) The peaks at 4.8-5.5 min (75 mg 99.5%) and 5.7-6.3 min (76 mg 98.6%) were collected. Combined yield 27%.

The stereochemistry of the first peak was putatively assigned “R” (comparison of the retention behaviour on the chiral HPLC with 2-2-1 and 2-2-2).

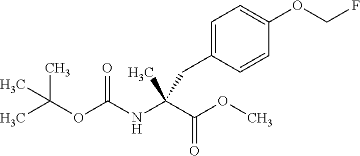 [SMILES: COC(=O)[C@@](C)(Cc1ccc(OCF)cc1)NC(=O)OC(C)(C)C]

### Methyl N-(tert-butoxycarbonyl)-O-(fluoromethyl)-alpha-methyl-D-tyrosinate 2-12-1

α_{D}+44.1° (MeOH, c=1, 589 nm).

MS (ESI⁺): m/e=364 (M+Na⁺), 342 (M+H⁺), 286 (M+H⁺−C₄H₈), 242 (M+H⁺−CO₂−C₄H₈).

¹⁹F NMR (376 MHz, DICHLOROMETHANE-d₂) δ ppm-149.0 (t, ²J_{HF}=55.1 Hz).

¹H NMR (DICHLOROMETHANE-d₂, 400 MHz): δ (ppm) 7.07 (d, J=8.5 Hz, 2H, Ar—H), 7.01 (d, J=8.5 Hz, 2H, Ar—H), 5.72 (d, ²J_{HF}=54.7 Hz, 2H, OCH₂F), 5.14 (br. s., 1H, NH), 3.75 (s, 3H, OCH₃), 3.34 (br. d, J=13.1 Hz, 1H, 3-H), 3.17 (d, J=13.8 Hz, 1H, 3-H), 1.54 (s, 3H, 2-CH₃), 1.48 (s, 9H, Boc).

¹³C NMR (101 MHz, DICHLOROMETHANE-d₂) δ ppm 174.3 (C-1), 155.7 (d, ³J_{CF}=2.7 Hz, 3C-4), 154.2 (2C-1), 131.8 (3C-1), 131.3 (3C-2)), 116.2 (d, ⁴J_{CF}=1.2 Hz, 3C-3), 101.0 (d, ¹J_{CF}=217.4 Hz, OCH₂F), 79.3 (br., 2C-2), 60.3 (C-2), 52.3 (1C-1), 40.7 (br., C-3), 28.1 (2C-3), 23.4 (2-CH₃).

The stereochemistry of the second peak was putatively assigned “S” (comparison of the retention behaviour on the chiral HPLC with 2-2-1 and 2-2-2).

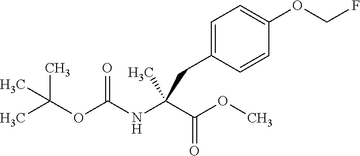 [SMILES: COC(=O)[C@](C)(Cc1ccc(OCF)cc1)NC(=O)OC(C)(C)C]

### Methyl N-(tert-butoxycarbonyl)-O-(fluoromethyl)-alpha-methyl-L-tyrosinate 2-12-2

α_{D}−45.8° (MeOH, c=1, 589 nm).

MS (ESI⁺): m/e=364 (M+Na⁺), 342 (M+H⁺), 286 (M+H⁺−C₄H₈), 242 (M+H⁺−CO₂−C₄H₈).

¹⁹F NMR (376 MHz, DICHLOROMETHANE-d₂) δ ppm-149.0 (t, ²J_{HF}=55.1 Hz).

¹H NMR (DICHLOROMETHANE-d₂, 400 MHz): δ (ppm) 7.04 (d, J=8.8 Hz, 2H, Ar—H), 6.98 (d, J=8.6 Hz, 2H, Ar—H), 5.70 (d, J=54.8 Hz, 2H, OCH₂F), 5.13 (br. s, 1H, NH), 3.73 (s, 3H, OCH₃), 3.33 (br. d, J=13.6 Hz, 1H, 3-H), 3.14 (d, J=13.6 Hz, 1H, 3-H), 1.51 (s, 3H, 2-CH₃), 1.45 (s, 9H, Boc).

¹³C NMR (101 MHz, DICHLOROMETHANE-d₂) δ ppm 174.3 (C-1), 155.7 (d, ³J_{CF}=2.4 Hz, 3C-4), 154.2 (2C-1), 131.7 (3C-1), 131.3 (3C-2)), 116.2 (3C-3), 100.9 (d, ¹J_{CF}=217.3 Hz, OCH₂F), 79.3 (br., 2C-2), 60.3 (C-2), 52.3 (1C-1), 40.7 (br., C-3), 28.1 (2C-3), 23.4 (2-CH₃).

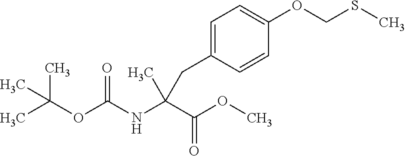 [SMILES: COC(=O)C(C)(Cc1ccc(OCSC)cc1)NC(=O)OC(C)(C)C]

### Methyl N-(tert-butoxycarbonyl)-alpha-methyl-O-[(methylsulfanyl)methyl]tyrosinate 3-12-2

A solution of 2.00 g (6.67 mmol) 3-12-1, 239 mg (0.67 mmol) tetrabutyl ammonium iodide in 20 ml N,N-dimethylformamide were cooled in an ice bath and a solution of 798 mg (7.11 mmol) potassium tert-butoxide in 7 ml tetrahydrofurane added. Subsequently 614 μl (7.44 mmol) chloromethyl methyl sulfide were added, whereupon the solution turned yellow. The ice bath was removed and the reaction stirred for 2 h at r.t. For work-up, ethyl acetate was added and the resulting solution washed with water. After phase separation, the aqueous phase was re-extracted with ethyl acetate. The combined organic phases were washed with 1N sodium hydrogen carbonate₃ and brine and then dried over sodium sulfate. Evaporation gave 2.56 g raw product. Chromatography on a Biotage Isolera system (SNAP 50 cartridge, 50 ml/min, n-hexane to n-hexane/ethyl acetate 6:4 in 12 CV) did not return pure product. Rechromatography of the product containing fractions on a Biotage system (C18HS 40+M cartridge, 40 ml/min, water to water/acetonitrile 1:1 in 12CV=1584 ml, water/acetonitrile 1:1 3CV=396 ml) gave 1.39 g (58%) 3-12-2.

MS (ESI⁺): m/e=392 (M+Na⁺), 370 (M+H⁺), 314 (M+H⁺−C₄H₈), 270 (M+H⁺−CO₂−C₄H₈).

¹H NMR (CHLOROFORM-d, 300 MHz): δ (ppm) 7.00 (d, J=8.5 Hz, 2H, Ar—H), 6.85 (d, J=8.7 Hz, 2H, Ar—H), 5.11 (s, 2H, OCH₂S), 3.75 (s, 3H, OCH₃), 3.31 (br. d, J=13.4 Hz, 1H, 3-H), 3.14 (d, J=13.8 Hz, 1H, 3-H), 2.25 (s, 3H, SCH₃), 1.54 (br. s., 3H, 2-CH₃), 1.46 (s, 9H, Boc).

¹³C NMR (CHLOROFORM-d, 75 MHz): δ (ppm) 174.4 (C-1), 156.0 (3C-4), 154.3 (2C-1), 131.0 (3C-2), 129.6 (br., 3C-1), 115.6 (3C-3), 79.5 (br., 2C-2), 72.4 (OCH₂S), 60.4 (C-2), 52.5 (0 CH₃), 40.9 (br., C-3), 28.4 (2C-3), 23.6 (2-CH₃), 14.6 (SCH₃).

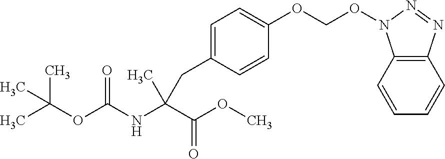 [SMILES: COC(=O)C(C)(Cc1ccc(OCOn2nnc3ccccc32)cc1)NC(=O)OC(C)(C)C]

### Methyl O-[(1H-benzotriazol-1-yloxy)methyl]-N-(tert-butoxycarbonyl)-alpha-methyltyrosinate 1-12

300 mg (0.81 mmol) of 3-12-2 were reacted as described for 1-2-1. The raw product was purified by chromatography on a Biotage Isolera system (SNAP 50 cartridge, 50 ml/min, n-Hexane, 1CV, n-Hexane to n-hexane/ethyl acetate 6:4 in 10 CV, n-hexane/ethyl acetate 6:4 4 CV) gave slightly impure material. Further purification was done by preparative HPLC (Dionex: Pump P 580, Gilson: Liquid Handler 215, Knauer: UV-Detector K-2501; Chiralpak IA 5 μm 250×20 mm; hexane/2-propanol 50:50; 12 ml/min; r.t. 170 mg/1.5 ml ethanol; 5×0.3 ml; UV 254 nm). The peak eluting 7.0-8.2 min was collected to give 137 mg (37%) of 1-12 with a purity of 99.7%. The material was not resolved into the enantiomers.

MS (ESI⁺): m/e=479 (M+Na⁺), 457 (M+H⁺), 401 (M+H⁺—C₄H₈), 357 (M+H⁺—CO₂−C₄H₈).

¹H NMR (DICHLOROMETHANE-d₂, 400 MHz): δ (ppm) 7.97 (d, J=8.3 Hz, 1H, Bt-H), 7.41 (ddd, J=8.1, 7.1, 0.8 Hz, 1H, Bt-H), 7.36 (ddd, J=8.3, 7.1, 1.3 Hz, 1H, Bt-H), 7.19 (d, J=8.1 Hz, 1H, Bt-H), 7.05 (d, J=8.8 Hz, 2H, Ar—H), 7.10 (d, J=8.8 Hz, 2H, Ar—H), 6.04 (s, 2H, OCH₂O), 5.17 (br. s., 1H, NH), 3.74 (s, 3H, OCH₃), 3.38 (d, br., J=13.4 Hz, 1H, 3-H), 3.17 (d, J=13.6 Hz, 1H, 3-H), 1.56 (s, 3H, 2-CH₃), 1.46 (s, 9H, Boc).

¹³C NMR (101 MHz, DICHLOROMETHANE-d₂) δ ppm 174.3 (C-1), 155.1 (2C-1), 154.2 (3C-4), 143.5 (Bt C-3a), 131.9 (3C-1), 131.5 (3C-2), 128.7 (Bt C-7a), 128.2 (Bt C-6), 124.6 (Bt C-5), 119.9 (Bt C-4), 115.8 (3C-3), 109.0 (Bt C-7), 98.9 (OCH₂O), 60.3 (C-2), 52.4 (1C-1), 40.7 (br., C-3), 28.1 (2C-3), 23.4 (br., 2-CH₃).

### Example 13

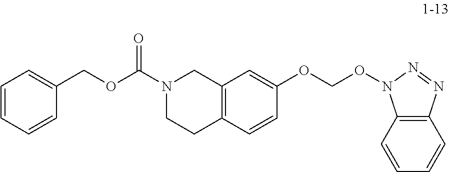 [SMILES: O=C(OCc1ccccc1)N1CCc2ccc(OCOn3nnc4ccccc43)cc2C1]

### Benzyl 7-[(1H-benzotriazol-1-yloxy)methoxy]-3,4-dihydroisoquinoline-2(1H)-carboxylate

1.00 g (6.53 mmol) N-hydroxy-1H-benzotriazol hydrate were dissolved in 6.63 ml 1 M KOH and stirred at ambient temperature over night. The solvent was removed i.vac. at 25° C. and the residue was dried under high vacuum at ambient temperature. 1.25 g (>100%) of the potassium salt of N-hydroxy-1H-benzotriazole was obtained as a white solid, which was used for further reactions.

100 mg (0.58 mmol) of the potassium salt of N-hydroxy-1H-benzotriazole prepared above were suspended in 6.5 ml THF and 319 mg (0.58 mmol) 7-chlormethoxy-3,4-dihydro-1H-isochinolin-2-carbonsäurebenzylester was added. The reaction was stirred over night at ambient temperature and then partitioned between with ethyl acetate and water. The organic phase was dried (sodium sulfate) and evaporated i. vac. Chromatography of the raw material on 10 g silica (hexane, hexane/ethyl acetate 8:2 and 6:4) gave 215 mg (87%) of an oil, which was further purified by preparative HPLC: (HPLC (Waters Autopurificationsystem: Pump 2545, Sample Manager 2767, CFO, DAD 2996, ELSD 2424, SQD 3001; XBrigde C18 5 μm 100×30 mm; A=H₂O+0.1% HCOOH; B=Acetonitrile, 0-1 min 1% B, 1-8 min 1-99% B, 8-10 min 99% B; 50 ml/min.) to give 10 mg (4%) of 1-13.

¹H NMR (CHLOROFORM-d, 400 MHz): δ (ppm) 8.00 (d, J=7.8 Hz, 1H, Bt 7-H), 7.31-7.45 (m, 7H, Bn-H, Bt H-5,6), 7.24 (d, J=8.3 Hz, 1H, Bt 4-H), 7.15 (d, J=8.3 Hz, 1H, Iq 4-H), 6.98 (dd, J=8.6, 2.3 Hz, 1H, Iq 5-H), 6.84-6.92 (m, 1H, Iq 7-H), 6.01 (s, 2H, OCH₂O), 5.20 (s, 2H, OCH₂Ph), 4.66 (s, br., 2H, Iq 2-H), 3.71-3.80 (m, br., 2H, Iq 3-H), 2.85 (s, Br., 2H, Iq 4-H).

### Example 14

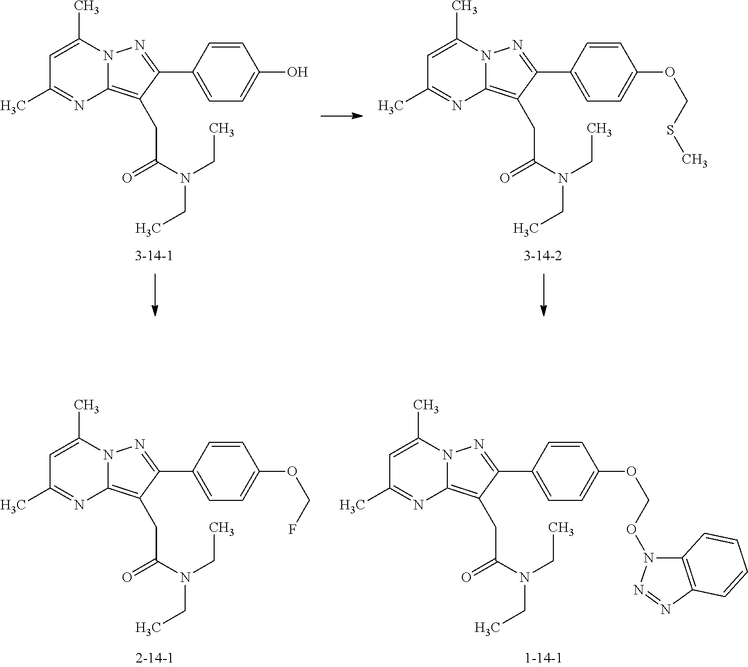

Compound 3-14-1 can be synthesized according to M. L. James et al., Bioorg. Med. Chem. 13 (2005), 6188.

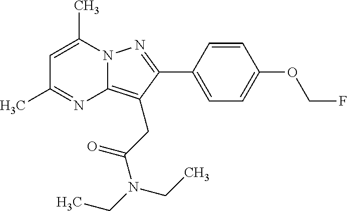 [SMILES: CCN(CC)C(=O)Cc1c(-c2ccc(OCF)cc2)nn2c(C)cc(C)nc12]

### N,N-Diethyl-2-{2-[4-(fluoromethoxy)phenyl]-5,7-dimethylpyrazolo[1,5-a]pyrimidin-3-yl}acetamide 2-14-1

A: 100 g (0.28 mmol) 3-14-1 were dissolved in 7 ml dry THF under an argon atmosphere and 17 mg (0.43 mmol) sodium hydride (60% in mineral oil) were added in one portion. The mixture was stirred for 5 min.

B: 25 ml dry THF were cooled to 0° C. and bromofluoromethane was bubbled into the solution. By weighing of the flask and of the steel container the amount of gas dissolved was determined. The solution can be stored in the refrigerator for some month.

3 ml of the solution of bromofluoromethane in THF were added to the solution prepared in A and the reaction was stirred at room temperature for 2 h. The mixture was poured in to ice water and extracted with dichloromethane three times. The combined organic phases were dried over sodium sulfate, and evaporated to give 119 mg raw product. Chromatography (Biotage Isolera System, Flash 12+M cartridge, CH₂Cl₂/MeOH 0-1% 15CV, 1-5% 10CV, 5-20% 10CV, 20-100% 10CV=540 ml) gave 97 mg 2-14-1 which was further purified by preparative HPLC (Agilent: Prep 1200, 2×Prep Pump, DLA, MWD, Prep FC, ESA: Corona; Chiralpak IC 5 μm 250×20 mm; Hexane/Ethanol 50:50; 15 ml/min; RT; 97 mg/1.5 ml EtOH/MeOH 1:1; 3×0.5 ml; UV 210 nm). The fractions eluting at 7.4-9.4 min were isolated to give 79 mg (72%) of 2-14-1 with >99% (210 nm) purity.

MS (ESI⁺): m/e=791 (2M+Na⁺), 769 (2M+H⁺), 385 (M+H⁺)

¹H NMR (DICHLOROMETHANE-d₂, 400 MHz): δ (ppm) 7.76-7.84 (m, 2H, Ph-H), 7.12-7.20 (m, 2H, Ph-H), 6.56 (q, J=1.0 Hz, 1H, 6-H), 5.77 (d, ¹J_{HF}=54.6 Hz, 2H, OCH₂F), 3.88 (s, 2H, CH₂), 3.51 (q, J=7.3 Hz, 2H, N CH₂), 3.38 (q, J=7.1 Hz, 2H, N CH₂), 2.72 (d, J=1.0 Hz, 3H, 5-CH₃), 2.53 (s, 3H, 7-CH₃), 1.22 (t, J=7.3 Hz, 3H, NCH₂CH₃), 1.10 (t, J=7.1 Hz, 3H, NCH₂CH₃).

¹³C NMR (101 MHz, DICHLOROMETHANE-d₂): δ (ppm) 169.7 (C═O), 157.8 (Pypy C-5), 156.8 (d, ³J_{CF}=3.2 Hz, Ph C-4), 153.9 (Pypy C-2), 147.7 (Pypy C-3a), 144.9 (Pypy C-7), 129.9 (Ph C-2/6), 129.4 (Ph C-1), 116.4 (d, ⁴J_{CF}=1.6 Hz, Ph C-3/5), 108.4 (Pypy C-6), 101.0 (Pypy C-3), 100.9 (d, ¹J_{CF}=218.1 Hz, OCH₂F), 42.3 (NCH₂ cis), 40.5 (NCH₂ trans), 28.0 (CH₂), 24.4 (5-CH₃), 16.6 (7-CH₃), 14.1 (NCH₂CH₃ trans), 12.9 (NCH₂CH₃ cis).

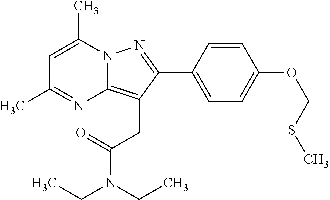

### 2-{5,7-Dimethyl-2-[4-(methylsulfanylmethoxy)phenyl]pyrazolo[1,5-a]pyrimidin-3-yl}-N,N-diethylacetamide 3-14-2

500 mg (1.42 mmol) 3-14-1 were dissolved in 35 ml dry DMF under an argon atmosphere and 85 mg (2.12 mmol) NaH (60% in mineral oil) were added. The mixture was stirred 5 min at room temperature and then 141 μl (1.70 mmol) chlordimethylsulfid added. The reaction was stirred over night, after which HPLC-MS indicated very little product formation. 52 mg (0.14 mmol) Tetrabutylammonium iodide were added and the reaction stirred for an additional 9 days. The mixture was poured into ice water and extracted three times with methylene chloride. The combined organic phases were dried over sodium sulfate, and evaporated to give 1.17 g raw material, which was purified (Biotage Isolera system, Flash 40+M cartridge, 40 ml/min, CH₂Cl₂ 3CV=396 ml, CH₂Cl₂/MeOH 0-80% 12CV=1584 ml) to give 620 mg (88%) of 3-16-2. HPLC-MS indicated the presence of double and triple alkylated species. 300 mg were subjected to a second chromatography (Biotage Isolera system, Flash 25+M cartridge, 25 ml/min, n-Hexane to Ethyl acetate in 10 CV, then Ethyl acetate 7 CV=880 ml) gave 220 mg 3-16-2, which still contained some 15% dialkylated species. Nevertheless, the material could be used in the next step.

MS (ESI⁺): m/e=412 (M⁺), 312 (M⁺−CONEt₂), 256 (M⁺−CONEt₂−C₂H₄S).

¹H NMR (DICHLOROMETHANE-d₂, 400 MHz): δ (ppm) 7.73-7.78 (m, 2H, Ph H2/6), 7.01-7.05 (m, 2H, Ph H3/5), 6.55 (br. s, 1H, Pypy H-5), 5.20 (s, 2H, SCH₂O), 3.89 (s, 2H, ArCH₂C0), 3.51 (q, J=7.3 Hz, 2H, NCH₂), 3.38 (q, J=7.1 Hz, 2H, NCH₂), 2.72 (d, J=0.8 Hz, 3H, 5-CH₃), 2.53 (s, 3H, 7-CH₃), 2.26 (s, 3H, SCH₃), 1.22 (t, J=7.1 Hz, 3H, NCH₂CH₃), 1.11 (t, J=7.1 Hz, 3H, NCH₂CH₃).

¹³C NMR (101 MHz, DICHLOROMETHANE-d₂): δ (ppm) 169.7 (C═O), 157.6 (Ph C-4), 157.3 (Pypy C-5), 154.3 (Pypy C-2), 147.6 (Pypy C-3a), 145.0 (Pypy C-7), 129.6 (Ph C-2/6), 127.5 (Ph C-1), 115.9 (Ph C-3/5), 108.2 (Pypy C-6), 100.9 (Pypy C-3), 72.5 (OCH₂S), 42.3 (NCH₂ cis), 40.5 (NCH₂ trans), 28.0 (CH₂), 24.3 (5-CH₃), 16.6 (7-CH₃), 14.4 (NCH₂CH₃ trans), 14.2 (NCH₂CH₃ cis), 12.9 (SCH₃).

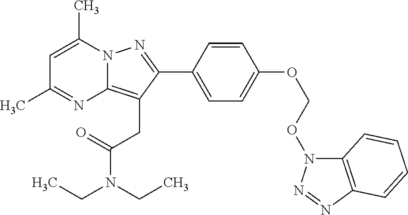 [SMILES: CCN(CC)C(=O)Cc1c(-c2ccc(OCOn3nnc4ccccc43)cc2)nn2c(C)cc(C)nc12]

### 2-(2-{4-[(1H-Benzotriazol-1-yloxy)methoxy]phenyl}-5,7-dimethylpyrazolo[1,5-a]-pyrimidin-3-yl)-N,N-diethylacetamide 1-14-1

A) 178 mg (1.17 mmol) N-Hydroxy-1H-benzotriazole hydrate were dissolved in 1.16 ml Tetrabutylammoniumhydroxide (1 mM in Methanol) and stirred for 30 minutes at room temperature. Methanol was then evaporated and the material stripped twice with toluene at max 40° C. bath temperature to give dry tetrabutylammonium N-hydroxy-1H-benzotriazolate.

B) 151 mg (0.37 mmol) 3-14-2 were dissolved in 2.5 ml dry methylene chloride, cooled to −15° C. and 54 mg (0.40 mmol) N-Chlorosuccinimide added. The mixture was stirred for 4 h, during which the reaction was slowly allowed to come to room temperature. Then the tetrabutylammonium N-hydroxy-1H-benzotriazolate prepared under A) was dissolved in 2.5 ml dry methylene chloride and added to the reaction and stirred for 30 min. The mixture was stored over night at −15° C. and then directly applied to the chromatography column. (Biotage Isolera System, SNAP 25 cartridge, 25 ml/min, A=Dichloromethane, B=Methanol, 100% A 3CV, 0% B to 30% B in 10CV, 30% B 3CV). 329 mg (>100%) material were obtained, which was subjected to preparative HPLC (Dionex: Pump P 580, Gilson: Liquid Handler 215, Knauer: UV-Detector K-2501; Chiralpak IA 5 μm 250×30 mm; Hexane/Ethanol 50:50; 40 ml/min; RT; 329 mg/3.5 ml EtOH; 7×0.5 ml; UV 254 nm) the fraction eluting at 10.3-11.5 min was collected to give 60 mg (33%) 1-14-1 with a purity of 99.9%.

MS (ESI⁺): m/e=500 (M+H⁺).

¹H NMR (CHLOROFORM-d, 400 MHz): δ (ppm) 7.99 (d, J=8.1 Hz, 1H, Bt-H), 7.89 (d, J=8.6 Hz, 2H, Ph-H), 7.42 (dd, J=8.1, 7.1 Hz, 1H, Bt-H), 7.37 (dd, J=8.3, 6.8 Hz, 1H, Bt-H), 7.30 (d, J=8.3 Hz, 1H, Bt-H), 7.25 (d, J=8.8 Hz, 2H, Ph-H), 6.58 (s, 1H, Pypy 6-H), 6.11 (s, 2H, OCH₂O), 3.93 (s, 2H, ArCH₂C), 3.53 (q, J=7.1 Hz, 2H, NCH₂), 3.39 (t, 2H, NCH₂), 2.75 (s, 2H, 5-CH₃), 2.55 (s, 2H, 7-CH₃), 1.24 (t, J=7.1 Hz, 3H, NCH₂CH₃), 1.11 (t, J=7.1 Hz, 2H, NCH₂CH₃).

¹³C NMR (101 MHz, DICHLOROMETHANE-d₂): δ (ppm) 170.0 (C═O), 158.1 (Ph C-4), 156.6 (Pypy C-5), 154.1 (Pypy C-2), 148.0 (Pypy C-3a), 145.2 (Pypy C-7), 143.8 (Bt C-3a), 130.4 (Ph C-2/6), 129.8 (Ph C-1), 129.1 (Bt C-7a), 128.6 (Bt C-6), 124.9 (Bt C-5), 120.1 (Bt C-4), 116.3 (Ph C-3/5), 109.3 (Bt C-7), 108.7 (Pypy C-6), 101.3 (Pypy C-3), 99.3 (OCH₂O), 42.6 (NCH₂ cis), 40.8 (NCH₂ trans), 28.3 (C-2), 24.7 (5-CH₃), 16.9 (7-CH₃), 14.4 (NCH₂CH₃ trans), 13.7 (NCH₂CH₃ cis),

### Example 15

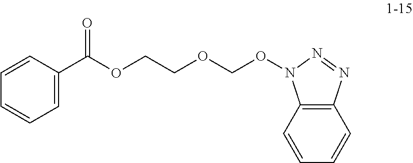 [SMILES: O=C(OCCOCOn1nnc2ccccc21)c1ccccc1]

### 2-[(1H-Benzotriazol-1-yloxy)methoxy]ethyl benzoate

As described in example 13, 100 mg (0.58 mmol) of the potassium salt of N-hydroxy-1H-benzotriazole prepared above and 124 mg (0.58 mmol) benzoyloxyethylchloromethylether were reacted. Chromatography of the raw material on 10 g silica (hexane, hexane/ethyl acetate 8:2) gave 30 mg (11%) of an oil, which was further purified by preparative HPLC: HPLC (Waters Autopurificationsystem: Pump 2545, Sample Manager 2767, CFO, DAD 2996, ELSD 2424, SQD 3001; XBrigde C18 5 μm 100×30 mm; A=H₂O+0.1% HCOOH; B=Acetonitril, 0-1 min 1% B, 1-8 min 1-99% B, 8-10 min 99% B; 50 ml/min.) to give 18 mg (9%) of 1-15.

¹H NMR (CHLOROFORM-d, 300 MHz): δ (ppm) 7.95-8.06 (m, 3H), 7.52-7.62 (m, 2H), 7.31-7.48 (m, 4H), 5.60 (s, 2H), 4.55 (t, J=4.5 Hz, 2H), 4.28 (t, J=4.5 Hz, 2H).

¹³C NMR (CHLOROFORM-d, 75 MHz): δ (ppm) 166.4 (C-1), 143.6 (Bt C-3a), 133.2 (C-5), 129.7 (C-3/7), 129.6 (C-2), 128.4 (C-4/6), 128.2 (Bt C-6), 128.2 (Bt C-7a), 124.6 (Bt C-5), 120.3 (Bt C-4), 108.5 (Bt C-7), 102.3 (OCH₂O), 68.6 (CH₂OC), 63.4 (CH₂OCO).

### Example 16

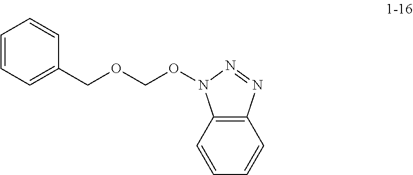 [SMILES: c1ccc(COCOn2nnc3ccccc32)cc1]

### 1-(Benzyloxymethoxy)-1H-benzotriazole

100 mg (0.58 mmol) of the potassium salt of N-hydroxy-1H-benzotriazole prepared in example 13 were suspended in 6.5 ml tetrahydrofuran and 81 μL (0.58 mmol) benzylchloromethylether was added. The reaction was stirred over night at ambient temperature and then partitioned between with ethyl acetate and water. The organic phase was dried (sodium sulfate) and evaporated i. vac. The residue was purified by chromatography and then by preparative HPLC (HPLC (Waters Autopurificationsystem: Pump 2545, Sample Manager 2767, CFO, DAD 2996, ELSD 2424, SQD 3001; XBrigde C18 5 μm 100×30 mm; A=H₂O+0.1% HCOOH; B=Acetonitril, 0-1 min 1% B, 1-8 min 1-99% B, 8-10 min 99% B; 50 ml/min.) to give 41 mg (27%) of 1-16.

¹H NMR (CHLOROFORM-d, 400 MHz): δ (ppm) 8.03 (d, J=8.3 Hz, 1H), 7.57 (d, J=8.6 Hz, 1H), 7.50 (ddd, J=8.3, 6.8, 0.5 Hz, 1H), 7.37-7.43 (ddd, J=8.3, 6.8, 1.0 Hz, 1H), 7.28-7.37 (m, 5H), 5.58 (s, 2H), 5.00 (s, 2H).

¹³C NMR (CHLOROFORM-d, 101 MHz): δ (ppm) 143.7 (Bt C-3a), 135.9 (Bn C-2), 128.7 (Bn C-3/7), 128.5 (Bt C-6), 128.3 (Bt C-7a), 128.2 (Bn C-4/6), 128.2 (Bn C-5), 124.7 (Bt C-5), 120.3 (Bt C-4), 108.8 (Bt C-7), 101.3 (OCH₂O), 72.0 (PhCH₂O).

### General Method for Radiofluorination

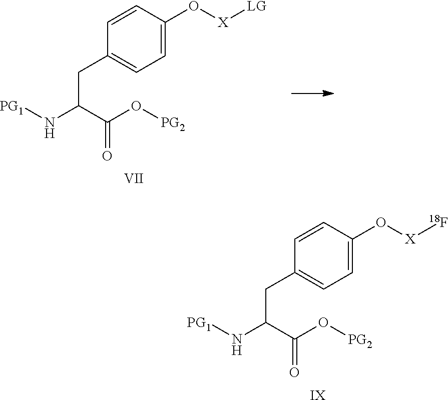

[¹⁸F]Fluoride was immobilized on a preconditioned QMA (Waters) cartridge (preconditioned by washing the cartridge with 5 ml 0.5 M potassium carbonate and 10 ml water), The [¹⁸F]fluoride was eluted using a solution of either:
I) potassium carbonate (1 mg) in 500 μl water and K₂₂₂ (5 mg) in 1500 μl acetonitrile
II) cesium carbonate (2.3 mg) in 500 μl water and K₂₂₂ (5 mg) in 1500 μl acetonitrile
III) 40% tetrabutylammonium hydroxide_{(aq)} (8 μl) in 500 ξL water and K₂₂₂ (5 mg) in 1500 μl acetonitrile

This solution was dried at 120° C. with a nitrogen flow of 150 ml/min. Additional acetonitrile (1 ml) was added and the drying step was repeated. This drying step was repeated once more. A solution of precursor (2 mg) in a solvent (300 μl) was added and heated at elevated temperature for a period of time (See Table 1 for details). The [¹⁸F]fluoride incorporation was analyzed by HPLC (ACE C18 3μ50×4.6 mm; Solvent A: 10 mM dipotassium phosphate in water, Solvent B: 10 mM dipotassium phosphate in acetonitrile:water (7:3); Gradient: 5% B to 95% B in 7 min, 95% B to 100% B in 6 sec, 100% B for 92 sec, 100% B to 5% B in 12 sec, 5% B for 3 min; flow: 2 ml/min). For compound 1-11-3 the [¹⁸F]fluoride incorporation was analyzed via a slightly modified HPLC method was used (ACE C18 3μ50×4.6 mm; Solvent A: 10 mM disodium phosphate in water pH 7.4, Solvent B: acetonitrile; Gradient: 5% B to 95% B in 7 min, 95% B to 100% B in 6 sec, 100% B for 92 sec, 100% B to 5% B in 12 sec, 5% B for 3 min; flow: 2 ml/min).

TABLE 1
Radiofluorination results using with different precursors and reaction conditions.

| | | | | | | Crypt- | | | Time | Incorpo- |
|---|---|---|---|---|---|---|---|---|---|---|
| Cpd | PG1 | PG2 | LG | X | Base | and | Solvent | Temp. | (min) | ration |
| 1-1-1 | Boc | tBu | OBt | CH₂ | TBAOH | na | MeCN | 140° C. | 15 | 38% |
| 1-1-1 | Boc | tBu | OBt | CH₂ | TBAOH | na | DMSO/MeCN | 140° C. | 15 | 40% |
| | | | | | | | (1:1) | | | |
| 1-1-2 | Boc | tBu | OAt | CH₂ | TBAOH | na | MeCN | 120° C. | 15 | 26% |
| 1-2-1 | Boc | dicyclopropyl | OBt | CH₂ | TBAOH | na | DMSO | 150° C. | 10 | 31% |
| 1-2-2 | Boc | dicyclopropyl | OBtCF₃ | CH₂ | Cs₂CO₃ | K222 | DMSO/MeCN | 140° C. | 15 | 52% |
| | | | | | | (5 mg) | (1:1) | | | |
| 1-2-3 | Boc | dicyclopropyl | OBtNO₂ | CH₂ | TBAOH | na | DMSO/MeCN | 140° C. | 15 | 12% |
| | | | | | | | (1:1) | | | |
| 1-3 | Boc | DMB | OBt | CH₂ | TBAOH | na | DMSO/MeCN | 140° C. | 15 | 18% |
| | | | | | | | (1:1) | | | |
| 1-3 | Boc | DMB | OBt | CH₂ | K₂CO₃ | K222 | DMSO/MeCN | 140° C. | 15 | 20% |
| | | | | | | (5 mg) | (1:1) | | | |
| 1-4-1 | Boc | cyclopropyl | OBt | CH₂ | TBAOH | na | DMSO/MeCN | 140° C. | 15 | 25% |
| | | | | | | | (1:1) | | | |
| 1-4-1 | Boc | cyclopropyl | OBt | CH₂ | K₂CO₃ | K222 | DMSO/MeCN | 140° C. | 15 | 35% |
| | | | | | | (5 mg) | (1:1) | | | |
| 1-4-2 | Boc | cyclopropyl | triazoleCO₂Et | CH₂ | TBAOH | na | DMSO | 120° C. | 10 | 12% |
| 1-4-2 | Boc | cyclopropyl | triazoleCO₂Et | CH₂ | Cs₂CO₃ | K222 | DMSO | 120° C. | 10 | 16% |
| | | | | | | (5 mg) | | | | |
| 1-5-1 | Boc | PMB | OBt | CH₂ | K₂CO₃ | K222 | DMSO/MeCN | 140° C. | 15 | 25% |
| | | | | | | (5 mg) | (1:1) | | | |
| 1-5-1 | Boc | PMB | OBt | CH₂ | TBAOH | na | DMSO/MeCN | 140° C. | 15 | 22% |
| | | | | | | | (1:1) | | | |
| 1-5-2 | Boc | PMB | OBt-Cl | CH₂ | K₂CO₃ | K222 | DMSO/MeCN | 140° C. | 15 | 33% |
| | | | | | | (5 mg) | (1:1) | | | |
| 1-5-3 | Boc | PMB | OBtCF₃ | CH₂ | K₂CO₃ | na | DMSO/MeCN | 140° C. | 15 | 25% |
| | | | | | | | (1:1) | | | |
| 1-5-4 | Boc | PMB | OBtCF₃ | CH₂ | Cs₂CO₃ | na | DMSO/MeCN | 140° C. | 15 | 60% |
| | | | | | | | (1:1) | | | |
| 1-6 | Boc | αMeBn | OBt | CH₂ | TBAOH | na | DMSO/MeCN | 140° C. | 15 | 13% |
| | | | | | | | (1:1) | | | |
| 1-6 | Boc | αMeBn | OBt | CH₂ | K₂CO₃ | K222 | DMSO/MeCN | 140° C. | 15 | 15% |
| | | | | | | (5 mg) | (1:1) | | | |
| 1-7 | Boc | cumyl | OBt | CH₂ | TBAOH | na | DMSO/MeCN | 140° C. | 15 | 21% |
| | | | | | | | (1:1) | | | |
| 1-7 | Boc | cumyl | OBt | CH₂ | K₂CO₃ | K222 | DMSO/MeCN | 140° C. | 15 | 20% |
| | | | | | | (5 mg) | (1:1) | | | |
| 1-8 | Trt | tBu | OBt | CH₂ | K₂CO₃ | K222 | DMSO/MeCN | 140° C. | 15 | 30% |
| | | | | | | (5 mg) | (1:1) | | | |
| 1-9 | Trt | PMB | OBt | CH₂ | TBAOH | na | DMSO/MeCN | 140° C. | 15 | 35% |
| | | | | | | | (1:1) | | | |
| 1-9 | Trt | PMB | OBt | CH₂ | K₂CO₃ | K222 | DMSO/MeCN | 140° C. | 15 | 32% |
| | | | | | | (5 mg) | (1:1) | | | |
| 1-10 | Boc | cyclopropyl | OBt | CD₂ | TBAOH | na | DMSO/MeCN | 140° C. | 15 | 38% |
| | | | | | | | (1:1) | | | |
| 1-10 | Boc | cyclopropyl | OBt | CD₂ | K₂CO₃ | K222 | DMSO/MeCN | 140° C. | 15 | 24% |
| | | | | | | (5 mg) | (1:1) | | | |
| 1-2-2 | Boc | dicyclopropyl | OBt | CH₂ | K₂CO₃ | K222 | DMSO/MeCN | 140° C. | 15 | 29% |
| | | | | | | (5 mg) | (1:1) | | | |
| 1-11-1 | Trt | Dmb | OBt | CH₂ | K₂CO₃ | K222 | DMSO/MeCN | 140° C. | 15 | 30% |
| | | | | | | (5 mg) | (1:1) | | | |
| 1-11-1 | Trt | Dmb | OBt | CH₂ | Cs₂CO₃ | K222 | DMSO/MeCN | 140° C. | 15 | 36% |
| | | | | | | (5 mg) | (1:1) | | | |
| 1-11-2 | Trt | Dmb | OBt-Cl | CH₂ | TBAOH | na | DMSO/MeCN | 140° C. | 15 | 27% |
| | | | | | | | (1:1) | | | |
| 1-11-2 | Trt | Dmb | OBt-Ct | CH₂ | K₂CO₃ | K222 | DMSO/MeCN | 140° C. | 15 | 28% |
| | | | | | | (5 mg) | (1:1) | | | |
| 1-11-3 | Trt | Dmb | OBt-CF3 | CH₂ | K₂CO₃ | K222 | DMSO/MeCN | 140° C. | 15 | 43% |
| | | | | | | (5 mg) | (1:1) | | | |
| 1-11-3 | Trt | Dmb | OBt-CF₃ | CH₂ | Cs₂CO₃ | K222 | DMSO/MeCN | 140° C. | 15 | 56% |
| | | | | | | (5 mg) | (1:1) | | | |
| 1-12 | Boc | Me (α-Me) | OBtl | CH₂ | Cs₂CO₃ | K222 | DMSO/MeCN | 140° C. | 15 | 40% |
| | | | | | | (5 mg) | (1:1) | | | |
| 1-12 | Boc | Me (α-Me) | OBt | CH₂ | K₂CO₃ | K222 | DMSO/MeCN | 140° C. | 15 | 31% |
| | | | | | | (5 mg) | (1:1) | | | |

### Synthesis of radioactive compounds

### Example 17

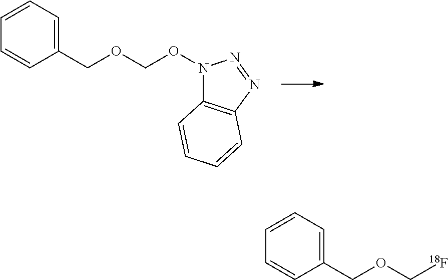 [SMILES: c1ccc(COCOn2nnc3ccccc32)cc1 ; [18F]COCc1ccccc1]

[¹⁸F]Fluoride was immobilized on a preconditioned QMA (Waters) cartridge (preconditioned by washing the cartridge with 5 ml 0.5 M K₂CO₃ and 10 ml water), The [¹⁸F]fluoride was eluted using a solution of either:
1) K₂CO₃ (1 mg) in 500 μl water and K₂₂₂ (5 mg) in 1500 μl acetonitrile
II) Cs₂CO₃ (2.3 mg) in 500 μl water and K₂₂₂ (5 mg) in 1500 μl acetonitrile
III) 40% TBAOH_{(aq)} (8 μl) in 500 μl water and K₂₂₂ (5 mg) in 1500 μl acetonitrile

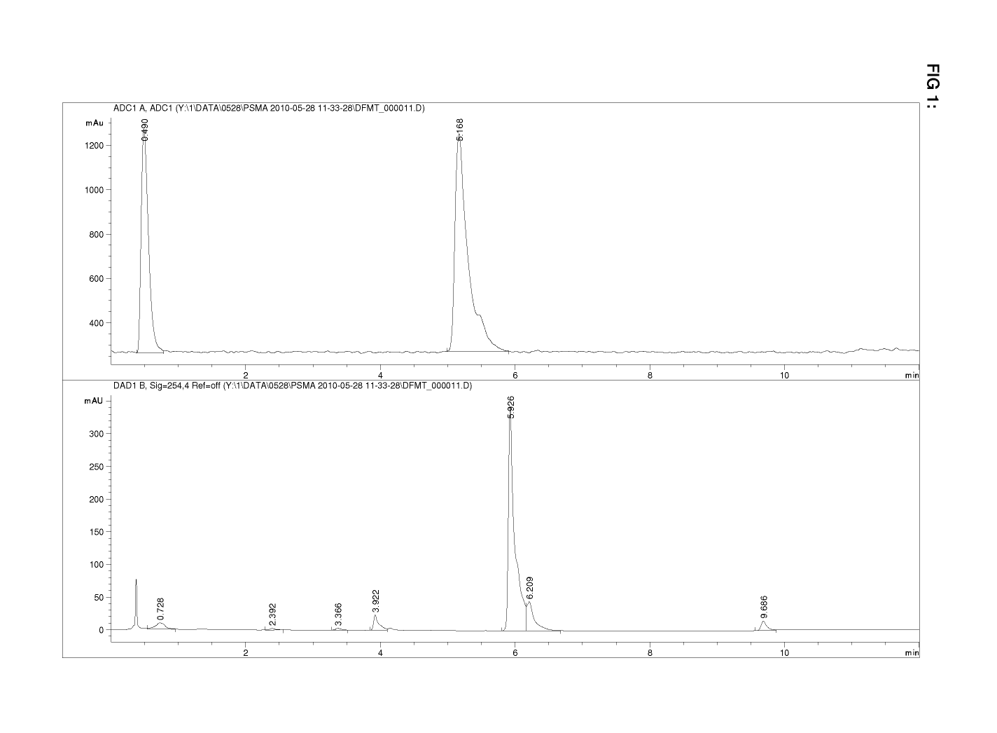

This solution was dried at 120° C. with a nitrogen flow of 150 ml/min. Additional acetonitrile (1 ml) was added and the drying step was repeated. This drying step was repeated once more. A solution of precursor (2 mg) in a DMSO:acetonitrile (1:1, 300 μl) was added and heated at 140° C. for 10 min. The [¹⁸F]fluoride incorporation was analyzed by HPLC (ACE C18 3μ50×4.6 mm; Solvent A: 10 mM K₂HPO₄ in water, Solvent B: 10 mM K₂HPO₄ in acetonitrile:water (7:3); Gradient: 5% B to 95% B in 7 min, 95% B to 100% B in 6 sec, 100% B for 92 sec, 100% B to 5% B in 12 sec, 5% B for 3 min; flow: 2 ml/min). The [¹⁸F]fluoride incorporation was:
I) K₂CO₃=78.5%
II) Cs₂CO₃=63.6% (For HPLC see FIG. 1)
III) TBAOH=74.4%

FIG. 1: HPLC above γ-trace and below UV detector.

### Example 18

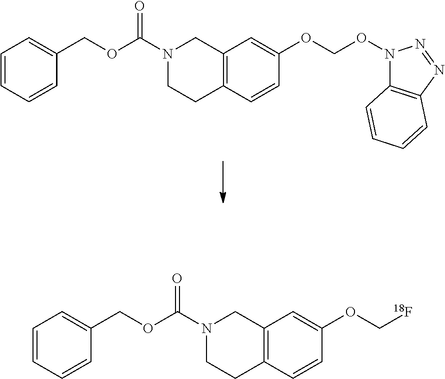 [SMILES: O=C(OCc1ccccc1)N1CCc2ccc(OCOn3nnc4ccccc43)cc2C1 ; O=C(OCc1ccccc1)N1CCc2ccc(OC[18F])cc2C1]

[¹⁸F]Fluoride was immobilized on a preconditioned QMA (Waters) cartridge (preconditioned by washing the cartridge with 5 ml 0.5 M K₂CO₃ and 10 ml water), The [¹⁸F]fluoride was eluted using a solution of either:
I) K₂CO₃ (1 mg) in 500 μl water and K₂₂₂ (5 mg) in 1500 μl acetonitrile
II) 40% TBAOH_{(aq)} (8 μl) in 500 μl water and K₂₂₂ (5 mg) in 1500 μl acetonitrile

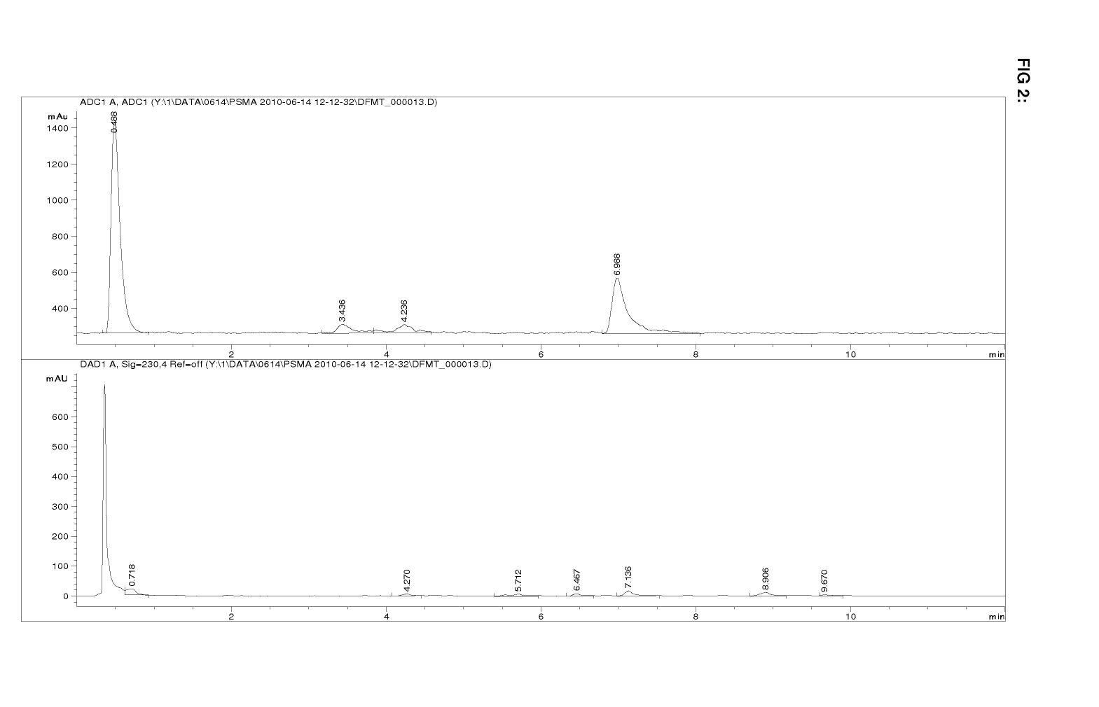

This solution was dried at 120° C. with a nitrogen flow of 150 ml/min. Additional acetonitrile (1 ml) was added and the drying step was repeated. This drying step was repeated once more. A solution of precursor (2 mg) in a DMSO:acetonitrile (1:1, 300 μl) was added and heated at 140° C. for 15 min. The [¹⁸F]fluoride incorporation was analyzed by HPLC (ACE C18 3μ50×4.6 mm; Solvent A: 10 mM K₂HPO₄ in water, Solvent B: 10 mM K₂HPO₄ in acetonitrile:water (7:3); Gradient: 5% B to 95% B in 7 min, 95% B to 100% B in 6 sec, 100% B for 92 sec, 100% B to 5% B in 12 sec, 5% B for 3 min; flow: 2 ml/min). The [¹⁸F]fluoride incorporation was:
I) K₂CO₃=28.5%
II) TBAOH=38.4% (for HPLC see FIG. 2)

FIG. 2: HPLC above γ-trace and below UV detector.

### Example 19

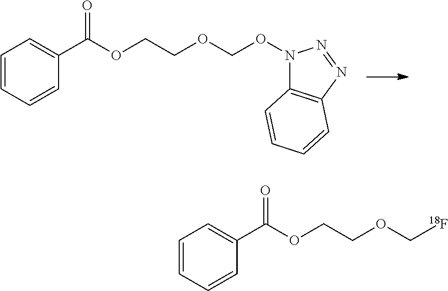 [SMILES: O=C(OCCOCOn1nnc2ccccc21)c1ccccc1 ; O=C(OCCOC[18F])c1ccccc1]

[¹⁸F]Fluoride was immobilized on a preconditioned QMA (Waters) cartridge (preconditioned by washing the cartridge with 5 ml 0.5 M K₂CO₃ and 10 ml water), The [¹⁸F]fluoride was eluted using a solution of either:
I) K₂CO₃ (1 mg) in 500 μl water and K₂₂₂ (5 mg) in 1500 μl acetonitrile
II) Cs₂CO₃ (2.3 mg) in 500 μl water and K₂₂₂ (5 mg) in 1500 μl acetonitrile
III) 40% TBAOH_{(aq)} (80) in 500 μl water and K₂₂₂ (5 mg) in 1500 μl acetonitrile

This solution was dried at 120° C. with a nitrogen flow of 150 ml/min. Additional acetonitrile (1 ml) was added and the drying step was repeated. This drying step was repeated once more. A solution of precursor (2 mg) in a DMSO:acetonitrile (1:1, 300 μl) was added and heated at 140° C. for 10 min. The [¹⁸F]fluoride incorporation was analyzed by HPLC (ACE C18 3μ50×4.6 mm; Solvent A: 10 mM K₂HPO₄ in water, Solvent B: 10 mM K₂HPO₄ in acetonitrile:water (7:3); Gradient: 5% B to 95% B in 7 min, 95% B to 100% B in 6 sec, 100% B for 92 sec, 100% B to 5% B in 12 sec, 5% B for 3 min; flow: 2 ml/min). The [¹⁸F]fluoride incorporation was:
I) K₂CO₃=26.9%
II) Cs₂CO₃=33.5%
III) TBAOH=33.9%

### Example 20

### DPA714

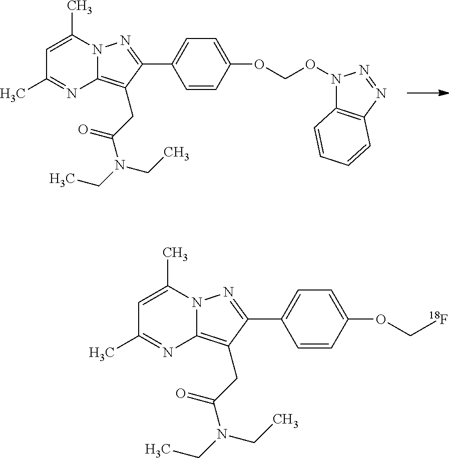 [SMILES: CCN(CC)C(=O)Cc1c(-c2ccc(OCOn3nnc4ccccc43)cc2)nn2c(C)cc(C)nc12 ; CCN(CC)C(=O)Cc1c(-c2ccc(OC[18F])cc2)nn2c(C)cc(C)nc12]

[¹⁸F]Fluoride was immobilized on a preconditioned QMA (Waters) cartridge (preconditioned by washing the cartridge with 5 ml 0.5 M K₂CO₃ and 10 ml water), The [¹⁸F]fluoride was eluted using a solution of K₂CO₃ (1 mg) in 500 it water and K₂₂₂ (5 mg) in 1500 μl acetonitrile. This solution was dried at 120° C. with a nitrogen flow of 150 ml/min. Additional acetonitrile (1 ml) was added and the drying step was repeated. This drying step was repeated once more. A solution of precursor (2 mg) in a DMSO:acetonitrile (1:1, 300 μl) was added and heated at 140° C. for 15 min. The [¹⁸F]fluoride incorporation was analyzed by HPLC (ACE C18 3μ50×4.6 mm; Solvent A: 10 mM K₂HPO₄ in water, Solvent B: 10 mM K₂HPO₄ in acetonitrile:water (7:3); Gradient: 5% B to 95% B in 7 min, 95% B to 100% B in 6 sec, 100% B for 92 sec, 100% B to 5% B in 12 sec, 5% B for 3 min; flow: 2 ml/min). The [¹⁸F]fluoride incorporation was 2%.

### Example 21

### Radiosynthesis of O—[¹⁸F]Fluoromethyl tyrosine (Precursor 1-2-1)

[¹⁸F]Fluoride (1.72 GBq) was immobilized on a preconditioned QMA (Waters) cartridge (preconditioned by washing the cartridge with 5 ml 0.5M potassium carbonate and 10 ml water), The [¹⁸F]fluoride was eluted using a solution of potassium carbonate (1 mg) in 250 μl water and K₂₂₂ (5 mg) in 1250 μl acetonitrile. This solution was dried at 120° C. with stirring under a nitrogen stream. Additional acetonitrile (1 ml) was added and the drying step was repeated. A solution of precursor 1-2-1 (2 mg) in dimethyl sulfoxide:acetonitrile (1:1; 300 μl) was added and heated at 140° C. for 15 min. The reaction was diluted with water (20 ml) and passed through a C18 Plus Light (preconditioned by washing the cartridge with 5 ml ethanol and 10 ml water). The solid phase extraction (SPE) cartridge was washed with water (10 ml) and eluted with acetonitrile (1 ml). The elution was concentrated at 70° C. with stirring under a nitrogen stream. To this was added dichloromethane:trifluoroacetic acid (1:2, 500 μl) and stirred at r.t. for 2 min. The reaction was concentrated under a nitrogen stream. To the residue was added pH2 water (4 ml, water pH adjusted to pH 2 with 0.1 M hydrochloric acid) and purified by HPLC (Synergi Hydro RP 4μ250×10 mm; 10% acetonitrile in water at pH 2; flow 5 ml/min). The product peak was collected, diluted with water (pH 2) and passed through a C18 Plus Environmental SPE (preconditioned by washing the cartridge with 5 ml ethanol and 10 ml water). The SPE cartridge was washed with water pH 2 (5 ml). The product was eluted with a 1:1 mixture of ethanol and water pH2 (3 ml). Starting from 1.72 GBq [¹⁸F]fluoride, 132 MBq (5.7% d.c.) of desired product were obtained in 103 min.

### Example 22

### Radiosynthesis of O—[¹⁸F]Fluoromethyl tyrosine (Precursor 1-3)

[¹⁸F]Fluoride (1.697 GBq) was immobilized on a preconditioned QMA (Waters) cartridge (preconditioned by washing the cartridge with 5 ml 0.5M potassium carbonate and 10 ml water), The [¹⁸F]fluoride was eluted using a solution of potassium carbonate (1 mg) in 500 μl water and K₂₂₂ (5 mg) in 1500 μl acetonitrile. This solution was dried at 120° C. with stirring under a nitrogen stream. Additional acetonitrile (1 ml) was added and the drying step was repeated. Additional acetonitrile (1 ml) was added and the drying step was repeated. A solution of precursor 1-3 (2 mg) in dimethyl sulfoxide:acetonitrile (1:1; 300 μl was added and heated at 140° C. for 15 min. The reaction was diluted with water (10 ml) and passed through a C18 Plus Light (preconditioned by washing the cartridge with 5 ml ethanol and 10 ml water). The SPE cartridge was washed with water (5 ml) and eluted with acetonitrile (1 ml). The elution was concentrated at 70° C. with stirring under a nitrogen stream. To this was added dichloromethane:trifluoroacetic acid (1:2, 500 μl and stirred at r.t. for 10 min. The reaction was concentrated under a nitrogen stream. To the residue was added pH2 water at (5 ml, water pH adjusted to pH 2 with 0.1 M hydrochloric acid) and purified by HPLC (Synergi Hydro RP 4μ250×10 mm; 10% acetonitrile in water at pH 2; flow 5 ml/min). The product peak was collected, diluted with water (pH 2) and passed through a C18 Plus Environmental SPE (preconditioned by washing the cartridge with 5 ml ethanol and 10 ml water). The SPE cartridge was washed with water pH 2 (5 ml). The product was eluted with a 1:1 mixture of ethanol and water pH2 (2 ml). Starting from 1.697 GBq [¹⁸F]fluoride, 5.7 MBq (0.8% d.c.) of desired product was isolated. The product was analyzed by analytical HPLC (ACE C18 3μ50×4.6 mm; Solvent A: 10 mM dipotassium phosphate in water, Solvent B: 10 mM dipotassium phosphate in acetonitrile:water (7:3); Gradient: 5% B to 95% B in 7 min, 95% B to 100% B in 6 sec, 100% B for 92 sec, 100% B to 5% B in 12 sec, 5% B for 3 min; flow: 2 ml/min).

### Example 23

### Radiosynthesis of O—[¹⁸F]Fluoromethyl-D-tyrosine (Precursor 1-11-1)

[¹⁸F]Fluoride (1063 MBq) was immobilized on a preconditioned QMA (Waters) cartridge (preconditioned by washing the cartridge with 5 ml 0.5M potassium carbonate and 10 ml water), The [¹⁸F]fluoride was eluted using a solution of potassium carbonate (1 mg) in 500 μl water and K₂₂₂ (5 mg) in 1500 μl acetonitrile. This solution was dried at 120° C. with stirring under a nitrogen stream. Additional acetonitrile (1 ml) was added and the drying step was repeated. Additional acetonitrile (1 ml) was added and the drying step was repeated. A solution of precursor 1-11-1 (2 mg) in dimethyl sulfoxide:acetonitrile (1:1; 300 μl) was added and heated at 140° C. for 15 min. The reaction was diluted with water (10 ml) and passed through a C18 Plus Light (preconditioned by washing the cartridge with 5 ml ethanol and 10 ml water). The SPE cartridge was washed with water (5 ml) and eluted with acetonitrile (1 ml). The elution was concentrated at 70° C. with stirring under a nitrogen stream. To this was added dichloromethane:trifluoroacetic acid (1:2, 500 μl and stirred at r.t. for 10 min. The reaction was concentrated under a nitrogen stream. To the residue was added pH2 water (5 ml, water pH adjusted to pH 2 with 0.1 M hydrochloric acid) and purified by HPLC (Synergi Hydro RP 4μ250×10 mm; 10% acetonitrile in water at pH 2; flow 5 ml/min). The product peak was collected, diluted with water (pH 2) and passed through a C18 Plus Environmental SPE (preconditioned by washing the cartridge with 5 ml ethanol and 10 ml water). The SPE cartridge was washed with water pH 2 (5 ml). The product was eluted with a 1:1 mixture of ethanol and water pH2 (2 ml). Starting from 1063 MBq [¹⁸F]fluoride, 1.7 MBq (0.4% d.c.) of D-FMT was isolated. The product was analyzed by analytical HPLC (ACE C18 3μ50×4.6 mm; Solvent A: 10 mM dipotassium phosphate in water, Solvent B: 10 mM dipotassium phosphate in acetonitrile:water (7:3); Gradient: 5% B to 95% B in 7 min, 95% B to 100% B in 6 sec, 100% B for 92 sec, 100% B to 5% B in 12 sec, 5% B for 3 min; flow: 2 ml/min) and using a chiral HPLC (Astec Chirobiotic T 250×4.6 mm; Solvent A: Water, Solvent B: Ethanol; Gradient: 50% B in A isocratic; flow: 5 ml/min).

### Example 24

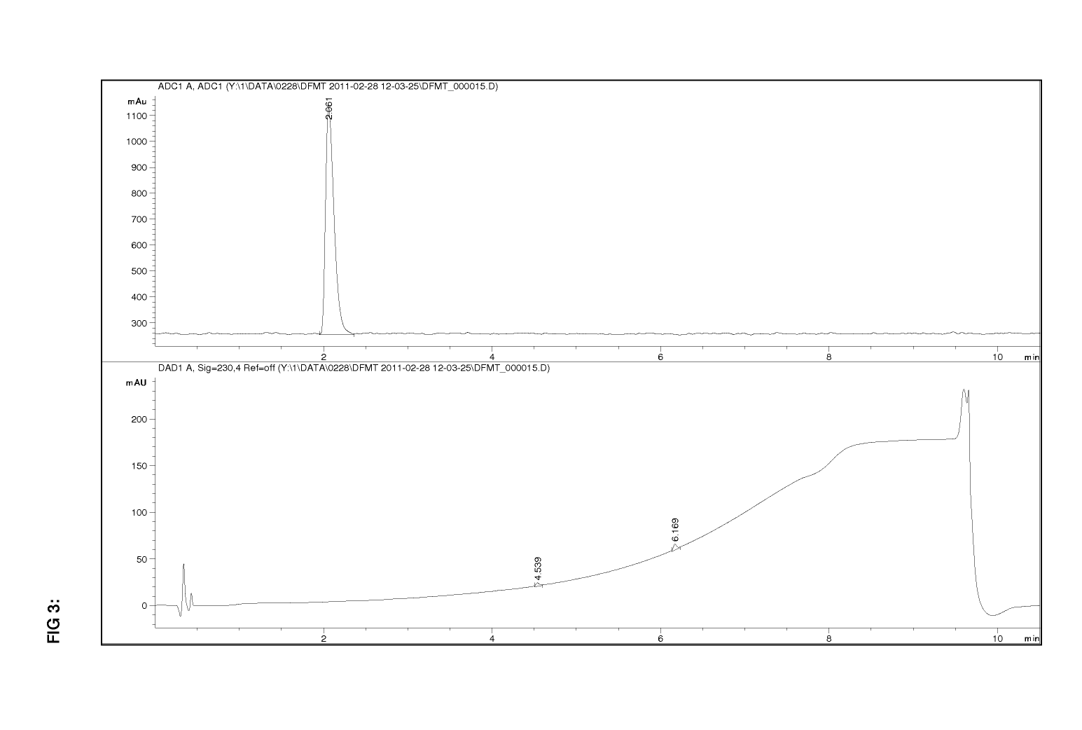

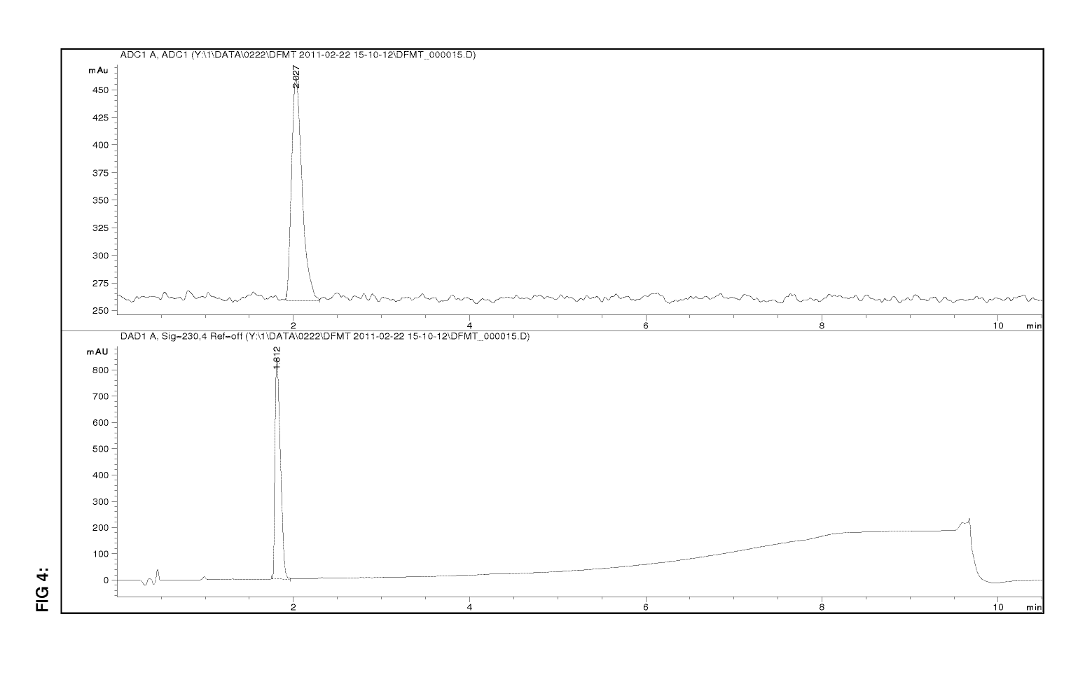

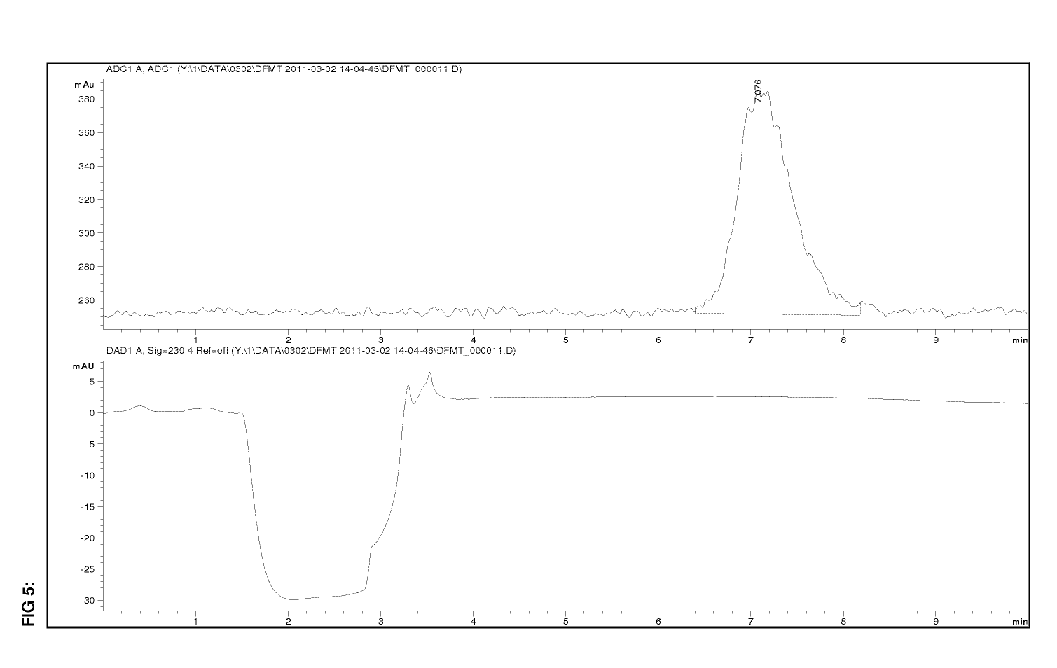

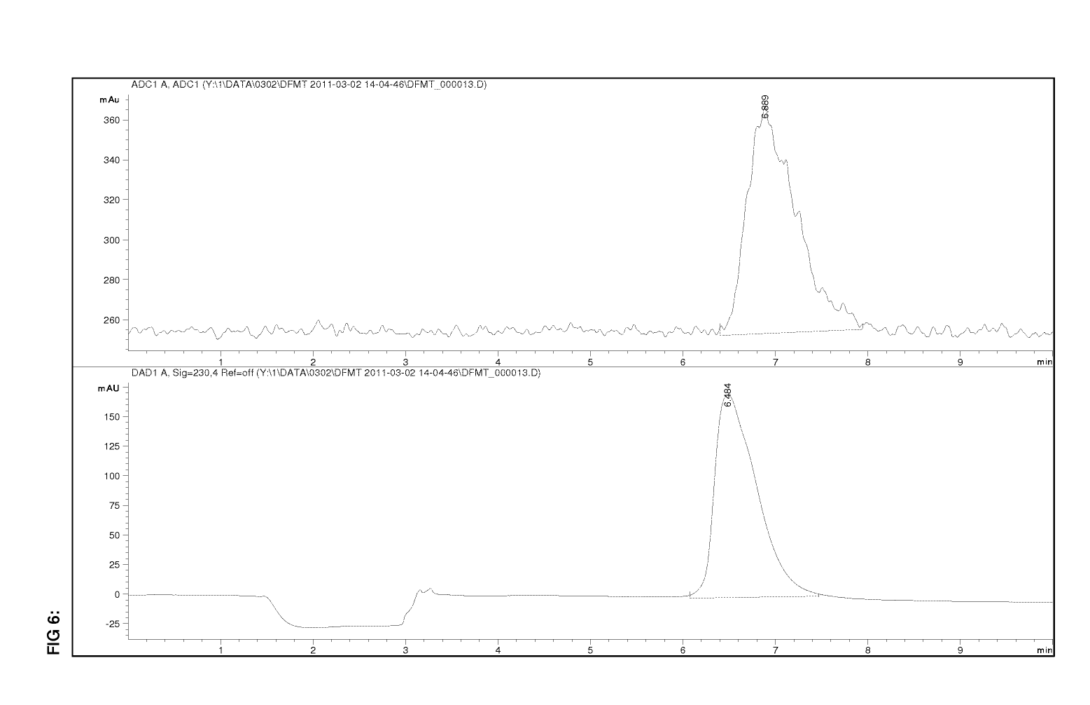

### Radiosynthesis of O—[¹⁸F]Fluoromethyl-D-tyrosine (Precursor 1-11-3)

[¹⁸F]Fluoride (2086 MBq) was immobilized on a preconditioned QMA (Waters) cartridge (preconditioned by washing the cartridge with 5 ml 0.5M potassium carbonate and 10 ml water), The [¹⁸F]fluoride was eluted using a solution of potassium carbonate (1 mg) in 500 μl water and K₂₂₂ (5 mg) in 1500 μl acetonitrile. This solution was dried at 120° C. with stirring under a nitrogen stream. Additional acetonitrile (1 ml) was added and the drying step was repeated. This azeotropic drying step was repeated twice more. A solution of precursor 1-11-3 (2 mg) in dimethyl sulfoxide:acetonitrile (1:1; 300 μl was added and heated at 140° C. for 15 min. The reaction mixture was diluted with 1.5 ml MeCN (1.5 ml) and passed through a Silica Plus SPE (preconditioned with 5 ml MeCN). The SPE was washed with MeCN (1.5 ml). This solution was purified by HPLC (ACE 5μ C18, 250×10 mm; 85% acetonitrile in water+0.% TFA; flow 5 ml/min). The product peak was collected, diluted with pH2 water (10 ml, water pH adjusted to pH 2 with 0.1 M hydrochloric acid) and stood for 10 min. This solution was passed through a SCX SPE (not preconditioned). The SPE cartridge was washed with pH2 water:MeCN (10 ml, 1:1). The SPE was kept wet for 2 min and then air (10 ml) was passed through. through. The SPE cartridge was washed with pH2 water (10 ml, 1:1). The SPE was kept wet for 2 min and then air (10 ml) was passed through. The desired product was eluted with a 10 ml buffer solution (7 g Na₂HPO₄ and 6 g NaCl in 1 L). Starting from 2086 MBq [¹⁸F]fluoride, 161.8 MBq (14.6% d.c.) of D-FMT was isolated. The product was analyzed by analytical HPLC (FIG. 3) (ACE C18 3μ50×4.6 mm; Solvent A: water+0.1% TFA, Solvent B: acetonitrile+0.1% TFA: Gradient: 5% B to 95% B in 7 min, 95% B to 100% B in 6 sec, 100% B for 92 sec, 100% B to 5% B in 12 sec, 5% B for 3 min; flow: 2 ml/min) and with co-injection of the cold standard (FIG. 4). The product was also analyzed using a chiral HPLC (FIG. 5) (Astec Chirobiotic T 250×4.6 mm; Solvent A: Water, Solvent B: Ethanol; Gradient: 50% B in A isocratic; flow: 5 ml/min) and with co-injection of the cold standard (FIG. 6).

FIGS. 2, 3, 4 and 5: HPLC Left UV-detector and Right γ-detector.

1-21. (canceled)

22. A radiolabelling method for converting compounds of formula I into compounds of formula II 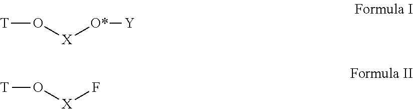
wherein
F is [¹⁸F] fluorine atom;
T is a small molecule having a molecular mass of about 150 daltons to about 1.500 daltons encompassing an aromatic or heteroaromatic moiety, wherein the —O—X—O*—Y group is covalently bond to the aromatic or heteroaromatic moiety;
X is CH₂, CHD or CD₂;
Y is a substituted heteroaromatic ring containing one to four nitrogen atoms with the proviso that the oxygen (O*) is directly bound to one of the nitrogens of the heteroaromatic ring and O*—Y acts as leaving group,
said method comprising reacting a compound of Formula I with a [¹⁸F]-Fluorination agent the step
Reacting compound of Formula I with a [¹⁸F]-Fluorination agent.

23. A radiolabelling method according to claim 22, further comprising deprotecting the obtained compound to obtain a deprotected compound of formula II.

24. A radiolabelling method according to claim 22, further comprising deprotecting the obtained compound to obtain a deprotected compound of formula II, and converting the deprotected compound of formula II into a suitable salt of inorganic or organic bases thereof, a hydrate thereof, a complex thereof, or a solvate thereof.

25. The method according to claim 22, wherein independently from each other
X is CH₂, or CD₂;
Y is 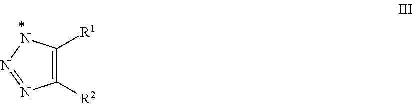
wherein * indicates the position of the covalent bond to the Oxygen (O*) in formula I;
R¹ is H, CN, or COOR⁴, and R² is H, CN, or COOR⁴, or
R¹ and R² together form a 6 membered aromatic ring, or
R¹ and R² together form a 6 membered aromatic ring which comprises 1 Nitrogen (N), or
R¹ and R² form together a 6 membered aromatic ring which comprises 1 Nitrogen (N) and 1 methine of the 6 membered ring that is substituted with Halogen, NO₂, CN, COOR^{S}, SO₂R³ or CF₃;
R³ is C₁-C₃ alkyl;
R⁴ is C₁-C₆ alkyl; and
T is a small molecule having a molecular mass of from about 150 daltons to about 1,500 daltons encompassing an aromatic or heteroaromatic moiety, wherein the —O—X—O*—Y group is covalently bond to the aromatic or heteroaromatic moiety.

26. The method according to claim 22, wherein independently from each other
Y is 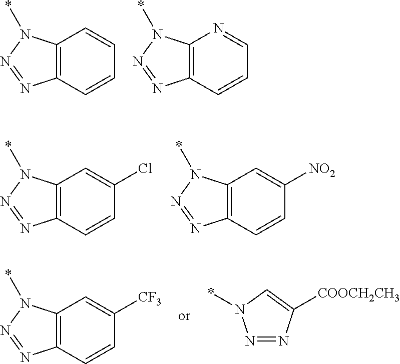
and
T is a small molecule having a molecular mass of from about 150 daltons to about 1,500 daltons, and a biological activity,
wherein said small molecule interacts with or has an effect on cell tissue or biological elements of mammal body, and encompasses an aromatic or heteroaromatic moiety wherein the —O—X—O*—Y and —O—X—F groups are covalently bond to the aromatic or heteroaromatic moiety.

27. The method according to claim 22, wherein independently from each other
Y is 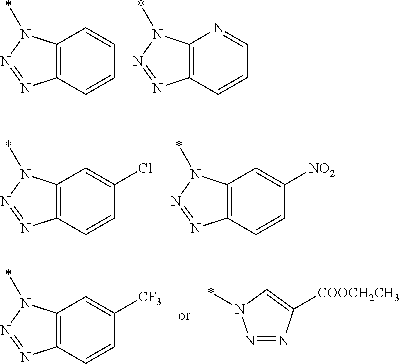
and
T is a small molecule having a molecular mass of from about 150 daltons to about 1,500 daltons, and a biological activity,
wherein said small molecule interacts with or has an effect on cell tissue or biological elements of mammal body, and encompasses an aromatic or heteroaromatic moiety wherein —O—X—O*—Y and —O—X—F groups are covalently bond to the aromatic or heteroaromatic moiety at the para position.

28. A compound of Formula Ia 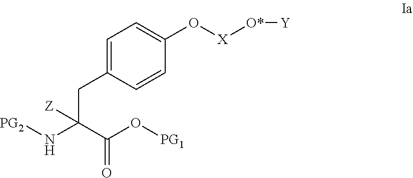
wherein:
X is CH₂, CHD or CD₂;
Y is a substituted or unsubstituted heteroaromatic ring containing one to four Nitrogen atoms (N) with the proviso that the oxygen (O*) is directly bound to one of the Nitrogen atoms (N) of the heteroaromatic ring and O*—Y acts as leaving group;
Z is Hydrogen or methyl;
PG1 is a carboxylic acid protecting group, containing up to 20 carbon atoms, or PG1 is a carboxylic acid protecting group, containing up to 20 carbon atoms containing independently one or more O, N or S atoms; and
PG2 is an amino protecting group, containing up to 20 carbon atoms, or PG2 is an amino protecting group, containing up to 20 carbon atoms containing one or more O, N or S atoms, or PG2 is an amino protecting group, containing up to 20 carbon atoms containing one or more O, N or S atoms substituted with one to three halogens.

29. The compound according to claim 28, wherein independently from each other
X is CH₂ or CD₂;
Y is a moiety of Formula III 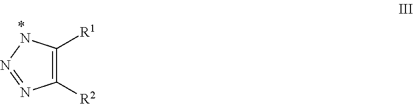
wherein * indicates the position of the covalent bond to the Oxygen (O*) in Formula Ia;
R¹ is H, CN, or COOR⁴, and R² is H, CN, or COOR⁴, or
R¹ and R² together form a 6 membered aromatic ring, or R¹ and R² form together a 6 membered aromatic ring which comprise 1 nitrogen atom (N) and 1 methine of the 6 membered ring is substituted with halogen, NO₂, CN, COOR^{S}, SO₂R³ or CF₃;
R³ is C₁-C₃ alkyl;
R⁴ is C₁-C₆ alkyl;
PG1 is
alkyl,
alkyl substituted with one phenyl or with one phenyl substituted with up to three C₁-C₃ alkyl, C₁-C₃ alkoxy or halogen,
alkyl substituted with one or two C₃-C₆ cycloalkyl,
alkyl substituted with one phenyl or with one phenyl substituted with up to three C₁-C₃ alkyl, C₁-C₃ alkoxy or halogen and one C₃-C₆ cycloalkyl, or
fluorenylmethyl;
alkyl is a branched or linear C₁-C₆ alkyl, or alkyl is a branched or linear C₁-C₆ alkyl substituted with C₁-C₃alkoxy; and
PG2 is Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) or 4-Methoxyphenyldiphenylmethyl (MMTr).

30. The compound according to claim 28, wherein
X is CH₂ or CD₂;
Y is 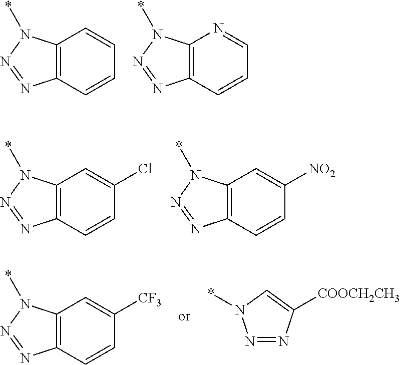
Z is hydrogen or methyl;
PG1 is dicyclopropylmethyl or 2,4-dimethoxybenzyl; and
PG2 is tert-Butoxycarbonyl (BOC) or Triphenylmethyl (trityl).

31. The compound according to claim 28, wherein said compound is a compound of Formula (Ib) 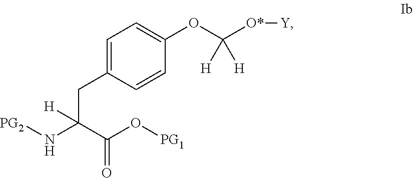
a compound of Formula (Ic) 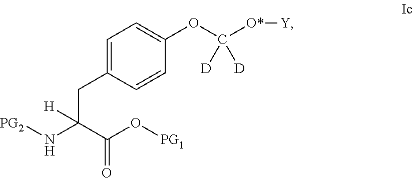
a compound of Formula (Id) 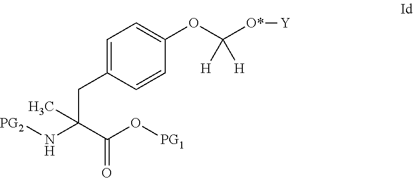
or
a compound of Formula (Ie) 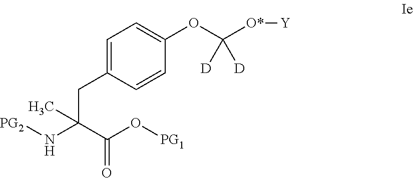
wherein X, Y, Z, PG1 and PG2 are as defined.

32. The compound according to claim 28, wherein said compound is a compound of Formula (D-Ia), (D-Ib), (D-Ic), (D-Id) or (D-Ie)

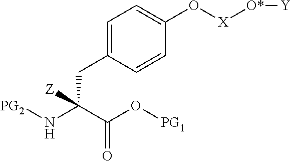

| Formula\Substituent | Z | X |
|---|---|---|
| D-Ia | H, CH₃ | CH₂, CD₂ |
| D-Ib | H | CH₂ |
| D-Ic | H | CD₂ |
| D-Id | CH₃ | CH₂ |
| D-Ie | CH₃ | CD₂ |

wherein Y, PG1 and PG2 are as defined in claim 28.

33. The compound according to claim 28, wherein said compound is:
tert-Butyl O-[(1H-benzotriazol-1-yloxy)methyl]-N-(tert-butoxycarbonyl)-D-tyrosinate 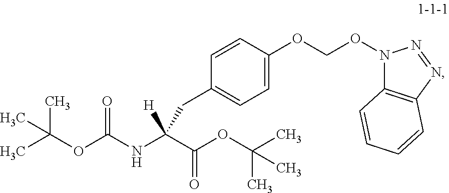 [SMILES: CC(C)(C)OC(=O)N[C@H](Cc1ccc(OCOn2nnc3ccccc32)cc1)C(=O)OC(C)(C)C]
tert-Butyl N-(tert-butoxycarbonyl)-O-[(1H-1,2,3-triazolo[5,4-b]pyridin-1-yloxy)methyl]-D-tyrosinate 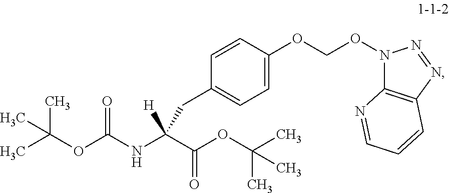 [SMILES: CC(C)(C)OC(=O)N[C@H](Cc1ccc(OCOn2nnc3cccnc32)cc1)C(=O)OC(C)(C)C]
Dicyclopropylmethyl O-[(1H-benzotriazol-1-yloxy)methyl]-N-(tert-butoxycarbonyl)-D-tyrosinate 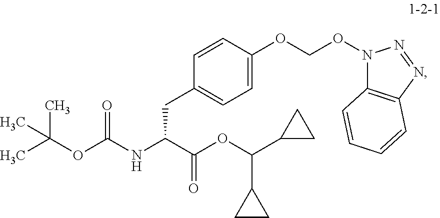 [SMILES: CC(C)(C)OC(=O)N[C@H](Cc1ccc(OCOn2nnc3ccccc32)cc1)C(=O)OC(C1CC1)C1CC1]
Dicyclopropylmethyl O-[(1H-benzotriazol-1-yloxy)methyl]-N-(tert-butoxycarbonyl)-L-tyrosinate 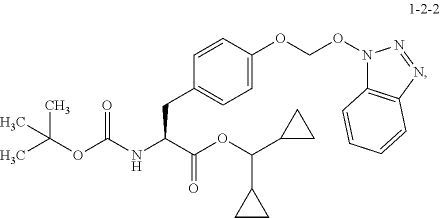 [SMILES: CC(C)(C)OC(=O)N[C@@H](Cc1ccc(OCOn2nnc3ccccc32)cc1)C(=O)OC(C1CC1)C1CC1]
Dicyclopropylmethyl O-[(6-nitro-1H-benzotriazol-1-yloxy)methyl]-N-(tert-butoxy carbonyl)-D-tyrosinate 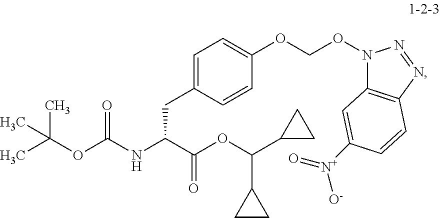 [SMILES: CC(C)(C)OC(=O)N[C@H](Cc1ccc(OCOn2nnc3ccc([N+](=O)[O-])cc32)cc1)C(=O)OC(C1CC1)C1CC1]
2,4-Dimethoxybenzyl O-[(1H-benzotriazol-1-yloxy)methyl]-N-(tert-butoxycarbonyl)-D-tyrosinate 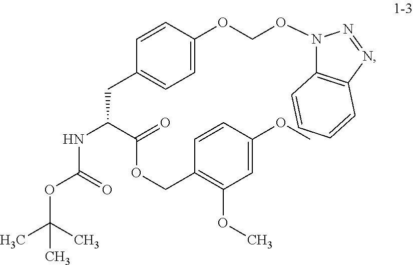 [SMILES: COc1ccc(COC(=O)[C@@H](Cc2ccc(OCOn3nnc4ccccc43)cc2)NC(=O)OC(C)(C)C)c(OC)c1]
Cyclopropylmethyl O-[(1H-benzotriazol-1-yloxy)methyl]-N-(tert-butoxycarbonyl)-D-tyrosinate 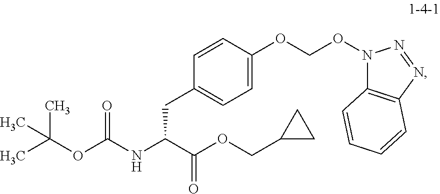 [SMILES: CC(C)(C)OC(=O)N[C@H](Cc1ccc(OCOn2nnc3ccccc32)cc1)C(=O)OCC1CC1]
Cyclopropylmethyl N-(tert-butoxycarbonyl)-O-({[4-(ethoxycarbonyl)-1H-1,2,3-triazol-1-yl]oxy}methyl)-D-tyrosinate 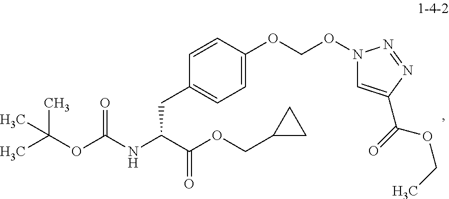 [SMILES: CCOC(=O)c1cn(OCOc2ccc(C[C@@H](NC(=O)OC(C)(C)C)C(=O)OCC3CC3)cc2)nn1]
4-Methoxybenzyl O-[(1H-benzotriazol-1-yloxy)methyl]-N-(tert-butoxycarbonyl)-D-tyrosinate 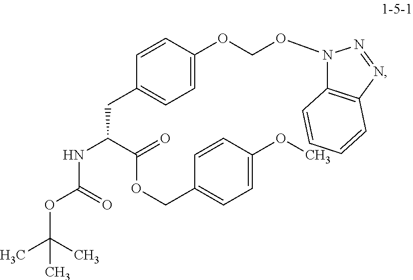 [SMILES: COc1ccc(COC(=O)[C@@H](Cc2ccc(OCOn3nnc4ccccc43)cc2)NC(=O)OC(C)(C)C)cc1]
4-Methoxybenzyl N-(tert-butoxycarbonyl)-O-{[(6-chloro-1H-benzotriazol-1-yl)oxy]methyl}-D-tyrosinate 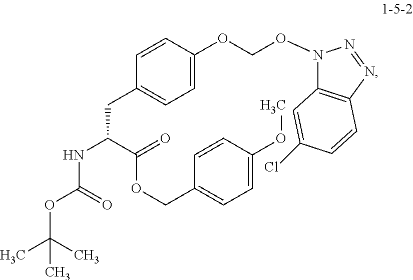 [SMILES: COc1ccc(COC(=O)[C@@H](Cc2ccc(OCOn3nnc4ccc(Cl)cc43)cc2)NC(=O)OC(C)(C)C)cc1]
4-Methoxybenzyl N-(tert-butoxycarbonyl)-O-[(6-trifluoromethyl-1H-benzotriazol-1-yloxy)methyl]-D-tyrosinate 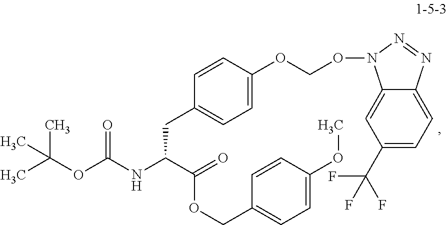 [SMILES: COc1ccc(COC(=O)[C@@H](Cc2ccc(OCOn3nnc4ccc(C(F)(F)F)cc43)cc2)NC(=O)OC(C)(C)C)cc1]
4-Methoxybenzyl O-[(6-trifluoromethyl-1H-benzotriazol-1-yloxy)methyl]-N-(tert-butoxycarbonyl)-L-tyrosinate. 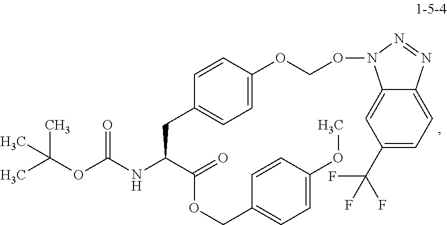 [SMILES: COc1ccc(COC(=O)[C@H](Cc2ccc(OCOn3nnc4ccc(C(F)(F)F)cc43)cc2)NC(=O)OC(C)(C)C)cc1]
alpha-Methylbenzyl O-[(1H-benzotriazol-1-yloxy)methyl]-N-(tert-butoxycarbonyl)-D-tyrosinate 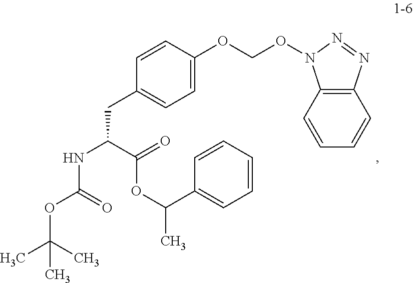 [SMILES: CC(OC(=O)[C@@H](Cc1ccc(OCOn2nnc3ccccc32)cc1)NC(=O)OC(C)(C)C)c1ccccc1]
alpha,alpha-Dimethylbenzyl O-[(1H-benzotriazol-1-yloxy)methyl]-N-(tert-butoxycarbonyl)-D-tyrosinate 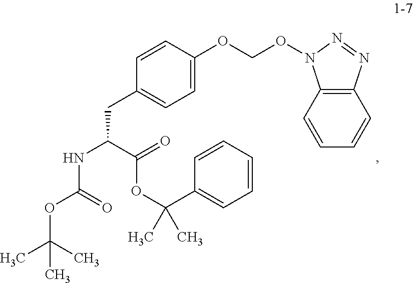 [SMILES: CC(C)(C)OC(=O)N[C@H](Cc1ccc(OCOn2nnc3ccccc32)cc1)C(=O)OC(C)(C)c1ccccc1]
tert-Butyl O-[(1H-benzotriazol-1-yloxy)methyl]-N-trityl-D-tyrosinate 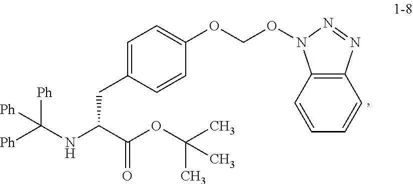 [SMILES: CC(C)(C)OC(=O)[C@@H](Cc1ccc(OCOn2nnc3ccccc32)cc1)NC(c1ccccc1)(c1ccccc1)c1ccccc1]
4-Methoxybenzyl O-[(1H-benzotriazol-1-yloxy)methyl]-N-trityl-D-tyrosinate 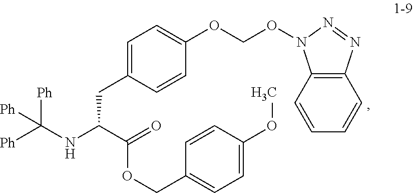 [SMILES: COc1ccc(COC(=O)[C@@H](Cc2ccc(OCOn3nnc4ccccc43)cc2)NC(c2ccccc2)(c2ccccc2)c2ccccc2)cc1]
Cyclopropylmethyl O-[(1H-benzotriazol-1-yloxy)[²H₂]methyl]-N-(tert-butoxy-carbonyl)-D-tyrosinate 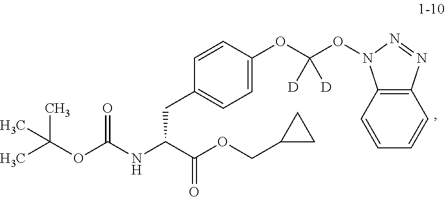 [SMILES: [2H]C([2H])(Oc1ccc(C[C@@H](NC(=O)OC(C)(C)C)C(=O)OCC2CC2)cc1)On1nnc2ccccc21]
2,4-Dimethoxybenzyl O-[(1H-benzotriazol-1-yloxy)methyl]-N-trityl-D-tyrosinate 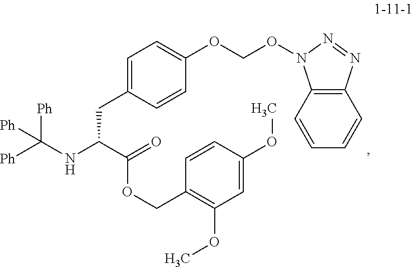 [SMILES: COc1ccc(COC(=O)[C@@H](Cc2ccc(OCOn3nnc4ccccc43)cc2)NC(c2ccccc2)(c2ccccc2)c2ccccc2)c(OC)c1]
2,4-Dimethoxybenzyl O—{[(6-chloro-1H-benzotriazol-1-yl)oxy]methyl}-N-trityl-D-tyrosinate 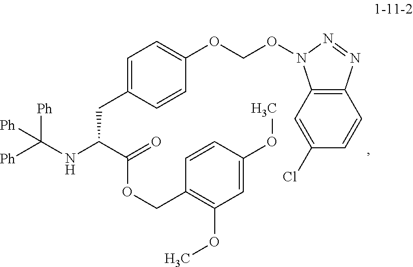 [SMILES: COc1ccc(COC(=O)[C@@H](Cc2ccc(OCOn3nnc4ccc(Cl)cc43)cc2)NC(c2ccccc2)(c2ccccc2)c2ccccc2)c(OC)c1]
2,4-Dimethoxybenzyl O—{[(6-trifluoromethyl-1H-benzotriazol-1-yl)oxy]methyl}-N-trityl-D-tyrosinate 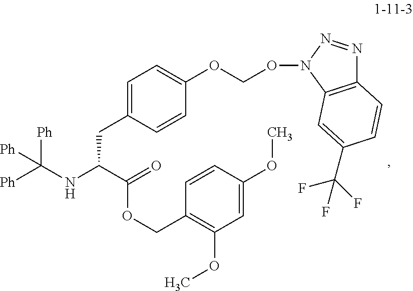 [SMILES: COc1ccc(COC(=O)[C@@H](Cc2ccc(OCOn3nnc4ccc(C(F)(F)F)cc43)cc2)NC(c2ccccc2)(c2ccccc2)c2ccccc2)c(OC)c1]
or
Methyl O-[(1H-benzotriazol-1-yloxy)methyl]-N-(tert-butoxycarbonyl)-alpha-methyltyrosinate

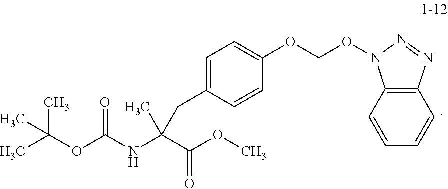 [SMILES: COC(=O)C(C)(Cc1ccc(OCOn2nnc3ccccc32)cc1)NC(=O)OC(C)(C)C]

34. A compound of Formula IIa 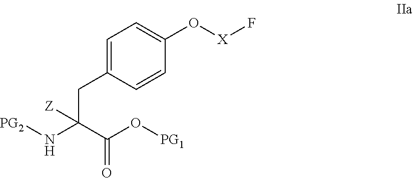
wherein:
X is CH₂, CHD or CD₂;
F is ¹⁸F or ¹⁹F;
Z is Hydrogen or methyl;
PG1 is a carboxylic protecting group, containing up to 20 carbon atoms with the proviso that PG1 is not methyl, or PG1 is a carboxylic protecting group, containing up to 20 carbon atoms containing independently one or more O, N or S atoms,
with the proviso that PG1 is not methyl; and
PG2 is an amino protecting group, containing up to 20 carbon atoms, or PG2 is an amino protecting group, containing up to 20 carbon atoms containing one or more O, N or S atoms, or PG2 is an amino protecting group, containing up to 20 carbon atoms containing one or more O, N or S atoms and are substituted with one or two halogens.

35. The compound according to claim 34, wherein independently from each other
X is CH₂ or CD₂;
F is ¹⁸F or ¹⁹F;
PG1 is
alkyl,
alkyl substituted with one phenyl or alkyl substituted with one phenyl substituted with up to three C₁-C₃ alkyl, C₁-C₃ alkoxy or halogen; with the proviso that PG1 is not methyl
alkyl substituted with one or two C₃-C₆ cycloalkyl,
alkyl substituted with one phenyl or alkyl substituted with one phenyl substituted with up to three C₁-C₃ alkyl, C₁-C₃ alkoxy or halogen and one C₃-C₆ cycloalkyl, or
fluorenylmethyl,
with the proviso that PG1 is not methyl;
alkyl is a branched or linear C₂-C₆ alkyl, or alkyl is a branched or linear C₂-C₆ alkyl substituted with C₁-C₃ alkoxy; and
PG2 is Carbobenzyloxy (Cbz), p-Methoxybenzyl carbonyl (Moz or MeOZ), tert-Butoxycarbonyl (BOC), 9-Fluorenylmethoxycarbonyl (FMOC), Triphenylmethyl (trityl), 4-Methylphenyl-diphenylmethyl (Mtt) or 4-Methoxyphenyldiphenylmethyl (MMTr).

36. The compound according to claim 34, wherein
X is CH₂ or CD₂;
F is ¹⁸F;
Z is hydrogen or methyl;
PG1 is dicyclopropylmethyl or 2,4-dimethoxybenzyl; and
PG2 is tert-Butoxycarbonyl (BOC) or Triphenylmethyl (trityl).

37. The compound according to claim 34, wherein said compound is a
compound of Formula (IIb) 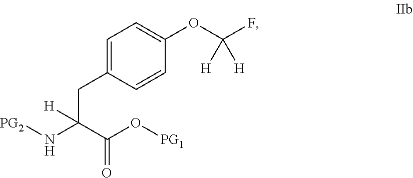
a compound of Formula (IIc) 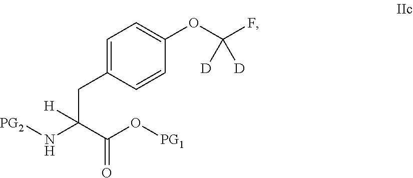
a compound of Formula (IId) 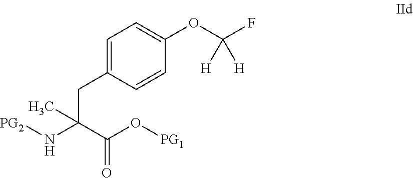
or
a compound of Formula (IIe) 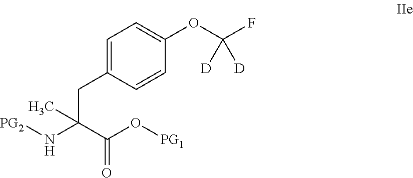
wherein X, F, Z, PG1 and PG2 are as defined.

38. The compound according to claim 34, wherein said compound is of Formula (D-IIa), (D-IIb), (D-IIc), (D-IId) or (D-IIe)

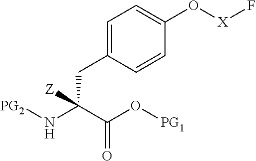

| Formula\Substituent | Z | X |
|---|---|---|
| D-IIa | H, CH₃ | CH₂, CD₂ |
| D-IIb | H | CH₂ |
| D-IIc | H | CD₂ |
| D-IId | CH₃ | CH₂ |
| D-IIe | CH₃ | CD₂ |

wherein F, PG1 and PG2 are as defined.

39. The compound according to claim 34, wherein said compound is:
tert-Butyl N-(tert-butoxycarbonyl)-O-(fluoromethyl)-D-tyrosinate 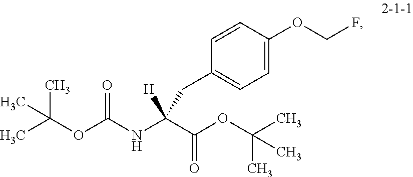 [SMILES: CC(C)(C)OC(=O)N[C@H](Cc1ccc(OCF)cc1)C(=O)OC(C)(C)C]
Dicyclopropylmethyl N-(tert-butoxycarbonyl)-O-(fluoromethyl)-D-tyrosinate 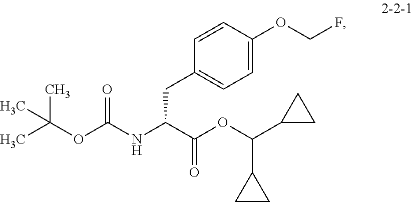 [SMILES: CC(C)(C)OC(=O)N[C@H](Cc1ccc(OCF)cc1)C(=O)OC(C1CC1)C1CC1]
Dicyclopropylmethyl N-(tert-butoxycarbonyl)-O-(fluoromethyl)-L-tyrosinate 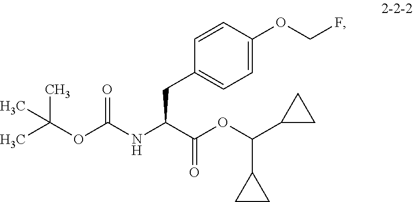 [SMILES: CC(C)(C)OC(=O)N[C@@H](Cc1ccc(OCF)cc1)C(=O)OC(C1CC1)C1CC1]
tert-Butyl O-(fluoromethyl)-N-trityl-D-tyrosinate 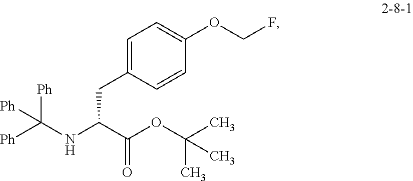 [SMILES: CC(C)(C)OC(=O)[C@@H](Cc1ccc(OCF)cc1)NC(c1ccccc1)(c1ccccc1)c1ccccc1]
2,4-Dimethoxybenzyl O-(fluoromethyl)-N-trityl-D-tyrosinate 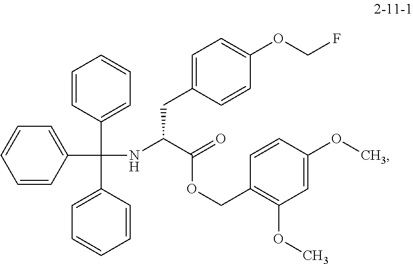 [SMILES: COc1ccc(COC(=O)[C@@H](Cc2ccc(OCF)cc2)NC(c2ccccc2)(c2ccccc2)c2ccccc2)c(OC)c1]
Methyl N-(tert-butoxycarbonyl)-O-(fluoromethyl)-alpha-methyl-D-tyrosinate 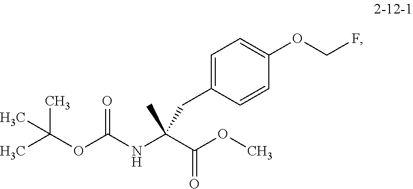 [SMILES: COC(=O)[C@@](C)(Cc1ccc(OCF)cc1)NC(=O)OC(C)(C)C]
or
Methyl N-(tert-butoxycarbonyl)-O-(fluoromethyl)-alpha-methyl-L-tyrosinate

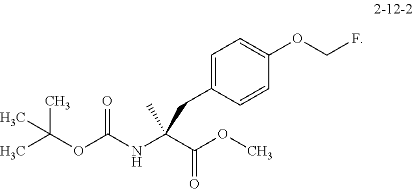 [SMILES: COC(=O)[C@](C)(Cc1ccc(OCF)cc1)NC(=O)OC(C)(C)C]

40. The compound according to claim 34, wherein said compound is:
tert-Butyl N-(tert-butoxycarbonyl)-O-([¹⁸F]fluoromethyl)-D-tyrosinate,
Dicyclopropylmethyl N-(tert-butoxycarbonyl)-O-([¹⁸F]fluoromethyl)-D-tyrosinate,
Dicyclopropylmethyl N-(tert-butoxycarbonyl)-O-([¹⁸F]fluoromethyl)-L-tyrosinate,
2,4-Dimethoxybenzyl N-(tert-butoxycarbonyl)-O— ([¹⁸F]fluoromethyl)-D-tyrosinate,
Cyclopropylmethyl N-(tert-butoxycarbonyl)-O-([¹⁸F]fluoromethyl)-D-tyrosinate,
4-Methoxybenzyl N-(tert-butoxycarbonyl)-O-([¹⁸F]fluoromethyl)-D-tyrosinate,
4-Methoxybenzyl N-(tert-butoxycarbonyl)-O-([¹⁸F]fluoromethyl)-L-tyrosinate,
alpha-Methylbenzyl N-(tert-butoxycarbonyl)-O-([¹⁸F]fluoromethyl)-D-tyrosinate,
alpha, alpha-Dimethylbenzyl N-(tert-butoxycarbonyl)-O-([¹⁸F]fluoromethyl)-D-tyrosinate,
tert-Butyl O-([¹⁸F]fluoromethyl)-N-trityl-D-tyrosinate,
4-Methoxybenzyl O-([¹⁸F]fluoromethyl)-N-trityl-D-tyrosinate,
Cyclopropylmethyl N-(tert-butoxycarbonyl)-O-([¹⁸F]fluoro[²H₂]methyl)-D-tyrosinate,
2,4-Dimethoxybenzyl O-([¹⁸F]fluoromethyl)-N-trityl-D-tyrosinate,
Labelling of 1-11-1, 1-11-2 and 1-11-3
Methyl N-(tert-butoxycarbonyl)-O-([¹⁸F]fluoromethyl)-alpha-methyl-DL-tyrosinate.

41. A composition comprising one or more compounds of Formulas Formula IIa, IIb, IIc, IId, IIe, (D-IIa), (D-IIb), (D-IIc), (D-IId) and or (D-IIe)

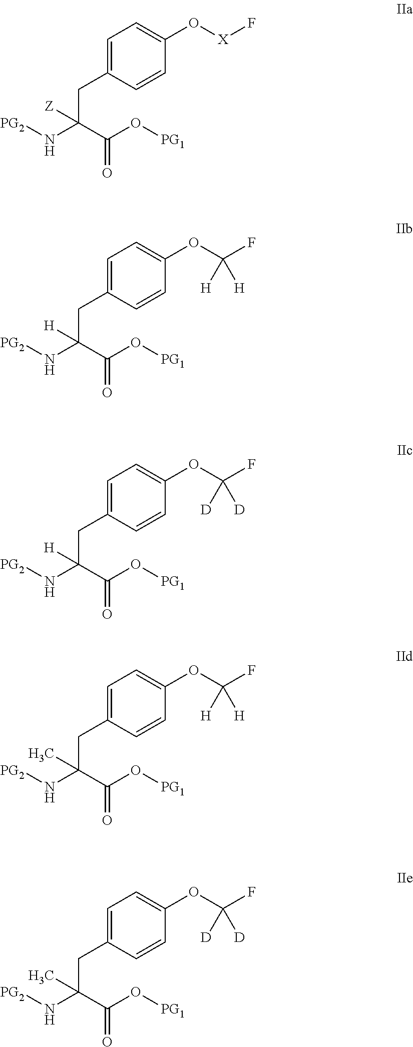

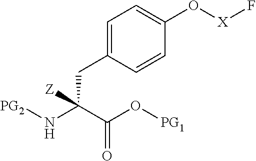

| Formula\Substituent | Z | X |
|---|---|---|
| D-IIa | H, CH₃ | CH₂, CD₂ |
| D-IIb | H | CH₂ |
| D-IIc | H | CD₂ |
| D-IId | CH₃ | CH₂ |
| D-IIe | CH₃ | CD₂ |

wherein, unless otherwise indicated,
X is CH₂, CHD or CD₂;
F is ¹⁸F or ¹⁹F;
Z is Hydrogen or methyl;
PG1 is a carboxylic protecting group, containing up to 20 carbon atoms with the proviso that PG1 is not methyl, or PG1 is a carboxylic protecting group, containing up to 20 carbon atoms containing independently one or more O, N or S atoms,
with the proviso that PG1 is not methyl; and
PG2 is an amino protecting group, containing up to 20 carbon atoms, or PG2 is an amino protecting group, containing up to 20 carbon atoms containing one or more O, N or S atoms, or PG2 is an amino protecting group, containing up to 20 carbon atoms containing one or more O, N or S atoms and are substituted with one or two halogens; and
one or more reagents suitable for deprotection of the amino group and the ester function of the tyrosine.

42. A composition comprising one or more compounds of Formulas Ia, Ib, Ic, Id, Ie, (D-Ia), (D-Ib), (D-Ic), (D-Id) and (D-Ie)

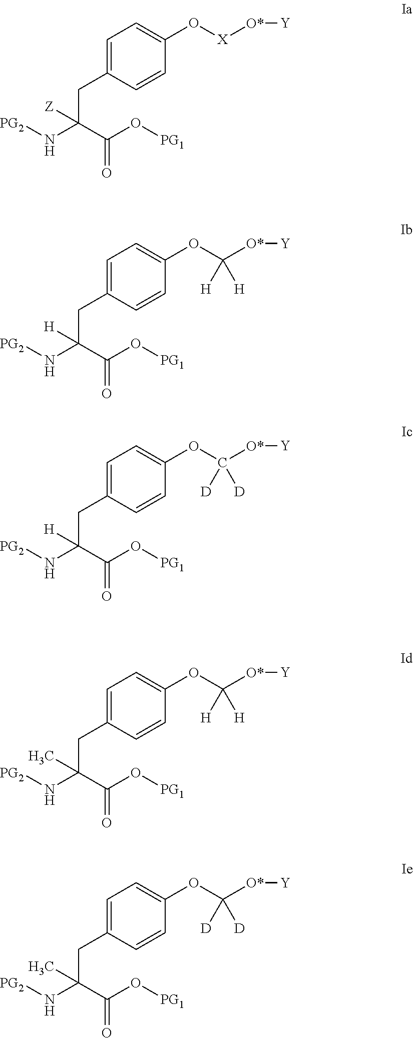

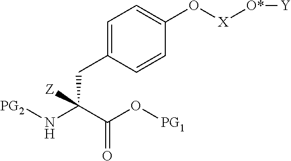

| Formula\Substituent | Z | X |
|---|---|---|
| D-Ia | H, CH₃ | CH₂, CD₂ |
| D-Ib | H | CH₂ |
| D-Ic | H | CD₂ |
| D-Id | CH₃ | CH₂ |
| D-Ie | CH₃ | CD₂ |

wherein, unless otherwise indicated,
X is CH₂, CHD or CD₂;
Y is a substituted or unsubstituted heteroaromatic ring containing one to four Nitrogen atoms (N) with the proviso that the oxygen (O*) is directly bound to one of the Nitrogen atoms (N) of the heteroaromatic ring and O*—Y acts as leaving group;
Z is Hydrogen or methyl;
PG1 is a carboxylic acid protecting group, containing up to 20 carbon atoms, or PG1 is a carboxylic acid protecting group, containing up to 20 carbon atoms containing independently one or more O, N or S atoms; and
PG2 is an amino protecting group, containing up to 20 carbon atoms, or PG2 is an amino protecting group, containing up to 20 carbon atoms containing one more O, N or S atoms, or PG2 is an amino protecting group, containing up to 20 carbon atoms containing one more O, N or S atoms substituted with one to three halogens; and
one or more reagents suitable for fluoro labelling.

43. A kit comprising a sealed vial containing a predetermined quantity of one or more compounds selected from Formulas Ia, Ib, Ic, Id, Ie, (D-Ia), (D-Ib), (D-Ic), (D-Id) and (D-Ie) and suitable salts of inorganic or organic acids, hydrates and solvates,

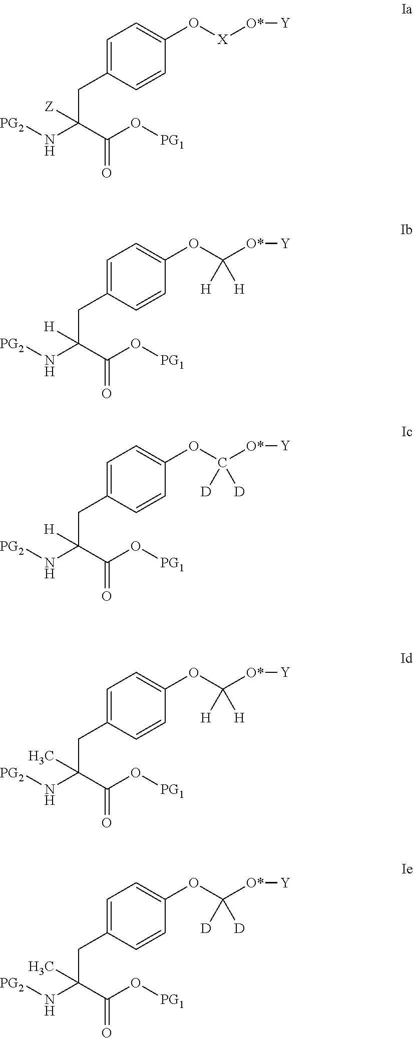

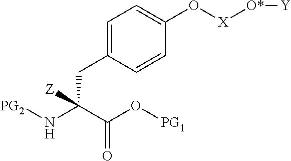

| Formula\Substituent | Z | X |
|---|---|---|
| D-Ia | H, CH₃ | CH₂, CD₂ |
| D-Ib | H | CH₂ |
| D-Ic | H | CD₂ |
| D-Id | CH₃ | CH₂ |
| D-Ie | CH₃ | CD₂ |

wherein, unless otherwise indicated,
X is CH₂, CHD or CD₂;
Y is a substituted or unsubstituted heteroaromatic ring containing one to four Nitrogen atoms (N) with the proviso that the oxygen (O*) is directly bound to one of the Nitrogen atoms (N) of the heteroaromatic ring and O*—Y acts as leaving group;
Z is Hydrogen or methyl;
PG1 is a carboxylic acid protecting group, containing up to 20 carbon atoms, or PG1 is a carboxylic acid protecting group, containing up to 20 carbon atoms containing independently one or more O, N or S atoms; and
PG2 is an amino protecting group, containing up to 20 carbon atoms, or PG2 is an amino protecting group, containing up to 20 carbon atoms containing one more O, N or S atoms, or PG2 is an amino protecting group, containing up to 20 carbon atoms containing one more O, N or S atoms substituted with one to three halogens.

44. A method for obtaining compounds of Formula Ia 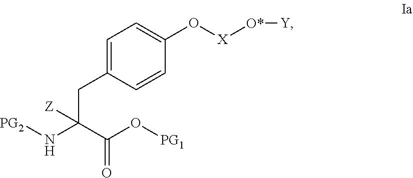
said method comprising:
reacting a compound of Formula V 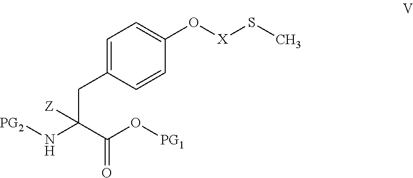
first with N-Chloro-succinimide (NCS) and then with anion of H—O*—Y to obtain a compound of Formula Ia,
wherein
Z, PG1, PG2, X, and Y are as defined according to claim 28.

45. A method for obtaining compounds of Formula IIa 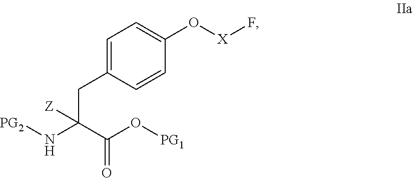
said method of comprising:
reacting a compound of Formula Ia 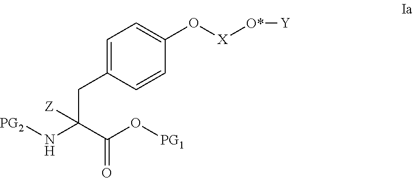
with a ¹⁸F-Fluorination agent
wherein
F, Z, PG1, PG2, X, and Y are as defined according to claim 28.

46. A method according to claim 45, further comprising
Converting the obtained compound into a salt of an inorganic or organic base thereof, a hydrate, a complex, or solvate thereof.
